(19) United States
(12) Patent Application Publication
(10) Pub. No.: US 2009/0149417 A1
(43) Pub. Date: Jun. 11, 2009

(54) Methods and compositions for the treatment of cancer using benzopyrone-type PARP inhibitors

(76) Inventors: Valeria Ossovskaya, San Francisco, CA (US); Barry M. Sherman, Hillsborough, CA (US)

Correspondence Address: WILSON SONSINI GOODRICH & ROSATI, 650 PAGE MILL ROAD, PALO ALTO, CA, 94304-1050, US

(21) Appl. No.: 12/288,329

(22) Filed: Oct. 17, 2008

Related U.S. Application Data
(60) Provisional application No. 60/981,436, filed on Oct. 19, 2007.
(60) Provisional application No. 61/096,282, filed on Sep. 11, 2008.

(51) Int. Cl. A61K 31/37; A61P 35/00; A61K 31/4025; A61K 31/4433; A61K 31/496; A61K 31/4178; A61K 31/7068

(52) U.S. Cl. 514/49; 514/457; 514/422; 514/337; 514/254.11; 514/389

(57) ABSTRACT

The present invention provides compositions of matter, kits and methods for their use in the treatment of cancer. In particular, the invention provides compositions and methods for treating cancer in a subject by inhibiting a poly-ADP-ribose polymerase, as well as providing formulations and modes of administering such compositions.

### CROSS REFERENCE

This application claims the benefit of U.S. Provisional Application No. 60/981,436, entitled “Treatment of Cancer with Benzopyrone-Type PARP Inhibitors” filed Oct. 19, 2007 (Attorney Docket No. 28825-745.101); and U.S. Provisional Application No. 61/096,282, entitled “Methods and Compositions for the Treatment of Cancer Using Benzopyrone PARP Inhibitors” filed Sep. 11, 2008 (Attorney Docket No. 28825-745.102), each of which applications is incorporated herein in its entirety by reference.

### BACKGROUND OF THE INVENTION

Cancer is a serious threat to modern society. Malignant cancerous growths, due to their unique characteristics, pose serious challenges for modern medicine. Their characteristics include uncontrollable cell proliferation resulting in unregulated growth of malignant tissue, an ability to invade local and even remote tissues, lack of differentiation, lack of detectable symptoms and most significantly, the lack of effective therapy and prevention.

Cancer can develop in any tissue of any organ at any age. The etiology of cancer is not clearly defined but mechanisms such as genetic susceptibility, chromosome breakage disorders, viruses, environmental factors and immunologic disorders have all been linked to a malignant cell growth and transformation. Cancer encompasses a large category of medical conditions, affecting millions of individuals worldwide. Cancer cells can arise in almost any organ and/or tissue of the body. Cancer develops when cells in a part of the body begin to grow or differentiate out of control. All cancer types begin with the out-of-control growth of abnormal cells.

There are many types of cancer, including, breast, lung, ovarian, bladder, prostate, pancreatic, cervical, and leukemia. Currently, some of the main treatments available are surgery, radiation therapy, and chemotherapy. Surgery is often a drastic measure and can have serious consequences. For example, all treatments for ovarian cancer may result in infertility. Some treatments for cervical cancer and bladder cancer may cause infertility and/or sexual dysfunction. Surgical procedures to treat pancreatic cancer may result in partial or total removal of the pancreas and can carry significant risks to the patient. Breast cancer surgery invariably involves removal of part of or the entire breast. Some surgical procedures for prostate cancer carry the risk of urinary incontinence and impotence. The procedures for lung cancer patients often have significant post-operative pain as the ribs must be cut through to access and remove the cancerous lung tissue. In addition, patients who have both lung cancer and another lung disease, such as emphysema or chronic bronchitis, typically experience an increase in their shortness of breath following the surgery.

Radiation therapy has the advantage of killing cancer cells but it also damages non-cancerous tissue at the same time. Chemotherapy involves the administration of various anti-cancer drugs to a patient but often is accompanied by adverse side effects.

Worldwide, more than 10 million people are diagnosed with cancer every year and it is estimated that this number will grow to 15 million new cases every year by 2020. Cancer causes six million deaths every year or 12% of the deaths worldwide. There remains a need for methods that can treat cancer. These methods can provide the basis for pharmaceutical compositions useful in the prevention and treatment of cancer in humans and other mammals.

A series of anti-tumor drugs have been identified. These drugs include nitro and nitroso compounds and their metabolites, which are the subject of U.S. Pat. No. 5,464,871 issued on Nov. 7, 1995 entitled “Aromatic Nitro and Nitroso Compounds and their Metabolites Useful as Anti-viral and Anti-tumor Agents,” Pat. No. 5,670,518 issued on Sep. 23, 1997 entitled “Aromatic Nitro and Nitroso Compounds and their Metabolites Useful as Anti-viral and Anti-tumor Agents,” Pat. No. 6,004,978 issued on Dec. 21, 1999 entitled “Methods of Treating Cancer with Aromatic Nitro and Nitroso Compounds and their Metabolites” the disclosures of which are incorporated herein by reference.

PARP (poly-ADP ribose polymerase) participates in a variety of DNA-related functions including cell proliferation, differentiation, apoptosis, DNA repair and also has effects on telomere length and chromosome stability (d'Adda di Fagagna et al, 1999, Nature Gen., 23(1): 76-80). Oxidative stress-induced over activation of PARP consumes NAD+ and consequently ATP, culminating in cell dysfunction or necrosis. This cellular suicide mechanism has been implicated in the pathomechanism of cancer, stroke, myocardial ischemia, diabetes, diabetes-associated cardiovascular dysfunction, shock, traumatic central nervous system injury, arthritis, colitis, allergic encephalomyelitis, and various other forms of inflammation. PARP has also been shown to associate with and regulate the function of several transcription factors. The multiple functions of PARP make it a target for a variety of serious conditions including various types of cancer and neurodegenerative diseases.

### SUMMARY OF THE INVENTION

In one aspect, the present invention provides a method of treating a cancer comprising administering to a subject in need thereof an effective amount of a compound of formula (I), or a metabolite, a pharmaceutically acceptable salt or prodrug thereof:

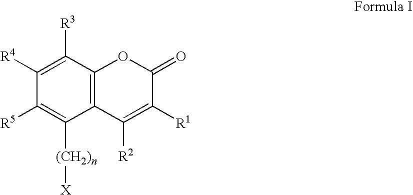

wherein n=0-10; R¹, R², R³, R⁴, R⁵ and X are independently selected from the group consisting of hydrogen, hydroxy, optionally substituted amine, amino, carboxyl, ester, nitroso, nitro, halogen, optionally substituted (C₁-C₆) alkyl, optionally substituted (C₁-C₆) alkoxy, optionally substituted (C₃-C₇) cycloalkyl, optionally substituted (C₃-C₇) heterocyclic, phenyl, and optionally substituted aryl; and wherein at least two of the R¹, R², R³, R⁴, and R⁵ substituents are always hydrogen;

wherein the cancer comprises adrenal cortical cancer, anal cancer, aplastic anemia, bile duct cancer, bladder cancer, bone cancer, bone metastasis, central nervous system (CNS) cancers, peripheral nervous system (PNS) cancers, Castleman's Disease, cervical cancer, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's disease, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, uterine cancer (e.g. uterine sarcoma), vaginal cancer, vulvar cancer, and Waldenstrom's macroglobulinemia.

Some embodiments of the present invention provide a method of treating a cancer comprising administering to a subject in need thereof an effective amount of a compound of formula (I), or a metabolite, a pharmaceutically acceptable salt or prodrug thereof:

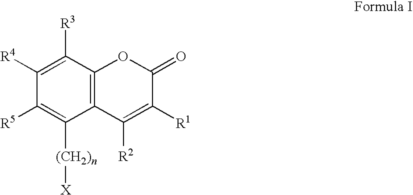

wherein n=0-10; R¹, R², R³, R⁴, R⁵ and X are independently selected from the group consisting of hydrogen, hydroxy, optionally substituted amine, amino, carboxyl, ester, nitroso, nitro, halogen, optionally substituted (C₁-C₆) alkyl, optionally substituted (C₁-C₆) alkoxy, optionally substituted (C₃-C₇) cycloalkyl, optionally substituted (C₃-C₇) heterocyclic, phenyl, and optionally substituted aryl; and wherein at least two of the R¹, R², R³, R⁴, and R⁵ substituents are always hydrogen;

wherein the cancer is a cancer formed at a different site of a body as a result of migration of a cell from a cancer selected from the group consisting of adrenal cortical cancer, anal cancer, aplastic anemia, bile duct cancer, bladder cancer, bone cancer, bone metastasis, central nervous system (CNS) cancers, peripheral nervous system (PNS) cancers, Castleman's Disease, cervical cancer, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's disease, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, uterine cancer (e.g. uterine sarcoma), vaginal cancer, vulvar cancer, and Waldenstrom's macroglobulinemia.

In some embodiments, the benzopyrone compound is of formula II or a metabolite, a pharmaceutically acceptable salt or prodrug thereof:

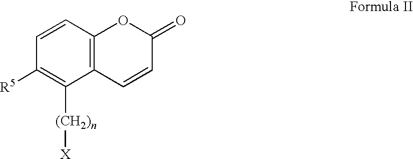

wherein R⁵ is selected from the group consisting of hydrogen, carboxyl, amino, nitroso, nitro and hydroxy; hydroxylamino, and X is selected from the group consisting of halogen, hydroxy, optionally substituted (C₁-C₇) alkyl, optionally substituted (C₁-C₆) alkoxy, optionally substituted (C₃-C₇) cycloalkyl, optionally substituted (C₃-C₇) heterocyclic, phenyl, and optionally substituted aryl.

In some embodiments, X is a halogen selected from the group consisting of F, Cl, Br and I. In some embodiments, X is iodine (I) and R⁵ is nitro, nitroso, hydroxylamino, hydroxy or amino. In some embodiments, n is 0. In some embodiments, the optionally substituted alkyl is substituted with a substituent selected from the group consisting of alkylamine, pyrrole, dihydropyrrole, and pyrrolidene.

In some embodiments, the compound is of the formula IIIa, IIIb, IIIc, IIId, IIIe, IIIf, IIIg, IIIh, IIIk, IIIl, IIIm, or IIIn, or one of their pharmaceutically acceptable salts or prodrugs:

In some embodiments, the compound is 5-iodo-6-nitro-benzopyrone of Formula IIIg, or a

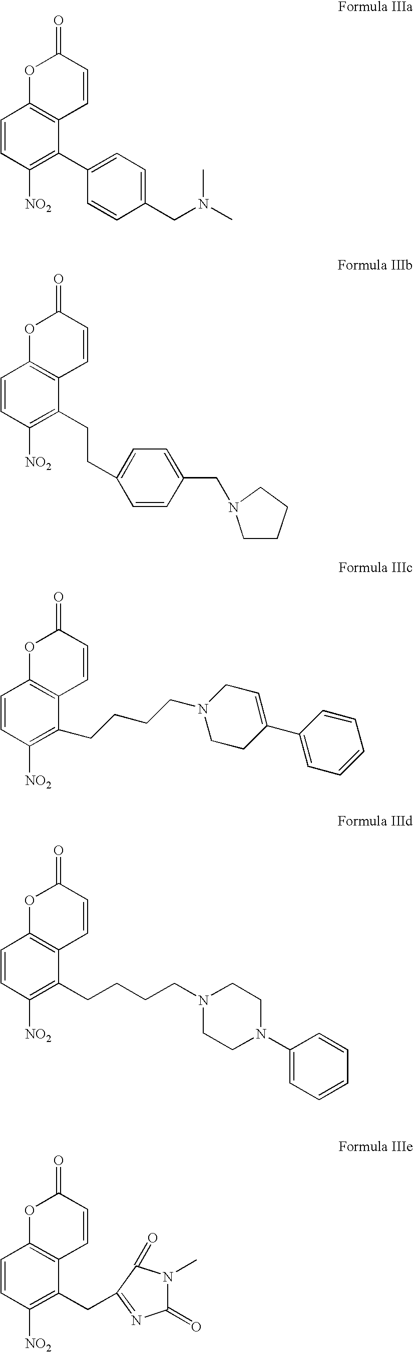

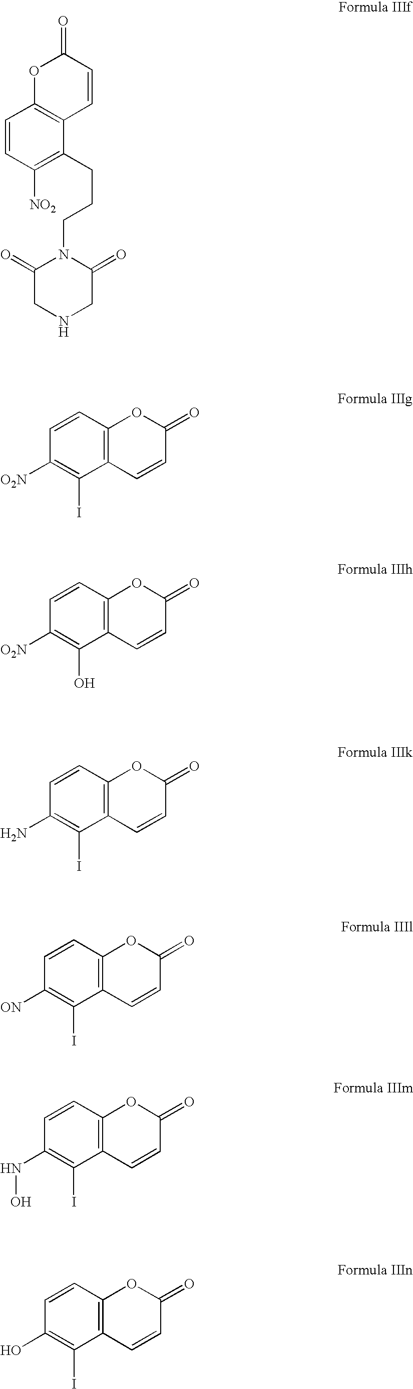

metabolite, a pharmaceutically acceptable salt or prodrug thereof. In some embodiments, the compound is 5-iodo-6-amino-benzopyrone of Formula IIIk, or a metabolite, a pharmaceutically acceptable salt or prodrug thereof. In some embodiments, the compound is 5-iodo-6-nitroso-benzopyrone of Formula IIIl, or a metabolite, a pharmaceutically acceptable salt or prodrug thereof. In some embodiments, the compound is 5-iodo-6-hydroxylamino-benzopyrone of Formula IIIm, or a metabolite, a pharmaceutically acceptable salt or prodrug thereof.

In some embodiments, the optionally substituted (C₃-C₇) heterocyclic is a five membered heterocyclic ring or a six membered heterocyclic ring. In some embodiments, the optionally substituted (C₃-C₇) heterocyclic contains at least one nitrogen. In some embodiments, the optionally substituted (C₃-C₇) heterocyclic is selected from the group consisting of azeridine, azetidine, pyrrole, dihydropyrrole, pyrrolidene, pyrazole, pyrazoline, pyrazolidine, imidazole, benzimidazole, triazole, tetrazole, oxazole, isoxazole, benzoxazole, oxadiazole, oxazoline, oxazolidine, thiazole, isothiazole, pyridine, dihydropyridine, tetrahydropyridine, quinazoline, pyrazine, pyrimidine, pyridazine, quinoline, isoquinoline, triazine, tetrazine, and piperazine. In some embodiments, the optionally substituted (C₃-C₇) heterocyclic is substituted with a substituent selected from the group consisting of optionally substituted (C₁-C₆) alkyl, optionally substituted (C₁-C₆) alkoxy, optionally substituted (C₃-C₇) cycloalkyl, optionally substituted (C₃-C₇) heterocyclic, and optionally substituted aryl.

In some embodiments, the methods of the present invention further comprise surgery, radiation therapy, chemotherapy, gene therapy, RNA therapy, nanotherapy, immunotherapy, or a combination thereof. In some embodiments, the methods of the present invention further comprise administering an effective amount of an anti-tumor agent. In some embodiments, the methods of the present invention further comprise administering an effective amount of an organoplatinum compound. In some embodiments, the methods of the present invention further comprise administering an effective amount of an antimetabolite. In some embodiments, the methods of the present invention further comprise administering an effective amount of oxaliplatin (OX). In some embodiments, the methods of the present invention further comprise administering an effective amount of gemcitabine (GEM). In some embodiments, the methods of the present invention further comprise administering an effective amount of an organoplatinum and an antimetabolite. In some embodiments, the methods of the present invention further comprise administering an effective amount of OX and GEM. In some embodiments, the administration is intravenous. In some embodiments, the administration is intraperitoneal. In some embodiments, the administration is orally. In some embodiments, a poly-ADP-ribose polymerase (PARP) is inhibited by the compound in the subject. In some embodiments, mono-ADP ribosylation and poly-ADP ribosylation are inhibited. In some embodiments, a tumor cell undergoes apoptosis, cell cycle arrest, and/or necrosis in the subject. In some embodiments, the subject expresses a detectable level of PARP protein. In some embodiments, the subject has a detectable level of mono-ADP ribosylation and poly-ADP ribosylation.

In some embodiments, the present invention provides a method of treating a cancer comprising administering to a subject in need thereof an effective amount of a compound of formula (I), or a metabolite, a pharmaceutically acceptable salt or prodrug thereof:

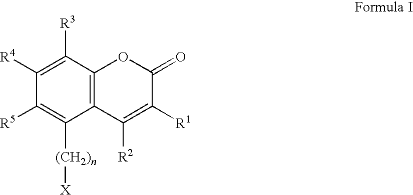

wherein n=0-10; R¹, R², R³, R⁴, R⁵ and X are independently selected from the group consisting of hydrogen, hydroxy, optionally substituted amine, amino, carboxyl, ester, nitroso, nitro, halogen, optionally substituted (C₁-C₆) alkyl, optionally substituted (C₁-C₆) alkoxy, optionally substituted (C₃-C₇) cycloalkyl, optionally substituted (C₃-C₇) heterocyclic, phenyl, and optionally substituted aryl; wherein at least two of the R¹, R², R³, R⁴, and R⁵ substituents are always hydrogen; and wherein at least one of the R¹, R², R³, R⁴, and R⁵ substituents is always a substituted cycloalkyl, a substituted heterocyclic, or a substituted phenyl;

wherein the cancer is selected from the group consisting of adrenal cortical cancer, anal cancer, aplastic anemia, bile duct cancer, bladder cancer, bone cancer, bone metastasis, central nervous system (CNS) cancers, peripheral nervous system (PNS) cancers, breast cancer, Castleman's Disease, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, uterine cancer (e.g. uterine sarcoma), vaginal cancer, vulvar cancer, and Waldenstrom's macroglobulinemia.

In some embodiments, the cancer is breast cancer, ovarian cancer, uterine cancer, pancreatic cancer, lung cancer, brain cancer, skin cancer, colon cancer, or a cancer derived from cancer stem cells. In some embodiments, the breast cancer is negative for at least one of: ER, PR or HER2. In some embodiments, the breast cancer is negative for at least one of: ER, PR or HER2; and wherein the breast cancer is positive for at least one of ER, PR or HER2. In some embodiments, the breast cancer is negative for two of: ER, PR or HER2. In some embodiments, the breast cancer is ER-negative and PR-negative. In some embodiments, the breast cancer is ER-negative and HER2-negative. In some embodiments, the breast cancer is PR-negative and HER2-negative. In some embodiments, the breast cancer is an ER-negative breast cancer. In some embodiments, the breast cancer is an HER²-negative breast cancer. In some embodiments, X is a halogen selected from the group consisting of F, Cl, Br and

I. In some embodiments, X is iodine (I) and R⁵ is nitro, nitroso, hydroxylamino, hydroxyl, or amino. In some embodiments, n is 0. In some embodiments, the optionally substituted alkyl is substituted with a substituent selected from the group consisting of alkylamine, pyrrole, dihydropyrrole, and pyrrolidene. In some embodiments, the compound is of the formula IIIa, IIIb, IIIc, IIId, IIIe, or IIIf, or one of their pharmaceutically acceptable salts or prodrugs:

In some embodiments, the optionally substituted (C₃-C₇) heterocyclic is a five membered

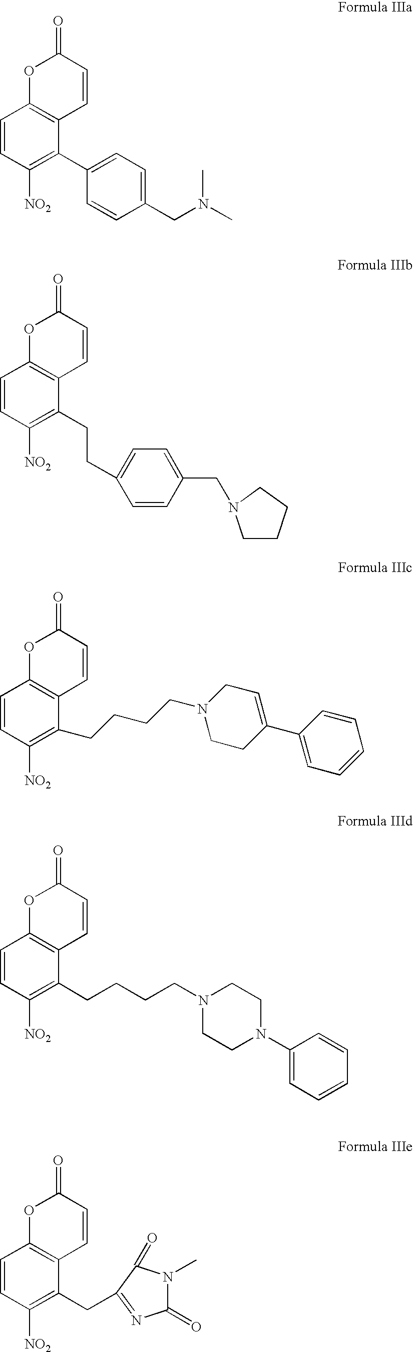

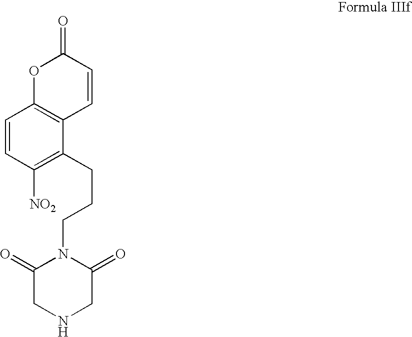

heterocyclic ring or a six membered heterocyclic ring. In some embodiments, the optionally substituted (C₃-C₇) heterocyclic contains at least one nitrogen. In some embodiments, the optionally substituted (C₃-C₇) heterocyclic is selected from the group consisting of azeridine, azetidine, pyrrole, dihydropyrrole, pyrrolidene, pyrazole, pyrazoline, pyrazolidine, imidazole, benzimidazole, triazole, tetrazole, oxazole, isoxazole, benzoxazole, oxadiazole, oxazoline, oxazolidine, thiazole, isothiazole, pyridine, dihydropyridine, tetrahydropyridine, quinazoline, pyrazine, pyrimidine, pyridazine, quinoline, isoquinoline, triazine, tetrazine, and piperazine. In some embodiments, the optionally substituted (C₃-C₇) heterocyclic is substituted with a substituent selected from the group consisting of optionally substituted (C₁-C₆) alkyl, optionally substituted (C₁-C₆) alkoxy, optionally substituted (C₃-C₇) cycloalkyl, optionally substituted (C₃-C₇) heterocyclic, and optionally substituted aryl.

In some embodiments, the method further comprises surgery, radiation therapy, chemotherapy, gene therapy, RNA therapy, immunotherapy, nanotherapy or a combination thereof. In some embodiments, the method further comprises administering an effective amount of an anti-tumor agent. In some embodiments, the method further comprises administering an effective amount of an organoplatinum compound. In some embodiments, the method further comprises administering an effective amount of an anti-metabolite compound. In some embodiments, the method further comprises administering an effective amount of oxaliplatin (OX). In some embodiments, the method further comprises administering an effective amount of gemcitabine (GEM). In some embodiments, the method further comprises administering an effective amount of OX and GEM. In some embodiments, the administration is intravenous or intraperitoneal. In some embodiments, the administration is orally. In some embodiments, a poly-ADP-ribose polymerase (PARP) is inhibited by the compound in the subject. In some embodiments, mono-ADP ribosylation and poly-ADP ribosylation are inhibited. In some embodiments, a tumor cell undergoes apoptosis, cell cycle arrest, and/or necrosis in the subject. In some embodiments, the subject expresses a detectable level of PARP protein. In some embodiments, the subject has a detectable level of mono-ADP ribosylation and poly-ADP ribosylation.

In some embodiments, the present invention provides a method of treating a cancer comprising administering to a subject in need thereof an effective amount of a composition comprising an anti-tumor agent and a compound of formula (I), or a metabolite, a pharmaceutically acceptable salt or prodrug thereof.

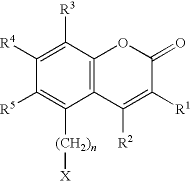

wherein n=0-10; R¹, R², R³, R⁴, R⁵ and X are independently selected from the group consisting of hydrogen, hydroxy, optionally substituted amine, amino, carboxyl, ester, nitroso, nitro, halogen, optionally substituted (C₁-C₆) alkyl, optionally substituted (C₁-C₆) alkoxy, optionally substituted (C₃-C₇) cycloalkyl, optionally substituted (C₃-C₇) heterocyclic, phenyl, and optionally substituted aryl; and wherein at least two of the R¹, R², R³, R⁴, and R⁵ substituents are always hydrogen.

In some embodiments, the compound is of formula II or a metabolite, a pharmaceutically acceptable salt or prodrug thereof:

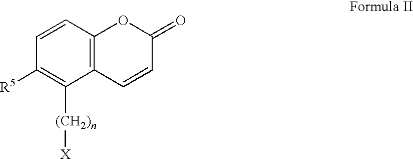

wherein R⁵ is selected from the group consisting of hydrogen, carboxyl, amino, nitroso, nitro and hydroxy; hydroxylamino, and X is selected from the group consisting of halogen, hydroxy, optionally substituted (C₁-C₇) alkyl, optionally substituted (C₁-C₆) alkoxy, optionally substituted (C₃-C₇) cycloalkyl, optionally substituted (C₃-C₇) heterocyclic, phenyl, and optionally substituted aryl.

In some embodiments, the compound is of the formula IIIa, IIIb, IIIc, IIId, IIIe, IIIf, IIIg, IIIh, IIIk, IIIl, IIIm, or IIIn, or one of their pharmaceutically acceptable salts or prodrugs:

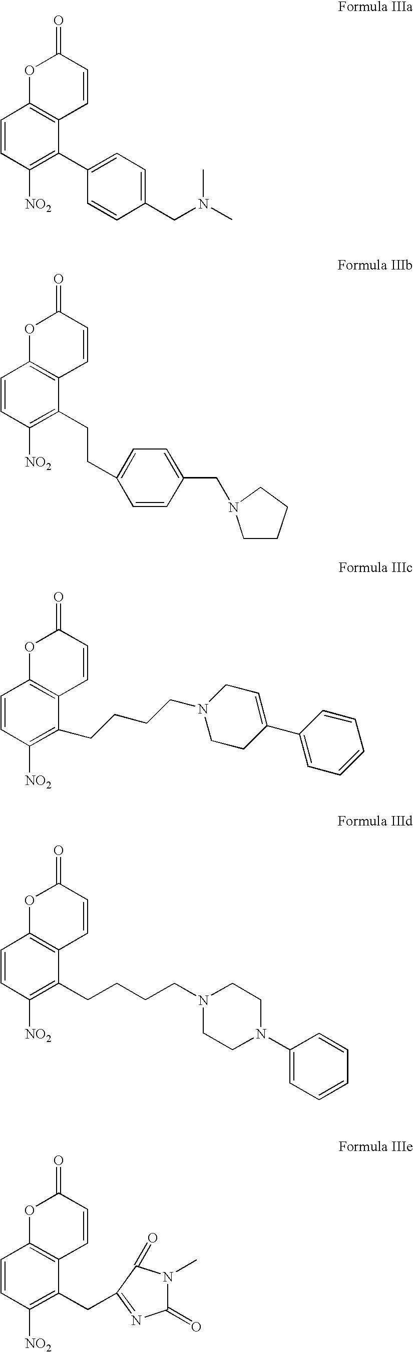

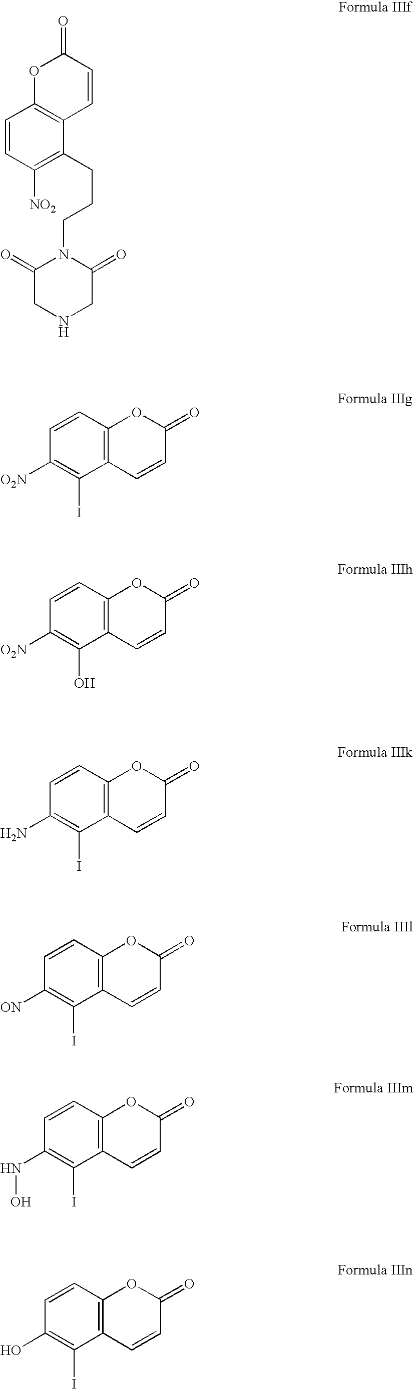

In some embodiments, the compound is 5-iodo-6-nitro-benzopyrone of Formula IIIg, or or a metabolite, a pharmaceutically acceptable salt or prodrug thereof. In some embodiments, the compound is 5-iodo-6-amino-benzopyrone of Formula IIIk, or a metabolite, a pharmaceutically acceptable salt or prodrug thereof. In some embodiments, the compound is 5-iodo-6-nitroso-benzopyrone of Formula IIIl, or a metabolite, a pharmaceutically acceptable salt or prodrug thereof. In some embodiments, the compound is 5-iodo-6-hydroxylamino-benzopyrone of Formula IIIm, or a metabolite, a pharmaceutically acceptable salt or prodrug thereof.

In some embodiments, the cancer that may be treated by a combination of an anti-tumor agent and a compound of formula (I) comprises adrenal cortical cancer, anal cancer, aplastic anemia, bile duct cancer, bladder cancer, bone cancer, bone metastasis, central nervous system (CNS) cancers, peripheral nervous system (PNS) cancers, breast cancer, Castleman's Disease, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, uterine cancer (e.g. uterine sarcoma), vaginal cancer, vulvar cancer, and Waldenstrom's macroglobulinemia.

In some embodiments, the cancer is breast cancer, ovarian cancer, uterine cancer, pancreatic cancer, lung cancer, brain cancer, skin cancer, colon cancer, or a cancer derived from cancer stem cells. In some embodiments, the breast cancer is negative for at least one of: ER, PR or HER2. In some embodiments, the breast cancer is negative for at least one of: ER, PR or HER2; and wherein the breast cancer is positive for at least one of ER, PR or HER2. In some embodiments, the breast cancer is negative for two of: ER, PR or HER2. In some embodiments, the breast cancer is ER-negative and PR-negative. In some embodiments, the breast cancer is ER-negative and HER2-negative. In some embodiments, the breast cancer is PR-negative and HER2-negative. In some embodiments, the breast cancer is an ER-negative breast cancer. In some embodiments, the breast cancer is an HER2-negative breast cancer.

In some embodiments, the anti-tumor agent comprises antitumor alkylating agents, antitumor antimetabolites, antitumor antibiotics, plant-derived antitumor agents, antitumor organoplatinum compounds, antitumor campthotecin derivatives, antitumor tyrosine kinase inhibitors, monoclonal antibodies, interferons, biological response modifiers, and other agents having antitumor activities, or a pharmaceutically acceptable salt thereof. In some embodiments, the antitumor alkylating agents comprise nitrogen mustard N-oxide, cyclophosphamide, ifosfamide, melphalan, busulfan, mitobronitol, carboquone, thiotepa, ranimustine, nimustine, temozolomide, and carmustine; the antitumor antimetabolites comprise methotrexate, 6-mercaptopurine riboside, mercaptopurine, 5-fluorouracil, tegafur, doxifluridine, carmofur, cytarabine, cytarabine ocfosfate, enocitabine, S-1, gemcitabine, fludarabine, and pemetrexed disodium; the antitumor antibiotics comprise actinomycin D, doxorubicin, daunorubicin, neocarzinostatin, bleomycin, peplomycin, mitomycin C, aclarubicin, pirarubicin, epirubicin, zinostatin stimalamer, idarubicin, sirolimus, and valrubicin; the plant-derived antitumor agents comprise vincristine, vinblastine, vindeshine, etoposide, sobuzoxane, docetaxel, paclitaxel, and vinorelbine; the antitumor platinum-complex compounds comprise cisplatin, carboplatin, nedaplatin, and oxaliplatin; the antitumor campthotecin derivatives comprise irinotecan, topotecan, and campthotecin; the antitumor tyrosine kinase inhibitors comprise gefitinib, imatinib, and erlotinib; the monoclonal antibodies comprise abciximab, adalimumab, alemtuzumab, basiliximab, bevacizumab, cetuximab, daclizumab, eculizumab, efalizumab, ibritumomab, tiuxetan, infliximab, muromonab-CD3, natalizumab, omalizumab, palivizumab, panitumumab, ranibizumab, gemtuzumab ozogamicin, rituximab, tositumomab, and trastuzumab; the interferons comprise interferon α, interferon α-2a, interferon α-2b, interferon β, interferon γ-1a, and interferon γ-n1, the biological response modifiers comprise krestin, lentinan, sizofuran, picibanil, or ubenimex, and the other antitumor agents comprise mitoxantrone, L-asparaginase, procarbazine, dacarbazine, hydroxycarbamide, pentostatin, tretinoin, alefacept, darbepoetin alfa, anastrozole, exemestane, bicalutamide, leuprorelin, flutamide, fulvestrant, pegaptanib octasodium, denileukin diftitox, aldesleukin, thyrotropin alfa, arsenic trioxide, bortezomib, capecitabine, and goserelin.

In some embodiments, the anti-tumor agent is an organoplatinum compound. In some embodiments, the anti-tumor agent is oxaliplatin (OX), cisplatin, or carboplatin. In some embodiments, the anti-tumor agent is oxaliplatin (OX). In some embodiments, the anti-tumor agent is an anti-metabolite agent. In some embodiments, the anti-tumor agent is gemcitabine (GEM). In some embodiments, the method further includes more than one anti-tumor agent. In some embodiments, the anti-tumor agents are an organoplatinum compound and an anti-metabolite agent. In some embodiments, the anti-tumor agents are OX and GEM.

In some embodiments, the method further comprises surgery, radiation therapy, gene therapy, RNA therapy, immunotherapy, nanotherapy, or a combination thereof. In some embodiments, the administration is intravenous. In some embodiments, the administration is intraperitoneal. In some embodiments, the administration is orally. In some embodiments, a poly-ADP-ribose polymerase (PARP) is inhibited by the compound in the subject. In some embodiments, a tumor cell undergoes apoptosis, cell cycle arrest, and/or necrosis in the subject.

In some embodiments, mono-ADP ribosylation and poly-ADP ribosylation are inhibited. In some embodiments, the subject expresses a detectable level of PARP protein. In some embodiments, the subject has a detectable level of mono or poly-ADP ribosylation. In some embodiments, the present invention discloses a method of treating a cancer comprising administering to a subject in need thereof an effective amount of a composition comprising an organoplatinum compound and a compound of formula (I), or a pharmaceutically acceptable salt or prodrug thereof. In some embodiments, the present invention discloses a method of treating a cancer comprising administering to a subject in need thereof an effective amount of a composition comprising oxaliplatin (OX) and a compound of formula (I), or a pharmaceutically acceptable salt or prodrug thereof. In some embodiments, the method further comprises administering an effective amount of OX and 5-iodo-6-nitro-benzopyrone (IIIg). In some embodiments, the compound is 5-iodo-6-nitro-benzopyrone of Formula IIIg, or one of its pharmaceutically acceptable salts or prodrugs. In some embodiments, the compound is 5-iodo-6-amino-benzopyrone of Formula IIIk, or one of its pharmaceutically acceptable salts or prodrugs. In some embodiments, the compound is 5-iodo-6-nitroso-benzopyrone of Formula IIIl, or one of its pharmaceutically acceptable salts or prodrugs. In some embodiments, the compound is 5-iodo-6-hydroxylamino-benzopyrone of Formula IIIm, or one of its pharmaceutically acceptable salts or prodrugs. In some embodiments, the method further comprises administering an effective amount of OX and 5-iodo-6-amino-benzopyrone (IIIk). In some embodiments, the method further comprises administering an effective amount of an antimetabolite. In some embodiments, the method further comprises administering an effective amount of gemcitabine (GEM). In some embodiments, the method further comprises surgery, radiation therapy, gene therapy, RNA therapy, immunotherapy, nanotherapy, or a combination thereof. In some embodiments, the administration is intravenous. In some embodiments, the administration is intraperitoneal. In some embodiments, the administration is orally. In some embodiments, a poly-ADP-ribose polymerase (PARP) is inhibited by the compound in the subject. In some embodiments, mono-ADP ribosylation and poly-ADP ribosylation are inhibited. In some embodiments, a tumor cell undergoes apoptosis, cell cycle arrest, and/or necrosis in the subject. In some embodiments, the subject expresses a detectable level of PARP protein. In some embodiments, the subject has a detectable level of mono or poly-ADP ribosylation.

In some embodiments, the present invention discloses a method of treating a cancer comprising administering to a subject in need thereof an effective amount of a composition comprising an antimetabolite and a compound of formula (I), or a pharmaceutically acceptable salt or prodrug thereof. In some embodiments, the present invention discloses a method of treating a cancer comprising administering to a subject in need thereof an effective amount of a composition comprising gemcitabine (GEM) and a compound of formula (I), or a pharmaceutically acceptable salt or prodrug thereof. In some embodiments, the compound is 5-iodo-6-nitro-benzopyrone of Formula IIIg, or one of its pharmaceutically acceptable salts or prodrugs. In some embodiments, the compound is 5-iodo-6-amino-benzopyrone of Formula IIIk, or one of its pharmaceutically acceptable salts or prodrugs. In some embodiments, the compound is 5-iodo-6-nitroso-benzopyrone of Formula IIIl, or one of its pharmaceutically acceptable salts or prodrugs. In some embodiments, the compound is 5-iodo-6-hydroxylamino-benzopyrone of Formula IIIm, or one of its pharmaceutically acceptable salts or prodrugs. In some embodiments, the method further comprises administering an effective amount of GEM and 5-iodo-6-nitro-benzopyrone (IIIg). In some embodiments, the method further comprises administering an effective amount of GEM and 5-iodo-6-amino-benzopyrone (IIIk). In some embodiments, the method further comprises administering an effective amount of an organoplatinum compound. In some embodiments, the method further comprises administering an effective amount of oxaliplatin (OX). In some embodiments, the method further comprises surgery, radiation therapy, gene therapy, RNA therapy, immunotherapy, nanotherapy, or a combination thereof. In some embodiments, the administration is intravenous. In some embodiments, the administration is intraperitoneal. In some embodiments, the administration is orally. In some embodiments, a poly-ADP-ribose polymerase (PARP) is inhibited by the compound in the subject. In some embodiments, mono-ADP ribosylation and poly-ADP ribosylation are inhibited. In some embodiments, a tumor cell undergoes apoptosis, cell cycle arrest, and/or necrosis in the subject. In some embodiments, the subject expresses a detectable level of PARP protein. In some embodiments, the subject has a detectable level of mono or poly-ADP ribosylation.

In some embodiments, the present invention discloses a method of treating a cancer comprising administering to a subject in need thereof an effective amount of a composition comprising an organoplatinum compound, an antimetabolite, and a compound of formula (I), or a pharmaceutically acceptable salt or prodrug thereof. In some embodiments, the present invention discloses a method of treating a cancer comprising administering to a subject in need thereof an effective amount of a composition comprising oxaliplatin (OX), gemcitabine (GEM) and a compound of formula (I), or a pharmaceutically acceptable salt or prodrug thereof. In some embodiments, the compound is 5-iodo-6-nitro-benzopyrone of Formula IIIg, or one of its pharmaceutically acceptable salts or prodrugs. In some embodiments, the compound is 5-iodo-6-amino-benzopyrone of Formula IIIk, or one of its pharmaceutically acceptable salts or prodrugs. In some embodiments, the compound is 5-iodo-6-nitroso-benzopyrone of Formula IIIl, or one of its pharmaceutically acceptable salts or prodrugs. In some embodiments, the compound is 5-iodo-6-hydroxylamino-benzopyrone of Formula IIIm, or one of its pharmaceutically acceptable salts or prodrugs. In some embodiments, the method further comprises administering an effective amount of OX, GEM and 5-iodo-6-nitro-benzopyrone (IIIg). In some embodiments, the method further comprises administering an effective amount of OX, GEM and 5-iodo-6-amino-benzopyrone (IIIk). In some embodiments, the method further comprises surgery, radiation therapy, gene therapy, RNA therapy, nanotherapy, immunotherapy, or a combination thereof. In some embodiments, the administration is intravenous. In some embodiments, the administration is intraperitoneal. In some embodiments, the administration is orally. In some embodiments, a poly-ADP-ribose polymerase (PARP) is inhibited by the compound in the subject. In some embodiments, mono-ADP ribosylation and poly-ADP ribosylation are inhibited. In some embodiments, a tumor cell undergoes apoptosis, cell cycle arrest, and/or necrosis in the subject. In some embodiments, the subject expresses a detectable level of PARP protein. In some embodiments, the subject has a detectable level of mono or poly-ADP ribosylation.

In another aspect, the present invention provides a composition for the treatment of a cancer, the composition comprising compound of formula I, or a metabolite, a pharmaceutically acceptable salt or prodrug thereof:

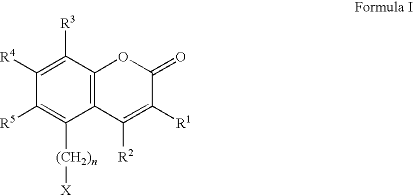

wherein n=0-10; R¹, R², R³, R⁴, R⁵ and X are independently selected from the group consisting of hydrogen, hydroxy, optionally substituted amine, amino, carboxyl, ester, nitroso, nitro, halogen, optionally substituted (C₁-C₆) alkyl, optionally substituted (C₁-C₆) alkoxy, optionally substituted (C₃-C₇) cycloalkyl, optionally substituted (C₃-C₇) heterocyclic, phenyl, and optionally substituted aryl; and wherein at least two of the R¹, R², R³, R⁴, and R⁵ substituents are always hydrogen; and wherein the compound is not one of the following:

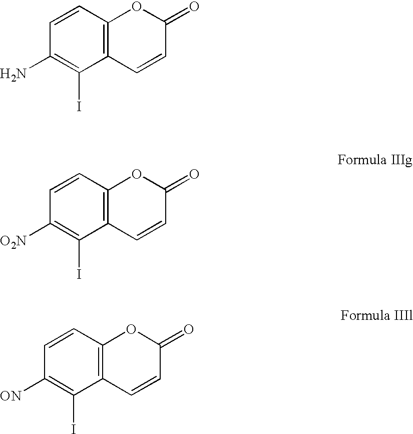

In some embodiments, the compound is of the formula IIIa, IIIb, IIIc, IIId, IIIe, IIIf, IIIh, IIIm, or IIIn, or a metabolite, a pharmaceutically acceptable salt or prodrug thereof:

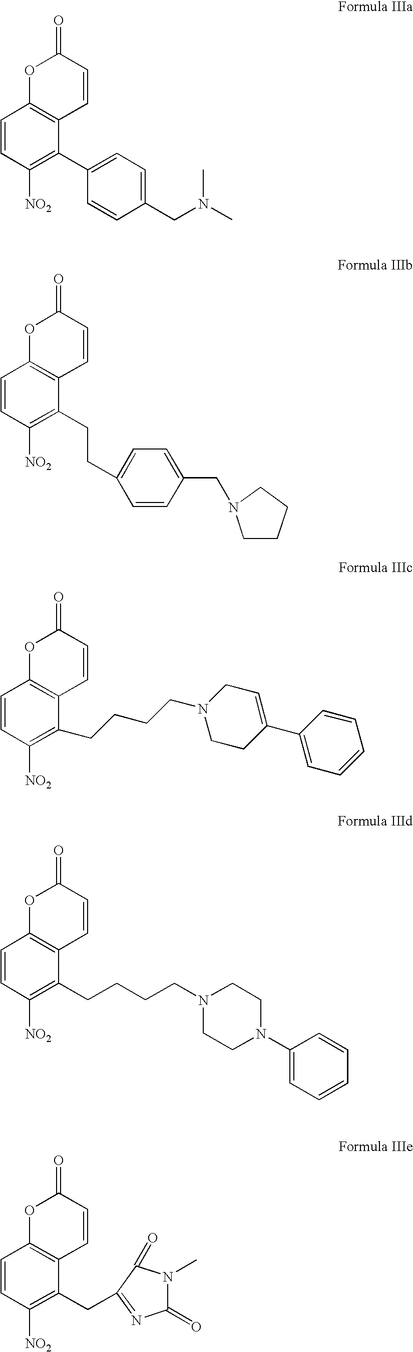

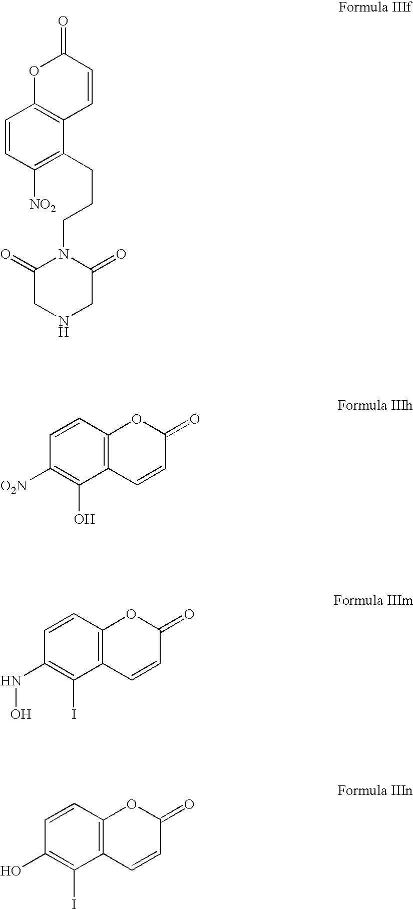

In some embodiments, the cancer is selected from the group consisting of adrenal cortical cancer, anal cancer, aplastic anemia, bile duct cancer, bladder cancer, bone cancer, bone metastasis, central nervous system (CNS) cancers, peripheral nervous system (PNS) cancers, Castleman's Disease, cervical cancer, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's disease, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, uterine cancer (e.g. uterine sarcoma), vaginal cancer, vulvar cancer, and Waldenstrom's macroglobulinemia.

In some embodiments of the composition, the cancer is breast cancer, ovarian cancer, uterine cancer, pancreatic cancer, lung cancer, brain cancer, skin cancer, colon cancer, or a cancer derived from cancer stem cells. In some embodiments, the breast cancer is negative for at least one of: ER, PR or HER2. In some embodiments, the breast cancer is negative for at least one of: ER, PR or HER2; and wherein the breast cancer is positive for at least one of ER, PR or HER2. In some embodiments, the breast cancer is negative for two of: ER, PR or HER2. In some embodiments, the breast cancer is ER-negative and PR-negative. In some embodiments, the breast cancer is ER-negative and HER2-negative. In some embodiments, the breast cancer is PR-negative and HER2-negative. In some embodiments, the breast cancer is an ER-negative breast cancer. In some embodiments, the breast cancer is an HER2-negative breast cancer.

In some embodiments, the composition further comprises an anti-tumor agent. In some embodiments, the anti-tumor agent is selected from the group consisting of antitumor alkylating agents, antitumor antimetabolites, antitumor antibiotics, plant-derived antitumor agents, antitumor organoplatinum compounds, antitumor campthotecin derivatives, antitumor tyrosine kinase inhibitors, monoclonal antibodies, interferons, biological response modifiers, and other agents having antitumor activities, or a pharmaceutically acceptable salt thereof. In some embodiments, the antitumor alkylating agents comprise nitrogen mustard N-oxide, cyclophosphamide, ifosfamide, melphalan, busulfan, mitobronitol, carboquone, thiotepa, ranimustine, nimustine, temozolomide, and carmustine; the antitumor antimetabolites comprise methotrexate, 6-mercaptopurine riboside, mercaptopurine, 5-fluorouracil, tegafur, doxifluridine, carmofur, cytarabine, cytarabine ocfosfate, enocitabine, S-1, gemcitabine, fludarabine, and pemetrexed disodium; the antitumor antibiotics comprise actinomycin D, doxorubicin, daunorubicin, neocarzinostatin, bleomycin, peplomycin, mitomycin C, aclarubicin, pirarubicin, epirubicin, zinostatin stimalamer, idarubicin, sirolimus, and valrubicin; the plant-derived antitumor agents comprise vincristine, vinblastine, vindeshine, etoposide, sobuzoxane, docetaxel, paclitaxel, and vinorelbine; the antitumor platinum-complex compounds comprise cisplatin, carboplatin, nedaplatin, and oxaliplatin; the antitumor campthotecin derivatives comprise irinotecan, topotecan, and campthotecin; the antitumor tyrosine kinase inhibitors comprise gefitinib, imatinib, and erlotinib; the monoclonal antibodies comprise cetuximab, bevacizumab, rituximab, bevacizumab, alemtuzumab, and trastuzumab; the interferons comprise interferon α, interferon α-2a, interferon α-2b, interferon β, interferon γ-1a, and interferon γ-n1, the biological response modifiers comprise krestin, lentinan, sizofuran, picibanil, or ubenimex, and the other antitumor agents comprise mitoxantrone, L-asparaginase, procarbazine, dacarbazine, hydroxycarbamide, pentostatin, tretinoin, alefacept, darbepoetin alfa, anastrozole, exemestane, bicalutamide, leuprorelin, flutamide, fulvestrant, pegaptanib octasodium, denileukin diftitox, aldesleukin, thyrotropin alfa, arsenic trioxide, bortezomib, capecitabine, and goserelin. In some embodiments, the anti-tumor agent is an organoplatinum compound. In some embodiments, the anti-tumor agent is oxaliplatin (OX), cisplatin, or carboplatin. In some embodiments, the anti-tumor agent is an anti-metabolite agent. In some embodiments, the anti-tumor agent is gemcitabine (GEM). In some embodiments, the composition further comprises more than one anti-tumor agent. In some embodiments, the anti-tumor agents are an organoplatinum compound and an anti-metabolite agent. In some embodiments, the anti-tumor agents are OX and GEM. In some embodiments, poly-ADP-ribose polymerase (PARP) is inhibited by the compound of formula (I). In some embodiments, mono-ADP ribosylation and poly-ADP ribosylation are inhibited. In some embodiments, a cancer cell expresses a detectable level of PARP protein. In some embodiments, the subject has a detectable level of mono or poly-ADP ribosylation.

In some embodiments, the present invention provides a kit for the treatment of a cancer. The kit comprises an effective amount of a compound of formula (I), or a pharmaceutically acceptable salt or prodrug thereof. In some embodiments, the kit can be used to treat cancer comprising adrenal cortical cancer, anal cancer, aplastic anemia, bile duct cancer, bladder cancer, bone cancer, bone metastasis, central nervous system (CNS) cancers, peripheral nervous system (PNS) cancers, Castleman's Disease, cervical cancer, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's disease, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, uterine cancer (e.g. uterine sarcoma), vaginal cancer, vulvar cancer, and Waldenstrom's macroglobulinemia.

In some embodiments, the present invention provides a composition for the treatment of a cancer, the composition comprising a combination of an anti-tumor agent and a compound of formula I, or a metabolite, a pharmaceutically acceptable salt or prodrug thereof:

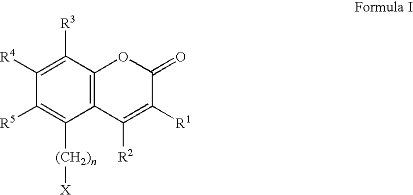

wherein n=0-10; R¹, R², R³, R⁴, R⁵ and X are independently selected from the group consisting of hydrogen, hydroxy, optionally substituted amine, amino, carboxyl, ester, nitroso, nitro, halogen, optionally substituted (C₁-C₆) alkyl, optionally substituted (C₁-C₆) alkoxy, optionally substituted (C₃-C₇) cycloalkyl, optionally substituted (C₃-C₇) heterocyclic, phenyl, and optionally substituted aryl; and wherein at least two of the R¹, R², R³, R⁴, and R⁵ substituents are always hydrogen.

In some embodiments, the compound is of formula II or a metabolite, a pharmaceutically acceptable salt or prodrug thereof:

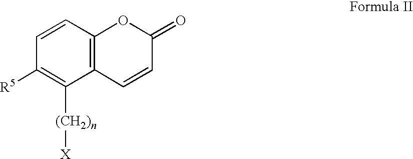

wherein R⁵ is selected from the group consisting of hydrogen, carboxyl, amino, nitroso, nitro and hydroxy; hydroxylamino, and X is selected from the group consisting of halogen, hydroxy, optionally substituted (C₁-C₇) alkyl, optionally substituted (C₁-C₆) alkoxy, optionally substituted (C₃-C₇) cycloalkyl, optionally substituted (C₃-C₇) heterocyclic, phenyl, and optionally substituted aryl.

In some embodiments, the compound is of the formula IIIa, IIIb, IIIc, IIId, IIIe, IIIf, IIIg, IIIh, IIIk, IIIl, IIIm, IIIn, or a metabolite, a pharmaceutically acceptable salt or prodrug thereof:

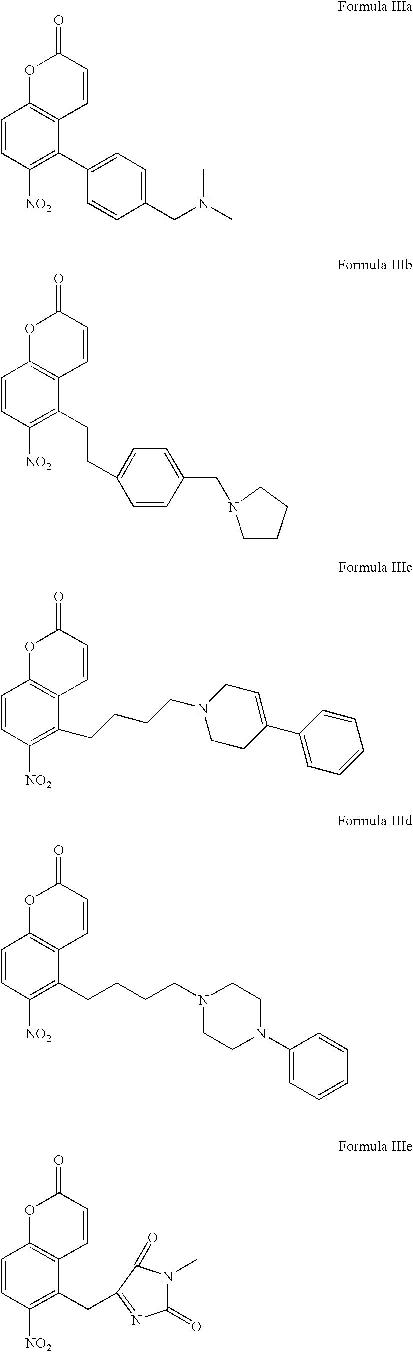

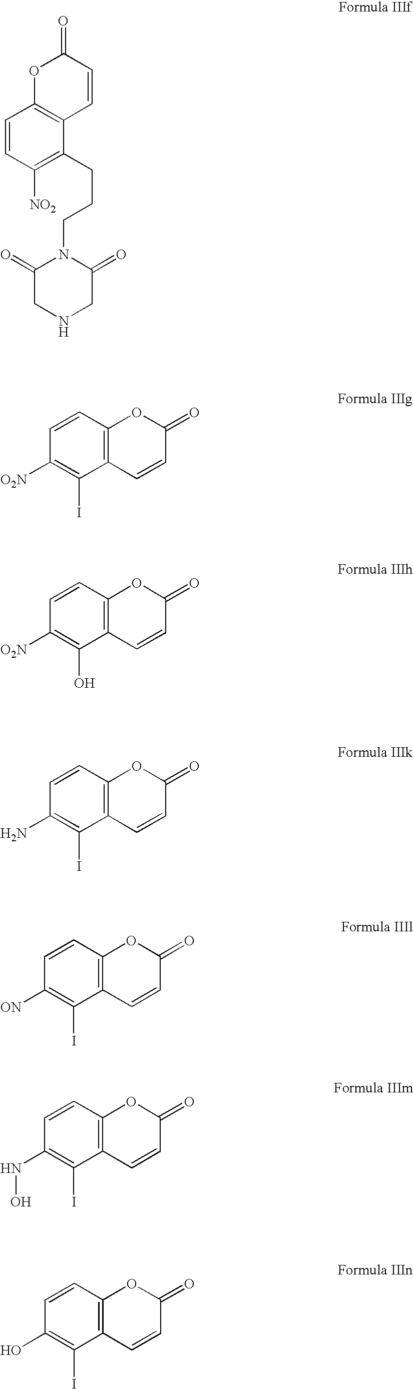

In some embodiments of the composition disclosed herein, the compound is 5-iodo-6-nitro-benzopyrone of Formula IIIg, or a metabolite, a pharmaceutically acceptable salt or prodrug thereof. In some embodiments, the compound is 5-iodo-6-amino-benzopyrone of Formula IIIk, or a metabolite, a pharmaceutically acceptable salt or prodrug thereof. In some embodiments of the composition disclosed herein, the compound is 5-iodo-6-nitroso-benzopyrone of Formula IIIl, or a metabolite, a pharmaceutically acceptable salt or prodrug thereof. In some embodiments, the compound is 5-iodo-6-hydroxylamino-benzopyrone of Formula IIIm, or a metabolite, a pharmaceutically acceptable salt or prodrug thereof.

In some embodiments of the composition, the cancer comprises adrenal cortical cancer, anal cancer, aplastic anemia, bile duct cancer, bladder cancer, bone cancer, bone metastasis, central nervous system (CNS) cancers, peripheral nervous system (PNS) cancers, breast cancer, Castleman's Disease, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, uterine cancer (e.g. uterine sarcoma), vaginal cancer, vulvar cancer, and Waldenstrom's macroglobulinemia.

In some embodiments of the composition, the cancer is breast cancer, ovarian cancer, uterine cancer, pancreatic cancer, lung cancer, brain cancer, skin cancer, colon cancer, or a cancer derived from cancer stem cells. In some embodiments, the breast cancer is negative for at least one of: ER, PR or HER2. In some embodiments, the breast cancer is negative for at least one of: ER, PR or HER2; and wherein the breast cancer is positive for at least one of ER, PR or HER2. In some embodiments, the breast cancer is negative for two of: ER, PR or HER2. In some embodiments, the breast cancer is ER-negative and PR-negative. In some embodiments, the breast cancer is ER-negative and HER2-negative. In some embodiments, the breast cancer is PR-negative and HER2-negative. In some embodiments, the breast cancer is an ER-negative breast cancer. In some embodiments, the breast cancer is an HER2-negative breast cancer.

In some embodiments of the composition disclosed herein, the anti-tumor agent comprises antitumor alkylating agents, antitumor antimetabolites, antitumor antibiotics, plant-derived antitumor agents, antitumor organoplatinum compounds, antitumor campthotecin derivatives, antitumor tyrosine kinase inhibitors, monoclonal antibodies, interferons, biological response modifiers, and other agents having antitumor activities, or a pharmaceutically acceptable salt thereof. In some embodiments, the antitumor alkylating agents comprise nitrogen mustard N-oxide, cyclophosphamide, ifosfamide, melphalan, busulfan, mitobronitol, carboquone, thiotepa, ranimustine, nimustine, temozolomide, and carmustine; the antitumor antimetabolites comprise methotrexate, 6-mercaptopurine riboside, mercaptopurine, 5-fluorouracil, tegafur, doxifluridine, carmofur, cytarabine, cytarabine ocfosfate, enocitabine, S-1, gemcitabine, fludarabine, and pemetrexed disodium; the antitumor antibiotics comprise actinomycin D, doxorubicin, daunorubicin, neocarzinostatin, bleomycin, peplomycin, mitomycin C, aclarubicin, pirarubicin, epirubicin, zinostatin stimalamer, idarubicin, sirolimus, and valrubicin; the plant-derived antitumor agents comprise vincristine, vinblastine, vindeshine, etoposide, sobuzoxane, docetaxel, paclitaxel, and vinorelbine; the antitumor platinum-complex compounds comprise cisplatin, carboplatin, nedaplatin, and oxaliplatin; the antitumor campthotecin derivatives comprise irinotecan, topotecan, and campthotecin; the antitumor tyrosine kinase inhibitors comprise gefitinib, imatinib, and erlotinib; the monoclonal antibodies comprise cetuximab, bevacizumab, rituximab, bevacizumab, alemtuzumab, and trastuzumab; the interferons comprise interferon α, interferon α-2a, interferon α-2b, interferon β, interferon γ-1a, and interferon γ-n1, the biological response modifiers comprise krestin, lentinan, sizofuran, picibanil, or ubenimex, and the other antitumor agents comprise mitoxantrone, L-asparaginase, procarbazine, dacarbazine, hydroxycarbamide, pentostatin, tretinoin, alefacept, darbepoetin alfa, anastrozole, exemestane, bicalutamide, leuprorelin, flutamide, fulvestrant, pegaptanib octasodium, denileukin diftitox, aldesleukin, thyrotropin alfa, arsenic trioxide, bortezomib, capecitabine, and goserelin.

In some embodiments, the anti-tumor agent is an organoplatinum compound. In some embodiments, the anti-tumor agent is oxaliplatin (OX), cisplatin, or carboplatin. In some embodiments, the anti-tumor agent is an anti-metabolite agent. In some embodiments, the anti-tumor agent is gemcitabine (GEM). In some embodiments, the composition further comprises more than one anti-tumor agent. In some embodiments, the anti-tumor agents are an organoplatinum compound and an anti-metabolite agent. In some embodiments, the anti-tumor agents are OX and GEM. In some embodiments, poly-ADP-ribose polymerase (PARP) is inhibited by the compound of formula (I). In some embodiments, mono-ADP ribosylation and poly-ADP ribosylation are inhibited. In some embodiments, a cancer cell expresses a detectable level of PARP protein.

In some embodiments, the present invention provides a kit for the treatment of a cancer, the kit comprising an effective amount of a compound of formula (I) in combination with an anti-tumor agent as disclosed herein, or a pharmaceutically acceptable salt or prodrug thereof. In some embodiments, the cancer that may be treated by the kit disclosed herein includes, but is not limited to, adrenal cortical cancer, anal cancer, aplastic anemia, bile duct cancer, bladder cancer, bone cancer, bone metastasis, central nervous system (CNS) cancers, peripheral nervous system (PNS) cancers, breast cancer, Castleman's Disease, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, uterine cancer (e.g. uterine sarcoma), vaginal cancer, vulvar cancer, and Waldenstrom's macroglobulinemia.

### INCORPORATION BY REFERENCE

All publications, patents, and patent applications mentioned in this specification are herein incorporated by reference to the same extent as if each individual publication, patent, or patent application is specifically and individually indicated to be incorporated by reference.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows Expression of PARP-1 protein in pancreatic tumor cell lines.

FIG. 2 shows the effect of IIIg and its analogs on COLO357FG and MiaPaCa2 pancreatic cancer cell proliferation in vitro.

FIG. 3 shows effect of IIIg on Colo357FG or L3.6pl pancreatic cancer growth in luciferase expressing pancreatic orthotopic cancer model in nude mice.

FIG. 4 shows effect of IIIg on tumor growth in nude mice bearing orthotopic COLO357FG and L3.6 pl pancreatic tumors.

FIG. 5 shows effect of IIIg on survival of athymic mice bearing orthotopic COLO357FG and L3.6pl pancreatic tumors.

FIG. 6 shows effect of different schedules of administration of IIIg on tumor growth and survival of nude mice bearing orthotopic COLO357FG pancreatic tumors.

FIG. 7 shows proliferation of uterine cancer Hela cells at 96 hours after treatment with IIIg.

FIG. 8 shows proliferation of lung carcinoma A549 cells at 96 hours after treatment with IIIg.

FIG. 9 shows the effect of 5-iodo-6-nitro-benzopyrone (IIIg) on proliferation of PARP-1+(A16) and PARP-1−/− fibroblasts (A12).

FIG. 10 shows the effect of IIIg as a single agent on proliferation of pancreatic tumor cell lines.

FIG. 11 shows PARP1 expression and PARP activity in pancreatic tumor cell lines and PARP-1+(A16) and PARP-1−/− fibroblasts (A12).

FIG. 12 shows effect of dose and schedule of IIIg on COLO357FG pancreatic cancer growth and survival of nude mice in luceferase expressing pancreatic orthotopic cancer model.

FIG. 13 shows effect of dose and schedule of oral administration of IIIg on COLO357FG pancreatic cancer growth in luceferase expressing pancreatic orthotopic cancer model in nude mice.

FIG. 14 shows effect of IIIg and its combination with oxaliplatin on proliferation of pancreatic tumor cells COLO357FG and MiaPaCa-2.

FIG. 15 shows that COLO357FG pancreatic tumors are more sensitive to the combination treatment of oxaliplatin with IIIg.

FIG. 16 shows anti-tumor activity of IIIg against human MX-1 breast carcinoma xenograft in nude mice.

FIG. 17 shows anti-tumor activity of IIIg against human SW620 colon carcinoma xenograft in nude mice.

FIG. 18 shows effect of IIIg in combination with γ-irradiation on proliferation of pancreatic cancer cells MIAPACA 2 and Colo 3.6.

FIG. 19 shows mass spectrometric (MS) analysis of 100 μM IIIg in 50% methanol.

FIG. 20 shows mass spectrometric (MS) analysis of 208 ion in 60 minute human whole blood sample.

FIG. 21 shows mass spectrometric (MS) analysis of 497 ion in 60 minute human whole blood sample.

### DETAILED DESCRIPTION OF THE INVENTION

In some embodiments, the present invention provides for the use of the aforesaid benzopyrone compounds for the treatment of cancer. In some embodiments, the present invention also provides the use of the aforesaid benzopyrone compounds for the treatment of cancers that are resulted from metastasis or migration of a primary tumor cell. The cancers that may be treated using the methods and compositions of the present invention include but are not limited to adrenal cortical cancer, anal cancer, aplastic anemia, bile duct cancer, bladder cancer, bone cancer, bone metastasis, central nervous system (CNS) cancers, peripheral nervous system (PNS) cancers, breast cancer, Castleman's Disease, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, uterine cancer (e.g. uterine sarcoma), vaginal cancer, vulvar cancer, and Waldenstrom's macroglobulinemia.

In other embodiments, the present invention provides the use of the aforesaid benzopyrone compounds in combination with one or more anti-tumor agents for the treatment of cancer including but not limited to adrenal cortical cancer, anal cancer, aplastic anemia, bile duct cancer, bladder cancer, bone cancer, bone metastasis, central nervous system (CNS) cancers, peripheral nervous system (PNS) cancers, breast cancer, Castleman's Disease, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, uterine cancer (e.g. uterine sarcoma), vaginal cancer, vulvar cancer, and Waldenstrom's macroglobulinemia. The anti-tumor agents that may be used in the present invention include but are not limited to antitumor alkylating agents, antitumor antimetabolites, antitumor antibiotics, plant-derived antitumor agents, antitumor platinum-complex compounds, antitumor campthotecin derivatives, antitumor tyrosine kinase inhibitors, monoclonal antibodies, interferons, biological response modifiers, and other agents that exhibit anti-tumor activities, or a pharmaceutically acceptable salt thereof.

In other preferred embodiments, the benzopyrone compounds of the present invention are used for the treatment of cancers derived from stem cells. In many malignancies described herein, a proportion of tumor cells—“cancer stem cells”—have the capacity for extensive proliferation and transferal of the tumor. An alteration in stem cell fate and growth may play a role in tumorigenesis. Epithelial stem cells have a life-span at least as long as that of the organism, and thus they are thought to be susceptible to multiple genetic hits which cumulatively may result in tumor formation. Many cancers, such as those of the skin and colon, arise in tissues that are constantly replenished with cells throughout life. But the crucial mutations that lead to the disease are likely to have occurred during the tissues' formative period, when cells are dividing exponentially.

The stem cell compartment, now identified virtually in every tissue, can be defined as a subset of rare cells, endowed with the exclusive prerogative of self-renewal and persistence throughout the organism's life, in contrast with differentiated cells, which form the tissue bulk, but usually feature a postmitotic behavior and a short lifespan. The fact that several mutations are necessary for a cell to become cancerous may suggest that in many tissues the mutations may accumulate in stem cells. As cancer stem cells self-renew, it follows that they may be derived either from self-renewing normal stem cells, or from more differentiated cells that acquire peculiar properties of stem cells. Consistently, a tumor can be conceived as a tissue, including both “differentiated” cells, and a subset of “cancer stem cells”, which maintain the tumor mass, and are likely responsible for formation of secondary tumors (metastasis). Hence, benzopyrone compounds of the present invention can be used to target cancers derived from stem cells.

The present invention discloses a nonclinical pharmacology of 5-iodo-6-nitro-benzopyrone (IIIg) in human tumor and normal primary cells and also in mice. In vitro IIIg inhibits the proliferation of a variety of human tumor cells including pancreatic, breast, uterine, lung, prostate, and ovarian cancer cells. In vivo IIIg effect is evaluated in several animal models of carcinogenesis. Intraperitoneal injection and oral administration of IIIg, either alone, or in combination with oxaliplatin, inhibits growth of human metastatic pancreatic cancer cells in vivo. Once-daily or twice-weekly administration of IIIg inhibits tumor growth in both human breast carcinoma xenograft model and human colon carcinoma xenograft model in nude mice, and positively affects the survival rate of animals exposed to the drug given daily or twice weekly. The twice weekly dosing of IIIg for 54 days is based on the results of the pre-clinical evaluation of the efficacy and safety of IIIg.

It has been reported that benzopyrone compounds, more specifically, 5-iodo-6-nitrobenzopyrone, have selective cytotoxicity upon malignant cancer cells but not upon nonmalignant cells (Kirsten E, Kun E, Int J Mol Med, 2000, 5(3):279-81). In one embodiment, the benzopyrone compounds utilized in the methods of the present invention may exhibit more selective toxicity towards tumor cells than non-tumor cells.

### Anti-Tumor Agents

Anti-tumor agents that may be used in the present invention include but are not limited to antitumor alkylating agents, antitumor antimetabolites, antitumor antibiotics, plant-derived antitumor agents, antitumor platinum-complex compounds, antitumor campthotecin derivatives, antitumor tyrosine kinase inhibitors, monoclonal antibodies, interferons, biological response modifiers, and other agents that exhibit anti-tumor activities, or a pharmaceutically acceptable salt thereof.

In some embodiments, the anti-tumor agent is an alkylating agent. The term “alkylating agent” herein generally refers to an agent giving an alkyl group in the alkylation reaction in which a hydrogen atom of an organic compound is substituted with an alkyl group. Examples of anti-tumor alkylating agents include but are not limited to nitrogen mustard N-oxide, cyclophosphamide, ifosfamide, melphalan, busulfan, mitobronitol, carboquone, thiotepa, ranimustine, nimustine, temozolomide or carmustine.

In some embodiments, the anti-tumor agent is an antimetabolite. The term “antimetabolite” used herein includes, in a broad sense, substances which disturb normal metabolism and substances which inhibit the electron transfer system to prevent the production of energy-rich intermediates, due to their structural or functional similarities to metabolites that are important for living organisms (such as vitamins, coenzymes, amino acids and saccharides). Examples of antimetabolites that have anti-tumor activities include but are not limited to methotrexate, 6-mercaptopurine riboside, mercaptopurine, 5-fluorouracil, tegafur, doxifluridine, carmofur, cytarabine, cytarabine ocfosfate, enocitabine, S-1, gemcitabine, fludarabine or pemetrexed disodium, and preferred are 5-fluorouracil, S-1, gemcitabine and the like.

In some embodiments, the anti-tumor agent is an antitumor antibiotic. Examples of antitumor antibiotics include but are not limited to actinomycin D, doxorubicin, daunorubicin, neocarzinostatin, bleomycin, peplomycin, mitomycin C, aclarubicin, pirarubicin, epirubicin, zinostatin stimalamer, idarubicin, sirolimus or valrubicin.

In some embodiments, the anti-tumor agent is a plant-derived antitumor agent. Examples of plant-derived antitumor agents include but are not limited to vincristine, vinblastine, vindesine, etoposide, sobuzoxane, docetaxel, paclitaxel and vinorelbine, and preferred and docetaxel and paclitaxel.

In some embodiments, the anti-tumor agent is a camptothecin derivative that exhibits anti-tumor activities. Examples of anti-tumor camptothecin derivatives include but are not limited to camptothecin, 10-hydroxycamptothecin, topotecan, irinotecan or 9-aminocamptothecin, with camptothecin, topotecan and irinotecan being preferred. Further, irinotecan is metabolized in vivo and exhibits antitumor effect as SN-38. The action mechanism and the activity of the camptothecin derivatives are believed to be virtually the same as those of camptothecin (e.g., Nitta, et al., Gan to Kagaku Ryoho, 14, 850-857 (1987)).

In some embodiments, the anti-tumor agent is an organoplatinum compound or a platinum coordination compound having antitumor activity. Organoplatinum compound herein refers to a platinum containing compound which provides platinum in ion form. Preferred organoplatinum compounds include but are not limited to cisplatin; cis-diamminediaquoplatinum (II)-ion; chloro(diethylenetriamine)-platinum (II) chloride; dichloro(ethylenediamine)-platinum (II); diammine(1,1-cyclobutanedicarboxylato) platinum (II) (carboplatin); spiroplatin; iproplatin; diammine(2-ethylmalonato)platinum (II); ethylenediaminemalonatoplatinum (II); aqua(1,2-diaminodicyclohexane)sulfatoplatinum (II); aqua(1,2-diaminodicyclohexane)malonatoplatinum (II); (1,2-diaminocyclohexane)malonatoplatinum (II); (4-carboxyphthalato)(1,2-diaminocyclohexane) platinum (II); (1,2-diaminocyclohexane)-(isocitrato)platinum (II); (1,2-diaminocyclohexane)oxalatoplatinum (II); ormaplatin; tetraplatin; carboplatin, nedaplatin and oxaliplatin, and preferred is carboplatin or oxaliplatin. Further, other antitumor organoplatinum compounds mentioned in the specification are known and are commercially available and/or producible by a person having ordinary skill in the art by conventional techniques.

In some embodiments, the anti-tumor agent is an antitumor tyrosine kinase inhibitor. The term “tyrosine kinase inhibitor” herein refers to a chemical substance inhibiting “tyrosine kinase” which transfers a λ-phosphate group of ATP to a hydroxyl group of a specific tyrosine in protein. Examples of anti-tumor tyrosine kinase inhibitors include but are not limited to gefitinib, imatinib or erlotinib.

In some embodiments, the anti-tumor agent is an antibody or a binding portion of an antibody that exhibits anti-tumor activity. In some embodiments, the anti-tumor agent is a monoclonal antibody. Examples thereof include but are not limited to abciximab, adalimumab, alemtuzumab, basiliximab, bevacizumab, cetuximab, daclizumab, eculizumab, efalizumab, ibritumomab, tiuxetan, infliximab, muromonab-CD3, natalizumab, omalizumab, palivizumab, panitumumab, ranibizumab, gemtuzumab ozogamicin, rituximab, tositumomab, trastuzumab, or any antibody fragments specific for antigens.

In some embodiments, the anti-tumor agent is an interferon. Such interferon has antitumor activity, and it is a glycoprotein which is produced and secreted by most animal cells upon viral infection. It has not only the effect of inhibiting viral growth but also various immune effector mechanisms including inhibition of growth of cells (in particular, tumor cells) and enhancement of the natural killer cell activity, thus being designated as one type of cytokine. Examples of anti-tumor interferons include but are not limited to interferon α, interferon α-2a, interferon α-2b, interferon β, interferon γ-1a and interferon γ-n1.

In some embodiments, the anti-tumor agent is a biological response modifier. It is generally the generic term for substances or drugs for modifying the defense mechanisms of living organisms or biological responses such as survival, growth or differentiation of tissue cells in order to direct them to be useful for an individual against tumor, infection or other diseases. Examples of the biological response modifier include but are not limited to krestin, lentinan, sizofuran, picibanil and ubenimex.

In some embodiments, the anti-tumor agents include but are not limited to mitoxantrone, L-asparaginase, procarbazine, dacarbazine, hydroxycarbamide, pentostatin, tretinoin, alefacept, darbepoetin alfa, anastrozole, exemestane, bicalutamide, leuprorelin, flutamide, fulvestrant, pegaptanib octasodium, denileukin diftitox, aldesleukin, thyrotropin alfa, arsenic trioxide, bortezomib, capecitabine, and goserelin.

The above-described terms “antitumor alkylating agent”, “antitumor antimetabolite”, “antitumor antibiotic”, “plant-derived antitumor agent”, “antitumor platinum coordination compound”, “antitumor camptothecin derivative”, “antitumor tyrosine kinase inhibitor”, “monoclonal antibody”, “interferon”, “biological response modifier” and “other antitumor agent” are all known and are either commercially available or producible by a person skilled in the art by methods known per se or by well-known or conventional methods. The process for preparation of gefitinib is described, for example, in U.S. Pat. No. 5,770,599; the process for preparation of cetuximab is described, for example, in WO 96/40210; the process for preparation of bevacizumab is described, for example, in WO 94/10202; the process for preparation of oxaliplatin is described, for example, in U.S. Pat. Nos. 5,420,319 and 5,959,133; the process for preparation of gemcitabine is described, for example, in U.S. Pat. Nos. 5,434,254 and 5,223,608; and the process for preparation of camptothecin is described in U.S. Pat. Nos. 5,162,532, 5,247,089, 5,191,082, 5,200,524, 5,243,050 and 5,321,140; the process for preparation of irinotecan is described, for example, in U.S. Pat. No. 4,604,463; the process for preparation of topotecan is described, for example, in U.S. Pat. No. 5,734,056; the process for preparation of temozolomide is described, for example, in JP-B No. 4-5029; and the process for preparation of rituximab is described, for example, in JP-W No. 2-503143.

The above-mentioned antitumor alkylating agents are commercially available, as exemplified by the following: nitrogen mustard N-oxide from Mitsubishi Pharma Corp. as Nitrorin (tradename); cyclophosphamide from Shionogi & Co., Ltd. as Endoxan (tradename); ifosfamide from Shionogi & Co., Ltd. as Ifomide (tradename); melphalan from GlaxoSmithKline Corp. as Alkeran (tradename); busulfan from Takeda Pharmaceutical Co., Ltd. as Mablin (tradename); mitobronitol from Kyorin Pharmaceutical Co., Ltd. as Myebrol (tradename); carboquone from Sankyo Co., Ltd. as Esquinon (tradename); thiotepa from Sumitomo Pharmaceutical Co., Ltd. as Tespamin (tradename); ranimustine from Mitsubishi Pharma Corp. as Cymerin (tradename); nimustine from Sankyo Co., Ltd. as Nidran (tradename); temozolomide from Schering Corp. as Temodar (tradename); and carmustine from Guilford Pharmaceuticals Inc. as Gliadel Wafer (tradename).

The above-mentioned antitumor antimetabolites are commercially available, as exemplified by the following: methotrexate from Takeda Pharmaceutical Co., Ltd. as Methotrexate (tradename); 6-mercaptopurine riboside from Aventis Corp. as Thioinosine (tradename); mercaptopurine from Takeda Pharmaceutical Co., Ltd. as Leukerin (tradename); 5-fluorouracil from Kyowa Hakko Kogyo Co., Ltd. as 5-FU (tradename); tegafur from Taiho Pharmaceutical Co., Ltd. as Futraful (tradename); doxyfluridine from Nippon Roche Co., Ltd. as Furutulon (tradename); carmofur from Yamanouchi Pharmaceutical Co., Ltd. as Yamafur (tradename); cytarabine from Nippon Shinyaku Co., Ltd. as Cylocide (tradename); cytarabine ocfosfate from Nippon Kayaku Co., Ltd. as Strasid(tradename); enocitabine from Asahi Kasei Corp. as Sanrabin (tradename); S-1 from Taiho Pharmaceutical Co., Ltd. as TS-1 (tradename); gemcitabine from Eli Lilly & Co. as Gemzar (tradename); fludarabine from Nippon Schering Co., Ltd. as Fludara (tradename); and pemetrexed disodium from Eli Lilly & Co. as Alimta (tradename).

The above-mentioned antitumor antibiotics are commercially available, as exemplified by the following: actinomycin D from Banyu Pharmaceutical Co., Ltd. as Cosmegen (tradename); doxorubicin from Kyowa Hakko Kogyo Co., Ltd. as adriacin (tradename); daunorubicin from Meiji Seika Kaisha Ltd. as Daunomycin; neocarzinostatin from Yamanouchi Pharmaceutical Co., Ltd. as Neocarzinostatin (tradename); bleomycin from Nippon Kayaku Co., Ltd. as Bleo (tradename); pepromycin from Nippon Kayaku Co, Ltd. as Pepro (tradename); mitomycin C from Kyowa Hakko Kogyo Co., Ltd. as Mitomycin (tradename); aclarubicin from Yamanouchi Pharmaceutical Co., Ltd. as Aclacinon (tradename); pirarubicin from Nippon Kayaku Co., Ltd. as Pinorubicin (tradename); epirubicin from Pharmacia Corp. as Pharmorubicin (tradename); zinostatin stimalamer from Yamanouchi Pharmaceutical Co., Ltd. as Smancs (tradename); idarubicin from Pharmacia Corp. as Idamycin (tradename); sirolimus from Wyeth Corp. as Rapamune (tradename); and valrubicin from Anthra Pharmaceuticals Inc. as Valstar (tradename).

The above-mentioned plant-derived antitumor agents are commercially available, as exemplified by the following: vincristine from Shionogi & Co., Ltd. as Oncovin (tradename); vinblastine from Kyorin Pharmaceutical Co., Ltd. as Vinblastine (tradename); vindesine from Shionogi & Co., Ltd. as Fildesin (tradename); etoposide from Nippon Kayaku Co., Ltd. as Lastet (tradename); sobuzoxane from Zenyaku Kogyo Co., Ltd. as Perazolin (tradename); docetaxel from Aventis Corp. as Taxsotere (tradename); paclitaxel from Bristol-Myers Squibb Co. as Taxol (tradename); and vinorelbine from Kyowa Hakko Kogyo Co., Ltd. as Navelbine (tradename).

The above-mentioned antitumor platinum coordination compounds are commercially available, as exemplified by the following: cisplatin from Nippon Kayaku Co., Ltd. as Randa (tradename); carboplatin from Bristol-Myers Squibb Co. as Paraplatin (tradename); nedaplatin from Shionogi & Co., Ltd. as Aqupla (tradename); and oxaliplatin from Sanofi-Synthelabo Co. as Eloxatin (tradename).

The above-mentioned antitumor camptothecin derivatives are commercially available, as exemplified by the following: irinotecan from Yakult Honsha Co., Ltd. as Campto (tradename); topotecan from GlaxoSmithKline Corp. as Hycamtin (tradename); and camptothecin from Aldrich Chemical Co., Inc., U.S.A.

The above-mentioned antitumor tyrosine kinase inhibitors are commercially available, as exemplified by the following: gefitinib from AstraZeneca Corp. as Iressa (tradename); imatinib from Novartis AG as Gleevec (tradename); and erlotinib from OSI Pharmaceuticals Inc. as Tarceva (tradename).

The above-mentioned monoclonal antibodies are commercially available, as exemplified by the following: cetuximab from Bristol-Myers Squibb Co. as Erbitux (tradename); bevacizumab from Genentech, Inc. as Avastin (tradename); rituximab from Biogen Idec Inc. as Rituxan (tradename); alemtuzumab from Berlex Inc. as Campath (tradename); and trastuzumab from Chugai Pharmaceutical Co., Ltd. as Herceptin (tradename).

The above-mentioned interferons are commercially available, as exemplified by the following: interferon α from Sumitomo Pharmaceutical Co., Ltd. as Sumiferon (tradename); interferon α-2a from Takeda Pharmaceutical Co., Ltd. as Canferon-A (tradename); interferon α-2b from Schering-Plough Corp. as Intron A (tradename); interferon β from Mochida Pharmaceutical Co., Ltd. as IFN.beta. (tradename); interferon γ-1a from Shionogi & Co., Ltd. as Immunomax-γ (tradename); and interferon γ-n1 from Otsuka Pharmaceutical Co., Ltd. as Ogamma (tradename).

The above-mentioned biological response modifiers are commercially available, as exemplified by the following: krestin from Sankyo Co., Ltd. as krestin (tradename); lentinan from Aventis Corp. as Lentinan (tradename); sizofuran from Kaken Seiyaku Co., Ltd. as Sonifuran (tradename); picibanil from Chugai Pharmaceutical Co., Ltd. as Picibanil (tradename); and ubenimex from Nippon Kayaku Co., Ltd. as Bestatin (tradename).

The above-mentioned other antitumor agents are commercially available, as exemplified by the following: mitoxantrone from Wyeth Lederle Japan, Ltd. as Novantrone (tradename); L-asparaginase from Kyowa Hakko Kogyo Co., Ltd. as Leunase (tradename); procarbazine from Nippon Roche Co., Ltd. as Natulan (tradename); dacarbazine from Kyowa Hakko Kogyo Co., Ltd. as Dacarbazine (tradename); hydroxycarbamide from Bristol-Myers Squibb Co. as Hydrea (tradename); pentostatin from Kagaku Oyobi Kessei Ryoho Kenkyusho as Coforin (tradename); tretinoin from Nippon Roche Co., Ltd. As Vesanoid (tradename); alefacept from Biogen Idec Inc. as Amevive (tradename); darbepoetin alfa from Amgen Inc. as Aranesp (tradename); anastrozole from AstraZeneca Corp. as Arimidex (tradename); exemestane from Pfizer Inc. as Aromasin (tradename); bicalutamide from AstraZeneca Corp. as Casodex (tradename); leuprorelin from Takeda Pharmaceutical Co., Ltd. as Leuplin (tradename); flutamide from Schering-Plough Corp. as Eulexin (tradename); fulvestrant from AstraZeneca Corp. as Faslodex (tradename); pegaptanib octasodium from Gilead Sciences, Inc. as Macugen (tradename); denileukin diftitox from Ligand Pharmaceuticals Inc. as Ontak (tradename); aldesleukin from Chiron Corp. as Proleukin (tradename); thyrotropin alfa from Genzyme Corp. as Thyrogen (tradename); arsenic trioxide from Cell Therapeutics, Inc. as Trisenox (tradename); bortezomib from Millennium Pharmaceuticals, Inc. as Velcade (tradename); capecitabine from Hoffmann-La Roche, Ltd. as Xeloda (tradename); and goserelin from AstraZeneca Corp. as Zoladex (tradename). The term “antitumor agent” as used in the specification includes the above-described antitumor alkylating agent, antitumor antimetabolite, antitumor antibiotic, plant-derived antitumor agent, antitumor platinum coordination compound, antitumor camptothecin derivative, antitumor tyrosine kinase inhibitor, monoclonal antibody, interferon, biological response modifier, and other antitumor agents.

One of the most promising anti-tumor agents in cancer therapy is oxaliplatin (OX). OX is a member of the organoplatinum family drugs, i.e. platinum-based chemotherapy drugs. Other examples of organoplatinum drugs include but are not limited to cisplatin and carboplatin. OX induces DNA single-strand breaks. It is typically administered in combination with fluorouracil and leucovorin in a combination known as FOLFOX for the treatment of colorectal cancer. The two amine groups of OX are replaced by cyclohexyldiamine for improved antitumour activity, and the chlorine ligands are replaced by the oxalato bidentate derived from oxalic acid to improve water solubility. The cytotoxicity of OX is thought to result from inhibition of DNA synthesis.

Gemcitabine (GEM) is a nucleoside analog in which the hydrogens on the 2′ carbons of deoxycytidine are replaced by fluorines. As with fluorouracil and other analogues of pyrimidines, the drug replaces one of the building blocks of nucleic acids, in this case cytidine, during DNA replication. The process arrests tumor growth, resulting in apoptosis. The invention also provides a method for treating cancer comprising the administration of a benzopyrone compound in combination with one or more anti-tumor agents including but not limited to OX and GEM.

In addition to OX and GEM, other anti-tumor agents or anti-neoplastic agents can be used in combination with benzopyrone compounds. Such suitable anti-tumor agents or anti-neoplastic agents include, but are not limited to, 13-cis-Retinoic Acid, 2-CdA, 2-Chlorodeoxyadenosine, 5-Azacitidine, 5-Fluorouracil, 5-FU, 6-Mercaptopurine, 6-MP, 6-TG, 6-Thioguanine, Abraxane, Accutane, Actinomycin-D, Adriamycin, Adrucil, Agrylin, Ala-Cort, Aldesleukin, Alemtuzumab, ALIMTA, Alitretinoin, Alkaban-AQ, Alkeran, All-transretinoic Acid, Alpha Interferon, Altretamine, Amethopterin, Amifostine, Aminoglutethimide, Anagrelide, Anandron, Anastrozole, Arabinosylcytosine, Ara-C, Aranesp, Aredia, Arimidex, Aromasin, Arranon, Arsenic Trioxide, Asparaginase, ATRA, Avastin, Azacitidine, BCG, BCNU, Bendamustine, Bevacizumab, Bexarotene, BEXXAR, Bicalutamide, BiCNU, Blenoxane, Bleomycin, Bortezomib, Busulfan, Busulfex, C225, Calcium Leucovorin, Campath, Camptosar, Camptothecin-11, Capecitabine, Carac, Carboplatin, Carmustine, Carmustine Wafer, Casodex, CC-5013, CCI-779, CCNU, CDDP, CeeNU, Cerubidine, Cetuximab, Chlorambucil, Cisplatin, Citrovorum Factor, Cladribine, Cortisone, Cosmegen, CPT-11, Cyclophosphamide, Cytadren, Cytarabine, Cytarabine Liposomal, Cytosar-U, Cytoxan, Dacarbazine, Dacogen, Dactinomycin, Darbepoetin Alfa, Dasatinib, Daunomycin, Daunorubicin, Daunorubicin Hydrochloride, Daunorubicin Liposomal, DaunoXome, Decadron, Decitabine, Delta-Cortef, Deltasone, Denileukin Diftitox, DepoCyt™, Dexamethasone, Dexamethasone Acetate, Dexamethasone Sodium Phosphate, Dexasone, Dexrazoxane, DHAD, DIC, Diodex, Docetaxel, Doxil, Doxorubicin, Doxorubicin Liposomal, Droxia™, DTIC, DTIC-Dome, Duralone, Efudex, Eligard, Ellence, Eloxatin, Elspar, Emcyt, Epirubicin, Epoetin Alfa, Erbitux, Erlotinib, Erwinia L-asparaginase, Estramustine, Ethyol, Etopophos, Etoposide, Etoposide Phosphate, Eulexin, Evista, Exemestane, Fareston, Faslodex, Femara, Filgrastim, Floxuridine, Fludara, Fludarabine, Fluoroplex, Fluorouracil, Fluorouracil (cream), Fluoxymesterone, Flutamide, Folinic Acid, FUDR, Fulvestrant, G-CSF, Gefitinib, Gemcitabine, Gemtuzumab ozogamicin, Gemzar & Gemzar Side Effects—Chemotherapy Drugs, Gleevec, Gliadel Wafer, GM-CSF, Goserelin, Granulocyte—Colony Stimulating Factor, Granulocyte Macrophage Colony Stimulating Factor, Halotestin, Herceptin, Hexadrol, Hexylen, Hexamethylmelamine, HMM, Hycamtin, Hydrea, Hydrocort Acetate, Hydrocortisone, Hydrocortisone Sodium Phosphate, Hydrocortisone Sodium Succinate, Hydrocortone Phosphate, Hydroxyurea, Ibritumomab, Ibritumomab Tiuxetan, Idamycin, Idarubicin, Ifex, IFN-alpha, Ifosfamide, IL-11, IL-2, Imatinib mesylate, Imidazole Carboxamide, Interferon alfa, Interferon Alfa-2b (PEG Conjugate), Interleukin-2, Interleukin-11, Intron A (interferon alfa-2b), Iressa, Irinotecan, Isotretinoin, Ixabepilone, Ixempra, Kidrolase (t), Lanacort, Lapatinib, L-asparaginase, LCR, Lenalidomide, Letrozole, Leucovorin, Leukeran, Leukine, Leuprolide, Leurocristine, Leustatin, Liposomal Ara-C, Liquid Pred, Lomustine, L-PAM, L-Sarcolysin, Lupron, Lupron Depot, Matulane, Maxidex, Mechlorethamine, Mechlorethamine Hydrochloride, Medralone, Medrol, Megace, Megestrol, Megestrol Acetate, Melphalan, Mercaptopurine, Mesna, Mesnex, Methotrexate, Methotrexate Sodium, Methylprednisolone, Meticorten, Mitomycin, Mitomycin-C, Mitoxantrone, M-Prednisol, MTC, MTX, Mustargen, Mustine, Mutamycin, Myleran, Mylocel, Mylotarg, Navelbine, Nelarabine, Neosar, Neulasta, Neumega, Neupogen, Nexavar, Nilandron, Nilutamide, Nipent, Nitrogen Mustard, Novaldex, Novantrone, Octreotide, Octreotide acetate, Oncospar, Oncovin, Ontak, Onxal, Oprevelkin, Orapred, Orasone, Oxaliplatin, Paclitaxel, Paclitaxel Protein-bound, Pamidronate, Panitumumab, Panretin, Paraplatin, Pediapred, PEG Interferon, Pegaspargase, Pegfilgrastim, PEG-INTRON, PEG-L-asparaginase, PEMETREXED, Pentostatin, Phenylalanine Mustard, Platinol, Platinol-AQ, Prednisolone, Prednisone, Prelone, Procarbazine, PROCRIT, Proleukin, Prolifeprospan 20 with Carmustine Implant, Purinethol, Raloxifene, Revlimid, Rheumatrex, Rituxan, Rituximab, Roferon-A (Interferon Alfa-2a), Rubex, Rubidomycin hydrochloride, Sandostatin, Sandostatin LAR, Sargramostim, Solu-Cortef, Solu-Medrol, Sorafenib, SPRYCEL, STI-571, Streptozocin, SU11248, Sunitinib, Sutent, Tamoxifen, Tarceva, Targretin, Taxol, Taxotere, Temodar, Temozolomide, Temsirolimus, Teniposide, TESPA, Thalidomide, Thalomid, TheraCys, Thioguanine, Thioguanine Tabloid, Thiophosphoamide, Thioplex, Thiotepa, TICE, Toposar, Topotecan, Toremifene, Torisel, Tositumomab, Trastuzumab, Tretinoin, Trexall™, Trisenox, TSPA, TYKERB, VCR, Vectibix, Vectibix, Velban, Velcade, VePesid, Vesanoid, Viadur, Vidaza, Vinblastine, Vinblastine Sulfate, Vincasar Pfs, Vincristine, Vinorelbine, Vinorelbine tartrate, VLB, VM-26, Vorinostat, VP-16, Vumon, Xeloda, Zanosar, Zevalin, Zinecard, Zoladex, Zoledronic acid, Zolinza, Zometa.

### Benzopyrone Compounds

In some embodiments, the compound used in the treatment of cancer is a benzopyrone compound of formula I, or a pharmaceutically acceptable salt or prodrug thereof:

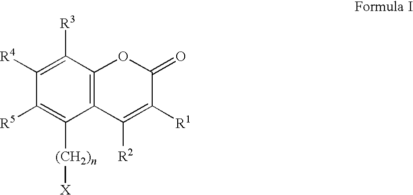

wherein n=0-10; R¹, R², R³, R⁴, R⁵ and X are independently selected from the group consisting of hydrogen, hydroxy, optionally substituted amine, amino, carboxyl, ester, nitroso, nitro, halogen, optionally substituted (C₁-C₆) alkyl, optionally substituted (C₁-C₆) alkoxy, optionally substituted (C₃-C₇) cycloalkyl, optionally substituted (C₃-C₇) heterocyclic, phenyl, and optionally substituted aryl; and wherein at least two of the R¹, R², R³, R⁴, and R⁵ substituents are always hydrogen.

In some embodiments, the benzopyrone compound is of formula II or its pharmaceutically acceptable salts or prodrugs:

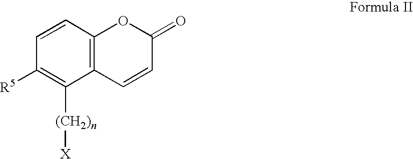

wherein R⁵ is selected from the group consisting of hydrogen, carboxyl, amino, nitroso, nitro, hydroxylamino, and hydroxy; and X is selected from the group consisting of halogen, hydroxy, optionally substituted (C₁-C₇) alkyl, optionally substituted (C₁-C₆) alkoxy, optionally substituted (C₃-C₇) cycloalkyl, optionally substituted (C₃-C₇) heterocyclic, phenyl, and optionally substituted aryl. In some embodiments, X is a halogen selected from the group consisting of F, Cl, Br and I. In some embodiments, X is iodine (I).

In some embodiments, the method comprises administering to a subject, preferably a human, in need thereof an effective amount of the compound of formula I or II. In some embodiments, X is I and R⁵ is nitro, nitroso, hydroxylamino, hydroxyl, or amino. In some embodiments, n is 0 and R⁵ is nitro. In some embodiments, n is 0 and R⁵ is amino. In other embodiments, n is 0, X is I, and R⁵ is nitro. In still other embodiments, n is 0, X is I, and R⁵ is amino. In some embodiments, the optionally substituted alkyl is substituted with a substituent selected from the group consisting of alkylamine, pyrrole, dihydropyrrole, and pyrrolidene. In some embodiments, the compound is of the formula IIIa, IIIb, IIIc, IIId, IIIe, IIIf, IIIg, IIIh, IIIk, IIIl, IIIm, IIIn, or one of their pharmaceutically acceptable salts or prodrugs:

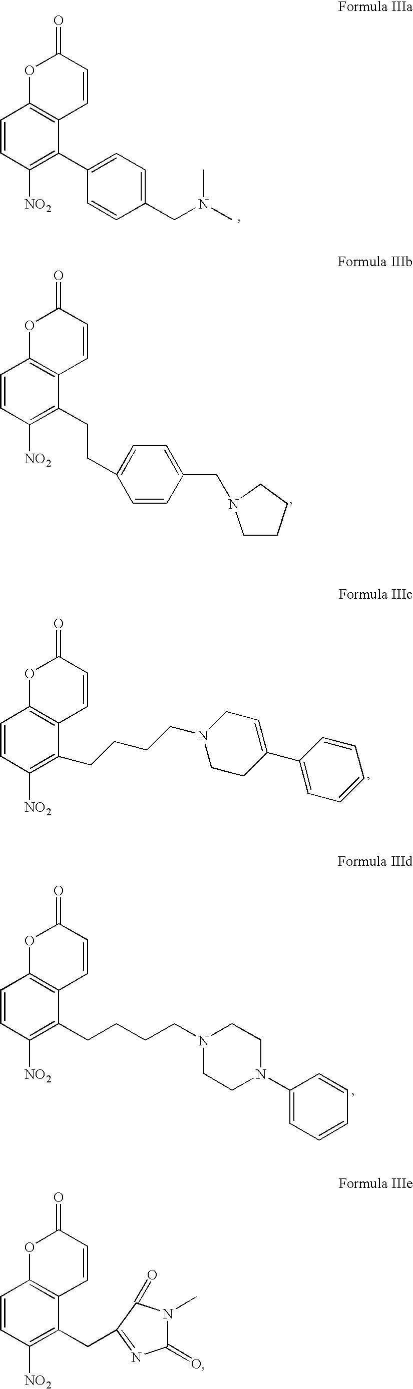

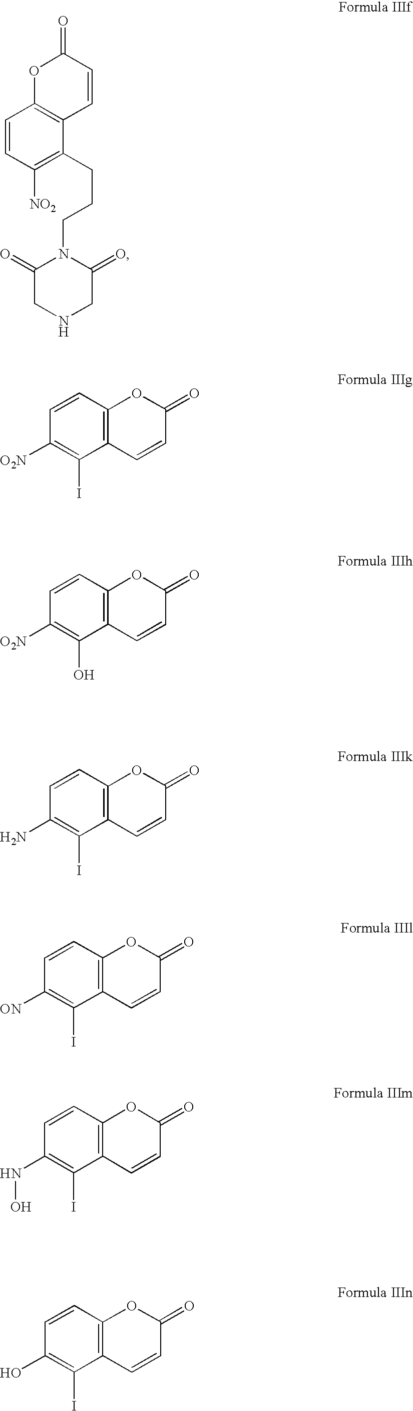

In some embodiments, the compound is of the formula IIIa or one of its pharmaceutically acceptable salts or prodrugs. In some embodiments, the compound is of the formula IIIb or one of its pharmaceutically acceptable salts or prodrugs. In some embodiments, the compound is of the formula IIIc or one of its pharmaceutically acceptable salts or prodrugs. In some embodiments, the compound is of the formula IIId or one of its pharmaceutically acceptable salts or prodrugs. In some embodiments, the compound is of the formula IIIe or one of its pharmaceutically acceptable salts or prodrugs. In some embodiments, the compound is of the formula IIIf or one of its pharmaceutically acceptable salts or prodrugs. In some embodiments, the compound is of the formula IIIg or one of its pharmaceutically acceptable salts or prodrugs. In some embodiments, the compound is of the formula IIIh or one of its pharmaceutically acceptable salts or prodrugs. In some embodiments, the compound is of the formula IIIk or one of its pharmaceutically acceptable salts or prodrugs. In some embodiments, the compound is of the formula IIIl or one of its pharmaceutically acceptable salts or prodrugs. In some embodiments, the compound is of the formula IIIm or one of its pharmaceutically acceptable salts or prodrugs. In some embodiments, the compound is of the formula IIIn or one of its pharmaceutically acceptable salts or prodrugs.

In some embodiments, the optionally substituted (C₃-C₇) heterocyclic is a five membered heterocyclic ring or a six membered heterocyclic ring. In some embodiments, the optionally substituted (C₃-C₇) heterocyclic contains at least one nitrogen. In some embodiments, the optionally substituted (C₃-C₇) heterocyclic is selected from the group consisting of azeridine, azetidine, pyrrole, dihydropyrrole, pyrrolidene, pyrazole, pyrazoline, pyrazolidine, imidazole, benzimidazole, triazole, tetrazole, oxazole, isoxazole, benzoxazole, oxadiazole, oxazoline, oxazolidine, thiazole, isothiazole, pyridine, dihydropyridine, tetrahydropyridine, quinazoline, pyrazine, pyrimidine, pyridazine, quinoline, isoquinoline, triazine, tetrazine, and piperazine. In some embodiments, the optionally substituted (C₃-C₇) heterocyclic is substituted with a substituent selected from the group consisting of optionally substituted (C₁-C₆) alkyl, optionally substituted (C₁-C₆) alkoxy, optionally substituted (C₃-C₇) cycloalkyl, optionally substituted (C₃-C₇) heterocyclic, and optionally substituted aryl.

In a preferred embodiment, the compositions disclosed herein relates to 5-iodo-6-nitro-benzopyrone (IIIg):

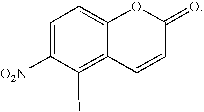

In another preferred embodiment, the compositions disclosed herein relates to 5-iodo-6-amino-benzopyrone (IIIk):

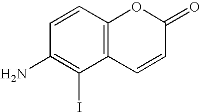

Typical salts are those of the inorganic ions, such as, for example, sodium, potassium, calcium, magnesium ions, and the like. Such salts include salts with inorganic or organic acids, such as hydrochloric acid, hydrobromic acid, phosphoric acid, nitric acid, sulfuric acid, methanesulfonic acid, p-toluenesulfonic acid, acetic acid, fumaric acid, succinic acid, lactic acid, mandelic acid, malic acid, citric acid, tartaric acid or maleic acid. In addition, if the compound(s) contain a carboxy group or other acidic group, it can be converted into a pharmaceutically acceptable addition salt with inorganic or organic bases. Examples of suitable bases include but are not limited to sodium hydroxide, potassium hydroxide, ammonia, cyclohexylamine, dicyclohexyl-amine, ethanolamine, diethanolamine, triethanolamine, and the like.

In some embodiments, the present invention provides a method of treating a cancer comprising administering to a subject in need thereof a therapeutically effective amount of a compound of formula (I) or (II), or a pharmaceutically acceptable salt or prodrug thereof. In some embodiments, the cancer includes, but is not limited to, adrenal cortical cancer, anal cancer, aplastic anemia, bile duct cancer, bladder cancer, bone cancer, bone metastasis, central nervous system (CNS) cancers, peripheral nervous system (PNS) cancers, breast cancer, Castleman's Disease, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, uterine cancer (e.g. uterine sarcoma), vaginal cancer, vulvar cancer, and Waldenstrom's macroglobulinemia.

In other embodiments, the cancer includes a cancer formed at a different site of a body as a result of migration of a cell from a cancer including but not limited to adrenal cortical cancer, anal cancer, aplastic anemia, bile duct cancer, bladder cancer, bone cancer, bone metastasis, central nervous system (CNS) cancers, peripheral nervous system (PNS) cancers, breast cancer, Castleman's Disease, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, uterine cancer (e.g. uterine sarcoma), vaginal cancer, vulvar cancer, and Waldenstrom's macroglobulinemia.

In some embodiments, the present invention also encompasses metabolites of a benzopyrone compound of formula (I). Metabolites are the intermediates and products of metabolism of a benzopyrone compound of formula (I). For example, 5-iodo-6-nitroso-1,2-benzopyrone is a metabolite of 5-iodo-6-amino-1,2-benzopyrones. Methods for identifying IIIg metabolites in whole blood samples are disclosed in Example 15. Any of the metabolites of benzopyrone compound of formula (I), in any form, is comtemplated by the present invention.

### Combination of Benzopyrone Compounds and Anti-Tumor Drugs

In some embodiments, the present invention provides a composition comprising a benzopyrone compound of formula (I) or (II) and an anti-tumor agent, or a pharmaceutically acceptable salt or prodrug thereof. In some embodiments, the anti-tumor agents include but are not limited to antitumor alkylating agents, antitumor antimetabolites, antitumor antibiotics, plant-derived antitumor agents, antitumor organoplatinum compounds, antitumor campthotecin derivatives, antitumor tyrosine kinase inhibitors, monoclonal antibodies, interferons, biological response modifiers, and other agents having antitumor activities, or a pharmaceutically acceptable salt thereof.

In some embodiments, the anti-tumor agent is an organoplatinum anti-cancer compound. In some embodiments, the anti-tumor agent is cisplatin, carboplatin or oxaliplatin. In some embodiments, the anti-tumor agent is oxaliplatin (OX). In some embodiments, the anti-tumor agent is gemcitabine (GEM). In some embodiments, the invention provides more than one anti-tumor agents. In some embodiments, the anti-tumor agents used in combination with a benzopyrone compound are OX and GEM. In some embodiments, the benzopyrone compound is of formula IIIg, i.e. 5-iodo-6-nitro-benzopyrone. In some embodiments, the benzopyrone compound is of formula IIIk, i.e. 5-iodo-6-amino-benzopyrone. In some embodiments, the composition comprises IIIg and OX. In some embodiments, the composition comprises IIIg and GEM. In some embodiments, the composition comprises IIIg, GEM and OX. In some embodiments, the composition comprises IIIk and OX. In some embodiments, the composition comprises IIIk and GEM. In some embodiments, the composition comprises IIIk, GEM and OX. In some embodiments, the combined effect of an anti-tumor agent and a benzopyrone compound is synergistic. In some embodiments, the combined effect of OX or GEM with IIIg (5-iodo-6-nitro-benzopyrone) is synergistic. In some embodiments, the combined effect of OX or GEM with IIIk (5-iodo-6-amino-benzopyrone) is synergistic.

In some embodiments, the present invention provides a method of treating a cancer comprising administering to a subject an effective amount of a composition comprising an anti-tumor agent and a compound of formula (I), or a pharmaceutically acceptable salt or prodrug thereof. In some embodiments, the cancer that may be treated using the method of the present invention includes, but is not limited to, adrenal cortical cancer, anal cancer, aplastic anemia, bile duct cancer, bladder cancer, bone cancer, bone metastasis, central nervous system (CNS) cancers, peripheral nervous system (PNS) cancers, breast cancer, Castleman's Disease, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, uterine cancer (e.g. uterine sarcoma), vaginal cancer, vulvar cancer, and Waldenstrom's macroglobulinemia.

### Mechanism of Benzopyrone Compounds

### Poly (ADP-Ribose) Polymerase (PARP) and PARP Inhibitors

Not intending to be limited by one mechanism of action, the compounds described herein are believed to have anti-cancer properties via the modulation of a poly (ADP-ribose) polymerase (PARP) enzyme. The drugs' mechanism of action is related to their ability to act as a ligand for the nuclear enzyme poly (ADP-ribose) polymerase (PARP-1). See Mendeleyev et al., supra, (1995). PARP-1 is expressed in the nucleus and catalyzes the conversion of β-nicotinamide adenine dinucleotide (NAD⁺) into nicotinamide and poly-ADP-ribose (PAR). PARP-1's role in homeostatic conditions seems to be limited to DNA transcription and repair. However, when cellular stress causes DNA damage, PARP-1 activity increases dramatically, which appears to be necessary for genomic integrity. Shall et al., Mutat Res. June 30; 460(1):1-15 (2000).

The poly (ADP-ribose) polymerase (PARP) is also known as poly (ADP-ribose) synthase and poly ADP-ribosyltransferase. PARP catalyzes the formation of poly (ADP-ribose) polymers which can attach to nuclear proteins (as well as to itself) and thereby modify the activities of those proteins. The enzyme plays a role in enhancing DNA repair, but more fundamentally there are indications that it plays a major role in regulating chromatin in the nuclei (for review see: D. D'Amours et al. “Poly (ADP-ribosylation reactions in the regulation of nuclear functions,” Biochem. J. 342: 249-268 (1999)).

More than 15 members of the PARP family of genes are present in the mammalian genome. PARP family proteins and poly(ADP-ribose) glycohydrolase (PARG), which degrades poly(ADP-ribose) to ADP-ribose, could be involved in a variety of cell regulatory functions including DNA damage response and transcriptional regulation and can be related to carcinogenesis and the biology of cancer in many respects.

Several PARP family proteins have been identified. Tankyrase has been found as an interacting protein of telomere regulatory factor 1 (TRF-1) and is involved in telomere regulation. Vault PARP (VPARP) is a component in the vault complex, which acts as a nuclear-cytoplasmic transporter. PARP-2, PARP-3 and 2,3,7,8-tetrachlorodibenzo-p-dioxin inducible PARP (TiPARP) have also been identified. Therefore, poly (ADP-ribose) metabolism could be related to a variety of cell regulatory functions.

The most studied member of this gene family is PARP-1. The PARP-1 gene product is expressed at high levels in the nuclei of cells and is dependent upon DNA damage for activation. Without being bound by any theory, it is believed that PARP-1 binds to DNA single or double stranded breaks through an amino terminal DNA binding domain. The binding activates the carboxy terminal catalytic domain and results in the formation of polymers of ADP-ribose on target molecules. PARP-1 is itself a target of poly ADP-ribosylation by virtue of a centrally located automodification domain. The ribosylation of PARP-1 causes dissociation of the PARP-1 molecules from the DNA. The entire process of binding, ribosylation, and dissociation occurs very rapidly. It has been suggested that this transient binding of PARP-1 to sites of DNA damage results in the recruitment of DNA repair machinery or can act to suppress the recombination long enough for the recruitment of repair machinery.

The source of ADP-ribose for the PARP reaction is nicotinamide adenosine dinucleotide (NAD). NAD is synthesized in cells from cellular ATP stores and thus high levels of activation of PARP activity can rapidly lead to depletion of cellular energy stores. It has been demonstrated that induction of PARP activity can lead to cell death that is correlated with depletion of cellular NAD and ATP pools. PARP activity is induced in many instances of oxidative stress or during inflammation. For example, during reperfusion of ischemic tissues reactive nitric oxide is generated and nitric oxide results in the generation of additional reactive oxygen species including hydrogen peroxide, peroxynitrate and hydroxyl radical. These latter species can directly damage DNA and the resulting damage induces activation of PARP activity. Frequently, it appears that sufficient activation of PARP activity occurs such that the cellular energy stores are depleted and the cell dies. A similar mechanism is believed to operate during inflammation when endothelial cells and pro-inflammatory cells synthesize nitric oxide which results in oxidative DNA damage in surrounding cells and the subsequent activation of PARP activity. The cell death that results from PARP activation is believed to be a major contributing factor in the extent of tissue damage that results from ischemia-reperfusion injury or from inflammation.

One of PARP-1's functions is to synthesize the biopolymer, poly (ADP-ribose). Both poly (ADP-ribose) and PARP-1 have been linked to the repair of DNA repair, apoptosis, the maintenance of genomic stability, and carcinogenesis. See Masutani et al., Genes, Chromosomes, and Cancer 38:339-348 (2003). PARP-1 plays a role in DNA repair, specifically base excision repair (BER). BER is a protection mechanism in mammalian cells for single-base DNA breakage. PARP-1 binds to the ends of DNA fragments through its zinc finger domains with great affinity and thereby acts as a DNA damage sensor. Gradwohl et al., Proc. Natl. Acad. Sci. USA 87:2990-2994 (1990); Murcia et al., Trends Biochem Sci 19: 172-176 (1994). A breakage in the DNA triggers a binding response by PARP-1 to the site of the break. PARP-1 then increases its catalytic activity several hundred fold (See Simonin et al., J Biol Chem 278: 13454-13461 (1993)) and begins to convert poly ADP-ribosylation of itself (Desmarais et al., Biochim Biophys Acta 1078: 179-186 (1991)) and BER proteins, such as DNA-PKcs and the molecular scaffold protein XRCC-1. See Ruscetti et al., J. Biol. Chem. June 5; 273(23):14461-14467 (1998) and Masson et al., Mol Cell Biol. June; 18(6):3563-71 (1998). BER proteins are rapidly recruited to the site of DNA damage. El-Kaminsy et al., Nucleic Acid Res. 31(19):5526-5533 (2003); Okano et al., Mol Cell Biol. 23(11):3974-3981 (2003). PARP-1's dissociates from the DNA breakage site but it remains in the vicinity of the DNA repair event.

Inhibition of PARP activity can be potentially useful in the treatment of cancer. De-inhibition of the DNAase (by PARP-1 inhibition) can initiate DNA breakdown that is specific for cancer cells and to only induce apoptosis in cancer cells. Small PARP molecule inhibitors can sensitize treated tumor cell lines to killing by ionizing radiation and by some DNA damaging chemotherapeutic drugs. A monotherapy by PARP inhibitors or a combination therapy of PARP inhibitors with an anti-tumor agent or radiation can be an effective treatment. Combination therapy with a chemotherapeutic can induce tumor regression at concentrations of the chemotherapeutic that are ineffective by themselves.

Inhibiting the activity of a PARP molecule includes reducing the activity of these molecules. The term “inhibits” and its grammatical conjugations, such as “inhibitory,” is not intended to require complete reduction in PARP activity. Such reduction is preferably by at least about 50%, at least about 75%, at least about 90%, and more preferably by at least about 95% of the activity of the molecule in the absence of the inhibitory effect, e.g., in the absence of an inhibitor, such as a benzopyrone compound of the invention. Most preferably, the term refers to an observable or measurable reduction in activity. In treatment scenarios, preferably the inhibition is sufficient to produce a therapeutic and/or prophylactic benefit in the condition being treated. The phrase “does not inhibit” and its grammatical conjugations does not require a complete lack of effect on the activity. For example, it refers to situations where there is less than about 20%, less than about 10%, and preferably less than about 5% of reduction in PARP activity in the presence of an inhibitor such as a benzopyrone compound of the invention.

The PARP inhibitors described herein can contain one or more asymmetric centers and thus occur as racemates and racemic mixtures, single enantiomers, individual diastereomers and diastereomeric mixtures. All such isomeric forms of these compounds are expressly included in the present invention. The PARP inhibitors described herein can also be represented in multiple tautomeric forms, all of which are included herein. The PARP inhibitors can also occur in cis- or trans- or E- or Z-double bond isomeric forms. All such isomeric forms of such inhibitors are expressly included in the present invention. All crystal forms of the PARP inhibitors described herein are expressly included in the present invention. The PARP inhibitors can also be present as their pharmaceutically acceptable salts, derivatives or prodrugs.

Other PARP inhibitors known in the art can also be used as known PARP inhibitors or candidate PARP inhibitors as disclosed in the present invention. The PARP inhibitors have been designed as analogs of benzamides, which bind competitively with the natural substrate NAD in the catalytic site of PARP. The PARP inhibitors include, but are not limited to, benzamides, quinolones and isoquinolones, benzopyrones, methyl 3,5-diiodo-4-(4′-methoxyphenoxy)benzoate, and 3,5-diiodo-4-(4′-methoxyphenoxy)acetophenone (U.S. Pat. No. 5,464,871, U.S. Pat. No. 5,670,518, U.S. Pat. No. 6,004,978, U.S. Pat. No. 6,169,104, U.S. Pat. No. 5,922,775, U.S. Pat. No. 6,017,958, U.S. Pat. No. 5,736,576, and U.S. Pat. No. 5,484,951, all incorporated herein in their entirety). The PARP inhibitors include a variety of cyclic benzamide analogs (i.e. lactams) which are potent inhibitors at the NAD site. Other PARP inhibitors include, but are not limited to, benzimidazoles and indoles (EP841924, EP1127052, U.S. Pat. No. 6,100,283, U.S. Pat. No. 6,310,082, US2002/156050, US2005/054631, WO05/012305, WO99/11628, and US2002/028815). Other PARP inhibitors known in the art can also be used as known PARP inhibitors or candidate PARP inhibitors as disclosed in the present invention (U.S. Application No. 60/804,563, filed on Jun. 12, 2006, incorporated herein by reference in its entirety).

### Synthesis PARP Inhibitors

The candidate PARP inhibitors as disclosed herein can be prepared by employing standard synthetic techniques known in the art and such techniques are within the scope of the present invention. Without limiting the scope of the present invention some of the synthesis schemes for the candidate PARP inhibitors are provided as below.

5-Iodo-6-nitro-benzopyrone (INBP or 5-iodo-6-nitrocoumarin) may be obtained as described in U.S. Pat. No. 5,484,951, which is incorporated herein by reference in its entirety. Alternatively, the INBP may be obtained according to the following reaction scheme:

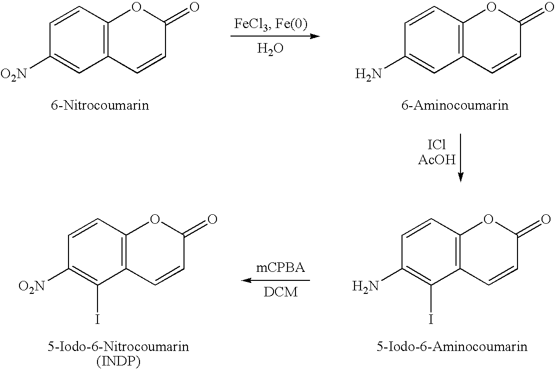

An example of a synthesis scheme for candidate PARP inhibitor of a compound of formula IIIa is as provided below. The (dimethylaminomethyl)phenol (CAS # 25338-55-0) is treated in step (i) with triflic anhydride (see D. Frantz et al., Org. Lett., 2002, 4, p. 4717-4718). Step (ii) forms a borate (H. Nakamura et al. J. Org. Chem. 1998, 63, p. 7529-7530) which reacts with iodonitrocoumarin to give compound of formula IIIa (W. Liu et al. Synthesis, 2006, p. 860-864).

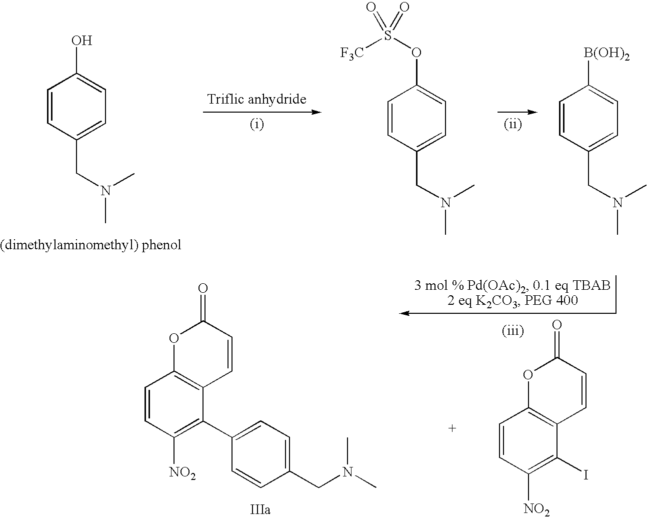

An alternative synthetic scheme for preparing a PARP inhibitor of formula IIIa comprises Suzuki coupling as shown in the following reaction scheme:

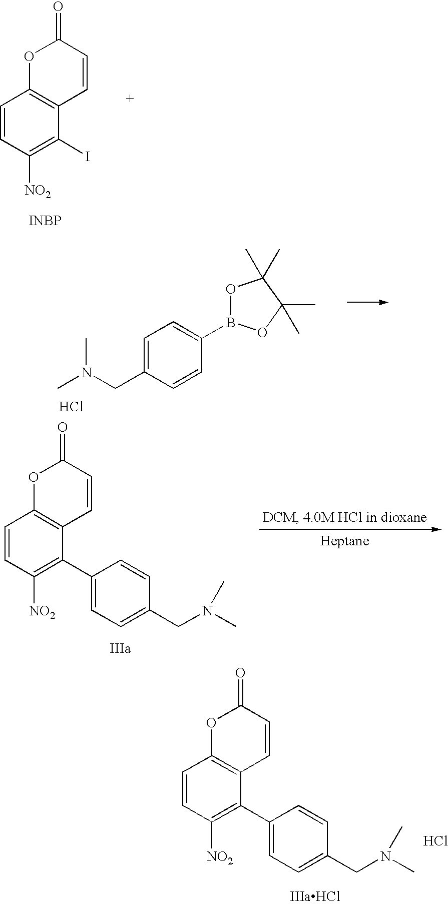

An example of a synthesis scheme for candidate PARP inhibitor of a compound of formula IIIb is as provided below (S. Huo, Org. Lett., 2003, 5, 423-425; T Baughman et al. Tetrahedron, 2004, 60, 10943-10948). Bromoethyl acetate (CAS # 927-68-4) is treated in step (i) with Zn dust to make its corresponding ZnBr, which is then treated with 1(4-iodobenzyl)pyrrolidine (CAS # 858676-60-5) in step (ii). In step (v) 5-iodo-6-nitrocoumarin is treated with a product of step (iv) to give a compound of formula IIIb.

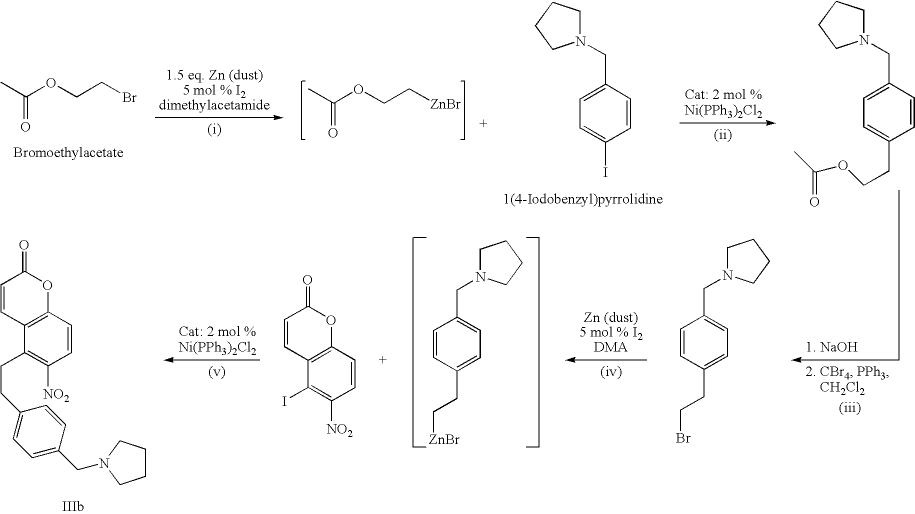

An alternative synthesis scheme for manufacturing IIIb is shown in the following scheme:

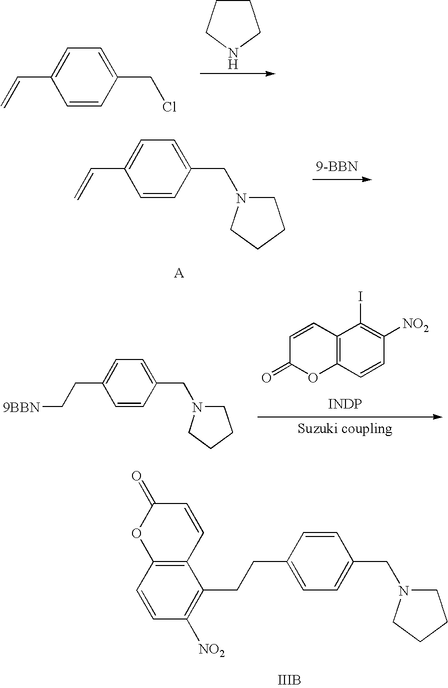

An example of a synthesis scheme for candidate PARP inhibitor of a compound of formula IIIc is as provided below (S. Huo, Org. Lett., 2003, 5, 423-425). 4-Phenyl-1,2,3,6-tetrahydropyridine (CAS # 43064-12-6) is treated with 1,4-dibromobutane (CAS # 110-52-1) in step (i). In step (iii) 5-iodo-6-nitrocoumarin is treated with a product of step (ii) to give a compound of formula IIIc.

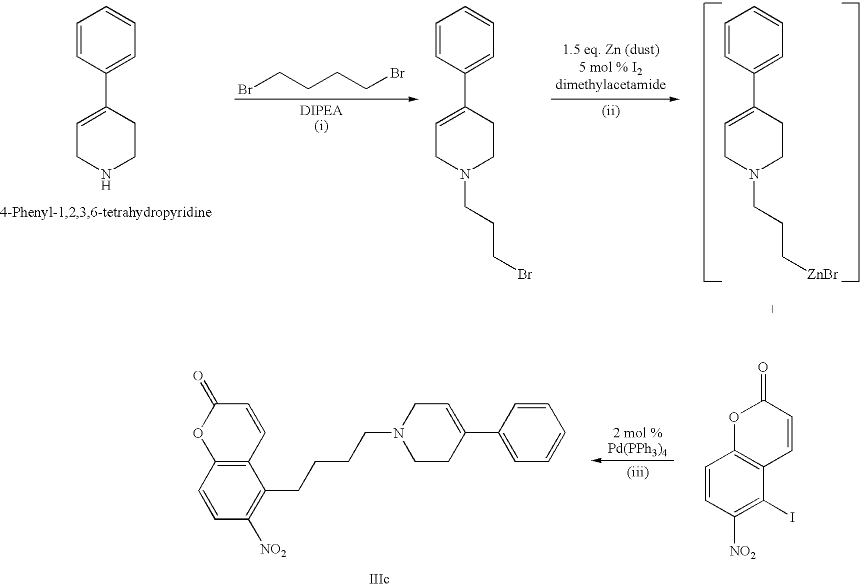

An alternative scheme for synthesizing IIIc is shown in the following scheme:

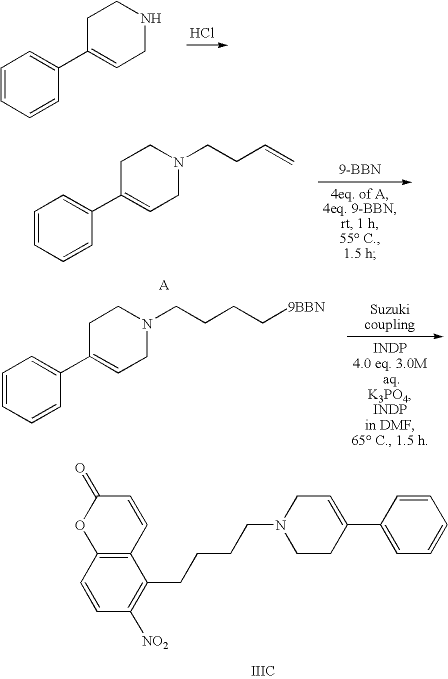

An example of a synthesis scheme for candidate PARP inhibitor of a compound of formula IIId is as provided below (S. Huo, Org. Lett., 2003, 5, 423-425). 1-Phenylpiperazine (CAS # 92-54-6) is treated with 1,4-dibrome butane (CAS # 110-52-1) in step (i). In step (iii) 5-iodo-6-nitrocoumarin is treated with a product of step (ii) to give a compound of formula IIId.

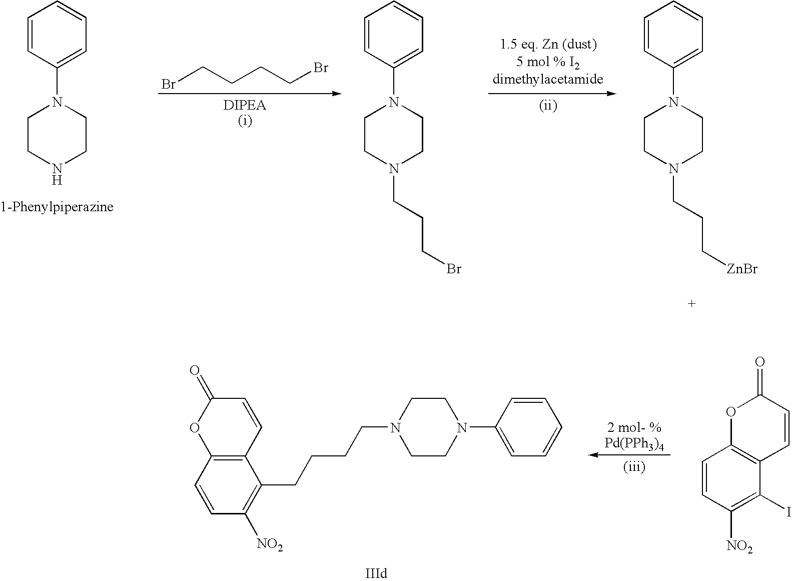

An alternative scheme for preparation of IIId is shown below:

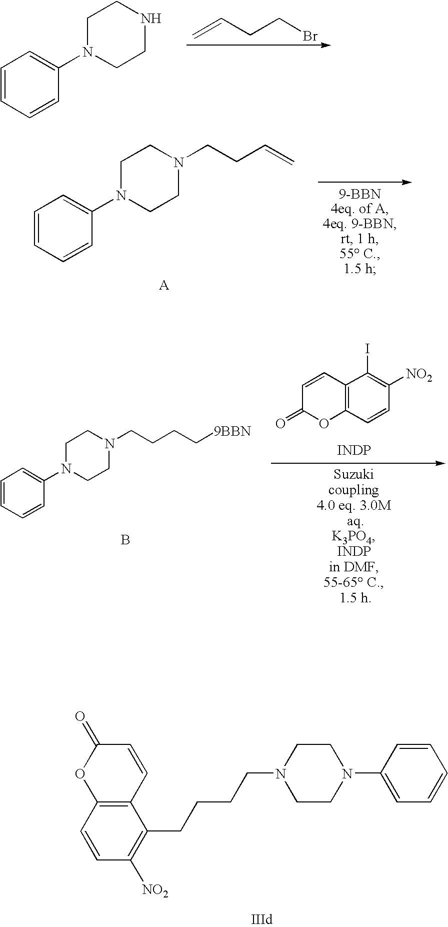

An example of a synthesis scheme for manufacturing IIIe is shown below:

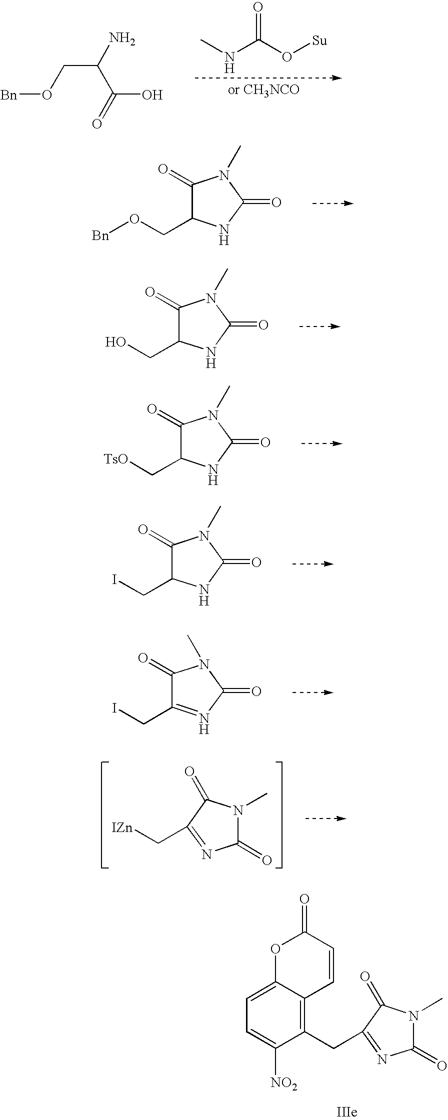

It is suspected, but unconfirmed, that IIIe may tautomerize to the enamine form as shown below. This could give rise to E/Z isomers.

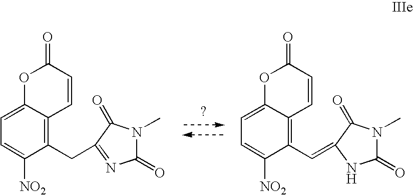

An example of a synthesis scheme for candidate PARP inhibitor of a compound of formula IIIf is as provided below (S. Huo, Org. Lett., 2003, 5, 423-425; T Baughman et al. Tetrahedron, 2004, 60, 10943-10948).

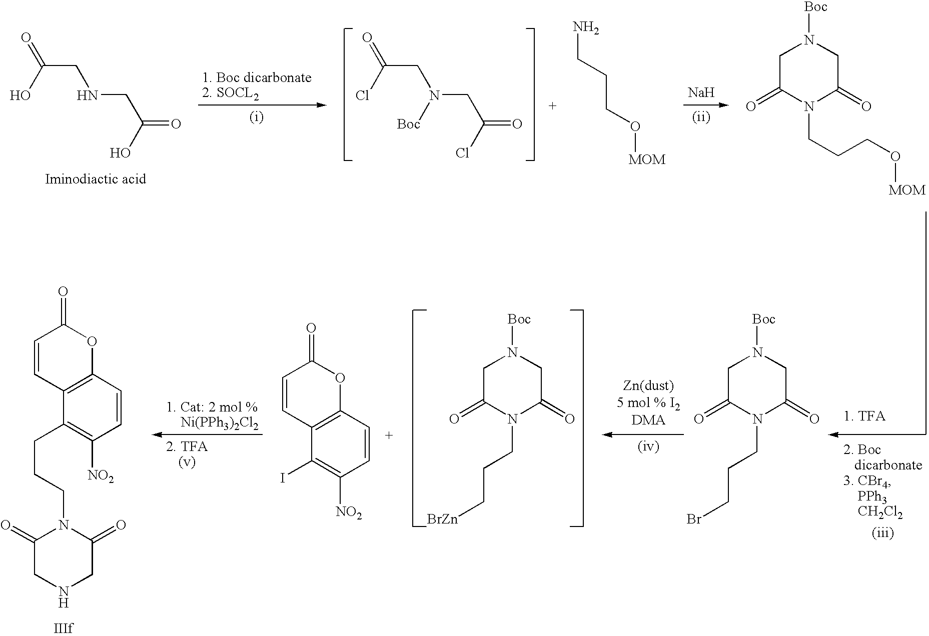

An alternative scheme for synthesis of IIIf is shown below:

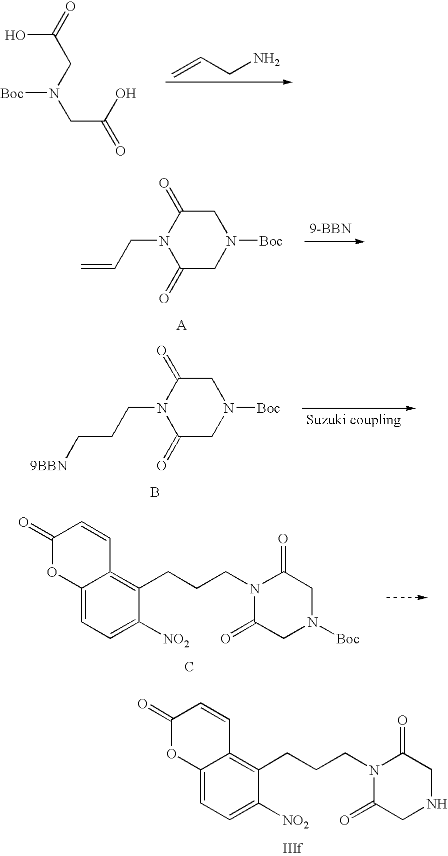

The compound of formula IIIh may be obtained from a compound of formula IIIg by electrophilic substitution of a hydroxyl moiety for the iodo on the benzo ring. In an alternative method, the compound of formula IIIh may be obtained as a metabolite from a sample collected from an animal or human after administration of the compound of formula IIIg to the animal or human. The compound of formula IIIh may be isolated from the biological sample (e.g. blood) by HPLC.

### Techniques for Analyzing PARP

The analysis of the PARP may include analysis of PARP gene expression, including an analysis of DNA, RNA, analysis of the level of PARP and/or analysis of the activity of PARP including a level of mono- and poly-ADP-ribozylation. Without limiting the scope of the present invention, any number of techniques known in the art can be employed for the analysis of PARP and they are all within the scope of the present invention. Some of the examples of such detection technique are given below but these examples are in no way limiting to the various detection techniques that can be used in the present invention.

Gene Expression Profiling: Methods of gene expression profiling include methods based on hybridization analysis of polynucleotides, polyribonucleotides methods based on sequencing of polynucleotides, polyribonucleotides and proteomics-based methods. The most commonly used methods known in the art for the quantification of mRNA expression in a sample include northern blotting and in situ hybridization (Parker & Barnes, Methods in Molecular Biology 106:247-283 (1999)); RNAse protection assays (Hod, Biotechniques 13:852-854 (1992)); and PCR-based methods, such as reverse transcription polymerase chain reaction (RT-PCR) (Weis et al., Trends in Genetics 8:263-264 (1992)). Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS), Comparative Genome Hybridisation (CGH), Chromatin Immunoprecipitation (ChIP), Single nucleotide polymorphism (SNP) and SNP arrays, Fluorescent in situ Hybridization (FISH), Protein binding arrays and DNA microarray (also commonly known as gene or genome chip, DNA chip, or gene array), RNAmicroarrays.

Reverse Transcriptase PCR (RT-PCR): One of the most sensitive and most flexible quantitative PCR-based gene expression profiling methods is RT-PCR, which can be used to compare mRNA levels in different sample populations, in normal and tumor tissues, with or without drug treatment, to characterize patterns of gene expression, to discriminate between closely related mRNAs, and to analyze RNA structure.

The first step is the isolation of mRNA from a target sample. For example, the starting material can be typically total RNA isolated from human tumors or tumor cell lines, and corresponding normal tissues or cell lines, respectively. Thus RNA can be isolated from a variety of normal and diseased cells and tissues, for example tumors, including breast, lung, colorectal, prostate, brain, liver, kidney, pancreas, spleen, thymus, testis, ovary, uterus, etc., or tumor cell lines. If the source of mRNA is a primary tumor, mRNA can be extracted, for example, from frozen or archived fixed tissues, for example paraffin-embedded and fixed (e.g. formalin-fixed) tissue samples. General methods for mRNA extraction are well known in the art and are disclosed in standard textbooks of molecular biology, including Ausubel et al., Current Protocols of Molecular Biology, John Wiley and Sons (1997).

In particular, RNA isolation can be performed using purification kit, buffer set and protease from commercial manufacturers, according to the manufacturer's instructions. RNA prepared from tumor can be isolated, for example, by cesium chloride density gradient centrifugation. As RNA cannot serve as a template for PCR, the first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. The derived cDNA can then be used as a template in the subsequent PCR reaction.

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and β-actin.

A more recent variation of the RT-PCR technique is the real time quantitative PCR, which measures PCR product accumulation through a dual-labeled fluorigenic probe. Real time PCR is compatible both with quantitative competitive PCR, where internal competitor for each target sequence is used for normalization, and with quantitative comparative PCR using a normalization gene contained within the sample, or a housekeeping gene for RT-PCR.

Fluorescence Microscopy: Some embodiments of the invention include fluorescence microscopy for analysis of PARP. Fluorescence microscopy enables the molecular composition of the structures being observed to be identified through the use of fluorescently-labeled probes of high chemical specificity such as antibodies. It can be done by directly conjugating a fluorophore to a protein and introducing this back into a cell. Fluorescent analogue may behave like the native protein and can therefore serve to reveal the distribution and behavior of this protein in the cell. Along with NMR, infrared spectroscopy, circular dichroism and other techniques, protein intrinsic fluorescence decay and its associated observation of fluorescence anisotropy, collisional quenching and resonance energy transfer are techniques for protein detection. The naturally fluorescent proteins can be used as fluorescent probes. The jellyfish aequorea victoria produces a naturally fluorescent protein known as green fluorescent protein (GFP). The fusion of these fluorescent probes to a target protein enables visualization by fluorescence microscopy and quantification by flow cytometry.

By way of example only, some of the probes are labels such as, fluorescein and its derivatives, carboxyfluoresceins, rhodamines and their derivatives, atto labels, fluorescent red and fluorescent orange: cy3/cy5 alternatives, lanthanide complexes with long lifetimes, long wavelength labels—up to 800 nm, DY cyanine labels, and phycobili proteins. By way of example only, some of the probes are conjugates such as, isothiocyanate conjugates, streptavidin conjugates, and biotin conjugates. By way of example only, some of the probes are enzyme substrates such as, fluorogenic and chromogenic substrates. By way of example only, some of the probes are fluorochromes such as, FITC (green fluorescence, excitation/emission=506/529 nm), rhodamine B (orange fluorescence, excitation/emission=560/584 nm), and nile blue A (red fluorescence, excitation/emission=636/686 nm). Fluorescent nanoparticles can be used for various types of immunoassays. Fluorescent nanoparticles are based on different materials, such as, polyacrylonitrile, and polystyrene etc. Fluorescent molecular rotors are sensors of microenvironmental restriction that become fluorescent when their rotation is constrained. Few examples of molecular constraint include increased dye (aggregation), binding to antibodies, or being trapped in the polymerization of actin. IEF (isoelectric focusing) is an analytical tool for the separation of ampholytes, mainly proteins. An advantage for IEF-gel electrophoresis with fluorescent IEF-marker is the possibility to directly observe the formation of gradient. Fluorescent IEF-marker can also be detected by UV-absorption at 280 nm (20° C.).

A peptide library can be synthesized on solid supports and, by using coloring receptors, subsequent dyed solid supports can be selected one by one. If receptors cannot indicate any color, their binding antibodies can be dyed. The method can not only be used on protein receptors, but also on screening binding ligands of synthesized artificial receptors and screening new metal binding ligands as well. Automated methods for HTS and FACS (fluorescence activated cell sorter) can also be used. A FACS machine originally runs cells through a capillary tube and separate cells by detecting their fluorescent intensities.

Immunoassays: Some embodiments of the invention include immunoassay for the analysis of PARP. In immunoblotting like the western blot of electrophoretically separated proteins a single protein can be identified by its antibody. Immunoassay can be competitive binding immunoassay where analyte competes with a labeled antigen for a limited pool of antibody molecules (e.g. radioimmunoassay, EMIT). Immunoassay can be non-competitive where antibody is present in excess and is labeled. As analyte antigen complex is increased, the amount of labeled antibody-antigen complex may also increase (e.g. ELISA). Antibodies can be polyclonal if produced by antigen injection into an experimental animal, or monoclonal if produced by cell fusion and cell culture techniques. In immunoassay, the antibody may serve as a specific reagent for the analyte antigen.

Without limiting the scope and content of the present invention, some of the types of immunoassays are, by way of example only, RIAs (radioimmunoassay), enzyme immunoassays like ELISA (enzyme-linked immunosorbent assay), EMIT (enzyme multiplied immunoassay technique), microparticle enzyme immunoassay (MEIA), LIA (luminescent immunoassay), and FIA (fluorescent immunoassay). These techniques can be used to detect biological substances in the nasal specimen. The antibodies—either used as primary or secondary ones—can be labeled with radioisotopes (e.g. 125I), fluorescent dyes (e.g. FITC) or enzymes (e.g. HRP or AP) which may catalyse fluorogenic or luminogenic reactions.

Biotin, or vitamin H is a co-enzyme which inherits a specific affinity towards avidin and streptavidin. This interaction makes biotinylated peptides a useful tool in various biotechnology assays for quality and quantity testing. To improve biotin/streptavidin recognition by minimizing steric hindrances, it can be necessary to enlarge the distance between biotin and the peptide itself. This can be achieved by coupling a spacer molecule (e.g., 6-nitrohexanoic acid) between biotin and the peptide.

The biotin quantitation assay for biotinylated proteins provides a sensitive fluorometric assay for accurately determining the number of biotin labels on a protein. Biotinylated peptides are widely used in a variety of biomedical screening systems requiring immobilization of at least one of the interaction partners onto streptavidin coated beads, membranes, glass slides or microtiter plates. The assay is based on the displacement of a ligand tagged with a quencher dye from the biotin binding sites of a reagent. To expose any biotin groups in a multiply labeled protein that are sterically restricted and inaccessible to the reagent, the protein can be treated with protease for digesting the protein.

EMIT is a competitive binding immunoassay that avoids the usual separation step. A type of immunoassay in which the protein is labeled with an enzyme, and the enzyme-protein-antibody complex is enzymatically inactive, allowing quantitation of unlabelled protein. Some embodiments of the invention include ELISA to analyze PARP. ELISA is based on selective antibodies attached to solid supports combined with enzyme reactions to produce systems capable of detecting low levels of proteins. It is also known as enzyme immunoassay or EIA. The protein is detected by antibodies that have been made against it, that is, for which it is the antigen. Monoclonal antibodies are often used.

The test may require the antibodies to be fixed to a solid surface, such as the inner surface of a test tube, and a preparation of the same antibodies coupled to an enzyme. The enzyme may be one (e.g., β-galactosidase) that produces a colored product from a colorless substrate. The test, for example, may be performed by filling the tube with the antigen solution (e.g., protein) to be assayed. Any antigen molecule present may bind to the immobilized antibody molecules. The antibody-enzyme conjugate may be added to the reaction mixture. The antibody part of the conjugate binds to any antigen molecules that are bound previously, creating an antibody-antigen-antibody “sandwich”. After washing away any unbound conjugate, the substrate solution may be added. After a set interval, the reaction is stopped (e.g., by adding 1 N NaOH) and the concentration of colored product formed is measured in a spectrophotometer. The intensity of color is proportional to the concentration of bound antigen.

ELISA can also be adapted to measure the concentration of antibodies, in which case, the wells are coated with the appropriate antigen. The solution (e.g., serum) containing antibody may be added. After it has had time to bind to the immobilized antigen, an enzyme-conjugated anti-immunoglobulin may be added, consisting of an antibody against the antibodies being tested for. After washing away unreacted reagent, the substrate may be added. The intensity of the color produced is proportional to the amount of enzyme-labeled antibodies bound (and thus to the concentration of the antibodies being assayed).

Some embodiments of the invention include radioimmunoassays to analyze PARP. Radioactive isotopes can be used to study in vivo metabolism, distribution, and binding of small amount of compounds. Radioactive isotopes of ¹H, ¹²C, ³¹P, ³²S, and ¹²⁷I in body are used such as ³H, ¹⁴C, ³²P, ³⁵S, and ¹²⁵I. In receptor fixation method in 96 well plates, receptors may be fixed in each well by using antibody or chemical methods and radioactive labeled ligands may be added to each well to induce binding. Unbound ligands may be washed out and then the standard can be determined by quantitative analysis of radioactivity of bound ligands or that of washed-out ligands. Then, addition of screening target compounds may induce competitive binding reaction with receptors. If the compounds show higher affinity to receptors than standard radioactive ligands, most of radioactive ligands would not bind to receptors and may be left in solution. Therefore, by analyzing quantity of bound radioactive ligands (or washed-out ligands), testing compounds' affinity to receptors can be indicated.

The filter membrane method may be needed when receptors cannot be fixed to 96 well plates or when ligand binding needs to be done in solution phase. In other words, after ligand-receptor binding reaction in solution, if the reaction solution is filtered through nitrocellulose filter paper, small molecules including ligands may go through it and only protein receptors may be left on the paper. Only ligands that strongly bound to receptors may stay on the filter paper and the relative affinity of added compounds can be identified by quantitative analysis of the standard radioactive ligands.

Some embodiments of the invention include fluorescence immunoassays for the analysis of PARP. Fluorescence based immunological methods are based upon the competitive binding of labeled ligands versus unlabeled ones on highly specific receptor sites. The fluorescence technique can be used for immunoassays based on changes in fluorescence lifetime with changing analyte concentration. This technique may work with short lifetime dyes like fluorescein isothiocyanate (FITC) (the donor) whose fluorescence may be quenched by energy transfer to eosin (the acceptor). A number of photoluminescent compounds may be used, such as cyanines, oxazines, thiazines, porphyrins, phthalocyanines, fluorescent infrared-emitting polynuclear aromatic hydrocarbons, phycobiliproteins, squaraines and organo-metallic complexes, hydrocarbons and azo dyes.

Fluorescence based immunological methods can be, for example, heterogenous or homogenous. Heterogenous immunoassays comprise physical separation of bound from free labeled analyte. The analyte or antibody may be attached to a solid surface. The technique can be competitive (for a higher selectivity) or noncompetitive (for a higher sensitivity). Detection can be direct (only one type of antibody used) or indirect (a second type of antibody is used). Homogenous immunoassays comprise no physical separation. Double-antibody fluorophore-labeled antigen participates in an equilibrium reaction with antibodies directed against both the antigen and the fluorophore. Labeled and unlabeled antigen may compete for a limited number of anti-antigen antibodies.

Some of the fluorescence immunoassay methods include simple fluorescence labeling method, fluorescence resonance energy transfer (FRET), time resolved fluorescence (TRF), and scanning probe microscopy (SPM). The simple fluorescence labeling method can be used for receptor-ligand binding, enzymatic activity by using pertinent fluorescence, and as a fluorescent indicator of various in vivo physiological changes such as pH, ion concentration, and electric pressure. TRF is a method that selectively measures fluorescence of the lanthanide series after the emission of other fluorescent molecules is finished. TRF can be used with FRET and the lanthanide series can become donors or acceptors. In scanning probe microscopy, in the capture phase, for example, at least one monoclonal antibody is adhered to a solid phase and a scanning probe microscope is utilized to detect antigen/antibody complexes which may be present on the surface of the solid phase. The use of scanning tunneling microscopy eliminates the need for labels which normally is utilized in many immunoassay systems to detect antigen/antibody complexes.

Protein identification methods: By way of example only, protein identification methods include low-throughput sequencing through Edman degradation, mass spectrometry techniques, peptide mass fingerprinting, de novo sequencing, and antibody-based assays. The protein quantification assays include fluorescent dye gel staining, tagging or chemical modification methods (i.e. isotope-coded affinity tags (ICATS), combined fractional diagonal chromatography (COFRADIC)). The purified protein may also be used for determination of three-dimensional crystal structure, which can be used for modeling intermolecular interactions. Common methods for determining three-dimensional crystal structure include x-ray crystallography and NMR spectroscopy. Characteristics indicative of the three-dimensional structure of proteins can be probed with mass spectrometry. By using chemical crosslinking to couple parts of the protein that are close in space, but far apart in sequence, information about the overall structure can be inferred. By following the exchange of amide protons with deuterium from the solvent, it is possible to probe the solvent accessibility of various parts of the protein.

In one embodiment, fluorescence-activated cell-sorting (FACS) is used to identify PARP expressing cells. FACS is a specialised type of flow cytometry. It provides a method for sorting a heterogenous mixture of biological cells into two or more containers, one cell at a time, based upon the specific light scattering and fluorescent characteristics of each cell. It provides quantitative recording of fluorescent signals from individual cells as well as physical separation of cells of particular interest. In yet another embodiment, microfluidic based devices are used to evaluate PARP expression.

Mass spectrometry can also be used to characterize PARP from patient samples. The two methods for ionization of whole proteins are electrospray ionization (ESI) and matrix-assisted laser desorption/ionization (MALDI). In the first, intact proteins are ionized by either of the two techniques described above, and then introduced to a mass analyser. In the second, proteins are enzymatically digested into smaller peptides using an agent such as trypsin or pepsin. Other proteolytic digest agents are also used. The collection of peptide products are then introduced to the mass analyser. This is often referred to as the “bottom-up” approach of protein analysis.

Whole protein mass analysis is conducted using either time-of-flight (TOF) MS, or Fourier transform ion cyclotron resonance (FT-ICR). The instrument used for peptide mass analysis is the quadrupole ion trap. Multiple stage quadrupole-time-of-flight and MALDI time-of-flight instruments also find use in this application.

Two methods used to fractionate proteins, or their peptide products from an enzymatic digestion. The first method fractionates whole proteins and is called two-dimensional gel electrophoresis. The second method, high performance liquid chromatography is used to fractionate peptides after enzymatic digestion. In some situations, it may be necessary to combine both of these techniques.

There are two ways mass spectroscopy can be used to identify proteins. Peptide mass uses the masses of proteolytic peptides as input to a search of a database of predicted masses that would arise from digestion of a list of known proteins. If a protein sequence in the reference list gives rise to a significant number of predicted masses that match the experimental values, there is some evidence that this protein is present in the original sample.

Tandem MS is also a method for identifying proteins. Collision-induced dissociation is used in mainstream applications to generate a set of fragments from a specific peptide ion. The fragmentation process primarily gives rise to cleavage products that break along peptide bonds.

A number of different algorithmic approaches have been described to identify peptides and proteins from tandem mass spectrometry (MS/MS), peptide de novo sequencing and sequence tag based searching. One option that combines a comprehensive range of data analysis features is PEAKS. Other existing mass spec analysis software include: Peptide fragment fingerprinting SEQUEST, Mascot, OMSSA and X!Tandem).

Proteins can also be quantified by mass spectrometry. Typically, stable (e.g. non-radioactive) heavier isotopes of carbon (C13) or nitrogen (N15) are incorporated into one sample while the other one is labelled with corresponding light isotopes (e.g. C12 and N14). The two samples are mixed before the analysis. Peptides derived from the different samples can be distinguished due to their mass difference. The ratio of their peak intensities corresponds to the relative abundance ratio of the peptides (and proteins). The methods for isotope labelling are SILAC (stable isotope labelling with amino acids in cell culture), trypsin-catalyzed 018 labeling, ICAT (isotope coded affinity tagging), ITRAQ (isotope tags for relative and absolute quantitation). “Semi-quantitative” mass spectrometry can be performed without labeling of samples. Typically, this is done with MALDI analysis (in linear mode). The peak intensity, or the peak area, from individual molecules (typically proteins) is here correlated to the amount of protein in the sample. However, the individual signal depends on the primary structure of the protein, on the complexity of the sample, and on the settings of the instrument.

N-terminal sequencing aids in the identification of unknown proteins, confirm recombinant protein identity and fidelity (reading frame, translation start point, etc.), aid the interpretation of NMR and crystallographic data, demonstrate degrees of identity between proteins, or provide data for the design of synthetic peptides for antibody generation, etc. N-terminal sequencing utilises the Edman degradative chemistry, sequentially removing amino acid residues from the N-terminus of the protein and identifying them by reverse-phase HPLC. Sensitivity can be at the level of 100s femtomoles and long sequence reads (20-40 residues) can often be obtained from a few 10 s picomoles of starting material. Pure proteins (>90%) can generate easily interpreted data, but insufficiently purified protein mixtures may also provide useful data, subject to rigorous data interpretation. N-terminally modified (especially acetylated) proteins cannot be sequenced directly, as the absence of a free primary amino-group prevents the Edman chemistry. However, limited proteolysis of the blocked protein (e.g. using cyanogen bromide) may allow a mixture of amino acids to be generated in each cycle of the instrument, which can be subjected to database analysis in order to interpret meaningful sequence information. C-terminal sequencing is a post-translational modification, affecting the structure and activity of a protein. Various disease situations can be associated with impaired protein processing and C-terminal sequencing provides an additional tool for the investigation of protein structure and processing mechanisms.

### Techniques for Measurement of PARP Inhibiting Activity of PARP Inhibitors

In some embodiments, a PARP inhibiting activity of the candidate PARP inhibitor is evaluated to characterize the ability of a candidate PARP inhibitor to bind to a PARP protein, and/or characterize the ability of the candidate PARP inhibitor to modify the activity of a PARP protein. There are various techniques known in the art to analyze PARP activity. Such techniques include without limitation, mass spectrometry, high performance liquid chromatography etc. In some embodiments, the technique used for evaluation is an assay technique. Both in vitro and in vivo assays can be used in accordance with the methods of the invention depending on the identity of the PARP protein being investigated. Appropriate activity or functional assays can be readily determined by the skilled artisan based on the disclosure herein. The candidate PARP inhibitors described herein can be used in assays, including radiolabeled, antibody detection and fluorometric assays, for the isolation, identification, or structural or functional characterization of the PARP protein.

The assay can be an enzyme inhibition assay utilizing a full length or truncated PARP protein. The PARP protein can be contacted with the candidate PARP inhibitor and a measurement of the binding affinity of the candidate PARP inhibitor against a standard is determined. Such assays are known to one of ordinary skill in the art and are within the scope of the present invention. The assay for evaluating PARP inhibiting activity of the candidate PARP inhibitor can be a cell-based assay. The candidate PARP inhibitor is contacted with a cell and a measurement of an inhibition of a standard marker produced in the cell is determined. Cells can be either isolated from an animal, including a transformed cultured cell, or can be in a living animal. Such assays are also known to one of ordinary skill in the art and are within the scope of the present invention.

An example of an assay for measuring PARP activity can proceed as follows. PARP-1 is purified from calf thymus as reported earlier (Molinet et al. (1993) EMBO J. 12:2109-2117). Alternatively recombinant PARP-1 is isolated from Sodoptera Fugiperda (Sf9) cells infected with recombinant baculovirus, expressing the human PARP-1 gene, constructed according to the instructions of Pharmingen. The cDNA of the amino acid exchange mutant R34G and R138 il of PARP-1 is created by the mega primer method (Kannann et al. (1989) Nucl Acids Res 17:5404). The mutated gene is cloned into the transfer vector pV 1392 and the recombinant virus is generated by the Baculogold technology of Pharmigen. The mutated proteins are expressed in Sf9 cells, purified and assayed as reported (Huang et al. (2004) Biochemistry 43:217-223; Kirsten et al. (2004) Methods in Molecular Biology 287, Epigenetics Protocols 137-149). Assays can be carried out as described in Kun et al. (2004) Biochemistry, 43:210-216.

The candidate PARP inhibitors of the present invention can be identified using, for example, immunoassays such as enzyme linked immunoabsorbent assays (ELISA) and radioimmunoassays (RIA) or binding assays such as Biacore assays. Binding assays can employ kinetic or thermodynamic methodology using a wide variety of techniques including, but not limited to, microcalorimetry, circular dichroism, capillary zone electrophoresis, nuclear magnetic resonance spectroscopy, fluorescence spectroscopy, and combinations thereof. Without limiting the scope of the present invention, some of the examples of the techniques for measurement of the bioactivity of the PARP inhibitors, are provided below.

Fluorescence Microscopy: Some embodiments of the invention include fluorescence microscopy for measuring the PARP inhibiting activity of the candidate PARP inhibitors of the present invention. Fluorescence microscopy enables the molecular composition of the structures being observed to be identified through the use of fluorescently-labeled probes of high chemical specificity such as antibodies. It can be done by directly conjugating a fluorophore to a PARP protein and introducing this back into a cell. Fluorescent analogue can behave like the native protein and can therefore serve to reveal the distribution and behavior of this PARP protein in the cell. Along with NMR, infrared spectroscopy, circular dichroism and other techniques, protein intrinsic fluorescence decay and its associated observation of fluorescence anisotropy, collisional quenching and resonance energy transfer are techniques for PARP detection. The naturally fluorescent proteins can be used as fluorescent probes. The jellyfish aequorea victoria produces a naturally fluorescent protein known as green fluorescent protein (GFP). The fusion of these fluorescent probes to a target protein enables visualization by fluorescence microscopy and quantification by flow cytometry.

By way of example only, some of the probes are labels such as, fluorescein and its derivatives, carboxyfluoresceins, rhodamines and their derivatives, atto labels, fluorescent red and fluorescent orange: cy3/cy5 alternatives, lanthanide complexes with long lifetimes, long wavelength labels—up to 800 nm, DY cyanine labels, and phycobili proteins. By way of example only, some of the probes are conjugates such as, isothiocyanate conjugates, streptavidin conjugates, and biotin conjugates. By way of example only, some of the probes are enzyme substrates such as, fluorogenic and chromogenic substrates. By way of example only, some of the probes are fluorochromes such as, FITC (green fluorescence, excitation/emission=506/529 nm), rhodamine B (orange fluorescence, excitation/emission=560/584 nm), and Nile blue A (red fluorescence, excitation/emission=636/686 nm). Fluorescent nanoparticles can be used for various types of immunoassays. Fluorescent nanoparticles are based on different materials, such as, polyacrylonitrile, and polystyrene etc. Fluorescent molecular rotors are sensors of microenvironmental restriction that become fluorescent when their rotation is constrained. Few examples of molecular constraint include increased dye (aggregation), binding to antibodies, or being trapped in the polymerization of actin. IEF (isoelectric focusing) is an analytical tool for the separation of ampholytes, mainly proteins. An advantage for IEF-gel electrophoresis with fluorescent IEF-marker is the possibility to directly observe the formation of gradient. Fluorescent IEF-marker can also be detected by UV-absorption at 280 nm (20° C.).

A peptide library can be synthesized on solid supports and, by using coloring receptors, subsequent dyed solid supports can be selected one by one. If receptors cannot indicate any color, their binding antibodies can be dyed. The method can not only be used on protein receptors, but also on screening binding ligands of synthesized artificial receptors and screening new metal binding ligands as well. Automated methods for HTS and FACS (fluorescence activated cell sorter) can also be used.

Immunoassays: Some embodiments of the invention include immunoassay for measuring the PARP inhibiting activity of the candidate PARP inhibitors of the present invention. In immunoblotting like the western blot of electrophoretically separated proteins a single protein can be identified by its antibody. Immunoassay can be competitive binding immunoassay where analyte competes with a labeled antigen for a limited pool of antibody molecules (e.g. radioimmunoassay, EMIT). Immunoassay can be non-competitive where antibody is present in excess and is labeled. As analyte antigen complex is increased, the amount of labeled antibody-antigen complex can also increase (e.g. ELISA). Antibodies can be polyclonal if produced by antigen injection into an experimental animal, or monoclonal if produced by cell fusion and cell culture techniques. In immunoassay, the antibody can serve as a specific reagent for the analyte antigen.

Without limiting the scope and content of the present invention, some of the types of immunoassays are, but not limited to, RIAs (radioimmunoassay), enzyme immunoassays like ELISA (enzyme-linked immunosorbent assay), EMIT (enzyme multiplied immunoassay technique), microparticle enzyme immunoassay (MEIA), LIA (luminescent immunoassay), and FIA (fluorescent immunoassay). The antibodies—either used as primary or secondary ones—can be labeled with radioisotopes (e.g. 125I), fluorescent dyes (e.g. FITC) or enzymes (e.g. HRP or AP) which can catalyze fluorogenic or luminogenic reactions.

Biotin, or vitamin H is a co-enzyme which inherits a specific affinity towards avidin and streptavidin. This interaction makes biotinylated peptides a useful tool in various biotechnology assays for quality and quantity testing. To improve biotin/streptavidin recognition by minimizing steric hindrances, it can be necessary to enlarge the distance between biotin and the peptide itself This can be achieved by coupling a spacer molecule (e.g., 6-aminohexanoic acid) between biotin and the peptide.

The biotin quantitation assay for biotinylated proteins provides a sensitive fluorometric assay for accurately determining the number of biotin labels on a protein. Biotinylated peptides are widely used in a variety of biomedical screening systems requiring immobilization of at least one of the interaction partners onto streptavidin coated beads, membranes, glass slides or microtiter plates. The assay is based on the displacement of a ligand tagged with a quencher dye from the biotin binding sites of a reagent. To expose any biotin groups in a multiply labeled protein that are sterically restricted and inaccessible to the reagent, the protein can be treated with protease for digesting the protein.

EMIT is a competitive binding immunoassay that avoids the usual separation step. A type of immunoassay in which the protein is labeled with an enzyme, and the enzyme-protein-antibody complex is enzymatically inactive, allowing quantitation of unlabelled protein. Some embodiments of the invention include ELISA to analyze PARP. ELISA is based on selective antibodies attached to solid supports combined with enzyme reactions to produce systems capable of detecting low levels of proteins. It is also known as enzyme immunoassay or EIA. The protein is detected by antibodies that have been made against it, that is, for which it is the antigen. Monoclonal antibodies are often used.

The test can require the antibodies to be fixed to a solid surface, such as the inner surface of a test tube, and a preparation of the same antibodies coupled to an enzyme. The enzyme can be one (e.g., β-galactosidase) that produces a colored product from a colorless substrate. The test, for example, can be performed by filling the tube with the antigen solution (e.g., protein) to be assayed. Any antigen molecule present can bind to the immobilized antibody molecules. The antibody-enzyme conjugate can be added to the reaction mixture. The antibody part of the conjugate binds to any antigen molecules that are bound previously, creating an antibody-antigen-antibody “sandwich”. After washing away any unbound conjugate, the substrate solution can be added. After a set interval, the reaction is stopped (e.g., by adding 1 N NaOH) and the concentration of colored product formed is measured in a spectrophotometer. The intensity of color is proportional to the concentration of bound antigen.

ELISA can also be adapted to measure the concentration of antibodies, in which case, the wells are coated with the appropriate antigen. The solution (e.g., serum) containing antibody can be added. After it has had time to bind to the immobilized antigen, an enzyme-conjugated anti-immunoglobulin can be added, consisting of an antibody against the antibodies being tested for. After washing away unreacted reagent, the substrate can be added. The intensity of the color produced is proportional to the amount of enzyme-labeled antibodies bound (and thus to the concentration of the antibodies being assayed).

Some embodiments of the invention include radioimmunoassays for measuring the PARP inhibiting activity of the candidate PARP inhibitors of the present invention. Radioactive isotopes can be used to study in vivo metabolism, distribution, and binding of small amount of compounds. Radioactive isotopes of ¹H, ¹²C, ³¹P, ³²S, and ¹²⁷I in body are used such as ³H, ¹⁴C, ³²P, ³⁵S, and ¹²⁵I. In receptor fixation method in 96 well plates, receptors can be fixed in each well by using antibody or chemical methods and radioactive labeled ligands can be added to each well to induce binding. Unbound ligands can be washed out and then the standard can be determined by quantitative analysis of radioactivity of bound ligands or that of washed-out ligands. Then, addition of screening target compounds can induce competitive binding reaction with receptors. If the compounds show higher affinity to receptors than standard radioactive ligands, most of radioactive ligands would not bind to receptors and can be left in solution. Therefore, by analyzing quantity of bound radioactive ligands (or washed-out ligands), testing compounds' affinity to receptors can be indicated.

The filter membrane method can be needed when receptors cannot be fixed to 96 well plates or when ligand binding needs to be done in solution phase. In other words, after ligand-receptor binding reaction in solution, if the reaction solution is filtered through nitrocellulose filter paper, small molecules including ligands can go through it and only protein receptors can be left on the paper. Only ligands that strongly bound to receptors can stay on the filter paper and the relative affinity of added compounds can be identified by quantitative analysis of the standard radioactive ligands.

Some embodiments of the invention include fluorescence immunoassays for measuring the PARP inhibiting activity of the candidate PARP inhibitors of the present invention. Fluorescence based immunological methods are based upon the competitive binding of labeled ligands versus unlabeled ones on highly specific receptor sites. The fluorescence technique can be used for immunoassays based on changes in fluorescence lifetime with changing analyte concentration. This technique can work with short lifetime dyes like fluorescein isothiocyanate (FITC) (the donor) whose fluorescence can be quenched by energy transfer to eosin (the acceptor). A number of photoluminescent compounds can be used, such as cyanines, oxazines, thiazines, porphyrins, phthalocyanines, fluorescent infrared-emitting polynuclear aromatic hydrocarbons, phycobiliproteins, squaraines and organo-metallic complexes, hydrocarbons and azo dyes.

Fluorescence based immunological methods can be, for example, heterogeneous or homogenous. Heterogeneous immunoassays comprise physical separation of bound from free labeled analyte. The analyte or antibody can be attached to a solid surface. Homogenous immunoassays comprise no physical separation. Double-antibody fluorophore-labeled antigen participates in an equilibrium reaction with antibodies directed against both the antigen and the fluorophore. Labeled and unlabeled antigen can compete for a limited number of anti-antigen antibodies.

Some of the fluorescence immunoassay methods include simple fluorescence labeling method, fluorescence resonance energy transfer (FRET), time resolved fluorescence (TRF), and scanning probe microscopy (SPM). The simple fluorescence labeling method can be used for receptor-ligand binding, enzymatic activity by using pertinent fluorescence, and as a fluorescent indicator of various in vivo physiological changes such as pH, ion concentration, and electric pressure.

Method of Treatment with Benzopyrone Compounds

In one aspect, the present invention relates to methods of treating cancer comprising administering to a subject in need thereof an effective amount of a PARP inhibitor, for example, a benzopyrone compound. The candidate PARP inhibitors comprising compounds of formula I-III where III includes IIIa, IIIb, IIIc, IIId, IIIe, IIIf, IIIg, IIIh, IIIk, IIIl, IIIm, and IIIn, for treatment of cancer.

In some embodiments, the invention provides a method of treating cancer by administering an effective amount of one or more benzopyrone compounds alone to a subject. Examples of cancers that may be treated include but not limited to acute lymphobalstic leukemia, adult acute lymphoblastic leukemia, childhood acute myeloid leukemia, adult acute myeloid leukemia, adrenocortical carcinoma, childhood adrenocortical carcinoma, AIDS-related cancers, AIDS-related lymphoma, anal cancer, appendix cancer, childhood cerebellar astrocytoma, childhood cerebral astrocytoma, basal cell carcinoma, extrahepatic bladder cancer, child hood bladder Cancer, osteosarcoma i.e. bone cancer, malignant fibrous histiocytoma, childhood brain stem glioma, brain tumor—cerebellar astrocytoma, brain tumor—cerebral astrocytoma/malignant glioma-childhood; brain tumor—ependymoma, brain tumor—medulloblastoma, brain tumor—supratentorial primitive neuroectodermal tumors, brain tumor—visual pathway and hypothalamic glioma, brain tumor—other, breast cancer, bronchial adenoma.carcinoids, Burkitt lymphoma, carcinoid tumor—childhood, carcinoid tumor—gastrointestinal, carcinoma of unknown primary, central nervous system lymphoma-primary, cervical cancer, childhood cancers, chronic lymphocytic leukemia; chronic myelogenous leukemia, chronic myeloproliferative disorders, colon cancer, colorectal cancer, cutaneous T-cell lymphoma; desmoplastic small round cell tumor, endometrial cancer, ependymoma, esophageal cancer, Ewing's sarcoma (in the Ewing family of tumors), extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor—extracranial, germ cell tumor—extragonadal, germ cell tumor-ovarian, gestational trophoblastic tumor, adult glioma, childhood brain stem glioma, childhood cerebral astrocytoma glioma, childhood visual pathway and hypothalamic glioma, gastric carcinoid; hairy cell leukemia, head and neck cancer, hepatocellular (liver) cancer, adult (Primary), hepatocellular (liver) cancer-childhood (primary), Hodgkin lymphoma-adult and childhood, Hodgkin lymphoma during pregnancy, hypopharyngeal cancer, hypothalamic and visual pathway glioma-childhood, intraocular melanoma, islet cell carcinoma (endocrine pancreas), Kaposi sarcoma, kidney (renal cell) cancer, childhood kidney cancer, laryngeal cancer, Leukemia, acute lymphoblastic, adult; Leukemia, acute lymphoblastic, childhood; Leukemia, acute myeloid, adult; Leukemia, acute myeloid, childhood; Leukemia, chronic lymphocytic; Leukemia, chronic myelogenous; Leukemia, hairy cell; lip and oral cavity cancer; liver cancer, adult (primary); liver cancer-childhood (primary); lung cancer, non-small cell; lung cancer—small cell; lymphoma, AIDS-related; Lymphoma, Burkitt; Lymphoma, cutaneous T-Cell; lymphoma, Hodgkin, adult; Lymphoma, hodgkin, childhood; Lymphoma, non-Hodgkin, adult; Lymphoma, Non-hodgkin, childhood; Lymphoma, non-Hodgkin during pregnancy; Lymphoma, Primary Central Nervous System; macroglobulinemia-Waldenstrφm, malignant fibrous histiocytoma of Bone/osteosarcoma; childhood medulloblastoma, melanoma, melanoma-intraocular (eye), Merkel cell carcinoma, adult malignant mesothelioma, childhood mesothelioma, metastatic squamous neck cancer with occult primary, mouth cancer, multiple endocrine neoplasia syndrome, multiple myeloma/pasma cell neoplasm, mycosis fungoides, myelodysplastic syndromes, myelodysplastic/myeloproliferative diseases, chronic myelogenous leukemia, adult acute myeloid leukemia, childhood acute myeloid leukemia, multiple myeloma (cancer of the bone-marrow), chronic myeloproliferative disorders, nasal cavity and paranasal sinus cancer, nasopharyngeal carcinoma, childhood nasopharyngeal cancer, neuroblastoma, non-Hodgkin lymphoma, adult; non-Hodgkin lymphoma, childhood; non-Hodgkin lymphoma during pregnancy, non-small cell lung cancer, oral cancer-childhood, oral cavity cancer-lip and oropharyngeal cancer; osteosarcoma/malignant fibrous histiocytoma of bone, childhood ovarian cancer, ovarian epithelial cancer, ovarian germ cell tumor, ovarian low malignant potential tumor, pancreatic cancer, childhood pancreatic cancer, islet cell pancreatic cancer, peripheral nervous system (PNS) cancers, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, pregnancy and breast cancer, pregnancy and Hodgkin lymphoma, pregnancy and Non-Hodgkin lymphoma, primary central nervous system lymphoma, prostate cancer, rectal cancer, renal cell (kidney) cancer, childhood renal Cell (kidney) cancer, renal pelvis and ureter-transitional cell cancer, retinoblastoma, childhood rhabdomyosarcoma, salivary gland cancer, childhood salivary gland cancer, sarcoma-Ewing family of tumors, Kaposi sarcoma, adult soft tissue sarcoma, childhood soft tissue sarcoma, uterine sarcoma, Sezary syndrome, skin cancer (nonmelanoma), childhood skin cancer, skin cancer (melanoma), Merkel cell skin carcinoma, small cell lung cancer, small intestine cancer, squamous cell carcinoma (nonmelanoma), metastatic squamous neck cancer with occult primary, stomach (gastric) cancer, childhood stomach (gastric) cancer, childhood supratentorial primitive neuroectodermal tumors, cutaneous T-cell lymphoma, testicular cancer, throat cancer, childhood thymoma, thymoma and thymic carcinoma, thyroid cancer, childhood thyroid cancer, transitional cell cancer of the renal pelvis and ureter, gestational trophoblastic tumor, unknown primary site carcinoma of adult, unknown primary site cancer of childhood, urethral cancer, endometrial uterine cancer, uterine sarcoma, vaginal cancer, childhood visual pathway and hypothalamic glioma, vulvar cancer, Waldenstrom macroglobulinemia, Wilms tumor, women's cancers.

In some preferred embodiments, the cancer is pancreatic cancer. In some embodiments, the method includes co-administering along with one of the compounds of formula I-III (e.g. one of formulae IIIa-IIIh, especially IIIg or IIIh or IIIk, and most especially IIIg) an anticancer drug. In some embodiments, the method includes co-administering one of the compounds of formulae I, II or III (e.g. one of formulae IIIa-IIIh, especially IIIg or IIIh or IIIk, and most especially IIIg) along with an anti-tumor agent. In some embodiments, the chemotherapeutic drug is cisplatin, carboplatin or oxaliplatin or a combination of two or more thereof. In some embodiments, the chemotherapeutic drug is oxaliplatin. In some embodiments, the chemotherapeutic drug is gemcitabine. In some embodiments, the chemotherapeutic drugs are oxaliplatin and gemcitabine.

In some embodiments, the present invention provides a method of treating cancer by administration of the benzopyrone compounds in combination with one or more anti-tumor agents, such as OX and/or GEM. Such combination can be used to treat cancers including, but not limited to, acute lymphobalstic leukemia, adult acute lymphoblastic leukemia, childhood acute myeloid leukemia, adult acute myeloid leukemia, adrenocortical carcinoma, childhood adrenocortical carcinoma, AIDS-related cancers, AIDS-related lymphoma, anal cancer, appendix cancer, childhood cerebellar astrocytoma, childhood cerebral astrocytoma, basal cell carcinoma, extrahepatic bladder cancer, child hood bladder Cancer, osteosarcoma i.e. bone cancer, malignant fibrous histiocytoma, childhood brain stem glioma, brain tumor—cerebellar astrocytoma, brain Tumor—cerebral astrocytoma/malignant glioma-childhood; brain tumor—ependymoma, brain tumor—medulloblastoma, brain tumor—supratentorial primitive neuroectodermal tumors, brain tumor-visual pathway and hypothalamic glioma, brain tumor—other, breast cancer, bronchial adenoma.carcinoids, Burkitt lymphoma, carcinoid tumor-childhood, carcinoid tumor-gastrointestinal, carcinoma of unknown primary, central nervous system lymphoma-primary, cervical cancer, childhood cancers, chronic lymphocytic leukemia; chronic myelogenous leukemia, chronic myeloproliferative disorders, colon cancer, colorectal cancer, cutaneous T-cell lymphoma; desmoplastic small round cell tumor, endometrial cancer, ependymoma, esophageal cancer, Ewing's sarcoma (in the Ewing family of tumors), extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor—extracranial, germ cell tumor-extragonadal, germ cell tumor-ovarian, gestational trophoblastic tumor, adult glioma, childhood brain stem glioma, childhood cerebral astrocytoma glioma, childhood visual pathway and hypothalamic glioma, gastric carcinoid; hairy cell leukemia, head and neck cancer, hepatocellular (liver) cancer, adult (Primary), hepatocellular (liver) cancer-childhood (primary), Hodgkin lymphoma-adult and childhood, Hodgkin lymphoma during pregnancy, hypopharyngeal cancer, hypothalamic and visual pathway glioma-childhood, intraocular melanoma, islet cell carcinoma (endocrine pancreas), Kaposi's sarcoma, kidney (renal cell) cancer, childhood kidney cancer, laryngeal cancer, Leukemia, acute lymphoblastic, adult; Leukemia, acute lymphoblastic, childhood; Leukemia, acute myeloid, adult; Leukemia, acute myeloid, childhood; Leukemia, chronic lymphocytic; Leukemia, chronic myelogenous; Leukemia, hairy cell; lip and oral cavity cancer; liver cancer, adult (primary); liver cancer-childhood (primary); lung cancer, non-small cell; lung cancer—small cell; lymphoma, AIDS-related; Lymphoma, Burkitt; Lymphoma, cutaneous T-Cell; lymphoma, Hodgkin, adult; Lymphoma, Hodgkin, childhood; Lymphoma, non-Hodgkin, adult; Lymphoma, Non-hodgkin, childhood; Lymphoma, non-Hodgkin during pregnancy; Lymphoma, Primary Central Nervous System; macroglobulinemia-Waldenstrom, malignant fibrous histiocytoma of Bone/osteosarcoma; childhood medulloblastoma, melanoma, melanoma-intraocular (eye), Merkel cell carcinoma, adult malignant mesothelioma, childhood mesothelioma, metastatic squamous neck cancer with occult primary, mouth cancer, multiple endocrine neoplasia syndrome, multiple myeloma/plasma cell neoplasm, mycosis fungoides, myelodysplastic syndromes, myelodysplastic/myeloproliferative diseases, chronic myelogenous leukemia, adult acute myeloid leukemia, childhood acute myeloid leukemia, multiple myeloma (cancer of the bone-marrow), chronic myeloproliferative disorders, nasal cavity and paranasal sinus cancer, nasopharyngeal carcinoma, childhood nasopharyngeal cancer, neuroblastoma, non-Hodgkin lymphoma, adult; non-Hodgkin lymphoma, childhood; non-Hodgkin lymphoma during pregnancy, non-small cell lung cancer, oral cancer-childhood, oral cavity cancer-lip and oropharyngeal cancer; osteosarcoma/malignant fibrous histiocytoma of bone, childhood ovarian cancer, ovarian epithelial cancer, ovarian germ cell tumor, ovarian low malignant potential tumor, pancreatic cancer, childhood pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, pregnancy and breast cancer, pregnancy and Hodgkin lymphoma, pregnancy and Non-Hodgkin lymphoma, primary central nervous system lymphoma, prostate cancer, rectal cancer, renal cell (kidney) cancer, childhood renal Cell (kidney) cancer, renal pelvis and ureter-transitional cell cancer, retinoblastoma, childhood rhabdomyosarcoma, salivary gland cancer, childhood salivary gland cancer, sarcoma-Ewing family of tumors, Kaposi sarcoma, adult soft tissue sarcoma, childhood soft tissue sarcoma, uterine sarcoma, Sézary syndrome, skin cancer (nonmelanoma), childhood skin cancer, skin cancer (melanoma), Merkel cell skin carcinoma, small cell lung cancer, small intestine cancer, squamous cell carcinoma (nonmelanoma), metastatic squamous neck cancer with occult primary, stomach (gastric) cancer, childhood stomach (gastric) cancer, childhood supratentorial primitive neuroectodermal tumors, cutaneous T-cell lymphoma, testicular cancer, throat cancer, childhood thymoma, thymoma and thymic carcinoma, thyroid cancer, childhood thyroid cancer, transitional cell cancer of the renal pelvis and ureter, gestational trophoblastic tumor, unknown primary site carcinoma of adult, unknown primary site cancer of childhood, urethral cancer, endometrial uterine cancer, uterine sarcoma, vaginal cancer, childhood visual Pathway and hypothalamic glioma, vulvar cancer, Waldenstrom macroglobulinemia, and Wilms tumor.

In some embodiments, the methods of the present invention also comprise the administration of candidate PARP inhibitors, e.g. benzopyrone compounds of formulae (I)-(III) in combination with other therapies. The choice of therapy that can be co-administered with the compositions of the invention will depend, in part, on the condition being treated. For example, for treating cancer, compound of some embodiments of the invention can be used in combination with radiation therapy, monoclonal antibody therapy, chemotherapy, bone marrow transplantation, or a combination thereof.

In other embodiments, the candidate PARP inhibitors in the present invention can be used to treat cancer, and to radiosensitize and/or chemosensitize tumor cells. The candidate PARP inhibitors of the present invention can be “anti-cancer agents,” which term also encompasses “anti-tumor cell growth agents” and “anti-neoplastic agents.” Radiosensitizers are known to increase the sensitivity of cancerous cells to the toxic effects of electromagnetic radiation. Many cancer treatment protocols currently employ radiosensitizers activated by the electromagnetic radiation of x-rays. Examples of x-ray activated radiosensitizers include, but are not limited to, the following: metronidazole, misonidazole, desmethylmisonidazole, pimonidazole, etanidazole, nimorazole, mitomycin C, RSU 1069, SR 4233, E09, RB 6145, nicotinamide, 5-bromodeoxyuridine (BUdR), 5-iododeoxyuridine (IUdR), bromodeoxycytidine, fluorodeoxyuridine (FudR), hydroxyurea, cisplatin, and therapeutically effective analogs and derivatives of the same.

Photodynamic therapy (PDT) of cancers employs visible light as the radiation activator of the sensitizing agent. Examples of photodynamic radiosensitizers include the following, but are not limited to: hematoporphyrin derivatives, photofrin, benzoporphyrin derivatives, NPe6, tin etioporphyrin SnET2, pheoborbide-α, bacteriochlorophyll-α, naphthalocyanines, phthalocyanines, zinc phthalocyanine, and therapeutically effective analogs and derivatives of the same.

Chemosensitizers are also known to increase the sensitivity of cancerous cells to the toxic effects of chemotherapeutic compounds. Exemplary anti-tumor agents that can be used in conjunction with PARP inhibitors include, but are not limited to, adriamycin, camptothecin, dacarbazine, carboplatin, cisplatin, daunorubicin, docetaxel, doxorubicin, interferon (alpha, beta, gamma), interleukin 2, innotecan, paclitaxel, streptozotocin, temozolomide, topotecan, and therapeutically effective analogs and derivatives of the same. In addition, other therapeutic agents which can be used in conjunction with a PARP inhibitors include, but are not limited to, 5-fluorouracil, leucovorin, 5′-amino-5′-deoxythymidine, oxygen, carbogen, red cell transfusions, perfluorocarbons (e.g., Fluosol-DA), 2,3-DPG, BW12C, calcium channel blockers, pentoxyfylline, antiangiogenesis compounds, hydralazine, and L-BSO.

The methods of treatment as disclosed herein can be via oral administration, transmucosal administration, buccal administration, nasal administration, inhalation, parental administration, intravenous, subcutaneous, intramuscular, sublingual, transdermal administration, and rectal administration.

### Examples of Cancer

### Her-2 Related Cancer

Her-2 disease is a type of breast cancer. Characterized by aggressive growth and a poor prognosis, it can be caused by the presence of excessive numbers of a gene called HER2 (human epidermal growth factor receptor-2) in tumor cells. Therapies that can used in combination with the PARP inhibitors as disclosed herein include, but are no limited to Her-2 antibodies such as herceptin, anti-hormones (e.g., selective oestrogen receptor modulator (SERM) tamoxifen), chemotherapy and radiotherapy, aromatase inhibitors (e.g. anastrazole, letrozole and exemestane) and anti-estrogens (e.g., fulvestrant (Faslodex)).

### Breast Cancer

In some embodiments, the invention provides a method of treating breast cancer comprising administering an effective amount of a combination of a benzopyrone compound with one or more anti-tumor agents.

Several types of breast cancer exist that may be treated by the methods provided by the invention. A lobular carcinoma in situ and a ductal carcinoma in situ are breast cancers that have developed in the lobules and ducts, respectively, but have not spread to the fatty tissue surrounding the breast or to other areas of the body. An infiltrating (or invasive) lobular and a ductal carcinoma are cancers that have developed in the lobules and ducts, respectively, and have spread to either the breast's fatty tissue and/or other parts of the body. Other cancers of the breast that would benefit from treatment by the methods provided by the invention are medullary carcinomas, colloid carcinomas, tubular carcinomas, and inflammatory breast cancer.

In some embodiments, the invention provides for treatment of so-called “triple negative” breast cancer. There are several subclasses of breast cancer identified by classic biomarkers such as estrogen receptor (ER) and/or progesterone receptor (PR) positive tumors, HER2-amplified tumors, and ER/PR/HER2-negative tumors. These three subtypes have been reproducibly identified by gene expression profiling in multiple breast cancer and exhibit basal-like subtype expression profiles and poor prognosis. Triple negative breast cancer is characterized by ER/PR/HER2-negative tumors.

Treatments available for breast cancer patients are surgery, immunotherapy, radiation therapy, chemotherapy, endocrine therapy, or a combination thereof. A lumpectomy and a mastectomy are two possible surgical procedures available for breast cancer patients.

Breast cancer is generally treated with a combination of surgery to remove the cancerous lesion and adjuvant therapy—radiation, chemotherapy or both—to attack any cancer cells that may be left after the surgery. Breast cancer can be classified broadly by the presence or absence of hormone receptors (HRs). Hormone receptor positive (HR+) cancer is characterized by the expression of one or both female hormone receptors—estrogen receptor (ER) or progesterone receptor (PR). Adjuvant therapy for ER+ breast cancer often includes chemotherapy with a selective estrogen receptor modulator (SERM), such as tamoxifen or raloxifene. Unfortunately, while about 70% of breast cancers are ER positive, the remaining 30% of breast cancers that are HR negative are not amenable to treatment with SERMs. Accordingly, other adjuvant chemotherapies, such as treatment with an anthracycline (alone or in combination with a taxane) have been tried on ER negative breast cancer. In particular, so-called triple negative metastatic breast cancer (i.e. breast cancer that is ER negative, PR negative and human epidermal growth factor receptor 2 (HER2) negative) is refractory to standard treatments and is entirely refractory to SERM chemotherapy.

Chemotherapy utilizes anti-tumor agents to prevent cancer cells from multiplying, invading, metastasizing and killing a patient. Several drugs are available to treat breast cancer, including cytotoxic drugs such as doxorubicin, cyclophosphamide, methotrexate, paclitaxel, thiotepa, mitoxantrone, vincristine, or combinations thereof. Endocrine therapy may be an effective treatment where the remaining breast tissue retains endocrine sensitivity. Agents administered for this therapy include tamoxifen, megestrol acetate, aminoglutethimide, fluoxymesterone, leuprolide, goserelin, and prednisone.

The methods provided by the invention can provide a beneficial effect for breast cancer patients, by administration of a benzopyrone compound or a combination of administration of a benzopyrone compound and surgery, radiation therapy, chemotherapy, or endocrine therapy.

### Ovarian Cancer

In another aspect, the invention provides a method of treating ovarian cancer, including epithelial ovarian tumors. In some embodiments, the method comprises administering benzopyrone compounds alone into a subject. In other embodiments, the method comprises administering benzopyrone compounds in combination with one or more anti-tumor agents as listed herein into a subject. Preferably, the invention provides a method of treating an ovarian cancer selected from the following: an adenocarcinoma in the ovary and an adenocarcinoma that has migrated from the ovary into the abdominal cavity. Surgery, immunotherapy, chemotherapy, hormone therapy, radiation therapy, or a combination thereof are some possible treatments available for ovarian cancer. Some possible surgical procedures include debulking, and a unilateral or bilateral oophorectomy and/or a unilateral or bilateral salpigectomy.

Anti-cancer drugs that may be used include cyclophosphamide, etoposide, altretamine, and ifosfamide. Hormone therapy with the drug tamoxifen may be used to shrink ovarian tumors. Radiation therapy may be external beam radiation therapy and/or brachytherapy.

The methods provided by the invention can provide a beneficial effect for ovarian cancer patients, by administration of a benzopyrone compound or a combination of administration of a benzopyrone compound and surgery, radiation therapy, chemotherapy endocrine therapy, or a combination thereof.

### Cervical Cancer

In another aspect, the invention provides a method of treating cervical cancer, preferably an adenocarcinoma in the cervix epithelial. In some embodiments, the method comprises administering benzopyrone compounds alone into a subject. In other embodiments, the method comprises administering benzopyrone compounds in combination with one or more anti-tumor agents as listed herein into a subject.

Two main types of this cancer exist: squamous cell carcinoma and adenocarcinomas. The former constitutes about 80-90% of all cervical cancers and develops where the ectocervix (portion closest to the vagina) and the endocervix (portion closest to the uterus) join. The latter develop in the mucous-producing gland cells of the endocervix. Some cervical cancers have characteristics of both of these and are called adenosquamous carcinomas or mixed carcinomas.

The chief treatments available for cervical cancer are surgery, immunotherapy, radiation therapy and chemotherapy. Some possible surgical options are cryosurgery, a hysterectomy, and a radical hysterectomy. Radiation therapy for cervical cancer patients includes external beam radiation therapy or brachytherapy. Anti-cancer drugs that may be administered as part of chemotherapy to treat cervical cancer include cisplatin, carboplatin, hydroxyurea, irinotecan, bleomycin, vincrinstine, mitomycin, ifosfamide, fluorouracil, etoposide, methotrexate, and combinations thereof.

The methods provided by the invention can provide a beneficial effect for cervical cancer patients, by administration of a benzopyrone compound or a combination of administration of a benzopyrone compound and surgery, radiation therapy, chemotherapy, or a combination thereof.

### Prostate Cancer

In one other aspect, the invention provides methods to treat prostate cancer, preferably a prostate cancer selected from the following: an adenocarcinoma or an adenocarinoma that has migrated to the bone. In some embodiments, the method comprises administering benzopyrone compounds alone into a subject. In other embodiments, the method comprises administering benzopyrone compounds in combination with one or more anti-tumor agents as listed herein into a subject.

Prostate cancer develops in the prostate organ in men, which surrounds the first part of the urethra. The prostate has several cell types but 99% of tumors are adenocarcinomas that develop in the glandular cells responsible for generating seminal fluid.

Surgery, immunotherapy, radiation therapy, cryosurgery, hormone therapy, and chemotherapy are some treatments available for prostate cancer patients. Possible surgical procedures to treat prostate cancer include radical retropubic prostatectomy, a radical perineal prostatectomy, and a laparscopic radical prostatectomy. Some radiation therapy options are external beam radiation, including three dimensional conformal radiation therapy, intensity modulated radiation therapy, and conformal proton beam radiation therapy. Brachytherapy (seed implantation or interstitial radiation therapy) is also an available method of treatment for prostate cancer. Cryosurgery is another possible method used to treat localized prostate cancer cells.

Hormone therapy, also called androgen deprivation therapy or androgen suppression therapy, may be used to treat prostate cancer. Several methods of this therapy are available including an orchiectomy in which the testicles, where 90% of androgens are produced, are removed. Another method is the administration of luteinizing hormone-releaseing hormone (LHRH) analogs to lower androgen levels. The LHRH analogs available include leuprolide, goserelin, triptorelin, and histrelin. An LHRH antagonist may also be administered, such as abarelix.

Treatment with an antiandrogen agent, which blocks androgen activity in the body, is another available therapy. Such agents include flutamide, bicalutamide, and nilutamide. This therapy is typically combined with LHRH analog administration or an orchiectomy, which is termed a combined androgen blockade (CAB).

Chemotherapy may be appropriate where a prostate tumor has spread outside the prostate gland and hormone treatment is not effective. Anti-cancer drugs such as doxorubicin, estramustine, etoposide, mitoxantrone, vinblastine, paclitaxel, docetaxel, carboplatin, and prednisone may be administered to slow the growth of prostate cancer, reduce symptoms and improve the quality of life.

The methods provided by the invention can provide a beneficial effect for prostate cancer patients, by administration of a benzopyrone compound or a combination of administration of a benzopyrone compound and surgery, radiation therapy, chemotherapy, hormone therapy, or a combination thereof.

### Pancreatic Cancer

In another aspect, the invention provides methods of treating pancreatic cancer, preferably a pancreatic cancer selected from the following: an epitheliod carcinoma in the pancreatic duct tissue and an adenocarcinoma in a pancreatic duct. In some embodiments, the method comprises administering benzopyrone compounds alone into a subject. In other embodiments, the method comprises administering benzopyrone compounds in combination with one or more anti-tumor agents as listed herein into a subject.

Pancreatic cancer is the fourth-leading cause of cancer mortality among adults in the United States. One of the most promising drugs in pancreatic cancer therapy is oxaliplatin, an organoplatinum molecule, that forms inter- and intrastrand DNA adducts/cross-links and induces a high proportion of DNA single strand breaks. However, the gemcitabine and oxaliplatin combination has failed to demonstrate a statistically significant advantage compared with single-agent gemcitabine. Development of novel agents and drug combinations are urgently needed. PARP-1 functions as a DNA damage sensor for both single- and double-stranded DNA breaks and plays a key role in many cellular processes including the regulation of DNA repair. PARP-1 also acts as a promoter-specific transcriptional coactivator of NF-κB, a transcription factor constitutively activated in most pancreatic cancer tissues and human pancreatic cancer cell lines. Some embodiments of the present invention describe the antitumor activity of the PARP-1 inhibitor IIIg alone and in combination with oxaliplatin in pancreatic cancer cell lines and its therapeutic efficacy in pancreatic cancer orthotopic nude mouse models.

The most common type of pancreatic cancer is an adenocarcinoma, which occurs in the lining of the pancreatic duct. The possible treatments available for pancreatic cancer are surgery, immunotherapy, radiation therapy, and chemotherapy. Possible surgical treatment options include a distal or total pancreatectomy and a pancreaticoduodenectomy (Whipple procedure).

Radiation therapy may be an option for pancreatic cancer patients, specifically external beam radiation where radiation is focused on the tumor by a machine outside the body. Another option is intraoperative electron beam radiation administered during an operation.

Chemotherapy may be used to treat pancreatic cancer patients. Appropriate anti-cancer drugs include 5-fluorouracil (5-FU), mitomycin, ifosfamide, doxorubicin, steptozocin, chlorozotocin, and combinations thereof.

The methods provided by the invention can provide a beneficial effect for pancreatic cancer patients, by administration of a benzopyrone compound or a combination of administration of a benzopyrone compound and surgery, radiation therapy, or chemotherapy.

### Bladder Cancer

In another aspect, the invention provides methods of treating bladder cancer, preferably a transitional cell carcinoma in urinary bladder. In some embodiments, the method comprises administering benzopyrone compounds alone into a subject. In other embodiments, the method comprises administering benzopyrone compounds in combination with one or more anti-tumor agents as listed herein into a subject.

Bladder cancers are urothelial carcinomas (transitional cell carcinomas) or tumors in the urothelial cells that line the bladder. The remaining cases of bladder cancer are squamous cell carcinomas, adenocarcinomas, and small cell cancers. Several subtypes of urothelial carcinomas exist depending on whether they are noninvasive or invasive and whether they are papillary, or flat. Noninvasive tumors are in the urothelium, the innermost layer of the bladder, while invasive tumors have spread from the urothelium to deeper layers of the bladder's main muscle wall. Invasive papillary urothelial carcinomas are slender finger-like projections that branch into the hollow center of the bladder and also grow outward into the bladder wall. Non-invasive papillary urothelial tumors grow towards the center of the bladder. While a non-invasive, flat urothelial tumor (also called a flat carcinoma in situ) is confined to the layer of cells closest to the inside hollow part of the bladder, an invasive flat urothelial carcinoma invades the deeper layer of the bladder, particularly the muscle layer.

To treat bladder cancer, surgery, radiation therapy, immunotherapy, chemotherapy, or a combination thereof may be applied. Some possible surgical options are a transurethral resection, a cystectomy, or a radical cystectomy. Radiation therapy for bladder cancer may include external beam radiation and brachytherapy.

Immunotherapy is another method that may be used to treat a bladder cancer patient. Typically this is accomplished intravesically, which is the administration of a treatment agent directly into the bladder by way of a catheter. One method is Bacillus Calmete-Guerin (BCG) where a bacterium sometimes used in tuberculosis vaccination is given directly to the bladder through a catheter. The body mounts an immune response to the bacterium, thereby attacking and killing the cancer cells.

Another method of immunotherapy is the administration of interferons, glycoproteins that modulate the immune response. Interferon alpha is often used to treat bladder cancer.

Anti-cancer drugs that may be used in chemotherapy to treat bladder cancer include thitepa, methotrexate, vinblastine, doxorubicin, cyclophosphamide, paclitaxel, carboplatin, cisplatin, ifosfamide, gemcitabine, or combinations thereof.

The methods provided by the invention can provide a beneficial effect for bladder cancer patients, by administration of a benzopyrone compound or a combination of administration of a benzopyrone compound and surgery, radiation therapy, immunotherapy, chemotherapy, or a combination thereof.

### B-Cell Lymphomas

Non-Hodgkin's Lymphomas caused by malignant (cancerous) B-Cell lymphocytes represent a large subset (about 85% in the US) of the known types of lymphoma (the other 2 subsets being T-Cell lymphomas and lymphomas where the cell type is the Natural Killer Cell or unknown). Cells undergo many changes in their life cycle dependent on complex signaling processes between cells and interaction with foreign substances in the body. Various types of lymphoma or leukemia can occur in the B-Cell life cycle.

### Acute Myeloid Leukemia

In another aspect, the invention provides methods of treating acute myeloid leukemia (AML), preferably acute promyleocytic leukemia in peripheral blood. In some embodiments, the method comprises administering benzopyrone compounds alone into a subject. In other embodiments, the method comprises administering benzopyrone compounds in combination with one or more anti-tumor agents as listed herein into a subject.

AML begins in the bone marrow but can spread to other parts of the body including the lymph nodes, liver, spleen, central nervous system, and testes. It is acute meaning it develops quickly and may be fatal if not treated within a few months. AML is characterized by immature bone marrow cells usually granulocytes or monocytes, which continue to reproduce and accumulate.

AML may be treated by immunotherapy, radiation therapy, chemotherapy, bone marrow or peripheral blood stem cell transplantation, or a combination thereof. Radiation therapy includes external beam radiation and may have side effects. Anti-cancer drugs that may be used in chemotherapy to treat AML include cytarabine, anthracycline, anthracenedione, idarubicin, daunorubicin, idarubicin, mitoxantrone, thioguanine, vincristine, prednisone, etoposide, or a combination thereof.

Monoclonal antibody therapy may be used to treat AML patients. Small molecules or radioactive chemicals may be attached to these antibodies before administration to a patient in order to provide a means of killing leukemia cells in the body. The monoclonal antibody, gemtuzumab ozogamicin, which binds CD33 on AML cells, may be used to treat AML patients unable to tolerate prior chemotherapy regimens. Bone marrow or peripheral blood stem cell transplantation may be used to treat AML patients. Some possible transplantation procedures are an allogenic or an autologous transplant.

The methods provided by the invention can provide a beneficial effect for leukemia patients, by administration of a benzopyrone compound or a combination of administration of a benzopyrone compound and surgery, radiation therapy, chemotherapy, or transplantation therapy.

There are other types of leukemia's that can also be treated by the methods provided by the invention including but not limited to, Acute Lymphocytic Leukemia, Acute Myeloid Leukemia, Chronic Lymphocytic Leukemia, Chronic Myeloid Leukemia, Hairy Cell Leukemia, Myelodysplasia, and Myeloproliferative Disorders.

### Lung Cancer

In another aspect, the invention provides methods to treat lung cancer. In some embodiments, the method comprises administering benzopyrone compounds alone into a subject. In other embodiments, the method comprises administering benzopyrone compounds in combination with one or more anti-tumor agents as listed herein into a subject.

The most common type of lung cancer is non-small cell lung cancer (NSCLC), which accounts for approximately 80-85% of lung cancers and is divided into squamous cell carcinomas, adenocarcinomas, and large cell undifferentiated carcinomas. Small cell lung cancer accounts for 15-20% of lung cancers.

Treatment options for lung cancer include surgery, immunotherapy, radiation therapy, chemotherapy, photodynamic therapy, or a combination thereof. Some possible surgical options for treatment of lung cancer are a segmental or wedge resection, a lobectomy, or a pneumonectomy. Radiation therapy may be external beam radiation therapy or brachytherapy.

Some anti-cancer drugs that may be used in chemotherapy to treat lung cancer include cisplatin, carboplatin, paclitaxel, docetaxel, gemcitabine, vinorelbine, irinotecan, etoposde, vinblastine, gefitinib, ifosfamide, methotrexate, or a combination thereof. Photodynamic therapy (PDT) may be used to treat lung cancer patients.

The methods provided by the invention can provide a beneficial effect for lung cancer patients, by administration of a benzopyrone compound or a combination of administration of a benzopyrone compound and surgery, radiation therapy, chemotherapy, photodynamic therapy, or a combination thereof.

### Skin Cancer

In another aspect, the invention provides methods to treat skin cancer. In some embodiments, the method comprises administering benzopyrone compounds alone into a subject. In other embodiments, the method comprises administering benzopyrone compounds in combination with one or more anti-tumor agents as listed herein into a subject.

There are several types of cancer that start in the skin. The most common types are basal cell carcinoma and squamous cell carcinoma, which are non-melanoma skin cancers. Actinic keratosis is a skin condition that sometimes develops into squamous cell carcinoma. Non-melanoma skin cancers rarely spread to other parts of the body. Melanoma, the rarest form of skin cancer, is more likely to invade nearby tissues and spread to other parts of the body. Different types of treatment are available for patients with non-melanoma and melanoma skin cancer and actinic keratosis including surgery, radiation therapy, chemotherapy and photodynamic therapy. Some possible surgical options for treatment of skin cancer are mohs micrographic surgery, simple excision, electrodesiccation and curettage, cryosurgery, laser surgery. Radiation therapy may be external beam radiation therapy or brachytherapy. Other types of treatments that are being tested in clinical trials are biologic therapy or immunotherapy, chemoimmunotherapy, topical chemotherapy with fluorouracil and photodynamic therapy.

The methods provided by the invention can provide a beneficial effect for skin cancer patients, by administration of a benzopyrone compound or a combination of administration of a benzopyrone compound and surgery, radiation therapy, chemotherapy, photodynamic therapy, or a combination thereof.

### Eye Cancer, Retinoblastoma

In another aspect, the invention provides methods to treat eye retinoblastoma. In some embodiments, the method comprises administering benzopyrone compounds alone into a subject. In other embodiments, the method comprises administering benzopyrone compounds in combination with one or more anti-tumor agents as listed herein into a subject.

Retinoblastoma is a malignant tumor of the retina. Although retinoblastoma may occur at any age, it most often occurs in younger children, usually before the age of 5 years. The tumor may be in one eye only or in both eyes. Retinoblastoma is usually confined to the eye and does not spread to nearby tissue or other parts of the body. Treatment options that attempt to cure the patient and preserve vision include enucleation (surgery to remove the eye), radiation therapy, cryotherapy, photocoagulation, immunotherapy, thermotherapy and chemotherapy. Radiation therapy may be external beam radiation therapy or brachytherapy.

The methods provided by the invention can provide a beneficial effect for eye retinoblastoma patients, by administration of a benzopyrone compound or a combination of administration of a benzopyrone compound and surgery, radiation therapy, cryotherapy, photocoagulation, thermotherapy and chemotherapy, or a combination thereof.

### Eye Cancer, Intraocular Melanoma

In another aspect, the invention provides methods to treat intraocular (eye) melanoma. In some embodiments, the method comprises administering benzopyrone compounds alone into a subject. In other embodiments, the method comprises administering benzopyrone compounds in combination with one or more anti-tumor agents as listed herein into a subject.

Intraocular melanoma, a rare cancer, is a disease in which cancer cells are found in the part of the eye called the uvea. The uvea includes the iris, the ciliary body, and the choroid. Intraocular melanoma occurs most often in people who are middle aged. Treatments for intraocular melanoma include surgery, immunotherapy, radiation therapy and laser therapy. Surgery is the most common treatment of intraocular melanoma. Some possible surgical options are iridectomy, iridotrabeculectomy, iridocyclectomy, choroidectomy, enucleation and orbital exenteration. Radiation therapy may be external beam radiation therapy or brachytherapy. Laser therapy may be an intensely powerful beam of light to destroy the tumor, thermotherapy or photocoagulation.

The methods provided by the invention can provide a beneficial effect for intraocular melanoma patients, by administration of a benzopyrone compound or a combination of administration of a benzopyrone compound and surgery, radiation therapy and laser therapy, or a combination thereof.

### Endometrium Cancer

In another aspect, the invention provides methods to treat endometrium cancer. In some embodiments, the method comprises administering benzopyrone compounds alone into a subject. In other embodiments, the method comprises administering benzopyrone compounds in combination with one or more anti-tumor agents as listed herein into a subject.

Endometrial cancer is a cancer that starts in the endometrium, the inner lining of the uterus. Some of the examples of the cancer of uterus and endometrium include, but are not limited to, adenocarcinomas, adenoacanthomas, adenosquamous carcinomas, papillary serous adenocarcinomas, clear cell adenocarcinomas, uterine sarcomas, stromal sarcomas, malignant mixed mesodermal tumors, and leiomyosarcomas.

The methods provided by the invention can provide a beneficial effect for endometrium cancer patients, by administration of a benzopyrone compound or a combination of administration of a benzopyrone compound and surgery, radiation therapy, chemotherapy, gene therapy, RNA therapy, photodynamic therapy, antiangiogenesis therapy, and immunotherapy, or a combination thereof.

### Liver Cancer

In another aspect, the invention provides methods to treat primary liver cancer (cancer that begins in the liver). In some embodiments, the method comprises administering benzopyrone compounds alone into a subject. In other embodiments, the method comprises administering benzopyrone compounds in combination with one or more anti-tumor agents as listed herein into a subject.

Primary liver cancer can occur in both adults and children. Different types of treatments are available for patients with primary liver cancer. These include surgery, immunotherapy, radiation therapy, chemotherapy and percutaneous ethanol injection. The types of surgery that may be used are cryosurgery, partial hepatectomy, total hepatectomy and radiofrequency ablation. Radiation therapy may be external beam radiation therapy, brachytherapy, radiosensitizers or radiolabel antibodies. Other types of treatment include hyperthermia therapy and immunotherapy.

The methods provided by the invention can provide a beneficial effect for liver cancer patients, by administration of a benzopyrone compound or a combination of administration of a benzopyrone compound and surgery, radiation therapy, chemotherapy, percutaneous ethanol injection, hyperthemia therapy and immunotherapy, or a combination thereof.

### Kidney Cancer

In another aspect, the invention provides methods to treat kidney cancer. In some embodiments, the method comprises administering benzopyrone compounds alone into a subject. In other embodiments, the method comprises administering benzopyrone compounds in combination with one or more anti-tumor agents as listed herein into a subject.

Kidney cancer (also called renal cell cancer or renal adenocarcinoma) is a disease in which malignant cells are found in the lining of tubules in the kidney. Kidney cancer may be treated by surgery, radiation therapy, chemotherapy and immunotherapy. Some possible surgical options to treat kidney cancer are partial nephrectomy, simple nephrectomy and radical nephrectomy. Radiation therapy may be external beam radiation therapy or brachytherapy. Stem cell transplant may be used to treat kidney cancer.

The methods provided by the invention can provide a beneficial effect for kidney cancer patients, by administration of a benzopyrone compound or a combination of administration of a benzopyrone compound and surgery, radiation therapy, chemotherapy, immunotherapy and stem cell transplant, or a combination thereof.

### Thyroid Cancer

In another aspect, the invention provides methods to treat thyroid cancer. In some embodiments, the method comprises administering benzopyrone compounds alone into a subject. In other embodiments, the method comprises administering benzopyrone compounds in combination with one or more anti-tumor agents as listed herein into a subject.

Thyroid cancer is a disease in which cancer (malignant) cells are found in the tissues of the thyroid gland. The four main types of thyroid cancer are papillary, follicular, medullary and anaplastic. Thyroid cancer may be treated by surgery, immunotherapy, radiation therapy, hormone therapy and chemotherapy. Surgery is the most common treatment of thyroid cancer. Some possible surgical options for treatment of thyroid cancer are lobectomy, near-total thyroidectomy, total thyroidectomy and lymph node dissection. Radiation therapy may be external radiation therapy or may required intake of a liquid that contains radioactive iodine. Hormone therapy uses hormones to stop cancer cells from growing. In treating thyroid cancer, hormones can be used to stop the body from making other hormones that might make cancer cells grow.

The methods provided by the invention can provide a beneficial effect for thyroid cancer patients, by administration of a benzopyrone compound or a combination of administration of a benzopyrone compound and surgery, surgery, radiation therapy, hormone therapy and chemotherapy, or a combination thereof.

### AIDS Related Cancers

### AIDS-Related Lymphoma

In another aspect, the invention provides methods to treat AIDS-related lymphomas. The method comprises administering a combination of benzopyrone compounds with one or more anti-tumor agents as listed herein into a subject.

AIDS-related lymphoma is a disease in which malignant cells form in the lymph system of patients who have acquired immunodeficiency syndrome (AIDS). AIDS is caused by the human immunodeficiency virus (HIV), which attacks and weakens the body's immune system. The immune system is then unable to fight infection and diseases that invade the body. People with HIV disease have an increased risk of developing infections, lymphoma, and other types of cancer. Lymphomas are cancers that affect the white blood cells of the lymph system. Lymphomas are divided into two general types: Hodgkin's lymphoma and non-Hodgkin's lymphoma. Both Hodgkin's lymphoma and non-Hodgkin's lymphoma may occur in AIDS patients, but non-Hodgkin's lymphoma is more common. When a person with AIDS has non-Hodgkin's lymphoma, it is called an AIDS-related lymphoma. Non-Hodgkin's lymphomas may be indolent (slow-growing) or aggressive (fast-growing). AIDS-related lymphoma is usually aggressive. The three main types of AIDS-related lymphoma are diffuse large B-cell lymphoma, B-cell immunoblastic lymphoma and small non-cleaved cell lymphoma.

Treatment of AIDS-related lymphoma combines treatment of the lymphoma with treatment for AIDS. Patients with AIDS have weakened immune systems and treatment can cause further damage. For this reason, patients who have AIDS-related lymphoma are usually treated with lower doses of drugs than lymphoma patients who do not have AIDS. Highly-active antiretroviral therapy (HAART) is used to slow progression of HIV. Medicine to prevent and treat infections, which can be serious, is also used. AIDS-related lymphomas may be treated by chemotherapy, immunotherapy, radiation therapy and high-dose chemotherapy with stem cell transplant. Radiation therapy may be external beam radiation therapy or brachytherapy. AIDS-related lymphomas can be treated by monoclonal antibody therapy.

The methods provided by the invention can provide a beneficial effect for AIDS-related lymphoma patients, by administration of a combination of benzopyrone with one or more anti-tumor agents or administration of a benzopyrone compound and radiation therapy, or a combination thereof.

### Kaposi's Sarcoma

In another aspect, the invention provides methods to treat Kaposi's sarcoma. The method comprises administering a combination of benzopyrone compounds with one or more anti-tumor agents as listed herein into a subject.

Kaposi's sarcoma is a disease in which cancer cells are found in the tissues under the skin or mucous membranes that line the mouth, nose, and anus. Classic Kaposi's sarcoma usually occurs in older men of Jewish, Italian, or Mediterranean heritage. This type of Kaposi's sarcoma progresses slowly, sometimes over 10 to 15 years. Kaposi's sarcoma may occur in people who are taking immunosuppressants. Kaposi's sarcoma in patients who have Acquired Immunodeficiency Syndrome (AIDS) is called epidemic Kaposi's sarcoma. Kaposi's sarcoma in people with AIDS usually spreads more quickly than other kinds of Kaposi's sarcoma and often is found in many parts of the body. Kaposi's sarcoma may be treated with surgery, chemotherapy, radiation therapy and immunotherapy. External radiation therapy is a common treatment of Kaposi's sarcoma. Some possible surgical options to treat Kaposi's Sarcome are local excision, electrodeiccation and curettage, and cryotherapy.

The methods provided by the invention can provide a beneficial effect for Kaposi's sarcoma, by administration of a benzopyrone compound or a combination of administration of a benzopyrone compound and surgery, chemotherapy, radiation therapy and immunotherapy, or a combination thereof.

### Viral-Induced Cancers

In another aspect, the invention provides methods to treat viral-induced cancers. Several common viruses are clearly or probable causal factors in the etiology of specific malignancies. These viruses either normally establish latency or few can become persistent infections. Oncogenesis is probably linked to an enhanced level of viral activation in the infected host, reflecting heavy viral dose or compromised immune control. The major virus-malignancy systems include hepatitis B virus (HBV), hepatitis C virus (HCV), and hepatocellular carcinoma; human lymphotropic virus-type 1 (HTLV-1) and adult T-cell leukemia/lymphoma; and human papilloma virus (HPV) and cervical cancer. In general, these malignancies occur relatively early in life, typically peaking in middle-age or earlier.

### Virus-Induced Hepatocellular Carcinoma

The causal relationship between both HBV and HCV and hepatocellular carcinoma or liver cancer is established through substantial epidemiologic evidence. Both appear to act via chronic replication in the liver by causing cell death and subsequent regeneration. Different types of treatments are available for patients with liver cancer. These include surgery, immunotherapy, radiation therapy, chemotherapy and percutaneous ethanol injection. The types of surgery that may be used are cryosurgery, partial hepatectomy, total hepatectomy and radiofrequency ablation. Radiation therapy may be external beam radiation therapy, brachytherapy, radiosensitizers or radiolabel antibodies. Other types of treatment include hyperthermia therapy and immunotherapy.

The methods provided by the invention can provide a beneficial effect for virus induce hepatocellular carcinoma patients, by administration of a combination of benzopyrone with one or more anti-tumor agents or administration of a benzopyrone compound and radiation therapy, or a combination thereof.

Viral-Induced Adult T cell Leukemia/Lymphoma

The association between HTLV-1 and Adult T cell leukemia (ATL) is firmly established. Unlike the other oncogenic viruses found throughout the world, HTLV-1 is highly geographically restricted, being found primarily in southern Japan, the Caribbean, west and central Africa, and the South Pacific islands. Evidence for causality includes the monoclonal integration of viral genome in almost all cases of ATL in carriers. The risk factors for HTLV-1-associated malignancy appear to be perinatal infection, high viral load, and being male sex.

Adult T cell leukemia is a cancer of the blood and bone marrow. The standard treatments for adult T cell leukemia/lymphoma are radiation therapy, immunotherapy, and chemotherapy. Radiation therapy may be external beam radiation therapy or brachytherapy. Other methods of treating adult T cell leukemia/lymphoma include immunotherapy and high-dose chemotherapy with stem cell transplantation.

The methods provided by the invention can provide a beneficial effect for Adult T cell leukemia patients, by administration of a benzopyrone compound or a combination of administration of a benzopyrone compound and radiation therapy, chemotherapy, immunotherapy and high-dose chemotherapy with stem cell transplantation, or a combination thereof.

### Viral-Induced Cervical Cancer

Infection of the cervix with human papillomavirus (HPV) is the most common cause of cervical cancer. Not all women with HPV infection, however, will develop cervical cancer. Cervical cancer usually develops slowly over time. Before cancer appears in the cervix, the cells of the cervix go through changes known as dysplasia, in which cells that are not normal begin to appear in the cervical tissue. Later, cancer cells start to grow and spread more deeply into the cervix and to surrounding areas. The standard treatments for cervical cancers are surgery, immunotherapy, radiation therapy and chemotherapy. The types of surgery that may be used are conization, total hysterectomy, bilateral salpingo-oophorectomy, radical hysterectomy, pelvic exenteration, cryosurgery, laser surgery and loop electrosurgical excision procedure. Radiation therapy may be external beam radiation therapy or brachytherapy.

The methods provided by the invention can provide a beneficial effect for adult cervical cancer, by administration of a benzopyrone compound or a combination of administration of a benzopyrone compound and radiation therapy, chemotherapy, or a combination thereof.

### CNS Cancers

In another aspect, the invention provides methods to treat central nervous system cancers. In some embodiments, the method comprises administering benzopyrone compounds alone into a subject. In other embodiments, the method comprises administering benzopyrone compounds in combination with one or more anti-tumor agents as listed herein into a subject.

Brain and spinal cord tumors are abnormal growths of tissue found inside the skull or the bony spinal column, which are the primary components of the central nervous system (CNS). Benign tumors are noncancerous, and malignant tumors are cancerous. The CNS is housed within rigid, bony quarters (i.e., the skull and spinal column), so any abnormal growth, whether benign or malignant, can place pressure on sensitive tissues and impair function. Tumors that originate in the brain or spinal cord are called primary tumors. Most primary tumors are caused by out-of-control growth among cells that surround and support neurons. In a small number of individuals, primary tumors may result from specific genetic disease (e.g., neurofibromatosis, tuberous sclerosis) or from exposure to radiation or cancer-causing chemicals. The cause of most primary tumors remains a mystery.

The first test to diagnose brain and spinal column tumors is a neurological examination. Special imaging techniques (computed tomography, and magnetic resonance imaging, positron emission tomography) are also employed. Laboratory tests include the EEG and the spinal tap. A biopsy, a surgical procedure in which a sample of tissue is taken from a suspected tumor, helps doctors diagnose the type of tumor.

Tumors are classified according to the kind of cell from which the tumor seems to originate. The most common primary brain tumor in adults comes from cells in the brain called astrocytes that make up the blood-brain barrier and contribute to the nutrition of the central nervous system. These tumors are called gliomas (astrocytoma, anaplastic astrocytoma, or glioblastoma multiforme) and account for 65% of all primary central nervous system tumors. Some of the tumors are, but not limited to, Oligodendroglioma, Ependymoma, Meningioma, Lymphoma, Schwannoma, and Medulloblastoma.

### Neuroepithelial Tumors of the CNS

Astrocytic tumors, such as astrocytoma; anaplastic (malignant) astrocytoma, such as hemispheric, diencephalic, optic, brain stem, cerebellar; glioblastoma multiforme; pilocytic astrocytoma, such as hemispheric, diencephalic, optic, brain stem, cerebellar; subependymal giant cell astrocytoma; and pleomorphic xanthoastrocytoma. Oligodendroglial tumors, such as oligodendroglioma; and anaplastic (malignant) oligodendroglioma. Ependymal cell tumors, such as ependymoma; anaplastic ependymoma; myxopapillary ependymoma; and subependymoma. Mixed gliomas, such as mixed oligoastrocytoma; anaplastic (malignant) oligoastrocytoma; and others (e.g. ependymo-astrocytomas). Neuroepithelial tumors of uncertain origin, such as polar spongioblastoma; astroblastoma; and gliomatosis cerebri. Tumors of the choroid plexus, such as choroid plexus papilloma; and choroid plexus carcinoma (anaplastic choroid plexus papilloma). Neuronal and mixed neuronal-glial tumors, such as gangliocytoma; dysplastic gangliocytoma of cerebellum (Lhermitte-Duclos); ganglioglioma; anaplastic (malignant) ganglioglioma; desmoplastic infantile ganglioglioma, such as desmoplastic infantile astrocytoma; central neurocytoma; dysembryoplastic neuroepithelial tumor; olfactory neuroblastoma (esthesioneuroblastoma. Pineal Parenchyma Tumors, such as pineocytoma; pineoblastoma; and mixed pineocytoma/pineoblastoma. Tumors with neuroblastic or glioblastic elements (embryonal tumors), such as medulloepithelioma; primitive neuroectodermal tumors with multipotent differentiation, such as medulloblastoma; cerebral primitive neuroectodermal tumor; neuroblastoma; retinoblastoma; and ependymoblastoma.

### Other CNS Neoplasms

Tumors of the Sellar Region, such as pituitary adenoma; pituitary carcinoma; and craniopharyngioma. Hematopoietic tumors, such as primary malignant lymphomas; plasmacytoma; and granulocytic sarcoma. Germ Cell Tumors, such as germinoma; embryonal carcinoma; yolk sac tumor (endodermal sinus tumor); choriocarcinoma; teratoma; and mixed germ cell tumors. Tumors of the Meninges, such as meningioma; atypical meningioma; and anaplastic (malignant) meningioma. Non-menigothelial tumors of the meninges, such as Benign Mesenchymal; Malignant Mesenchymal; Primary Melanocytic Lesions; Hemopoietic Neoplasms; and Tumors of Uncertain Histogenesis, such as hemangioblastoma (capillary hemangioblastoma). Tumors of Cranial and Spinal Nerves, such as schwannoma (neurinoma, neurilemoma); neurofibroma; malignant peripheral nerve sheath tumor (malignant schwannoma), such as epithelioid, divergent mesenchymal or epithelial differentiation, and melanotic. Local Extensions from Regional Tumors; such as paraganglioma (chemodectoma); chordoma; chodroma; chondrosarcoma; and carcinoma. Metastatic tumours, Unclassified Tumors and Cysts and Tumor-like Lesions, such as Rathke cleft cyst; Epidermoid; dermoid; colloid cyst of the third ventricle; enterogenous cyst; neuroglial cyst; granular cell tumor (choristoma, pituicytoma); hypothalamic neuronal hamartoma; nasal glial herterotopia; and plasma cell granuloma.

Chemotherapeutics available are, but not limited to, alkylating agents such as, Cyclophosphamide, Ifosphamide, Melphalan, Chlorambucil, BCNU, CCNU, Decarbazine, Procarbazine, Busulfan, and Thiotepa; antimetabolites such as, Methotraxate, 5-Fluorouracil, Cytarabine, Gemcitabine (Gemzar®), 6-mercaptopurine, 6-thioguanine, Fludarabine, and Cladribine; anthracyclins such as, daunorubicin. Doxorubicin, Idarubicin, Epirubicin and Mitoxantrone; antibiotics such as, Bleomycin; camptothecins such as, irinotecan and topotecan; taxanes such as, paclitaxel and docetaxel; and platinums such as, Cisplatin, carboplatin, and Oxaliplatin.

The treatments are surgery, radiation therapy, immunotherapy, hyperthermia, gene therapy, RNA therapy, chemotherapy, and combination of radiation and chemotherapy. Doctors also may prescribe steroids to reduce the swelling inside the CNS.

The methods provided by the invention can provide a beneficial effect for CNS cancer, by administration of a benzopyrone compound or a combination of administration of a benzopyrone compound and radiation therapy, chemotherapy, or a combination thereof.

### Colon Cancer and Rectal Cancer

In another aspect, the invention provides methods to treat colorectal cancers. In some embodiments, the method comprises administering benzopyrone compounds alone into a subject. In other embodiments, the method comprises administering benzopyrone compounds in combination with one or more anti-tumor agents as listed herein into a subject.

Colorectal cancer includes cancerous growths in the colon, rectum and appendix. Many colorectal cancers are thought to arise from adenomatous polyps in the colon. Colorectal cancer originates from the epithelial cells lining the gastrointestinal tract. Hereditary or somatic mutations in specific DNA sequences, among which are included DNA replication or DNA repair genes, and also the APC, K-Ras, NOD2 and p53 genes, lead to unrestricted cell division. Therapy is usually through surgery, which in many cases is followed by chemotherapy. Bacillus Calmette-Guérin (BCG) is being investigated as an adjuvant mixed with autologous tumor cells in immunotherapy for colorectal cancer.

Over 20% of patients present with metastatic (stage 1V) colorectal cancer at the time of diagnosis, and up to 25% of this group have isolated liver metastasis that is potentially resectable. Patients with colon cancer and metastatic disease to the liver may be treated in either a single surgery or in staged surgeries depending upon the fitness of the patient for prolonged surgery, the difficulty expected with the procedure with either the colon or liver resection, and the comfort of the surgery performing potentially complex hepatic surgery.

The methods provided by the invention can provide a beneficial effect for colorectal cancer patients, by administration of a benzopyrone compound or a combination of administration of a benzopyrone compound and anti-tumor agents, and radiation therapy, immunotherapy, or a combination thereof.

### Stomach Cancer

In another aspect, the invention provides methods to treat stomach cancers. In some embodiments, the method comprises administering benzopyrone compounds alone into a subject. In other embodiments, the method comprises administering benzopyrone compounds in combination with one or more anti-tumor agents as listed herein into a subject.

Stomach or gastric cancer can develop in any part of the stomach and may spread throughout the stomach and to other organs; particularly the esophagus and the small intestine. There are three main types of stomach cancers: lymphomas, gastric stromal tumors, and carcinoid tumors. Lymphomas are cancers of the immune system tissue that are sometimes found in the wall of the stomach. Gastric stromal tumors develop from the tissue of the stomach wall. Carcinoid tumors are tumors of hormone-producing cells of the stomach. Infection with the bacterium H. pylori is the main risk factor in about 80% or more of gastric cancers. It is more common in men. The causes of stomach cancer continue to be debated. A combination of heredity and environment (diet, smoking, etc) are all thought to play a part.

Common approaches to the treatment include surgery, immunotherapy, chemotherapy, radiation therapy, combination of chemotherapy and radiation therapy or biological therapy. Stomach cancer is difficult to cure unless it is found in an early stage (before it has begun to spread). New treatment approaches such as biological therapy and improved ways of using current methods are being studied in clinical trials.

The methods provided by the invention can provide a beneficial effect for stomach cancer patients, by administration of a benzopyrone compound or a combination of administration of a benzopyrone compound and anti-tumor agents, and radiation therapy, immunotherapy, or a combination thereof.

### Gallbladder Cancer

In another aspect, the invention provides methods to treat gallbladder cancers. In some embodiments, the method comprises administering benzopyrone compounds alone into a subject. In other embodiments, the method comprises administering benzopyrone compounds in combination with one or more anti-tumor agents as listed herein into a subject.

Gallbladder cancer is a rare cancer in which malignant cells are found in the tissues of the gallbladder. The gallbladder stores bile, a fluid made by the liver to digest fat. The wall of the gallbladder has 3 main layers of tissue: mucosal (innermost) layer, muscularis (middle, muscle) layer, and serosal (outer) layer. Between these layers is supporting connective tissue. Primary gallbladder cancer starts in the innermost layer and spreads through the outer layers as it grows. Gallbladder cancer can be cured only if it is found before it has spread, when it can be removed by surgery. If the cancer has spread, palliative treatment can improve the patient's quality of life by controlling the symptoms and complications of this disease.

The methods provided by the invention can provide a beneficial effect for gallbladder cancer patients, by administration of a benzopyrone compound or a combination of administration of a benzopyrone compound and anti-tumor agents, and radiation therapy, immunotherapy, or a combination thereof.

### Esophageal Cancer

In another aspect, the invention provides methods to treat esophageal cancers. In some embodiments, the method comprises administering benzopyrone compounds alone into a subject. In other embodiments, the method comprises administering benzopyrone compounds in combination with one or more anti-tumor agents as listed herein into a subject.

Esophageal cancer is malignancy of the esophagus. There are various subtypes. Most tumors of the esophagus are malignant. A very small proportion (under 10%) is leiomyoma (smooth muscle tumor) or gastrointestinal stromal tumor (GIST). Malignant tumors are generally adenocarcinomas, squamous cell carcinomas, and occasionally small-cell carcinomas. The latter share many properties with small-cell lung cancer, and are relatively sensitive to chemotherapy compared to the other types.

Small and localized tumors are treated surgically with curative intent. Larger tumors tend not to be operable and hence cannot be cured; their growth can still be delayed with chemotherapy, radiotherapy or a combination of the two. In some cases chemo- and radiotherapy can render these larger tumors operable.

The methods provided by the invention can provide a beneficial effect for esophageal cancer patients, by administration of a benzopyrone compound or a combination of administration of a benzopyrone compound and anti-tumor agents, and radiation therapy, immunotherapy, or a combination thereof.

### PNS Cancers

In another aspect, the invention provides methods to treat peripheral nervous system (PNS) cancers. In some embodiments, the method comprises administering benzopyrone compounds alone into a subject. In other embodiments, the method comprises administering benzopyrone compounds in combination with one or more anti-tumor agents as listed herein into a subject.

The peripheral nervous system consists of the nerves that branch out from the brain and spinal cord. These nerves form the communication network between the CNS and the body parts. The peripheral nervous system is further subdivided into the somatic nervous system and the autonomic nervous system. The somatic nervous system consists of nerves that go to the skin and muscles and is involved in conscious activities. The autonomic nervous system consists of nerves that connect the CNS to the visceral organs such as the heart, stomach, and intestines. It mediates unconscious activities.

Acoustic neuromas are benign fibrous growths that arise from the balance nerve, also called the eighth cranial nerve or vestibulocochlear nerve. These tumors are non-malignant, meaning that they do not spread or metastasize to other parts of the body. The location of these tumors is deep inside the skull, adjacent to vital brain centers in the brain stem. As the tumors enlarge, they involve surrounding structures which have to do with vital functions. In the majority of cases, these tumors grow slowly over a period of years.

The malignant peripheral nerve sheath tumor (MPNST) is the malignant counterpart to benign soft tissue tumors such as neurofibromas and schwannomas. It is most common in the deep soft tissue, usually in close proximity of a nerve trunk. The most common sites include the sciatic nerve, brachial plexus, and sarcal plexus. The most common symptom is pain which usually prompts a biopsy. It is a rare, aggressive, and lethal orbital neoplasm that usually arises from sensory branches of the trigeminal nerve in adults. Malignant PNS tumor spreads along nerves to involve the brain, and most patients die within 5 years of clinical diagnosis. The MPNST may be classified into three major categories with epithelioid, mesenchymal or glandular characteristics. Some of the MPNST include but not limited to, Subcutaneous malignant epithelioid schwannoma with cartilaginous differentiation, Glandular malignant schwannoma, Malignant peripheral nerve sheath tumor with perineurial differentiation, Cutaneous epithelioid malignant nerve sheath tumor with rhabdoid features, Superficial epithelioid MPNST, Triton Tumor (MPNST with rhabdomyoblastic differentiation), Schwannoma with rhabdomyoblastic differentiation. Rare MPNST cases contain multiple sarcomatous tissue types, especially osteosarcoma, chondrosarcoma and angiosarcoma. These have sometimes been indistinguishable from the malignant mesenchymoma of soft tissue.

Other types of PNS cancers include but not limited to, malignant fibrous cytoma, malignant fibrous histiocytoma, malignant meningioma, malignant mesothelioma, and malignant mixed Müllerian tumor.

The treatments are surgery, radiation therapy, immunotherapy, chemotherapy, and combination of radiation and chemotherapy.

The methods provided by the invention can provide a beneficial effect for PNS cancer patients, by administration of a benzopyrone compound or a combination of administration of a benzopyrone compound and anti-tumor agents, and radiation therapy, immunotherapy, or a combination thereof.

### Head and Neck, Oral Cavity and Oropharyngeal Cancer

In another aspect, the invention provides methods to treat head and neck cancers including cancers of the lip, oral cavity, nasal cavity, paranasal sinuses, pharynx, and larynx. In some embodiments, the method comprises administering benzopyrone compounds alone into a subject. In other embodiments, the method comprises administering benzopyrone compounds in combination with one or more anti-tumor agents as listed herein into a subject.

Cancers such as, hypopharyngeal cancer, laryngeal cancer, nasopharyngeal cancer, oropharyngeal cancer, and the like, have been treated with surgery, immunotherapy, chemotherapy, combination of chemotherapy and radiation therapy. Etoposide and actinomycin D, two commonly used oncology agents that inhibit topoisomerase II, fail to cross the blood-brain barrier in useful amounts.

The methods provided by the invention can provide a beneficial effect for oral cavity and oropharyngeal cancer, by administration of a benzopyrone compound or a combination of administration of a benzopyrone compound and anti-tumor agents, and radiation therapy, immunotherapy, or a combination thereof.

### Testicular Cancer

In another aspect, the invention provides methods to treat testicular cancer. In some embodiments, the method comprises administering benzopyrone compounds alone into a subject. In other embodiments, the method comprises administering benzopyrone compounds in combination with one or more anti-tumor agents as listed herein into a subject.

Testicular cancer is cancer that typically develops in one or both testicles in young men. Cancers of the testicle develop in certain cells known as germ cells. The 2 main types of germ cell tumors (GCTs) that occur in men are seminomas (60%) and nonseminomas (40%). Tumors can also arise in the supportive and hormone-producing tissues, or stroma, of the testicles. Such tumors are known as gonadal stromal tumors. The 2 main types are Leydig cell tumors and Sertoli cell tumors. Secondary testicular tumors are those that start in another organ and then spread to the testicle. Lymphoma is the most common secondary testicular cancer.

Common approaches to the treatment include surgery, immunotherapy, chemotherapy, radiation therapy, combination of chemotherapy and radiation therapy or biological therapy. Several drugs are typically used to treat testicular cancer: Platinol (cisplatin), Vepesid or VP-16 (etoposide) and Blenoxane (bleomycin sulfate). Additionally, Ifex (ifosamide), Velban (vinblastine sulfate) and others may be used.

The methods provided by the invention can provide a beneficial effect for stomach cancer, by administration of a benzopyrone compound or a combination of administration of a benzopyrone compound and radiation therapy, chemotherapy, or a combination thereof.

### Thymus Cancer

In another aspect, the invention provides methods to treat thymus cancer. In some embodiments, the method comprises administering benzopyrone compounds alone into a subject. In other embodiments, the method comprises administering benzopyrone compounds in combination with one or more anti-tumor agents as listed herein into a subject.

The thymus is a small organ located in the upper/front portion of your chest, extending from the base of the throat to the front of the heart. The thymus contains 2 main types of cells, thymic epithelial cells and lymphocytes. Thymic epithelial cells can give origin to thymomas and thymic carcinomas. Thymomas are epithelial tumors of the thymus, which may or may not be extensively infiltrated by normeoplastic lymphocytes. The term thymoma is customarily used to describe neoplasms that show no overt atypia of the epithelial component. A thymic epithelial tumor that exhibits clear-cut cytologic atypia and histologic features no longer specific to the thymus is known as a thymic carcinoma (also known as type C thymoma). Lymphocytes, whether in the thymus or in the lymph nodes, can become malignant and develop into cancers called Hodgkin disease and non-Hodgkin lymphomas. The thymus also contains another much less common type of cells called Kulchitsky cells, or neuroendocrine cells, which normally release certain hormones. These cells can give rise to cancers, called carcinoids or carcinoid tumors that often release the same type of hormones, and are similar to other tumors arising from neuroendocrine cells elsewhere in the body.

Common approaches to the treatment include surgery, immunotherapy, chemotherapy, radiation therapy, combination of chemotherapy and radiation therapy or biological therapy. Anticancer drugs that have been used in the treatment of thymomas and thymic carcinomas are doxorubicin (Adriamycin), cisplatin, ifosfamide, and corticosteroids (prednisone). Often, these drugs are given in combination to increase their effectiveness. Combinations used to treat thymic cancer include cisplatin, doxorubicin, etoposide and cyclophosphamide, and the combination of cisplatin, doxorubicin, cyclophosphamide, and vincristine.

The methods provided by the invention can provide a beneficial effect for stomach cancer, by administration of a benzopyrone compound or a combination of administration of a benzopyrone compound and radiation therapy, chemotherapy, or a combination thereof.

### Urethral Cancer

In another aspect, the invention provides methods to treat urethral cancer. In some embodiments, the method comprises administering benzopyrone compounds alone into a subject. In other embodiments, the method comprises administering benzopyrone compounds in combination with one or more anti-tumor agents as listed herein into a subject.

Urethral cancer is a rare cancer that occurs more often in women than in men. There are different types of urethral cancer that begin in cells that line the urethra. These cancers are named for the types of cells that become malignant: Squamous cell carcinoma is the most common type of urethral cancer. It forms in cells in the part of the urethra near the bladder in women, and in the lining of the urethra in the penis in men. Transitional cell carcinoma forms in the area near the urethral opening in women, and in the part of the urethra that goes through the prostate gland in men. Adenocarcinoma forms in glands near the urethra in both men and women.

Treatment of urethral cancer depends on the stage of the cancer and where it is in the urethra; the patient's sex and general health; and whether the cancer has just been diagnosed or has recurred.

The methods provided by the invention can provide a beneficial effect for urethral cancer patients, by administration of a benzopyrone compound or a combination of administration of a benzopyrone compound and anti-tumor agents, and radiation therapy, immunotherapy, or a combination thereof.

Sarcomas Other than Kaposi's Sarcoma

In another aspect, the invention provides methods to treat sarcomas other than Kaposi's sarcoma. In some embodiments, the method comprises administering benzopyrone compounds alone into a subject. In other embodiments, the method comprises administering benzopyrone compounds in combination with one or more anti-tumor agents as listed herein into a subject.

There are several subtypes of sarcomas, based on the type of tissue from which they arise. For example, osteosarcoma arises from bone, chondrosarcoma arises from cartilage, and leiomyosarcoma arises from smooth muscle. Soft tissue sarcomas, such as leiomyosarcoma, chondrosarcoma, and gastrointestinal stromal tumor (GIST), are more common in adults than in children. Bone sarcomas, such as osteosarcoma and Ewing's sarcoma, are more common in children than in adults. These tumors most commonly strike adolescents and young adults between the ages of 12 and 25. In addition to being named based on the tissue of origin, sarcomas are also assigned a grade, such as low grade or high grade. Low grade sarcomas are usually treated surgically, although sometimes radiation therapy or chemotherapy is used. High grade sarcomas are more frequently treated with chemotherapy. Since these tumors are more likely to undergo metastasis, these tumors are treated more aggressively. Childhood sarcomas are almost always treated with a combination of surgery and chemotherapy, and radiation is frequently used as well. The recognition that childhood sarcomas are sensitive to chemotherapy has dramatically improved the survival of patients.

### Vaginal Cancer

Vaginal cancer is a disease in which malignant cells form in the vagina. Carcinomas of the vagina include squamous cell carcinoma, adenocarcinoma, melanoma and sarcoma. Squamous cell vaginal carcinoma spreads slowly and usually stays near the vagina, but may spread to the lungs and liver. Adenocarcinoma begins in glandular (secretory) cells. Adenocarcinoma is more likely than squamous cell cancer to spread to the lungs and lymph nodes.

### Cancer Stem Cells:

Methods and compositions of the present invention may be used to treat cancers derived from cancer stem cells. Cancer stem cells (CSCs) are a sub-population of cancer cells found within tumors or hematological cancers that possess characteristics normally associated with stem cells. CSCs are believed to be tumorigenic, in contrast to the bulk of cancer cells, which are thought to be non-tumorigenic. CSCs have stem cell properties such as self-renewal and the ability to differentiate into multiple cell types. CSCs are also capable of forming heterogeneous tumors in immunodeficient mice at high frequency. It has been suggested that CSCs persist in tumors as a distinct population and cause relapse and metastasis by giving rise to new tumors. Most human tumors have now been shown to contain a sub-population of cells that display cancer stem cell characteristics. The types of cancer include but are not limited to leukemia, breast cancer, melanoma, lung cancer, brain cancers, colon cancers, pancreatic cancer, and ovarian cancer.

The existence of cancer stem cells has several implications in terms of cancer treatment and therapies. Normal stem cells are naturally resistant to chemotherapeutic agents because they have various pumps (such as MDR) that pump out drugs. Stem cells also have DNA repair proteins and a slow rate of cell turnover. Cancer stem cells, being the mutated counterparts of normal stem cells, may also have similar functions which allow them to survive various therapies. By selectively targeting cancer stem cells, it would be possible to treat patients with aggressive tumors, as well as preventing the tumor from metastasizing. References on cancer stem cells and cancer stem cell targeted agents include Trumpp A, Wiestler OD. Mechanisms of Disease: cancer stem cells—targeting the evil twin. Nat Clin Pract Oncol. 2008 June; 5(6):337-47. Epub 2008 Apr. 22. Chumsri S, Burger A M. Cancer stem cell targeted agents: therapeutic approaches and consequences. Curr Opin Mol. Ther. 2008 August; 10(4):323-33, both of which are herein incorporated by reference in their entireties.

The methods provided by the invention can provide a beneficial effect for cancer patients, by administration of a benzopyrone compound or a combination of administration of a benzopyrone compound and anti-tumor agents, and radiation therapy, RNA therapy, nanotherapy, gene therapy, immunotherapy, or a combination thereof.

### Combination Therapy

One aspect of the invention provides methods for treating cancer using different combinations of treatment regimens. For example, such combinations may include, but are not limited to, the use of one or more of the benzopyrone compounds in conjunction with one or more various antineoplastic anti-tumor agents, chemopreventative agents, and/or side-effect limiting agents.

### Antineoplastic Chemotherapeutic Agents

Suitable antineoplastic anti-tumor agents to be used in the present invention include, but are not limited to, alkylating agents, antimetabolites, natural antineoplastic agents, hormonal antineoplastic agents, angiogenesis inhibitors, differentiating reagents, RNA inhibitors, antibodies or immunotherapeutic agents, gene therapy agents, small molecule enzymatic inhibitors, biological response modifiers, and anti-metastatic agents.

### Alkylating Agents

Alkylating agents are known to act through the alkylation of macromolecules such as the DNA of cancer cells, and are usually strong electrophiles. This activity can disrupt DNA synthesis and cell division. Examples of alkylating reagents suitable for use herein include nitrogen mustards and their analogues and derivatives including, cyclophosphamide, ifosfamide, chlorambucil, estramustine, mechlorethamine hydrochloride, melphalan, and uracil mustard. Other examples of alkylating agents include alkyl sulfonates (e.g. busulfan), nitrosoureas (e.g. carmustine, lomustine, and streptozocin), triazenes (e.g. dacarbazine and temozolomide), ethylenimines/methylmelamines (e.g. altretamine and thiotepa), and methylhydrazine derivatives (e.g. procarbazine). Included in the alkylating agent group are the alkylating-like platinum-containing drugs comprising carboplatin, cisplatin, and oxaliplatin.

### Antimetabolites

Antimetabolic antineoplastic agents structurally resemble natural metabolites, and are involved in normal metabolic processes of cancer cells such as the synthesis of nucleic acids and proteins. They differ enough from the natural metabolites so that they interfere with the metabolic processes of cancer cells. Suitable antimetabolic antineoplastic agents to be used in the present invention can be classified according to the metabolic process they affect, and can include, but are not limited to, analogues and derivatives of folic acid, pyrimidines, purines, and cytidine. Members of the folic acid group of agents suitable for use herein include, but are not limited to, methotrexate (amethopterin), pemetrexed and their analogues and derivatives. Pyrimidine agents suitable for use herein include, but are not limited to, cytarabine, floxuridine, fluorouracil (5-fluorouracil), capecitabine, gemcitabine, and their analogues and derivatives. Purine agents suitable for use herein include, but are not limited to, mercaptopurine (6-mercaptopurine), pentostatin, thioguanine, cladribine, and their analogues and derivatives. Cytidine agents suitable for use herein include, but are not limited to, cytarabine (cytosine arabinodside), azacitidine (5-azacytidine) and their analogues and derivatives.

### Natural Antineoplastic Agents

Natural antineoplastic agents comprise antimitotic agents, antibiotic antineoplastic agents, camptothecin analogues, and enzymes. Antimitotic agents suitable for use herein include, but are not limited to, vinca alkaloids like vinblastine, vincristine, vindesine, vinorelbine, and their analogues and derivatives. They are derived from the Madagascar periwinkle plant and are usually cell cycle-specific for the M phase, binding to tubulin in the microtubules of cancer cells. Other antimitotic agents suitable for use herein are the podophyllotoxins, which include, but are not limited to etoposide, teniposide, and their analogues and derivatives. These reagents predominantly target the G2 and late S phase of the cell cycle.

Also included among the natural antineoplastic agents are the antibiotic antineoplastic agents. Antibiotic antineoplastic agents are antimicrobial drugs that have anti-tumor properties usually through interacting with cancer cell DNA. Antibiotic antineoplastic agents suitable for use herein include, but are not limited to, belomycin, dactinomycin, doxorubicin, idarubicin, epirubicin, mitomycin, mitoxantrone, pentostatin, plicamycin, and their analogues and derivatives.

The natural antineoplastic agent classification also includes camptothecin analogues and derivatives which are suitable for use herein and include camptothecin, topotecan, and irinotecan. These agents act primarily by targeting the nuclear enzyme topoisomerase I. Another subclass under the natural antineoplastic agents is the enzyme, L-asparaginase and its variants. L-asparaginase acts by depriving some cancer cells of L-asparagine by catalyzing the hydrolysis of circulating asparagine to aspartic acid and ammonia.

### Hormonal Antineoplastic Agents

Hormonal antineoplastic agents act predominantly on hormone-dependent cancer cells associated with prostate tissue, breast tissue, endometrial tissue, ovarian tissue, lymphoma, and leukemia. Such tissues may be responsive to and dependent upon such classes of agents as glucocorticoids, progestins, estrogens, and androgens. Both analogues and derivatives that are agonists or antagonists are suitable for use in the present invention to treat tumors. Examples of glucocorticoid agonists/antagonists suitable for use herein are dexamethasone, cortisol, corticosterone, prednisone, mifepristone (RU486), their analogues and derivatives. The progestin agonist/antagonist subclass of agents suitable for use herein includes, but is not limited to, hydroxyprogesterone, medroxyprogesterone, megestrol acetate, mifepristone (RU486), ZK98299, their analogues and derivatives. Examples from the estrogen agonist/antagonist subclass of agents suitable for use herein include, but are not limited to, estrogen, tamoxifen, toremifene, RU58668, SR16234, ZD164384, ZK191703, fulvestrant, their analogues and derivatives. Examples of aromatase inhibitors suitable for use herein, which inhibit estrogen production, include, but are not limited to, androstenedione, formestane, exemestane, aminoglutethimide, anastrozole, letrozole, their analogues and derivatives. Examples from the androgen agonist/antagonist subclass of agents suitable for use herein include, but are not limited to, testosterone, dihydrotestosterone, fluoxymesterone, testolactone, testosterone enanthate, testosterone propionate, gonadotropin-releasing hormone agonists/antagonists (e.g. leuprolide, goserelin, triptorelin, buserelin), diethylstilbestrol, abarelix, cyproterone, flutamide, nilutamide, bicalutamide, their analogues and derivatives.

### Angiogenesis Inhibitors

Angiogenesis inhibitors work by inhibiting the vascularization of tumors. Angiogenesis inhibitors encompass a wide variety of agents including small molecule agents, antibody agents, and agents that target RNA function. Examples of angiogenesis inhibitors suitable for use herein include, but are not limited to, ranibizumab, bevacizumab, SU11248, PTK787, ZK222584, CEP-7055, angiozyme, dalteparin, thalidomide, suramin, CC-5013, combretastatin A4 Phosphate, LY317615, soy isoflavones, AE-941, interferon alpha, PTK787/ZK 222584, ZD6474, EMD 121974, ZD6474, BAY 543-9006, celecoxib, halofuginone hydrobromide, bevacizumab, their analogues, variants, or derivatives.

### Differentiating Reagents

Differentiating agents inhibit tumor growth through mechanisms that induce cancer cells to differentiate. One such subclass of these agents suitable for use herein includes, but is not limited to, vitamin A analogues or retinoids, and peroxisome proliferator-activated receptor agonists (PPARs). Retinoids suitable for use herein include, but are not limited to, vitamin A, vitamin A aldehyde (retinal), retinoic acid, fenretinide, 9-cis-retinoid acid, 13-cis-retinoid acid, all-trans-retinoic acid, isotretinoin, tretinoin, retinyl palmitate, their analogues and derivatives. Agonists of PPARs suitable for use herein include, but are not limited to, troglitazone, ciglitazone, tesaglitazar, their analogues and derivatives.

### Antibodies/Immunotherapeutic Agents

Antibody agents bind targets selectively expressed in cancer cells and can either utilize a conjugate to kill the cell associated with the target, or elicit the body's immune response to destroy the cancer cells. Immunotherapeutic agents can either be comprised of polyclonal or monoclonal antibodies. The antibodies may be comprised of non-human animal (e.g. mouse) and human components, or be comprised of entirely human components (“humanized antibodies”). Examples of monoclonal immunotherapeutic agents suitable for use herein include, but are not limited to, rituximab, tosibtumomab, ibritumomab which target the CD-20 protein. Other examples suitable for use herein include trastuzumab, edrecolomab, bevacizumab, cetuximab, carcinoembryonic antigen antibodies, gemtuzumab, alemtuzumab, mapatumumab, panitumumab, EMD 72000, TheraCIM hR3, 2C4, HGS-TR2J, and HGS-ETR2.

### Gene Therapy Agents

Gene therapy agents insert copies of genes into a specific set of a patient's cells, and can target both cancer and non-cancer cells. The goal of gene therapy can be to replace altered genes with functional genes, to stimulate a patient's immune response to cancer, to make cancer cells more sensitive to chemotherapy, to place “suicide” genes into cancer cells, or to inhibit angiogenesis. Genes may be delivered to target cells using viruses, liposomes, or other carriers or vectors. This may be done by injecting the gene-carrier composition into the patient directly, or ex vivo, with infected cells being introduced back into a patient. Such compositions are suitable for use in the present invention.

### Nanotherapy

Nanometer-sized particles have novel optical, electronic, and structural properties that are not available from either individual molecules or bulk solids. When linked with tumor-targeting moieties, such as tumor-specific ligands or monoclonal antibodies, these nanoparticles can be used to target cancer-specific receptors, tumor antigens (biomarkers), and tumor vasculatures with high affinity and precision. The formuation and manufacturing process for cancer nanotherapy is disclosed in patent U.S. Pat. No. 7,179,484, and article M. N. Khalid, P. Simard, D. Hoarau, A. Dragomir, J. Leroux, Long Circulating Poly(Ethylene Glycol)Decorated Lipid Nanocapsules Deliver Docetaxel to Solid Tumors, Pharmaceutical Research, 23(4), 2006, all of which are herein incorporated by reference in their entireties.

### RNA Therapy

RNA including but not limited to siRNA, shRNA, microRNA may be used to modulate gene expression and treat cancers. Double stranded oligonucleotides are formed by the assembly of two distinct oligonucleotide sequences where the oligonucleotide sequence of one strand is complementary to the oligonucleotide sequence of the second strand; such double stranded oligonucleotides are generally assembled from two separate oligonucleotides (e.g., siRNA), or from a single molecule that folds on itself to form a double stranded structure (e.g., shRNA or short hairpin RNA). These double stranded oligonucleotides known in the art all have a common feature in that each strand of the duplex has a distinct nucleotide sequence, wherein only one nucleotide sequence region (guide sequence or the antisense sequence) has complementarity to a target nucleic acid sequence and the other strand (sense sequence) comprises nucleotide sequence that is homologous to the target nucleic acid sequence.

MicroRNAs (miRNA) are single-stranded RNA molecules of about 21-23 nucleotides in length, which regulate gene expression. miRNAs are encoded by genes that are transcribed from DNA but not translated into protein (non-coding RNA); instead they are processed from primary transcripts known as pri-miRNA to short stem-loop structures called pre-miRNA and finally to functional miRNA. Mature miRNA molecules are partially complementary to one or more messenger RNA (mRNA) molecules, and their main function is to downregulate gene expression.

Certain RNA inhibiting agents may be utilized to inhibit the expression or translation of messenger RNA (“mRNA”) that is associated with a cancer phenotype. Examples of such agents suitable for use herein include, but are not limited to, short interfering RNA (“siRNA”), ribozymes, and antisense oligonucleotides. Specific examples of RNA inhibiting agents suitable for use herein include, but are not limited to, Cand5, Sirna-027, fomivirsen, and angiozyme.

### Small Molecule Enzymatic Inhibitors

Certain small molecule therapeutic agents are able to target the tyrosine kinase enzymatic activity or downstream signal transduction signals of certain cell receptors such as epidermal growth factor receptor (“EGFR”) or vascular endothelial growth factor receptor (“VEGFR”). Such targeting by small molecule therapeutics can result in anti-cancer effects. Examples of such agents suitable for use herein include, but are not limited to, imatinib, gefitinib, erlotinib, lapatinib, canertinib, ZD6474, sorafenib (BAY 43-9006), ERB-569, and their analogues and derivatives.

### Biological Response Modifiers

Certain protein or small molecule agents can be used in anti-cancer therapy through either direct anti-tumor effects or through indirect effects. Examples of direct-acting agents suitable for use herein include, but are not limited to, differentiating reagents such as retinoids and retinoid derivatives. Indirect-acting agents suitable for use herein include, but are not limited to, agents that modify or enhance the immune or other systems such as interferons, interleukins, hematopoietic growth factors (e.g. erythropoietin), and antibodies (monoclonal and polyclonal).

### Anti-Metastatic Agents

The process whereby cancer cells spread from the site of the original tumor to other locations around the body is termed cancer metastasis. Certain agents have anti-metastatic properties, designed to inhibit the spread of cancer cells. Examples of such agents suitable for use herein include, but are not limited to, marimastat, bevacizumab, trastuzumab, rituximab, erlotinib, MMI-166, GRN163L, hunter-killer peptides, tissue inhibitors of metalloproteinases (TIMPs), their analogues, derivatives and variants.

### Chemopreventative Agents

Certain pharmaceutical agents can be used to prevent initial occurrences of cancer, or to prevent recurrence or metastasis. Administration with such chemopreventative agents in combination with one or more other anticancer agents including the benzopyrone compounds can act to both treat and prevent the recurrence of cancer. Examples of chemopreventative agents suitable for use herein include, but are not limited to, tamoxifen, raloxifene, tibolone, bisphosphonate, ibandronate, estrogen receptor modulators, aromatase inhibitors (letrozole, anastrozole), luteinizing hormone-releasing hormone agonists, goserelin, vitamin A, retinal, retinoic acid, fenretinide, 9-cis-retinoid acid, 13-cis-retinoid acid, all-trans-retinoic acid, isotretinoin, tretinoid, vitamin B6, vitamin B12, vitamin C, vitamin D, vitamin E, cyclooxygenase inhibitors, non-steroidal anti-inflammatory drugs (NSAIDs), aspirin, ibuprofen, celecoxib, polyphenols, polyphenol E, green tea extract, folic acid, glucaric acid, interferon-alpha, anethole dithiolethione, zinc, pyridoxine, finasteride, doxazosin, selenium, indole-3-carbinal, alpha-difluoromethylomithine, carotenoids, beta-carotene, lycopene, antioxidants, coenzyme Q10, flavonoids, quercetin, curcumin, catechins, epigallocatechin gallate, N-acetylcysteine, indole-3-carbinol, inositol hexaphosphate, isoflavones, glucanic acid, rosemary, soy, saw palmetto, and calcium. An additional example of chemopreventative agents suitable for use in the present invention is cancer vaccines. These can be created through immunizing a patient with all or part of a cancer cell type that is targeted by the vaccination process.

### Side-Effect Limiting Agents

Treatment of cancer with benzopyrone compounds alone or in combination with other antineoplastic compounds may be accompanied by administration of pharmaceutical agents that can alleviate the side effects produced by the antineoplastic agents. Such agents suitable for use herein include, but are not limited to, anti-emetics, anti-mucositis agents, pain management agents, infection control agents, and anti-anemia/anti-thrombocytopenia agents. Examples of anti-emetics suitable for use herein include, but are not limited to, 5-hydroxytryptamine 3 receptor antagonists, metoclopramide, steroids, lorazepam, ondansetron, cannabinoids, their analogues and derivatives. Examples of anti-mucositis agents suitable for use herein include, but are not limited to, palifermin (keratinocyte growth factor), glucagon-like peptide-2, teduglutide, L-glutamine, amifostin, and fibroblast growth factor 20. Examples of pain management agents suitable for use herein include, but are not limited to, opioids, opiates, and non-steroidal anti-inflammatory compounds. Examples of agents used for control of infection suitable for use herein include, but are not limited to, antibacterials such as aminoglycosides, penicillins, cephalosporins, tetracyclines, clindamycin, lincomycin, macrolides, vancomycin, carbapenems, monobactams, fluoroquinolones, sulfonamides, nitrofurantoins, their analogues and derivatives. Examples of agents that can treat anemia or thrombocytopenia associated with chemotherapy suitable for use herein include, but are not limited to, erythropoietin, and thrombopoietin.

Several other suitable therapies for use in combination with the benzopyrone compounds and other compounds described herein are also available. For example, see Goodman & Gilman's The Pharmacological Basis of Therapeutics 11th ed. Brunton LL, Lazo JS, and Parker KL, ed. McGraw-Hill, New York, 2006.

### Formulations, Routes of Administration, and Effective Doses

Another aspect of the present invention relates to formulations and routes of administration for pharmaceutical compositions comprising a benzopyrone compound. In some embodiments, the pharmaceutical composition comprises one or more benzopyrone compounds. In other embodiments, the pharmaceutical composition comprises one or more benzopyrone compounds in combination with one or more antineoplastic anti-tumor agents. Such pharmaceutical compositions can be used to treat cancer in the methods described in detail above.

The compounds of formula II, for example, 6-nitro-5-iodo-benzopyrone, may be provided for in vivo administration. Either the benzopyrone form or pharmaceutically acceptable salts may be used in developing a formulation for use in the present invention. Further, in some embodiments, the compound may be used in combination with one or more other compounds or in one or more other forms. For example a formulation may comprise both the benzopyrone compound and acid forms in particular proportions, depending on the relative potencies of each and the intended indication. The two forms may be formulated together, in the same dosage unit e.g. in one cream, suppository, tablet, capsule, or packet of powder to be dissolved in a beverage; or each form may be formulated in a separate unit, e.g., two creams, two suppositories, two tablets, two capsules, a tablet and a liquid for dissolving the tablet, a packet of powder and a liquid for dissolving the powder, etc.

In compositions comprising combinations of a benzopyrone compound and another active agent, for example, an antineoplastic anti-tumor agent, can be effective. The two compounds and/or forms of a compound may be formulated together, in the same dosage unit e.g. in one cream, suppository, tablet, capsule, or packet of powder to be dissolved in a beverage; or each form may be formulated in separate units, e.g., two creams, suppositories, tablets, two capsules, a tablet and a liquid for dissolving the tablet, a packet of powder and a liquid for dissolving the powder, etc.

The term “pharmaceutically acceptable salt” means those salts which retain the biological effectiveness and properties of the compounds used in the present invention, and which are not biologically or otherwise undesirable. For example, a pharmaceutically acceptable salt does not interfere with the beneficial effect of the compound of the invention in treating a cancer.

Typical salts are those of the inorganic ions, such as, for example, sodium, potassium, calcium and magnesium ions. Such salts include salts with inorganic or organic acids, such as hydrochloric acid, hydrobromic acid, phosphoric acid, nitric acid, sulfuric acid, methanesulfonic acid, p-toluenesulfonic acid, acetic acid, fumaric acid, succinic acid, lactic acid, mandelic acid, malic acid, citric acid, tartaric acid or maleic acid. In addition, if the compounds used in the present invention contain a carboxy group or other acidic group, it may be converted into a pharmaceutically acceptable addition salt with inorganic or organic bases. Examples of suitable bases include sodium hydroxide, potassium hydroxide, ammonia, cyclohexylamine, dicyclohexyl-amine, ethanolamine, diethanolamine and triethanolamine.

For oral administration, the compounds can be formulated readily by combining the active compound(s) with pharmaceutically acceptable carriers well known in the art. Such carriers enable the compounds of the invention to be formulated as tablets, including chewable tablets, pills, dragees, capsules, lozenges, hard candy, liquids, gels, syrups, slurries, powders, suspensions, elixirs, wafers, and the like, for oral ingestion by a patient to be treated. Such formulations can comprise pharmaceutically acceptable carriers including solid diluents or fillers, sterile aqueous media and various non-toxic organic solvents. Generally, the compounds of the invention will be included at concentration levels ranging from about 0.5%, about 5%, about 10%, about 20%, or about 30% to about 50%, about 60%, about 70%, about 80% or about 90% by weight of the total composition of oral dosage forms, in an amount sufficient to provide a desired unit of dosage.

Aqueous suspensions may contain a benzopyrone compound with pharmaceutically acceptable excipients, such as a suspending agent (e.g., methyl cellulose), a wetting agent (e.g., lecithin, lysolecithin and/or a long-chain fatty alcohol), as well as coloring agents, preservatives, flavoring agents, and the like.

In some embodiments, oils or non-aqueous solvents may be required to bring the compounds into solution, due to, for example, the presence of large lipophilic moieties. Alternatively, emulsions, suspensions, or other preparations, for example, liposomal preparations, may be used. With respect to liposomal preparations, any known methods for preparing liposomes for treatment of a condition may be used. See, for example, Bangham et al., J. Mol. Biol, 23: 238-252 (1965) and Szoka et al., Proc. Natl. Acad. Sci. 75: 4194-4198 (1978), incorporated herein by reference. Ligands may also be attached to the liposomes to direct these compositions to particular sites of action. Compounds of this invention may also be integrated into foodstuffs, e.g., cream cheese, butter, salad dressing, or ice cream to facilitate solubilization, administration, and/or compliance in certain patient populations.

Pharmaceutical preparations for oral use may be obtained as a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; flavoring elements, cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl pyrrolidone (PVP). If desired, disintegrating agents may be added, such as the cross-linked polyvinyl pyrrolidone, agar, or alginic acid or a salt thereof such as sodium alginate. The compounds may also be formulated as a sustained release preparation.

Dragee cores can be provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for administration.

For injection, the inhibitors of the present invention may be formulated in aqueous solutions, preferably in physiologically compatible buffers such as Hank's solution, Ringer's solution, or physiological saline buffer. Such compositions may also include one or more excipients, for example, preservatives, solubilizers, fillers, lubricants, stabilizers, albumin, and the like. Methods of formulation are known in the art, for example, as disclosed in Remington's Pharmaceutical Sciences, latest edition, Mack Publishing Co., Easton P. These compounds may also be formulated for transmucosal administration, buccal administration, for administration by inhalation, for parental administration, for transdermal administration, and rectal administration.

In addition to the formulations described previously, the compounds may also be formulated as a depot preparation. Such long acting formulations may be administered by implantation or transcutaneous delivery (for example subcutaneously or intramuscularly), intramuscular injection or use of a transdermal patch. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Pharmaceutical compositions suitable for use in the present invention include compositions wherein the active ingredients are present in an effective amount, i.e., in an amount effective to achieve therapeutic and/or prophylactic benefit in at least one of the cancers described herein. The actual amount effective for a particular application will depend on the condition or conditions being treated, the condition of the subject, the formulation, and the route of administration, as well as other factors known to those of skill in the art. Determination of an effective amount of a benzopyrone compound is well within the capabilities of those skilled in the art, in light of the disclosure herein, and will be determined using routine optimization techniques.

### Clinical Efficacy

Clinical efficacy may be measured by any method known in the art. In some embodiments, clinical efficacy of the combination of benzopyrone compounds and anti-neoplastic anti-tumor agents (e.g. gemcitabine, and oxaliplatin) may be determined by measuring the clinical benefit rate (CBR). In other embodiments, clinical efficacy of the benzopyrone compounds may be determined by measuring the clinical benefit rate (CBR).

The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD≧6 months. The CBR for combination therapy with gemcitabine and oxaliplatin is 55% (Demols A, et. al., British J. of Cancer, vol. 94, 2006). Thus, the CBR for triple combination therapy with a benzopyrone, (e.g. GEM, OX and 6-nitro-5-iodo-benzopyrone) may be compared to that of the double combination therapy with GEM and OX. In some embodiments, CBR for triple combination therapy is at least about 60%.

In some embodiments disclosed herein, the methods include pre-determining that a cancer is treatable by PARP modulators. Some such methods comprise identifying a level of PARP in a pancreatic cancer sample of a patient, determining whether the level of PARP expression in the sample is greater than a pre-determined value, and, if the PARP expression is greater than said predetermined value, treating the patient with a combination of a benzopyrone compound with or without one or more anti-tumor agents (e.g. OX and/or GEM).

Germline mutations in the tumor suppressor genes breast cancer antigen gene (BRCA)1 and BRCA2 have been proven to portend a drastically increased lifetime risk of breast and ovarian cancers in the individuals who carry them. A number of studies have shown that the third most common cancer associated with these mutations is pancreatic cancer. Pancreatic tumors in people who inherit faults in either the BRCA1 or BRCA2 genes occur because the tumor cells have lost a specific mechanism that repair damaged DNA. BRCA1 and BRCA2 are important for DNA double-strand break repair by homologous recombination, and mutations in these genes predispose to uterine and other cancers. PARP is involved in base excision repair, a pathway in the repair of DNA single-strand breaks. BRCA1 or BRCA2 dysfunction sensitizes cells to the inhibition of PARP enzymatic activity, resulting in chromosomal instability, cell cycle arrest and subsequent apoptosis. In vivo study has demonstrated a significantly reduced tumor weight and increased survival up to 40 days after inoculation of human pancreatic cancer cells with combination therapy of PARP inhibitor 3-aminobenzamide (3-ABA) and gemcitabine, as compared to animals treated with PBS, gemcitabine or 3-ABA alone (Dietmar A, et. al, Journal of gastroenterology and hepatology, 2007, vol. 22).

PARP inhibitors kill cells where this form of DNA repair is absent; and thus are effective in killing BRCA deficient tumor cells and other similar tumor cells. Normal cells may be unaffected by the drug as they may still possess this DNA repair mechanism. This treatment might also be applicable to other forms of uterine cancer that behave like BRCA deficient cancer. Typically, uterine cancer patients are treated with drugs that kill tumor cells but also damage normal cells. It is damage to normal cells that can lead to distressing side effects, like nausea and hair loss. In some embodiments, an advantage of treating with PARP inhibitors is that it is targeted therapy: tumor cells are killed while normal cells appear unaffected. This is because PARP inhibitors exploit the specific genetic make-up of some tumor cells.

Patients deficient in BRCA genes have up-regulated levels of PARP. PARP up-regulation may be an indicator of other defective DNA-repair pathways and unrecognized BRCA-like genetic defects. Assessment of PARP-1 gene expression is an indicator of tumor sensitivity to PARP inhibitor. Hence, in some embodiments, treatment of uterine cancer can be enhanced not only by determining the HR and/or HER2 status of the cancer, but also by identifying early onset of cancer in BRCA deficient patients by measuring the level of PARP. The BRCA deficient patients treatable by PARP inhibitors can be identified if PARP is up-regulated. Further, such BRCA deficient patients can be treated with PARP inhibitors.

In some embodiments, a sample is collected from a patient having a pancreatic lesion suspected of being cancerous. While such sample may be any available biological tissue, in most cases the sample will be a portion of the suspected pancreatic lesion, whether obtained by laparoscopy or open surgery (e.g. hysterectomy). PARP expression may then be analyzed and, if the PARP expression is above a predetermined level (e.g. is up-regulated vis-à-vis normal tissue) the patient may be treated with a PARP-1 inhibitor, such as a benzopyrone compound, either alone or in combination with one or more anti-tumor agents. It is thus to be understood that, while embodiments described herein are directed to treatment of negative pancreatic cancers, in some embodiments the pancreatic cancer need not be negative so long as the threshold PARP up-regulation is satisfied.

In some embodiments, tumors that are homologous recombination deficient are identified by evaluating levels of PARP expression. If up-regulation of PARP is observed, such tumors can be treated with PARP inhibitors. Another embodiment is a method for treating a homologous recombination deficient cancer comprising evaluating level of PARP expression and, if overexpression is observed, the cancer is treated with a PARP inhibitor.

Ovarian tumors in women who inherit faults in either the BRCA1 or BRCA2 genes occur because the tumor cells have lost a specific mechanism that repair damaged DNA. BRCA1 and BRCA2 are important for DNA double-strand break repair by homologous recombination, and mutations in these genes predispose to uterine and other cancers. PARP is involved in base excision repair, a pathway in the repair of DNA single-strand breaks. BRCA1 or BRCA2 dysfunction sensitizes cells to the inhibition of PARP enzymatic activity, resulting in chromosomal instability, cell cycle arrest and subsequent apoptosis.

PARP inhibitors kill cells where this form of DNA repair is absent; and thus are effective in killing BRCA deficient tumor cells and other similar tumor cells. Normal cells may be unaffected by the drug as they may still possess this DNA repair mechanism. This treatment might also be applicable to other forms of uterine cancer that behave like BRCA deficient cancer. Typically, uterine cancer patients are treated with drugs that kill tumor cells but also damage normal cells. It is damage to normal cells that can lead to distressing side effects, like nausea and hair loss. In some embodiments, an advantage of treating with PARP inhibitors is that it is targeted therapy: tumor cells are killed while normal cells appear unaffected. This is because PARP inhibitors exploit the specific genetic make-up of some tumor cells.

Patients deficient in BRCA genes have up-regulated levels of PARP. PARP up-regulation may be an indicator of other defective DNA-repair pathways and unrecognized BRCA-like genetic defects. Assessment of PARP-1 gene expression is an indicator of tumor sensitivity to PARP inhibitor. Hence, in some embodiments, treatment of ovarian cancer can be enhanced not only by determining the HR and/or HER2 status of the cancer, but also by identifying early onset of cancer in BRCA deficient patients by measuring the level of PARP. The BRCA deficient patients treatable by PARP inhibitors can be identified if PARP is up-regulated. Further, such BRCA deficient patients can be treated with PARP inhibitors.

In some embodiments, a sample is collected from a patient having an ovarian lesion suspected of being cancerous. While such sample may be any available biological tissue, in most cases the sample will be a portion of the suspected ovarian lesion, whether obtained by laparoscopy or open surgery (e.g. hysterectomy). PARP expression may then be analyzed and, if the PARP expression is above a predetermined level (e.g. is up-regulated vis-à-vis normal tissue) the patient may be treated with a PARP-1 inhibitor, such as a benzopyrone compound, either alone or in combination with one or more anti-tumor agents such as OX and GEM. It is thus to be understood that, while embodiments described herein are directed to treatment of negative ovarian cancer, in some embodiments the ovarian cancer need not be negative so long as the threshold PARP up-regulation is satisfied.

In some embodiments, tumors that are homologous recombination deficient are identified by evaluating levels of PARP expression. If up-regulation of PARP is observed, such tumors can be treated with PARP inhibitors. Another embodiment is a method for treating a homologous recombination deficient cancer comprising evaluating level of PARP expression and, if overexpression is observed, the cancer is treated with a PARP inhibitor.

Uterine tumors in women who inherit faults in either the BRCA1 or BRCA2 genes occur because the tumor cells have lost a specific mechanism that repair damaged DNA. BRCA1 and BRCA2 are important for DNA double-strand break repair by homologous recombination, and mutations in these genes predispose to uterine and other cancers. PARP is involved in base excision repair, a pathway in the repair of DNA single-strand breaks. BRCA1 or BRCA2 dysfunction sensitizes cells to the inhibition of PARP enzymatic activity, resulting in chromosomal instability, cell cycle arrest and subsequent apoptosis.

PARP inhibitors kill cells where this form of DNA repair is absent; and thus are effective in killing BRCA deficient tumor cells and other similar tumor cells. Normal cells may be unaffected by the drug as they may still possess this DNA repair mechanism. This treatment might also be applicable to other forms of uterine cancer that behave like BRCA deficient cancer. Typically, uterine cancer patients are treated with drugs that kill tumor cells but also damage normal cells. It is damage to normal cells that can lead to distressing side effects, like nausea and hair loss. In some embodiments, an advantage of treating with PARP inhibitors is that it is targeted therapy: tumor cells are killed while normal cells appear unaffected. This is because PARP inhibitors exploit the specific genetic make-up of some tumor cells.

Patients deficient in BRCA genes have up-regulated levels of PARP. PARP up-regulation may be an indicator of other defective DNA-repair pathways and unrecognized BRCA-like genetic defects. Assessment of PARP-1 gene expression is an indicator of tumor sensitivity to PARP inhibitor. Hence, in some embodiments, treatment of uterine cancer can be enhanced not only by determining the HR and/or HER2 status of the cancer, but also by identifying early onset of cancer in BRCA deficient patients by measuring the level of PARP. The BRCA deficient patients treatable by PARP inhibitors can be identified if PARP is up-regulated. Further, such BRCA deficient patients can be treated with PARP inhibitors.

In some embodiments, a sample is collected from a patient having a uterine lesion suspected of being cancerous. While such sample may be any available biological tissue, in most cases the sample will be a portion of the suspected uterine lesion, whether obtained by laparoscopy or open surgery (e.g. hysterectomy). PARP expression may then be analyzed and, if the PARP expression is above a predetermined level (e.g. is up-regulated vis-à-vis normal tissue) the patient may be treated with a PARP-1 inhibitor, such as a benzopyrone compound, either alone or in combination with one or more anti-tumor agents such as OX and GEM. It is thus to be understood that, while embodiments described herein are directed to treatment of so-called triple negative metastatic uterine cancer, in some embodiments the uterine cancer need not be triple negative so long as the threshold PARP up-regulation is satisfied.

In some embodiments, tumors that are homologous recombination deficient are identified by evaluating levels of PARP expression. If up-regulation of PARP is observed, such tumors can be treated with PARP inhibitors. Another embodiment is a method for treating a homologous recombination deficient cancer comprising evaluating level of PARP expression and, if overexpression is observed, the cancer is treated with a PARP inhibitor.

Breast tumors in women who inherit faults in either the BRCA 1 or BRCA2 genes occur because the tumor cells have lost a specific mechanism that repair damaged DNA. BRCA1 and BRCA2 are important for DNA double-strand break repair by homologous recombination, and mutations in these genes predispose to breast and other cancers. PARP is involved in base excision repair, a pathway in the repair of DNA single-strand breaks. BRCA1 or BRCA2 dysfunction sensitizes cells to the inhibition of PARP enzymatic activity, resulting in chromosomal instability, cell cycle arrest and subsequent apoptosis.

PARP inhibitors kill cells where this form of DNA repair is absent; and thus are effective in killing BRCA deficient tumor cells and other similar tumor cells. Normal cells may be unaffected by the drug as they may still possess this DNA repair mechanism. This treatment might also be applicable to other forms of breast cancer that behave like BRCA deficient cancer. Typically, breast cancer patients are treated with drugs that kill tumor cells but also damage normal cells. It is damage to normal cells that can lead to distressing side effects, like nausea and hair loss. In some embodiments, an advantage of treating with PARP inhibitors is that it is targeted therapy: tumor cells are killed while normal cells appear unaffected. This is because PARP inhibitors exploit the specific genetic make-up of some tumor cells.

Patients deficient in BRCA genes have up-regulated levels of PARP. PARP up-regulation may be an indicator of other defective DNA-repair pathways and unrecognized BRCA-like genetic defects. Assessment of PARP-1 gene expression is an indicator of tumor sensitivity to PARP inhibitor. Hence, in some embodiments, treatment of metastatic breast cancer can be enhanced not only by determining the HR and/or HER2 status of the cancer, but also by identifying early onset of cancer in BRCA deficient patients by measuring the level of PARP. The BRCA deficient patients treatable by PARP inhibitors can be identified if PARP is up-regulated. Further, such BRCA deficient patients can be treated with PARP inhibitors.

In some embodiments, a sample is collected from a patient having a breast lesion suspected of being cancerous. While such sample may be any available biological tissue, in most cases the sample will be a portion of the suspected breast lesion, whether obtained by minimally invasive biopsy or by therapeutic surgery (e.g. lumpectomy, mastectomy, partial or modified mastectomy or radical mastectomy). Such sample may also include all or part of one or more lymph nodes extracted during mastectomy. PARP expression may then be analyzed and, if the PARP expression is above a predetermined level (e.g. is up-regulated vis-à-vis normal tissue) the patient may be treated with a PARP-1 inhibitor, such as a benzopyrone, in combination with one or more anti-tumor agents such as OX and GEM. It is thus to be understood that, while embodiments described herein are directed to treatment of so-called triple negative metastatic breast cancer, in some embodiments the breast cancer need not be triple negative so long as the threshold PARP up-regulation is satisfied.

In some embodiments, tumors that are homologous recombination deficient are identified by evaluating levels of PARP expression. If up-regulation of PARP is observed, such tumors can be treated with PARP inhibitors. Another embodiment is a method for treating a homologous recombination deficient cancer comprising evaluating level of PARP expression and, if overexpression is observed, the cancer is treated with a PARP inhibitor.

### Sample Collection, Preparation and Separation

Biological samples may be collected from a variety of sources from a patient including a body fluid sample, or a tissue sample. Samples collected can be human normal and tumor samples, nipple aspirants. The samples can be collected from individuals repeatedly over a longitudinal period of time (e.g., about once a day, once a week, once a month, biannually or annually). Obtaining numerous samples from an individual over a period of time can be used to verify results from earlier detections and/or to identify an alteration in biological pattern as a result of, for example, disease progression, drug treatment, etc.

Sample preparation and separation can involve any of the procedures, depending on the type of sample collected and/or analysis of PARP. Such procedures include, by way of example only, concentration, dilution, adjustment of pH, removal of high abundance polypeptides (e.g., albumin, gamma globulin, and transferin, etc.), addition of preservatives and calibrants, addition of protease inhibitors, addition of denaturants, desalting of samples, concentration of sample proteins, extraction and purification of lipids.

The sample preparation can also isolate molecules that are bound in non-covalent complexes to other protein (e.g., carrier proteins). This process may isolate those molecules bound to a specific carrier protein (e.g., albumin), or use a more general process, such as the release of bound molecules from all carrier proteins via protein denaturation, for example using an acid, followed by removal of the carrier proteins.

Removal of undesired proteins (e.g., high abundance, uninformative, or undetectable proteins) from a sample can be achieved using high affinity reagents, high molecular weight filters, ultracentrifugation and/or electrodialysis. High affinity reagents include antibodies or other reagents (e.g. aptamers) that selectively bind to high abundance proteins. Sample preparation could also include ion exchange chromatography, metal ion affinity chromatography, gel filtration, hydrophobic chromatography, chromatofocusing, adsorption chromatography, isoelectric focusing and related techniques. Molecular weight filters include membranes that separate molecules on the basis of size and molecular weight. Such filters may further employ reverse osmosis, nanofiltration, ultrafiltration and microfiltration.

Ultracentrifugation is a method for removing undesired polypeptides from a sample. Ultracentrifugation is the centrifugation of a sample at about 15,000-60,000 rpm while monitoring with an optical system the sedimentation (or lack thereof) of particles. Electrodialysis is a procedure which uses an electromembrane or semipermable membrane in a process in which ions are transported through semi-permeable membranes from one solution to another under the influence of a potential gradient. Since the membranes used in electrodialysis may have the ability to selectively transportions having positive or negative charge, reject ions of the opposite charge, or to allow species to migrate through a semipermable membrane based on size and charge, it renders electrodialysis useful for concentration, removal, or separation of electrolytes.

Separation and purification in the present invention may include any procedure known in the art, such as capillary electrophoresis (e.g., in capillary or on-chip) or chromatography (e.g., in capillary, column or on a chip). Electrophoresis is a method which can be used to separate ionic molecules under the influence of an electric field. Electrophoresis can be conducted in a gel, capillary, or in a microchannel on a chip. Examples of gels used for electrophoresis include starch, acrylamide, polyethylene oxides, agarose, or combinations thereof. A gel can be modified by its cross-linking, addition of detergents, or denaturants, immobilization of enzymes or antibodies (affinity electrophoresis) or substrates (zymography) and incorporation of a pH gradient. Examples of capillaries used for electrophoresis include capillaries that interface with an electrospray.

Capillary electrophoresis (CE) is preferred for separating complex hydrophilic molecules and highly charged solutes. CE technology can also be implemented on microfluidic chips. Depending on the types of capillary and buffers used, CE can be further segmented into separation techniques such as capillary zone electrophoresis (CZE), capillary isoelectric focusing (CIEF), capillary isotachophoresis (cITP) and capillary electrochromatography (CEC). An embodiment to couple CE techniques to electrospray ionization involves the use of volatile solutions, for example, aqueous mixtures containing a volatile acid and/or base and an organic such as an alcohol or acetonitrile.

Capillary isotachophoresis (cITP) is a technique in which the analytes move through the capillary at a constant speed but are nevertheless separated by their respective mobilities. Capillary zone electrophoresis (CZE), also known as free-solution CE (FSCE), is based on differences in the electrophoretic mobility of the species, determined by the charge on the molecule, and the frictional resistance the molecule encounters during migration which is often directly proportional to the size of the molecule. Capillary isoelectric focusing (CIEF) allows weakly-ionizable amphoteric molecules, to be separated by electrophoresis in a pH gradient. CEC is a hybrid technique between traditional high performance liquid chromatography (HPLC) and CE.

Separation and purification techniques used in the present invention include any chromatography procedures known in the art. Chromatography can be based on the differential adsorption and elution of certain analytes or partitioning of analytes between mobile and stationary phases. Different examples of chromatography include, but not limited to, liquid chromatography (LC), gas chromatography (GC), high performance liquid chromatography (HPLC) etc.

### Identifying Level of PARP

The poly (ADP-ribose) polymerase (PARP) is also known as poly (ADP-ribose) synthase and poly ADP-ribosyltransferase. PARP catalyzes the formation of poly (ADP-ribose) polymers which can attach to nuclear proteins (as well as to itself) and thereby modify the activities of those proteins. The enzyme plays a role in enhancing DNA repair, but it also plays a role in regulating chromatin in the nuclei (for review see: D. D'amours et al. “Poly (ADP-ribosylation reactions in the regulation of nuclear functions,” Biochem. J. 342: 249-268 (1999)).

PARP-1 comprises an N-terminal DNA binding domain, an automodification domain and a C-terminal catalytic domain and various cellular proteins interact with PARP-1. The N-terminal DNA binding domain contains two zinc finger motifs. Transcription enhancer factor-1 (TEF-1), retinoid X receptor α, DNA polymerase α, X-ray repair cross-complementing factor-1 (XRCC1) and PARP-1 itself interact with PARP-1 in this domain. The automodification domain contains a BRCT motif, one of the protein-protein interaction modules. This motif is originally found in the C-terminus of BRCA1 (uterine cancer susceptibility protein 1) and is present in various proteins related to DNA repair, recombination and cell-cycle checkpoint control. POU-homeodomain-containing octamer transcription factor-1 (Oct-1), Yin Yang (YY)1 and ubiquitin-conjugating enzyme 9 (ubc9) could interact with this BRCT motif in PARP-1.

More than 15 members of the PARP family of genes are present in the mammalian genome. PARP family proteins and poly(ADP-ribose) glycohydrolase (PARG), which degrades poly(ADP-ribose) to ADP-ribose, could be involved in a variety of cell regulatory functions including DNA damage response and transcriptional regulation and may be related to carcinogenesis and the biology of cancer in many respects.

Several PARP family proteins have been identified. Tankyrase has been found as an interacting protein of telomere regulatory factor 1 (TRF-1) and is involved in telomere regulation. Vault PARP (VPARP) is a component in the vault complex, which acts as a nuclear-cytoplasmic transporter. PARP-2, PARP-3 and 2,3,7,8-tetrachlorodibenzo-p-dioxin inducible PARP (TiPARP) have also been identified. Therefore, poly (ADP-ribose) metabolism could be related to a variety of cell regulatory functions.

A member of this gene family is PARP-1. The PARP-1 gene product is expressed at high levels in the nuclei of cells and is dependent upon DNA damage for activation. Without being bound by any theory, it is believed that PARP-1 binds to DNA single or double stranded breaks through an amino terminal DNA binding domain. The binding activates the carboxy terminal catalytic domain and results in the formation of polymers of ADP-ribose on target molecules. PARP-1 is itself a target of poly ADP-ribosylation by virtue of a centrally located automodification domain. The ribosylation of PARP-1 causes dissociation of the PARP-1 molecules from the DNA. The entire process of binding, ribosylation, and dissociation occurs very rapidly. It has been suggested that this transient binding of PARP-1 to sites of DNA damage results in the recruitment of DNA repair machinery or may act to suppress the recombination long enough for the recruitment of repair machinery.

The source of ADP-ribose for the PARP reaction is nicotinamide adenosine dinucleotide (NAD). NAD is synthesized in cells from cellular ATP stores and thus high levels of activation of PARP activity can rapidly lead to depletion of cellular energy stores. It has been demonstrated that induction of PARP activity can lead to cell death that is correlated with depletion of cellular NAD and ATP pools. PARP activity is induced in many instances of oxidative stress or during inflammation. For example, during reperfusion of ischemic tissues reactive nitric oxide is generated and nitric oxide results in the generation of additional reactive oxygen species including hydrogen peroxide, peroxynitrate and hydroxyl radical. These latter species can directly damage DNA and the resulting damage induces activation of PARP activity. Frequently, it appears that sufficient activation of PARP activity occurs such that the cellular energy stores are depleted and the cell dies. A similar mechanism is believed to operate during inflammation when endothelial cells and pro-inflammatory cells synthesize nitric oxide which results in oxidative DNA damage in surrounding cells and the subsequent activation of PARP activity. The cell death that results from PARP activation is believed to be a major contributing factor in the extent of tissue damage that results from ischemia-reperfusion injury or from inflammation.

In some embodiments, the level of PARP in a sample from a patient is compared to predetermined standard sample. The sample from the patient is typically from a diseased tissue, such as cancer cells or tissues. The standard sample can be from the same patient or from a different subject. The standard sample is typically a normal, non-diseased sample. However, in some embodiments, such as for staging of disease or for evaluating the efficacy of treatment, the standard sample is from a diseased tissue. The standard sample can be a combination of samples from several different subjects. In some embodiments, the level of PARP from a patient is compared to a pre-determined level. This pre-determined level is typically obtained from normal samples. As described herein, a “pre-determined PARP level” may be a level of PARP used to, by way of example only, evaluate a patient that may be selected for treatment, evaluate a response to a PARP inhibitor treatment, evaluate a response to a combination of a PARP inhibitor and a second therapeutic agent treatment, and/or diagnose a patient for cancer, inflammation, pain and/or related conditions. A pre-determined PARP level may be determined in populations of patients with or without cancer. The pre-determined PARP level can be a single number, equally applicable to every patient, or the pre-determined PARP level can vary according to specific subpopulations of patients. For example, men might have a different pre-determined PARP level than women; non-smokers may have a different pre-determined PARP level than smokers. Age, weight, and height of a patient may affect the pre-determined PARP level of the individual. Furthermore, the pre-determined PARP level can be a level determined for each patient individually. The pre-determined PARP level can be any suitable standard. For example, the pre-determined PARP level can be obtained from the same or a different human for whom a patient selection is being assessed. In one embodiment, the pre-determined PARP level can be obtained from a previous assessment of the same patient. In such a manner, the progress of the selection of the patient can be monitored over time. In addition, the standard can be obtained from an assessment of another human or multiple humans, e.g., selected groups of humans. In such a manner, the extent of the selection of the human for whom selection is being assessed can be compared to suitable other humans, e.g., other humans who are in a similar situation to the human of interest, such as those suffering from similar or the same condition(s).

In some embodiments of the present invention the change of PARP from the pre-determined level is about 0.5 fold, about 1.0 fold, about 1.5 fold, about 2.0 fold, about 2.5 fold, about 3.0 fold, about 3.5 fold, about 4.0 fold, about 4.5 fold, or about 5.0 fold. In some embodiments is fold change is less than about 1, less than about 5, less than about 10, less than about 20, less than about 30, less than about 40, or less than about 50. In other embodiments, the changes in PARP level compared to a predetermined level is more than about 1, more than about 5, more than about 10, more than about 20, more than about 30, more than about 40, or more than about 50. Preferred fold changes from a pre-determined level are about 0.5, about 1.0, about 1.5, about 2.0, about 2.5, and about 3.0.

The analysis of PARP levels in patients is particularly valuable and informative, as it allows the physician to more effectively select the best treatments, as well as to utilize more aggressive treatments and therapy regimens based on the up-regulated or down-regulated level of PARP. More aggressive treatment, or combination treatments and regimens, can serve to counteract poor patient prognosis and overall survival time. Armed with this information, the medical practitioner can choose to provide certain types of treatment such as treatment with PARP inhibitors, and/or more aggressive therapy.

In monitoring a patient's PARP levels, over a period of time, which may be days, weeks, months, and in some cases, years, or various intervals thereof, the patient's body fluid sample, e.g., serum or plasma, can be collected at intervals, as determined by the practitioner, such as a physician or clinician, to determine the levels of PARP, and compared to the levels in normal individuals over the course or treatment or disease. For example, patient samples can be taken and monitored every month, every two months, or combinations of one, two, or three month intervals according to the invention. In addition, the PARP levels of the patient obtained over time can be conveniently compared with each other, as well as with the PARP values, of normal controls, during the monitoring period, thereby providing the patient's own PARP values, as an internal, or personal, control for long-term PARP monitoring.

### Compositions

In another aspect, the present invention provides a composition for the treatment of a cancer, the composition comprising compound of formula I, or a pharmaceutically acceptable salt or prodrug thereof:

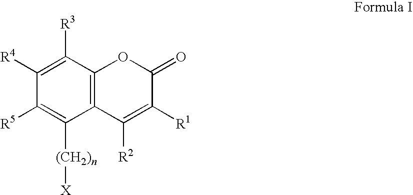

wherein n=0-10; R¹, R², R³, R⁴, R⁵ and X are independently selected from the group consisting of hydrogen, hydroxy, optionally substituted amine, amino, carboxyl, ester, nitroso, nitro, halogen, optionally substituted (C₁-C₆) alkyl, optionally substituted (C₁-C₆) alkoxy, optionally substituted (C₃-C₇) cycloalkyl, optionally substituted (C₃-C₇) heterocyclic, phenyl, and optionally substituted aryl; and wherein at least two of the R¹, R², R³, R⁴, and R⁵ substituents are always hydrogen; and

wherein the compound is not one of the following:

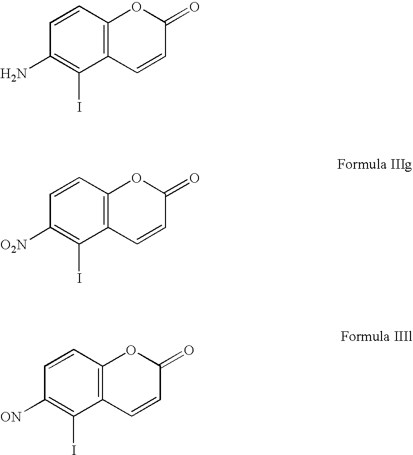

In some embodiments, the compound is of the formula IIIa, IIIb, IIIc, IIId, IIIe, IIIf, IIIh, IIIm, or IIIn, or one of their pharmaceutically acceptable salts or prodrugs:

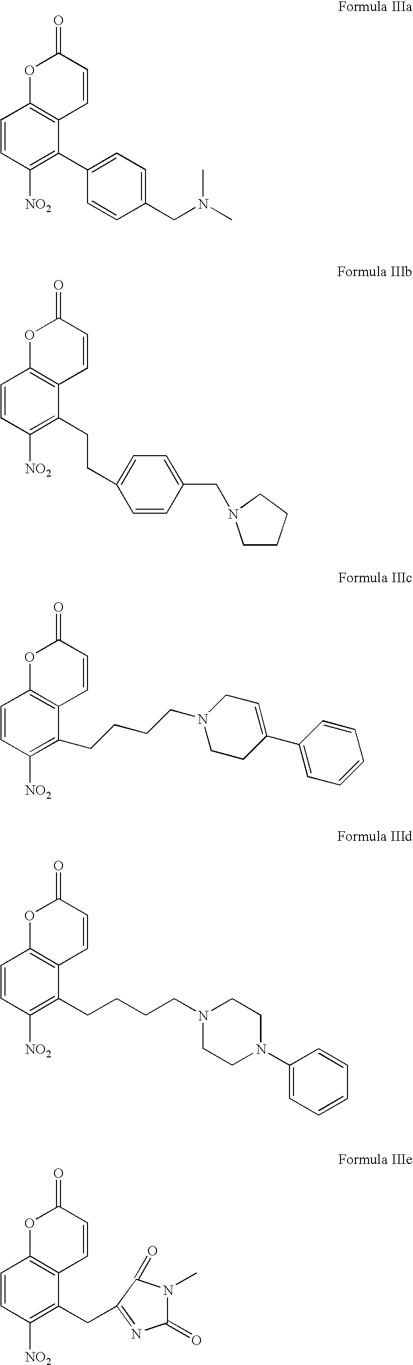

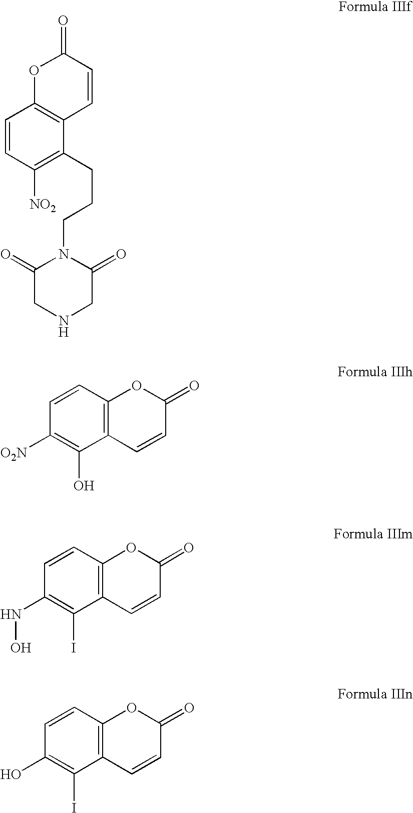

In some embodiments, the cancers that may be treated with this composition include but are not limited adrenal cortical cancer, anal cancer, aplastic anemia, bile duct cancer, bladder cancer, bone cancer, bone metastasis, central nervous system (CNS) cancers, peripheral nervous system (PNS) cancers, Castleman's Disease, cervical cancer, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's disease, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, uterine cancer (e.g. uterine sarcoma), vaginal cancer, vulvar cancer, and Waldenstrom's macroglobulinemia.

In some embodiments, the composition further includes an anti-tumor agent. The anti-tumor agents include but are not limited to antitumor alkylating agents, antitumor antimetabolites, antitumor antibiotics, plant-derived antitumor agents, antitumor organoplatinum compounds, antitumor campthotecin derivatives, antitumor tyrosine kinase inhibitors, monoclonal antibodies, interferons, biological response modifiers, and other agents having antitumor activities, or a pharmaceutically acceptable salt thereof.

In some embodiments, the antitumor alkylating agents are nitrogen mustard N-oxide, cyclophosphamide, ifosfamide, melphalan, busulfan, mitobronitol, carboquone, thiotepa, ranimustine, nimustine, temozolomide, and carmustine; the antitumor antimetabolites are methotrexate, 6-mercaptopurine riboside, mercaptopurine, 5-fluorouracil, tegafur, doxifluridine, carmofur, cytarabine, cytarabine ocfosfate, enocitabine, S-1, gemcitabine, fludarabine, and pemetrexed disodium; the antitumor antibiotics are actinomycin D, doxorubicin, daunorubicin, neocarzinostatin, bleomycin, peplomycin, mitomycin C, aclarubicin, pirarubicin, epirubicin, zinostatin stimalamer, idarubicin, sirolimus, and valrubicin; the plant-derived antitumor agents are vincristine, vinblastine, vindeshine, etoposide, sobuzoxane, docetaxel, paclitaxel, and vinorelbine; the antitumor platinum-complex compounds are cisplatin, carboplatin, nedaplatin, and oxaliplatin; the antitumor campthotecin derivatives are irinotecan, topotecan, and campthotecin; the antitumor tyrosine kinase inhibitors are gefitinib, imatinib, and erlotinib; the monoclonal antibodies are cetuximab, bevacizumab, rituximab, bevacizumab, alemtuzumab, and trastuzumab; the interferons are interferon α, interferon α-2a, interferon α-2b, interferon β, interferon γ-1a, and interferon γ-n1, the biological response modifiers are krestin, lentinan, sizofuran, picibanil, or ubenimex, and the other antitumor agents are mitoxantrone, L-asparaginase, procarbazine, dacarbazine, hydroxycarbamide, pentostatin, tretinoin, alefacept, darbepoetin alfa, anastrozole, exemestane, bicalutamide, leuprorelin, flutamide, fulvestrant, pegaptanib octasodium, denileukin diftitox, aldesleukin, thyrotropin alfa, arsenic trioxide, bortezomib, capecitabine, and goserelin.

In some embodiments, the anti-tumor agent is an organoplatinum compound including but not limited to oxaliplatin (OX), cisplatin, or carboplatin. In some embodiments, the anti-tumor agent is an anti-metabolite agent including but not limited togemcitabine (GEM). In some embodiments, the composition further includes more than one anti-tumor agent, for example, an organoplatinum compound and an anti-metabolite agent. In some embodiments, the anti-tumor agents are OX and GEM.

Other embodiments provide a composition for the treatment of a cancer, the composition comprising an effective amount of a combination of an anti-tumor agent and a compound of formula (I), or a pharmaceutically acceptable salt or prodrug thereof:

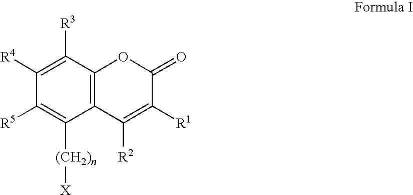

wherein n=0-10; R¹, R², R³, R⁴, R⁵ and X are independently selected from the group consisting of hydrogen, hydroxy, optionally substituted amine, carboxyl, ester, nitroso, nitro, amino, halogen, optionally substituted (C₁-C₆) alkyl, optionally substituted (C₁-C₆) alkoxy, optionally substituted (C₃-C₇) cycloalkyl, optionally substituted (C₃-C₇) heterocyclic, phenyl, and optionally substituted aryl; and wherein at least two of the R¹, R², R³, R⁴, and R⁵ substituents are always hydrogen.

In some embodiments, the benzopyrone compound is of formula II or its pharmaceutically acceptable salts or prodrugs:

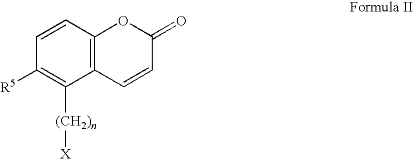

wherein R⁵ is selected from the group consisting of hydrogen, carboxyl, nitroso, nitro, amino, and hydroxy; and X is selected from the group consisting of halogen, hydroxy, optionally substituted (C₁-C₇) alkyl, optionally substituted (C₁-C₆) alkoxy, optionally substituted (C₃-C₇) cycloalkyl, optionally substituted (C₃-C₇) heterocyclic, phenyl, and optionally substituted aryl. In some embodiments, the compound is of the formula IIIa, IIIb, IIIc, IIId, IIIe, IIIf, IIIg, IIIh, IIIk, IIIl, IIIm, IIIn, or one of their pharmaceutically acceptable salts or prodrugs:

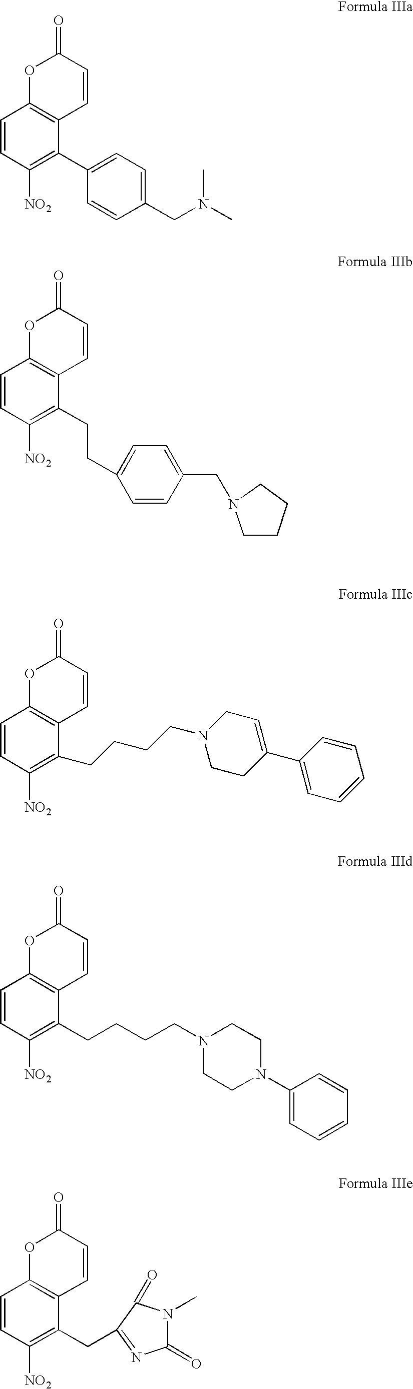

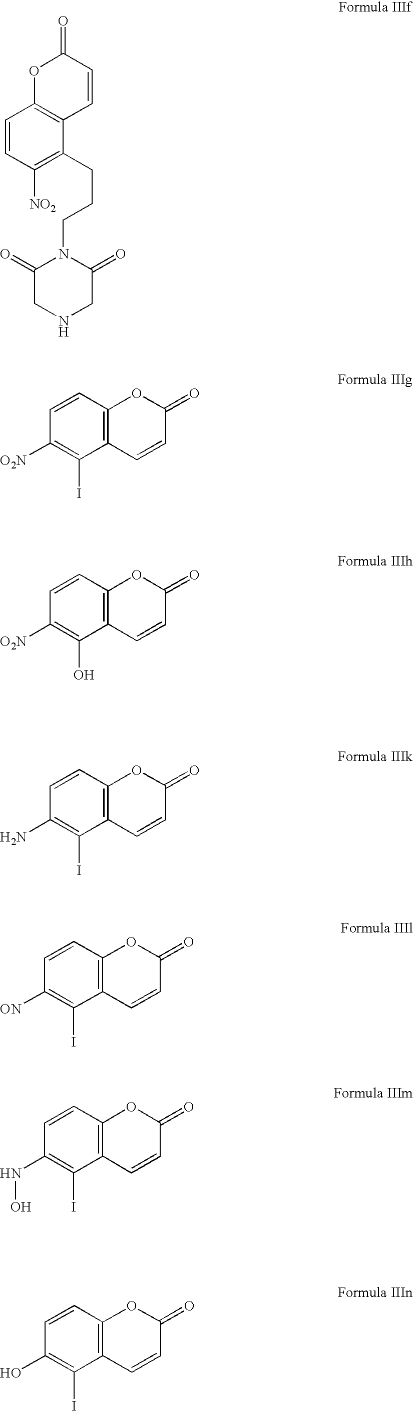

In some embodiments, the anti-tumor agent is an organoplatinum anti-cancer compound. In some embodiments, the anti-tumor agent is cisplatin, carboplatin or oxaliplatin. In some embodiments, the anti-tumor agent is oxaliplatin (OX). In some embodiments, the anti-tumor agent is gemcitabine (GEM). In some embodiments, the invention provides more than one anti-tumor agent. In some embodiments, the anti-tumor agents used in combination with a benzopyrone compound are OX and GEM. In some embodiments, the benzopyrone compound is of formula IIIg, i.e. 5-iodo-6-nitro-benzopyrone. In some embodiments, the benzopyrone compound is of formula IIIk, i.e. 5-iodo-6-amino-benzopyrone. In some embodiments, the composition comprises IIIg and OX. In some embodiments, the composition comprises IIIg and GEM. In some embodiments, the composition comprises IIIg, GEM and OX. In some embodiments, the composition comprises IIIk and OX. In some embodiments, the composition comprises IIIk and GEM. In some embodiments, the composition comprises IIIk, GEM and OX. In some embodiments, the combined effect of an anti-tumor agent and a benzopyrone compound is synergistic. In some embodiments, the combined effect of OX or GEM with IIIg (5-iodo-6-nitro-benzopyrone) is synergistic. In some embodiments, the combined effect of OX or GEM with IIIk (5-iodo-6-amino-benzopyrone) is synergistic.

Some embodiments provide use of a composition set forth herein for preparation of a medicament for the treatment of cancer including but not limited to adrenal cortical cancer, anal cancer, aplastic anemia, bile duct cancer, bladder cancer, bone cancer, bone metastasis, CNS tumors, peripheral CNS cancer, breast cancer, Castleman's Disease, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, uterine cancer (e.g. uterine sarcoma), vaginal cancer, vulvar cancer, and Waldenstrom's macroglobulinemia. In some preferred embodiments, the cancer is pancreatic cancer.

Compositions according to the invention may include salts or free-base forms of the compounds of one of formulae I-III (e.g. one of compounds of formulae IIIa-IIIh, in particular IIIg or IIIh or IIIk, and most particularly IIIg) and salts or free base forms of an anti-tumor agent, such as an organoplatinum drug including but not limited to cisplatin, carboplatin or oxaliplatin, or gemcitabine. Compositions may be in the form of an oral, intravenous, intraperitoneal, or other pharmaceutically acceptable dosage form. In some embodiments, the composition is administered orally and the dosage form is a tablet, capsule, caplet or other orally available form. In some embodiments, the composition is parenteral, e.g. intravenous, and is administered by means of a solution containing both the compound of one of formulae I-III (e.g. one of compounds of formulae IIIa-IIIh, in particular IIIg or IIIh or IIIk, and most particularly IIIg) and salts or free base forms of an anti-tumor agent, such as an organoplatinum drug including but not limited to cisplatin, carboplatin or oxaliplatin, or gemcitabine.

Pharmaceutical compositions of the candidate PARP inhibitors of the present invention, include compositions wherein the active ingredient is contained in a therapeutically or prophylactically effective amount, i.e., in an amount effective to achieve therapeutic or prophylactic benefit. The actual amount effective for a particular application will depend, inter alia, on the condition being treated and the route of administration. Determination of an effective amount is well within the capabilities of those skilled in the art. The pharmaceutical compositions comprise the candidate PARP inhibitor, one or more pharmaceutically acceptable carriers, diluents or excipients, and optionally additional therapeutic agents. The compositions can be formulated for sustained or delayed release.

A preferred therapeutic composition of the present invention also includes an excipient, an adjuvant and/or carrier. Suitable excipients include compounds that the subject to be treated can tolerate. Examples of such excipients include water, saline, Ringer's solution, dextrose solution, Hank's solution, and other aqueous physiologically balanced salt solutions. Nonaqueous vehicles, such as fixed oils, sesame oil, ethyl oleate, or triglycerides can also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosal, o-cresol, formalin and benzyl alcohol. Standard formulations can either be liquid injectables or solids which can be taken up in a suitable liquid as a suspension or solution for injection. Thus, in a non-liquid formulation, the excipient can comprise dextrose, human serum albumin, preservatives, etc., to which sterile water or saline can be added prior to administration. In one embodiment of the present invention, a therapeutic composition can include a carrier. Carriers include compounds that increase the half-life of a therapeutic composition in the treated subject. Suitable carriers include, but are not limited to, polymeric controlled release vehicles, biodegradable implants, liposomes, bacteria, viruses, other cells, oils, esters, and glycols.

The oral form in which the therapeutic agent is administered can include powder, tablet, capsule, solution, or emulsion. The effective amount can be administered in a single dose or in a series of doses separated by appropriate time intervals, such as hours. Pharmaceutical compositions can be formulated in conventional manner using one or more physiologically acceptable carriers comprising excipients and auxiliaries which facilitate processing of the active compounds into preparations which can be used pharmaceutically. Proper formulation is dependent upon the route of administration chosen. Suitable techniques for preparing pharmaceutical compositions of the therapeutic agents of the present invention are well known in the art.

It will be appreciated that appropriate dosages of the active compounds, and compositions comprising the active compounds, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular candidate PARP inhibitor, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g. in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. In some embodiments, the dosage ranges from about 1 to about 100 mg/m² or about 1 to about 250 mg/kg of body weight for human subjects. Single or multiple administrations can be carried out with the dose level and pattern being selected by the treating physician.

### Kits

In some embodiments, the present invention also provides a kit for the treatment of a cancer. The kit includes an effective amount of a compound of formula (I) or a pharmaceutically acceptable salt or prodrug thereof. The kit may be used to treat cancers including but not limited to adrenal cortical cancer, anal cancer, aplastic anemia, bile duct cancer, bladder cancer, bone cancer, bone metastasis, central nervous system (CNS) cancers, peripheral nervous system (PNS) cancers, Castleman's Disease, cervical cancer, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's disease, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, uterine cancer (e.g. uterine sarcoma), vaginal cancer, vulvar cancer, and Waldenstrom's macroglobulinemia.

Other embodiments provide a kit for the treatment of a cancer, the kit comprising a composition of a combination of an anti-tumor agent and a compound of formula (I) as disclosed hereinabove, and optionally instructions for the use of the composition for the treatment of a cancer including but not limited to adrenal cortical cancer, anal cancer, aplastic anemia, bile duct cancer, bladder cancer, bone cancer, bone metastasis, CNS tumors, peripheral CNS cancer, breast cancer, Castleman's Disease, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, uterine cancer (e.g. uterine sarcoma), vaginal cancer, vulvar cancer, and Waldenstrom's macroglobulinemia.

Still other embodiments provide a use of the composition disclosed herein for preparation of a kit for the treatment of a cancer. Such kit may also include information, such as scientific literature references, package insert materials, clinical trial results, and/or summaries of these and the like, which indicate or establish the activities and/or advantages of the benzopyrone composition. Kits described herein can be provided, marketed and/or promoted to health providers, including physicians, nurses, pharmacists, formulary officials, and the like.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### Example 1

### In Vitro Efficacy of the Compound of Formula IIIg (5-iodo-6-nitrocoumarin)

### Materials and Methods

Test Agents—Cells are treated 24 hours after plating (Day 1) with vehicle or test agents at concentrations of 0.1 μM, 0.3 μM, 1 μM, 3 μM, 10 μM, 30 μM and 100 μM.

Tumor Cell Lines—Tumor cell lines (Table 1) are obtained from the American Type Cell Collection (ATCC, Rockville, Md.) or NCI-DCTD Tumor Repository (Bethesda, Md.) and maintained in proper growth media supplemented with 10% fetal bovine serum (FBS, NovaTech), and are maintained in specified growth media. Cells are propagated at 37° C. in a humidified atmosphere containing 5% carbon dioxide.

Cell proliferation assay—Cell proliferation is determined using BrdU chemiluminescent assay, which measures incorporation of 5-bromo-2′-deoxyuridine (BrdU) into the genomic DNA of proliferating cells. Briefly, cells are added with BrdU for 4 hours, during which time it incorporates into the DNA of dividing cells in place of thymidine. Following labeling, cells are fixed and DNA denatured in one step by adding a FixDenat® denaturizing solution. After removing FixDenat, an anti-BrdU-peroxidase conjugated antibody is added, which binds to BrdU incorporated into newly-synthesized cellular DNA. Following antibody incubation, cells are washed 3 times with PBS and subjected to luminescent analysis. The reaction product is quantified by measuring light emission using a scanning multi-well luminometer (luminescence ELISA reader.)

Experimental Design—Tumor cells (Table 1) are grown to 70% confluency, trypsinized, counted and seeded in 96 well flat-bottom plates at final concentrations of 7.5×103-3×104 cells/well (Day 0); treatment with IIIg begins on Day 1 and continues for 68 hours (Table 2). Doses for both compounds range between 0.1 M and 100 μM. At the 68 hour time point, viable cell number is measured by BrdU assay as described above. Experiments are repeated at least twice at the same doses to determine inhibition of cell proliferation. Results from these studies are used to calculate an IC₅₀ value (drug concentration that results in half-maximal response) for IIIg in each line.

Pancreatic Cancer Cell Line and Culture Conditions

The human pancreatic cancer cell lines (Table 1) are maintained in minimal essential medium supplemented with 10% fetal bovine serum (FBS), sodium pyruvate, non-essential amino acids, 1-glutamine, a two-fold vitamin solution (Life Technologies, Grand Island, N.Y.), and a penicillin-streptomycin mixture (Flow Laboratories, Rockville, Md.). The cultures are free of mycoplasma and the following pathogenic murine viruses: reovirus type 3, pneumonia virus, K virus, Theiler's encephalitis virus, Sendai virus, minute virus, mouse adenovirus, mouse hepatitis virus, lymphocytic choriomeningitis virus, ectromelia virus, and lactate dehydrogenase virus (assayed by Science Applications International Corp., Frederick, Md.).

TABLE 1
Tumur Cell Lines

| Cell Line | Tissue Type | Histology |
|---|---|---|
| PANC-1 | Pancreas | Ductal Carcinoma |
| MX-1 | Breast | Adenocarcinoma |
| PC-3 | Prostate | Adenocarcinoma |
| MDA-MB-231 | Breast | Adenocarcinoma |
| NIH: OVCAR-3 | Ovary | Adenocarcinoma |

TABLE 2
Treatment Schedule

| | | | Day 3 | |
|---|---|---|---|---|
| Day 0 | Day 1 (0 Hr.) | Day 2 (24 Hr.) | (38 Hr.) | Day 4 (72 Hr.) |
| Plate Cells | Treatment Begins | → | → | BrdU Assay |

Data Collection and Statistical Analysis

For single studies, data from each experiment is collected and expressed as % Inhibition of Cell Proliferation (% ICP) using the following calculation.

% ICP=100%×[(1−(RLUₜₑₛₜ/RLU_{vehicle})]

Where RLUₜₑₛₜ is the relative light unit of the tested sample, and RLU_{vehicle} is the relative light unit of the vehicle in which the drug is dissolved. An IC₅₀ value is calculated from % ICP (PRISM® GraphPad software) using the formula:

y=min+(max−min)/(1+10^{(Log(IC50)-x)×Hillslope})

Where (x) is the logarithm of agonist concentration, (y) is the response, and (min) and (max) are the variable lower and upper plateaus, respectively. The variable Hillslope characterizes the slope of the curve at its midpoint. The IC₅₀ is the drug concentration for y halfway between min and max.

Results

Single agent activity of a compound of Formula IIIg is evaluated after 72 hour treatment in a panel of human tumor cells including human breast (MX-1, MDA-MB0231), pancrease (PANC-1), ovarian (NIH: OVCAR-3), prostate (PC-3). IC₅₀ values for these experiments are summarized in Table 3, below.

TABLE 3
Mean IC₅₀ Values for compound IIIg (5-iodo-6-nitrocoumarin)

| Cell Line | | IIIg |
|---|---|---|
| PANC-1 | Exp1 | 22.0 μM |
| | Exp2 | 27.0 μM |
| | Mean | 24.5 μM |
| | IC₅₀ | |
| MX-1 | Exp1 | 27.0 μM |
| | Exp2 | 27.0 μM |
| | Mean | 27.0 μM |
| | IC₅₀ | |
| PC-3 | Exp1 | 22.0 μM |
| | Exp2 | 25.3 μM |
| | Mean | 23.7 μM |
| | IC₅₀ | |
| MDA-MB-231 | Exp1 | 24.0 μM |
| | Exp2 | 24.3 μM |
| | Mean | 24.2 μM |
| | IC₅₀ | |
| NIH:OVCAR-3 | Exp1 | 30.4 μM |
| | Exp2 | 29.6 μM |
| | Mean | 30.0 μM |
| | IC₅₀ | |

TABLE 4
Mean Maximum % Response for IIIg

| Cell Line | Max % Response | Max Concentration |
|---|---|---|
| PANC-1 | 99 | 100 μM |
| MX-1 | 99 | 100 μM |
| PC-3 | 100 | 100 μM |
| MDA-MB-231 | 99 | 100 μM |
| NIH: OVCAR-3 | 98 | 100 μM |

### Example 2

### Evaluation of IIIg Against the Human Carcinoma Xenografts in Nude Mice

### Methods and Materials

Mice

Female athymic nude mice (nu/nu, Harlan) are 9-10 weeks old, and have a body weigh (BW) range of 18.1-27.0 g on D1 of the study. The animals are fed ad libitu water (reverse osmosis, 1 ppm Cl) and NIH 31 Modified and Irradiated Lab Diet® consisting of 18.0% crude protein, 5.0% crude fat, and 5.0% crude fiber. The mice are housed on irradiated ALPHA-dri® bed-o-cobs® Laboratory Animal Bedding in static microisolators on a 12-hour light cycle at 21-22° C. (70-72° F.) and 40-60% humidity. PRC specifically complies with the recommendations of the Guide for Care and Use of Laboratory Animals with respect to restraint, husbandry, surgical procedures, feed and fluid regulation, and veterinary care. The animal program at PRC is accredited by AAALAC International, which assures compliance with accepted standards for the care and use of laboratory animals.

Tumor Implantation

For experiments with SW620, the human SW620 colon adenocarcinoma utilized in the study is maintained in nude mice by serial engraftment.

For experiments with MX-1, the human MX-1 breast adenocarcinoma utilized in the study is maintained in nude mice by serial engraftment.

For experiments with PANC-1, the human PANC-1 pancreatic carcinoma utilized in the study is maintained in nude mice by serial engraftment.

A tumor fragment (1 mm³) is implanted s.c. into the right flank of each test mouse. Tumors are monitored twice weekly and then daily as their volumes approached 80-120 mm³. On D1 of the study, animals are sorted into treatment groups with tumor sizes of 63-144 mm³ and group mean tumor sizes of approximately 98 mm³. Tumor size, in mm³, is calculated from the following equation:

Tumor Volume=(w²×l)/2

where w=width and l=length in mm of the tumor. Tumor weight may be estimated with the assumption that 1 mg is equivalent to 1 mm³ of tumor volume.

Test Articles

IIIg dosing solutions are prepared fresh weekly by dissolving IIIg in 100% dimethylsulfoxide (DMSO). The compound and dosing solutions are stored at 4° C. 5-FU (positive control) (Adrucil®, Roche Diagnostics, 50 mg/mL, Lot # 200826) is diluted with 5% dextrose in water, pH ˜4.8. A fresh 5-FU dosing solution is prepared for each dose.

Treatment

Mice are sorted into six groups (n=10), and treated in accordance with the protocol in Table 1. Control Group 1 mice receive DMSO, the LK4 vehicle, by i.p. injection once daily for the duration of the study (qd to end). Group 2 mice receive 100 mg/kg 5-FU i.p. once weekly for three weeks (qwk×3). In Group 2, the dosing volume of 0.2 mL/20 g mouse is scaled to the BW of each animal. Groups 3 and 4 receive LK4 i.p. at 1.5 and 1 mg/mouse, respectively, qd to end. Groups 5 and 6 receive LK4 i.p. at 2 and 1 mg/mouse, respectively, biweekly for the duration of the study (biwk to end). In Groups 1 and 3-6, the dosing volume of 0.05 mL/mouse is not BW-adjusted.

Endpoint

Tumors are calipered twice weekly for the duration of the study. Each animal is euthanized when its neoplasm reaches the predetermined endpoint size (1200 mm³). The time to endpoint (TTE) for each mouse is calculated by the following equation:

TTE=[Log₁₀(endpoint volume)−b]/m,

where TTE is expressed in days, endpoint volume is in mm³, b is the intercept, and m is the slope of the line obtained by linear regression of a log-transformed tumor growth data set.

The data set comprises the first observation that exceeds the study endpoint volume and the three consecutive observations that immediately precede the attainment of the endpoint volume. The calculated TTE is usually less than the day on which an animal is euthanized for tumor size. Animals that do not reach the endpoint are euthanized at the end of the study, and assigned a TTE value equal to the designated last day of the study (54 days). An animal classified as having died from treatment-related (TR) causes or non-treatment-related metastasis (NTRm) causes is assigned a TTE value equal to the day of death. An animal classified as having died from non-treatment related (NTR) causes is excluded from TTE calculations. Treatment efficacy is determined from tumor growth delay (TGD), which is defined as the increase in the median TTE for a treatment group compared to the control group:

TGD=T−C,

expressed in days, or as a percentage of the median TTE of the control group:

% TGD=%100×[(T−C)/C]

where:
T=median TTE for a treatment group,
C=median TTE for control Group 1.

MTV and Criteria for Regression Responses

Treatment efficacy is also determined from the tumor volumes of animals remaining in the study on the last day, and from the number of regression responses. The MTV(n) is defined as the median tumor volume on D54 in the number of animals remaining, n, whose tumors have not attained the endpoint volume. Treatment may cause a partial regression (PR) or a complete regression (CR) of the tumor in an animal. A PR indicates that the tumor volume is 50% or less of its D1 volume for three consecutive measurements during the course of the study, and equal to or greater than 13.5 mm³ for one or more of these three measurements. A CR indicates that the tumor volume is less than 13.5 mm³ for three consecutive measurements during the course of the study. An animal with a CR at the termination of a study is additionally classified as a tumor-free survivor (TFS).

Statistical and Graphical Analyses

Statistical and graphical analyses are conducted using Prism 3.03 (GraphPad) for Windows. The Kiruskal-Wallis test, and post hoc analysis with Dunn's multiple comparison test, are employed to analyze for differences among the treatment groups, and between two treatment groups, respectively. The Kiruskal-Wallis, which tests for differences in distribution functions, is an extension of the Mann-Whitney test, and an analog of the F-test used in ANOVAs. The logrank test is employed to analyze for differences between the overall survival experiences of two groups. The Kiruskal-Wallis and logrank tests utilize the TTE data for all animals in a group, except the NTR deaths. The two-tailed statistical analyses are conducted at P=0.05.

Kaplan-Meier plots show the percentage of animals remaining in the study versus time. Kaplan-Meier plots use the same TTE data as the Kiruskal-Wallis and logrank tests. The tumor growth curves show the group median tumor volume as a function of time. When an animal exits the study due to tumor size or TR death, the final tumor volume recorded for the animal is included with the data used to calculate the median volume at subsequent time points. Therefore, the final median tumor volume shown by the curve may differ from the MTV, which is the median tumor volume for mice remaining in the study on the last day (excluding all with tumors that have attained the endpoint). If more than one TR death occurs in a group, the median tumor growth curve is customarily truncated at the time of the last measurement that precedes the second TR death. Tumor growth curves are also truncated when the tumors in more than 50% of the assessable animals in a group have attained the endpoint volume.

Western Blot

Cells are lysed on ice in 150 mM NaCl, 1% NP-40, 0.5% sodium deoxycholate, 0.1% SDS, and 50 mM Tris-HCl, pH 7.4 containing a protease inhibitor cocktail (Roche Diagnostics, Indianapolis, Ind.) as specified by the manufacturer. Protein concentration is determined using the BCA assay (Pierce Chemical, Rockford, Ill.). Samples are boiled with 2× Laemmli buffer and electrophoresed on 10% polyacrylamide gels containing 0.1% SDS, followed by transfer to Immobilon-P membranes (Millipore, Billerica, Mass.), and then they are incubated with specific primary antibodies. Proteins of interest are detected with appropriate horseradish peroxidase-conjugated secondary antibodies and enhanced chemiluminescence substrate (Pierce Chemical).

The expression of PARP-1 protein is analyzed in thirteen pancreatic cancer cell lines by western blotting.

The effect of IIIg alone and in combination with oxaliplatin on the proliferation of eight pancreatic cancer cells is determined using a BrdU-ELISA assay.

Assessment of synergy is performed according to the method described by Chou and Talalay.

To determine whether the PARP-1 activity inhibition by IIIg has any inhibitory effects on NF-κB signaling, NF-κB DNA binding activity is evaluated using an electrophoretic mobility shift assay.

The therapeutic efficacy of IIIg on tumor growth and survival is evaluated in different luciferase-expressing pancreatic cancer orthotopic nude mouse models using an IVIS100 Imaging System, allowing a quantitative real time monitoring of tumor growth.

Mice are monitored daily for signs of toxicity including weight loss, diarrhea, inactivity, and general appearance.

Results

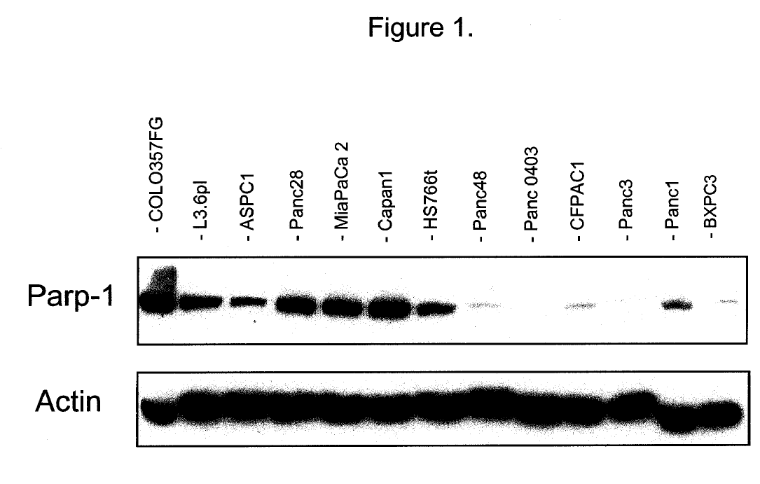

PARP-1 overexpression is detected in 8 of 13 human pancreatic cancer cell lines (FIG. 1).

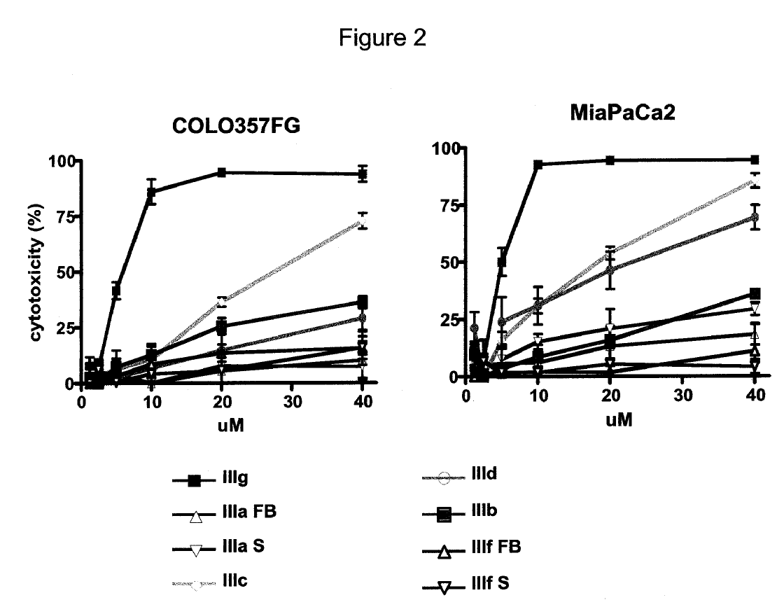

In vitro, IIIg alone significantly inhibits the growth of eight PC cell lines, with an IC₅₀ ranging from 5 to 10 μM (FIG. 2).

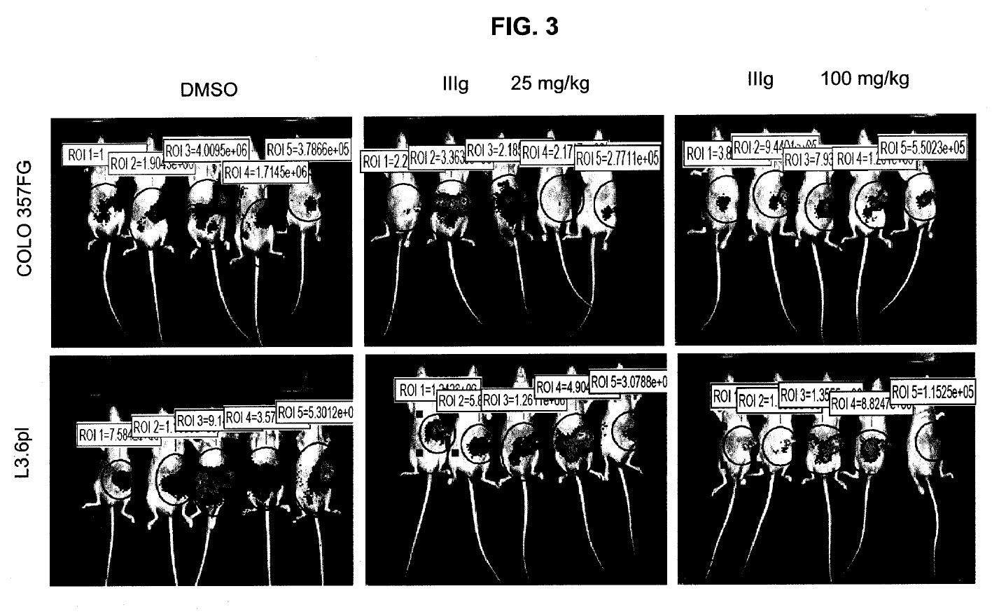

In nude mice orthotopically injected with luciferase-expressing Colo357FG or L3.6 μl PC cells, IIIg at dose of 100 mg/kg/day 2/week×4 weeks significantly reduces the tumor burden and prolongs survival without signs of toxicity (FIG. 3).

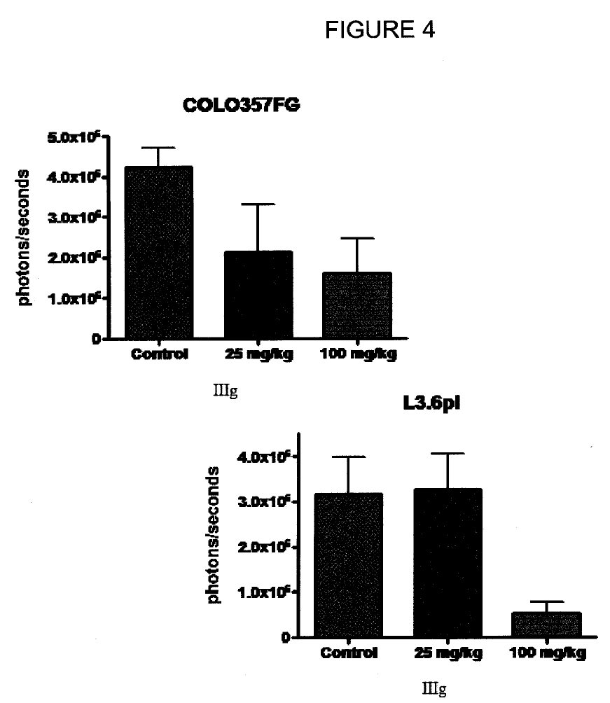

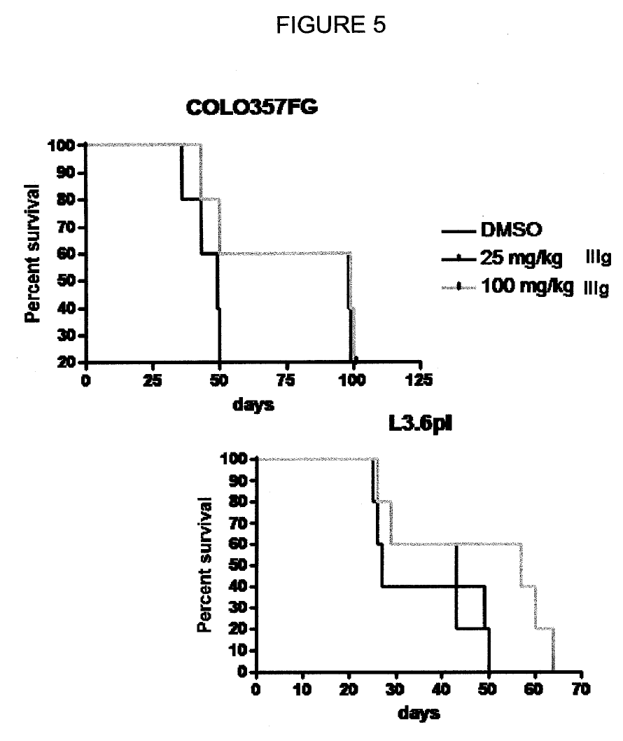

FIG. 4 shows mice bearing Colo357FG and L3.6 μl human pancreatic cancer xenografts treated with 0.25 and 100 mg/kg IIIg; FIG. 5 shows the mean survival data for mice bearing Colo357FG and L3.6 μl human pancreatic cancer xenografts treated with 0.25 and 100 mg/kg IIIg.

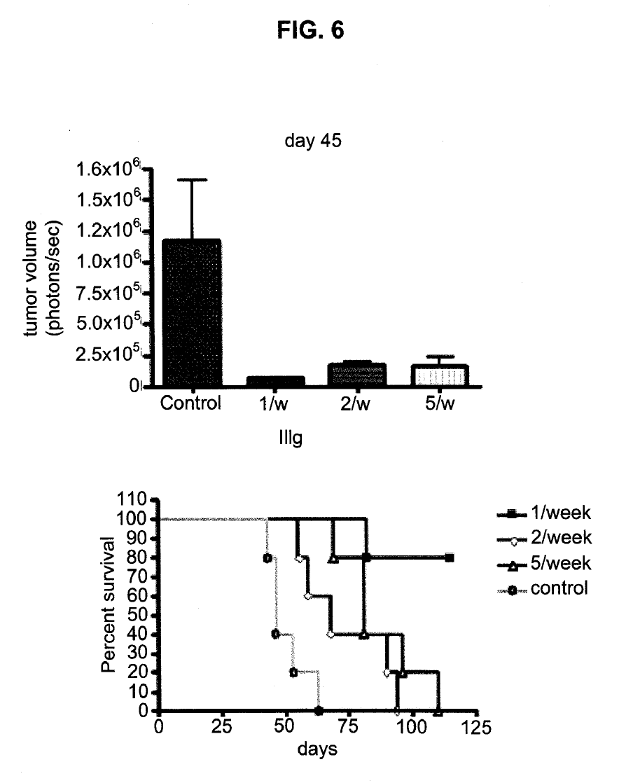

Preliminary results indicate a greater in vivo antitumor effect for IIIg 200 mg/kg/week if administered once a week in comparison with a 2 times/week or daily schedule (FIG. 6).

Conclusions

The PARP inhibitor 5-iodo-6-nitrocoumarin (IIIg) shows a potent in vitro and in vivo antitumor activity as a single agent and potentiates oxaliplatin cytotoxicity in different pancreatic cancer cell models.

### Example 3

### Effect of IIIg on Pancreatic Cancer Cell Lines

Pancreatic Cancer Cell Line and Culture Conditions

The human pancreatic cancer cell lines are maintained in minimal essential medium supplemented with 10% fetal bovine serum (FBS), sodium pyruvate, non-essential amino acids, 1-glutamine, a two-fold vitamin solution (Life Technologies, Grand Island, N.Y.), and a penicillin-streptomycin mixture (Flow Laboratories, Rockville, Md.). The cultures are free of mycoplasma and the following pathogenic murine viruses: reovirus type 3, pneumonia virus, K virus, Theiler's encephalitis virus, Sendai virus, minute virus, mouse adenovirus, mouse hepatitis virus, lymphocytic choriomeningitis virus, ectromelia virus, and lactate dehydrogenase virus (assayed by Science Applications International Corp., Frederick, Md.).

Animals and Orthotopic Implantation of Tumor Cells

Male athymic nude mice (NCI-nu) are purchased from the Animal Production Area of the National Cancer Institute Frederick Cancer Research and Development Center (Frederick, Md.). The mice are housed under specific pathogen-free conditions in facilities approved by the American Association for Accreditation of Laboratory Animal Care and in accordance with current regulations and standards of the U.S. Department of Agriculture, U.S. Department of Health and Human Services, and the National Institutes of Health. The mice are used in accordance with institutional guidelines when they are 8 to 12 weeks old.

To produce pancreatic tumors, subconfluent cultures are harvested by a brief exposure to 0.25% trypsin and 0.02% EDTA. Trypsinization is stopped with medium containing 10% FBS, and the cells are washed once in serum-free medium and resuspended in Hanks' balanced salt solution (HBSS). Only suspensions consisting of single cells with greater than 90% viability are used for the injections. One million cells suspended in 50 μl of HBSS are injected into the pancreas of nude mice as described previously (Baker C H, Solorzano C C, Fidler I J. Cancer Res. 2002; 62:1996-2003; Hwang R F, et. al. Clin Cancer Res. 2003; 9:6534-6544).

Treatment of Nude Mice with Established Orthotopic Human Pancreatic Cancer

Fourteen days after the injection of tumor cells into the pancreas, all mice are randomized to one of the following four treatment groups (n=5).

IIIg is diluted in 100% DMSO and administrated bi-weekly (biw):

25 mg/kg biw

50 mg/kg biw

100 mg/kg biw

Necropsy Procedure and Histologic Studies

Before necropsy, mice are weighed, and tumors are then excised and weighed. For histology and immunohistochemical (1HC) analyses, one part of the tumor tissue is fixed in formalin and embedded in paraffin, and another is embedded in OCT compound (Miles, Inc., Elkhart, Ind.), rapidly frozen in liquid nitrogen, and stored at −70° C.

### Example 4

### Effects of 5-iodo-6-nitro-benzopyrone (IIIg), Oxaliplatin (OxaliPt), and a Combination of IIIg with Oxaliplatin on Cell Cycle Distribution in Colo375FG Metastatic Pancreatic Adenocarcinoma Cells

Colo375FG metastatic pancreatic adenocarcinoma cells are cultured in Dulbecco Modified Eagle Medium with 10% fetal bovine serum. Cells are plated at 10⁵ per P100 or at 10⁴ per P60 (for assays requiring up to 3 days of culture), in the presence of different concentrations of BiPar compounds and other agents, or DMSO control. Following treatment, the number of attached cells is measured using Coulter counter, and by staining with 1% methylene blue. Methylene blue is dissolved in 50%-50% mixture of Methanol and water. Cells are plated in 24- or 96-well plates and treated with compounds as indicated, then media are aspirated, cells are washed with PBS, fixed in methanol for 5-10 min, washed and the plates are allowed to dry completely. Methylene blue solution is added to wells and plates are incubated for 5 min. Staining solution is removed and plates are washed with dH₂O. After plates are completely dry, a small amount of 1N HCl is added to each well to extract the methylene blue. The OD readout at 600 nm and a calibration curve are used to determine cell number.

### Compounds

IIIg (6-nitro-5-iodo-benzopyrone) is dissolved directly from dry powder to 10 mM stock solution in DMSO for each separate experiment, and then the entire volume of the stock solution is used to prepare working concentrations in cell culture medium to avoid any possibility of precipitation and the corresponding loss of compound. Control experiments are carried out with the matching volume/concentration of the vehicle (DMSO). In these controls, the cells showed no changes in their growth or cell cycle distribution.

### PI Exclusion, Cell Cycle and TUNEL Assays

After the addition of drugs and incubation, cells are trypsinized and aliquots of the samples are taken for counting and PI (Propidium Iodide) exclusion assay. One part of the cells is centrifuged and resuspended in 0.5 ml ice-cold PBS containing 5 μg/ml of PI. The other part of the cells is fixed in ice-cold 70% ethanol and stored in a freezer overnight. For cell cycle analysis, cells are stained with propidium iodide (PI) by standard procedures. Cellular DNA content is determined by flow cytometry using BD LSRII FACS, and the percentages of cells in G1, S or G2/M are determined using ModFit software.

Cells are labeled for apoptosis with the “In Situ Cell Death Detection Kit, Fluorescein” (Roche Diagnostics Corporation, Roche Applied Science, Indianapolis, Ind.). Briefly, fixed cells are centrifuged and washed once in phosphate-buffered saline (PBS) containing 1% bovine serum albumin (BSA), then resuspended in 2 ml permeabilization buffer (0.1% Triton X-100 and 0.1% sodium citrate in PBS) for 25 min at room temperature and washed twice in 0.2 ml PBS/1% BSA. The cells are resuspended in 50 μl TUNEL reaction mixture (TdT enzyme and labeling solution) and incubated for 60 min at 37° C. in a humidified dark atmosphere in an incubator. The labeled cells are washed once in PBS/1% BSA, then resuspended in 0.5 ml ice-cold PBS containing 1 μg/ml 4′,6-diamidino-2-phenylindole (DAPI) for at least 30 min. All cell samples are analyzed with a BD LSR II (BD Biosciences, San Jose, Calif.).

### Bromodeoxyuridine (BrdU) Labeling Assay

50 μl of BrdU (Sigma Chemical Co., St. Louis, Mo.) stock solution (1 mM) is added to give 10 μM BrdU final concentration. The cells are incubated for 30 min at 37° C. and fixed in ice-cold 70% ethanol and stored in a cold room (4° C.) overnight. Fixed cells are centrifuged and washed once in 2 ml PBS, then resuspended in 0.7 ml of denaturation solution (0.2 mg/ml pepsin in 2 N HCl) for 15 min at 37° C. in the dark and suspended with 1.04 ml 1M Tris buffer (Trizma base, Sigma Chemical Co.) to terminate the hydrolysis and washed in 2 ml PBS.

Cells are resuspended in 100-μl anti-BrdU antibody (DakoCytomation, Carpinteria, Calif.) with 1:100 dilution in TBFP permeable buffer (0.5% Tween-20, 1% bovine serum albumin and 1% fetal bovine serum in PBS) and incubated for 25 min at room temperature in the dark and washed in 2 ml PBS. The primary antibody-labeled cells are resuspended in 100 μl Alexa Fluor F(ab′)2 fragment of goat anti-mouse IgG (H+L) (2 mg/mL) (Molecular Probes, Eugene, Oreg.) with 1:200 dilution in TBFP permeable buffer and incubated for 25 min at room temperature in the dark and washed in 2 ml PBS, then resuspended in 0.5 ml ice-cold PBS containing 1 μg/ml 4′,6-diamidino-2-phenylindole (DAPI) for at least 30 min. All cell samples are analyzed with a BD LSR II (BD Biosciences, San Jose, Calif.).

The results of this cell cycle analysis using IIIg, oxaliplatin, or a combination of IIIg and oxaliplatin, are shown in Table 5. The combination of IIIg and oxaliplatin significantly reduces the percentage of live cells and inhibits the proliferation of Colo375FG pancreatic tumor cells as reflected by the percentage of BrdU positive cells.

TABLE 5

| | | OxaliPt | | TUNEL | S-phase % |
|---|---|---|---|---|---|
| | IIIg (μM) | (μM) | Live Cell (%) | (%) | BrdU+ |
| OxaliPt | 0 | 0 | 100 | 2.6 | 94.38 |
| | 0 | 10 | 54 | 21 | 81.74 |
| | 0 | 20 | 29 | 48.3 | 63.12 |
| IIIg | 2.5 | 0 | 100 | 3.3 | |
| | | 0 | 118 | 2.5 | 94.89 |
| | | 10 | 62 | 19 | 8631 |
| | | 20 | 27 | 42 | 65.32 |
| | 12.5 | 0 | 100 | 3.3 | |
| | | 0 | 49 | 21.6 | 93.1 |
| | | 10 | 22 | 57.3 | 67.5 |
| | | 20 | 13 | 85.7 | 33 |
| | 25 | 0 | 100 | 3.3 | |
| | | 0 | 20 | 77.7 | 75.18 |
| | | 10 | 11 | 89.5 | 24.69 |
| | | 20 | 7 | 94.2 | 33.85 |

### Example 5

### Effect of IIIg as a Single Active Agent on Proliferation of Tumor Cells In Vitro

Proliferation of various tumor cell lines including uterine cancer Hela cells, lung carcinoma A549 cells, PARP1+/+A16 and PARP1−/− A12 fibroblasts, and several human pancreatic tumor cell lines, COLO357FG, MiaPaCa-2, AsPC-1, L3.6pl, and Panc28, is measured by BrdU incorporation. In some experiments, tumor cell proliferation is measured after 96 hours. The concentration of IIIg is titrated to show the effect of IIIg on tumor cell growth in vitro.

BrdU assay is well known in the art. Briefly, cells are cultured in the presence of the respective test substances in an appropriate 96-well MP at 37° C. for a certain period of time (1 to 5 days, depending on the individual assay system). Subsequently, BrdU is added to the cells and the cells are reincubated (usually 2-24 h). During this labeling period, the pyrimidine analogue BrdU is incorporated in place of thymidine into the DNA of proliferating cells. After removing the culture medium the cells are fixed and the DNA is denatured in one step by adding FixDenat (the denaturation of the DNA is necessary to improve the accessibility of the incorporated BrdU for detection by the antibody). The anti-BrdU-POD antibody is added and the antibody binds to the BrdU incorporated in newly synthesized, cellular DNA. The immune complexes are detected by the subsequent substrate reaction via chemiluminescent detection (based on Cell Proliferation ELISA, BrdU Chemiluminescence Protocol from Roche).

Experiments and analyses are performed according to Shaw G and Prowse DM, “Inhibition of androgen-independent prostate cancer cell growth is enhanced by combination therapy targeting Hedgehog and ErbB signaling” Cancer Cell Int. 2008 Mar. 18; 8:3.

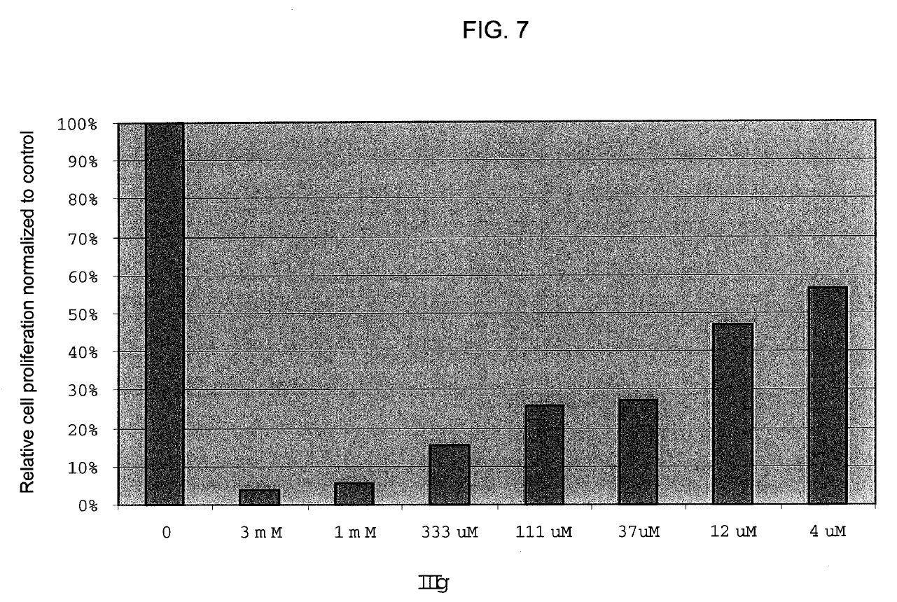

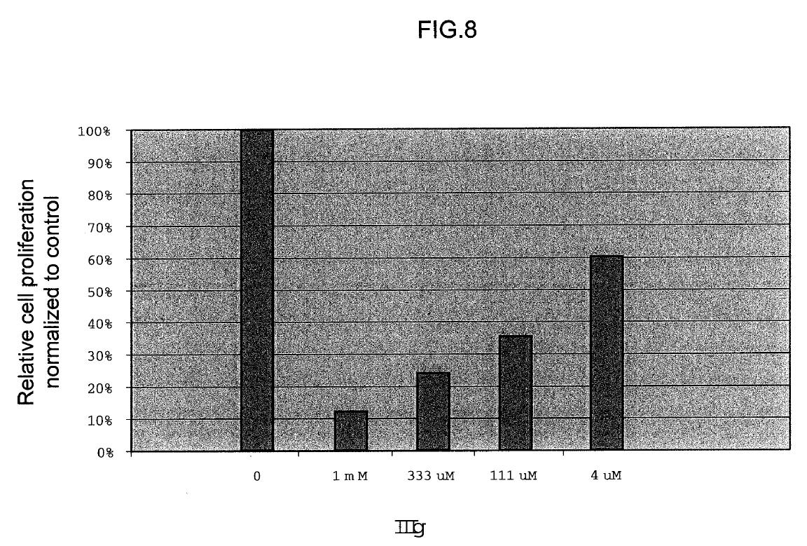

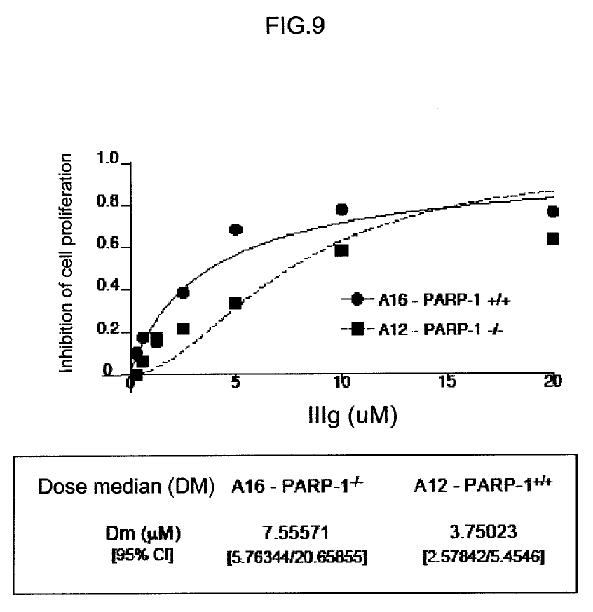

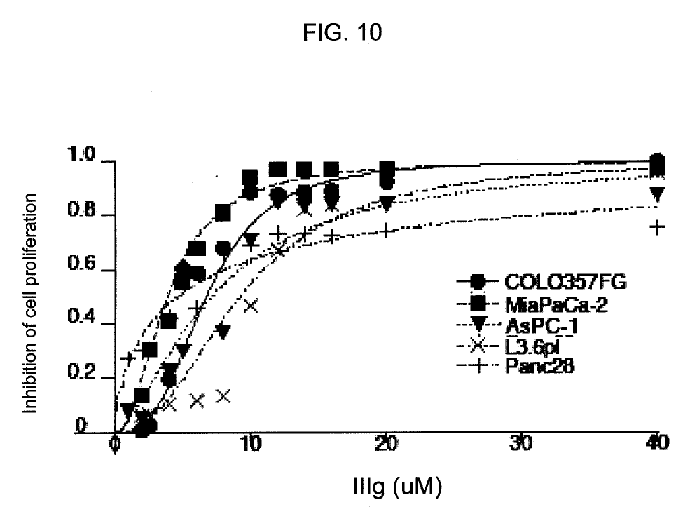

Results of these proliferation experiments are shown in FIGS. 7-10. IIIg selectively inhibits the growth of wildtype but not PARP-1−/− fibroblasts. IIIg also significantly inhibits cell growth of several human pancreatic tumor cell lines.

### Example 6

### PARP1 Expression and PARP Activity in Pancreatic Tumor Cell Lines and PARP1+(A16) and PARP1−/− Fibroblasts (A12)

PARP1 expression in various pancreatic tumor cell lines is assessed by Western Blot. Briefly, cells are lysed on ice in 150 mM NaCl, 1% NP-40, 0.5% sodium deoxycholate, 0.1% SDS, and 50 mM Tris-HCl, pH 7.4 containing a protease inhibitor cocktail (Roche Diagnostics, Indianapolis, Ind.) as specified by the manufacturer. Protein concentration is determined using the BCA assay (Pierce Chemical, Rockford, Ill.). Samples are boiled with 2× Laemmli buffer and electrophoresed on 10% polyacrylamide gels containing 0.1% SDS, followed by transfer to Immobilon-P membranes (Millipore, Billerica, Mass.), and then they are incubated with specific primary antibodies. Proteins of interest are detected with appropriate horseradish peroxidase-conjugated secondary antibodies and enhanced chemiluminescence substrate (Pierce Chemical).

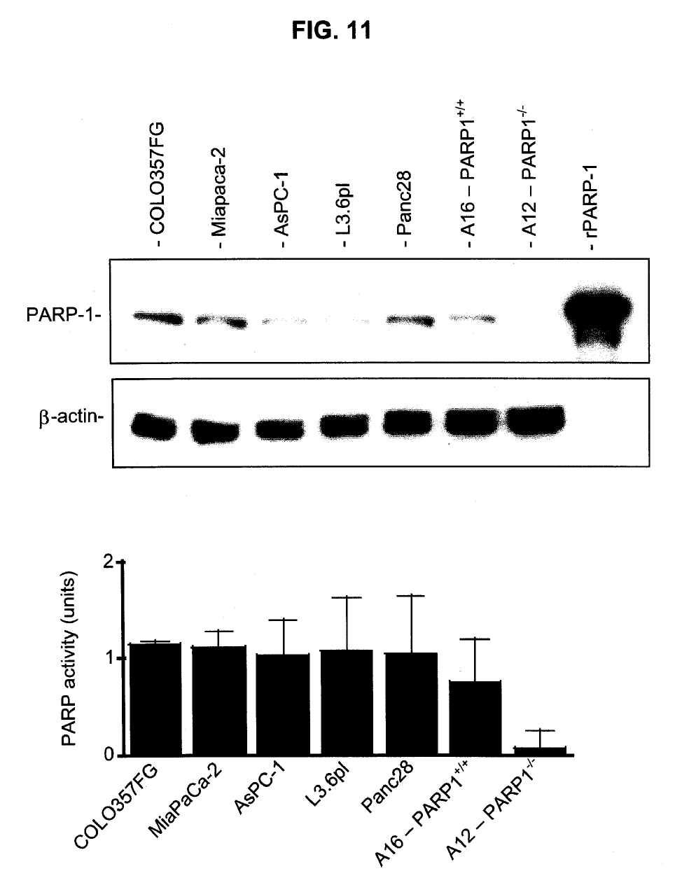

Results of the experiments are shown in FIG. 11

### Example 7

### Effect of IIIg as a Single Active Agent on Pancreatic Tumor Cells In Vivo

The objective of this study is to evaluate the effect of IIIg alone on pancreatic tumor cell growth in vivo. Experiments are carried out to evaluate the treatment of established human pancreatic carcinoma tumors growing in the pancreas of athymic nude mice.

### Materials and Methods

Three days after the orthotopic implantation of 1.0×10⁶ COL0357FG tumor cells in 50 μL of HBSS, when bioluminescence imaging confirmed that tumors are well established, in one experiment, mice are randomly allocated into three groups (n=10 mice per group) to receive one of the following treatments. (a) Vehicle solution for 50 μL of sterile saline; (b) IIIg (25 mg/kg) biweekly i.p.; (c) IIIg (100 mg/kg) biweekly i.p. Treatments are continued for 4 weeks. All mice are weighed weekly and observed for tumor growth. Tumor diameter is assessed with a Vernier caliper, and tumor volume (mm³) is calculated as d²×D/2, wherein d and D represent the shortest and longest diameters, respectively. Bulky disease is considered present when tumor burden is prominent in the mouse abdomen (tumor volume, >=2,000 mm³).

In another experiment, mice are randomly allocated into four groups (n=10 mice per group) to receive one of the following treatments. (a) Vehicle solution for 50 μL of sterile saline; (b) IIIg (200 mg/kg) once weekly i.p.; (c) IIIg (100 mg/kg) biweekly i.p; (d) IIIg (40 mg/kg) once daily i.p. Treatments are continued for 4 weeks. All mice are weighed weekly and observed for tumor growth. Tumor diameter is assessed with a Vernier caliper, and tumor volume (mm³) is calculated as d²×D/2, wherein d and D represent the shortest and longest diameters, respectively. Bulky disease is considered present when tumor burden is prominent in the mouse abdomen (tumor volume, >=2,000 mm³).

In another experiment, mice are randomly allocated into eight groups to receive one of the following treatments via oral or ip administration. (a) Vehicle solution for 50 μL of sterile saline (oral); (b) Gemcitabine (25 mg/kg) biweekly for 4 weeks (ip); (c) IIIg (200 mg/kg) once weekly (oral); (d) IIIg (200 mg/kg) biweekly (oral); (e) IIIg (200 mg/kg) once daily (oral); (f) IIIg (400 mg/kg) once weekly (oral); (g) IIIg (400 mg/kg) biweekly (oral); (h) IIIg (400 mg/kg) once daily (oral). In all treatments, IIIg is given orally. Treatments are continued for 4 weeks. All mice are weighed weekly and observed for tumor growth. Tumor diameter is assessed with a Vernier caliper, and tumor volume (mm³) is calculated as d²×D/2, wherein d and D represent the shortest and longest diameters, respectively. Bulky disease is considered present when tumor burden is prominent in the mouse abdomen (tumor volume, >=2,000 mm³).

When at least 6 of 10 mice in a treatment group presented with bulky disease, the median survival duration for that group is considered to be reached. At the median survival duration of the control group, the tumor growth in mice in all groups is evaluated using the bioluminescence emitted by the tumor cells. Bioluminescence imaging is conducted using a cryogenically cooled IVIS 100 imaging system coupled to a data acquisition computer running Living Image software (Xenogen). The mice are sacrificed by carbon dioxide inhalation when evidence of advanced bulky disease is present. The day of sacrifice is considered the day of death for survival evaluation.

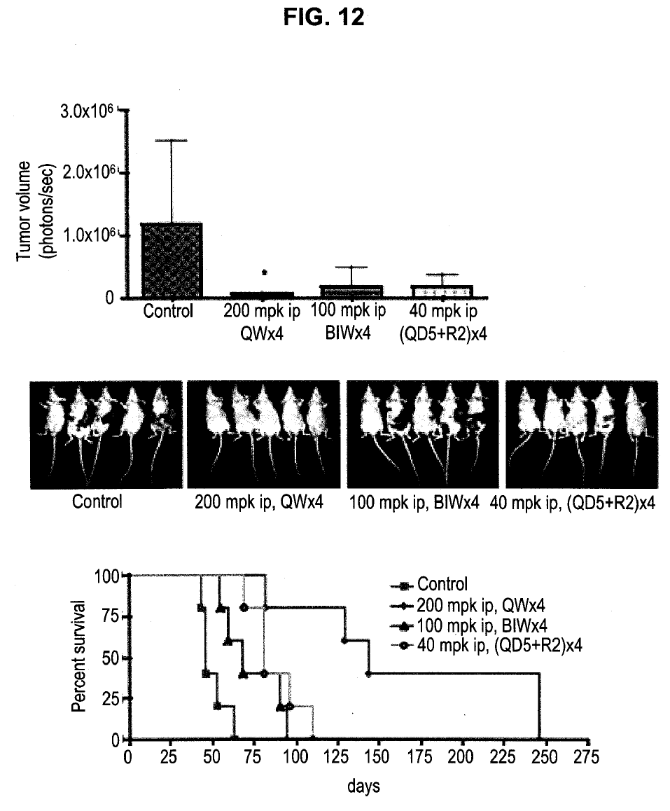

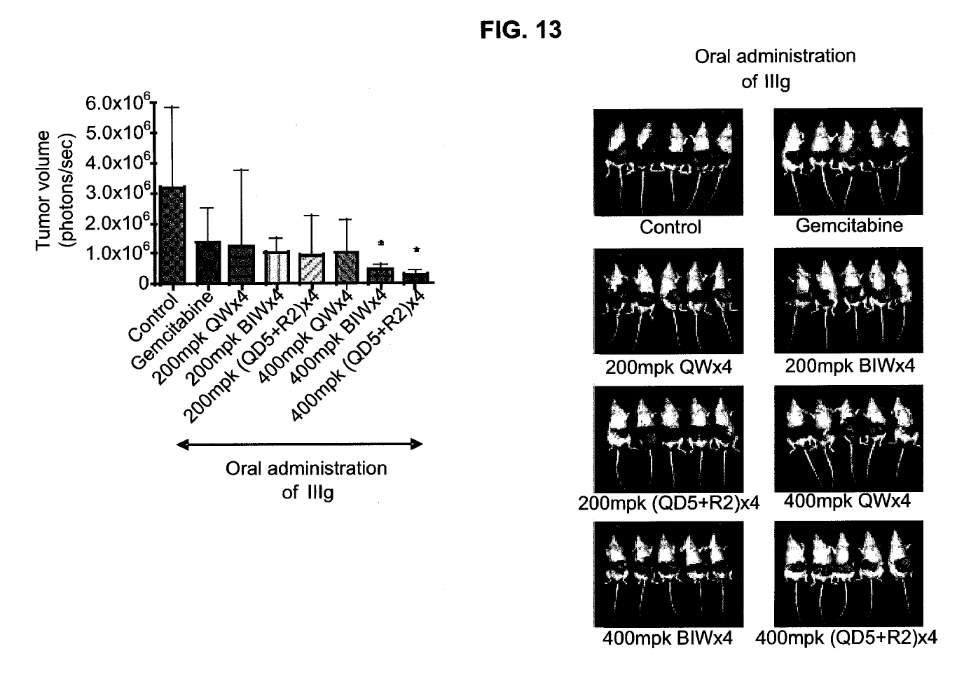

The results of the experiments are shown in FIGS. 12 and 13. Intraperitoneal injection of IIIg (200 mg/kg QW×4) potently reduces the tumor burden and prolongs the survival of the mice. Oral administration of IIIg (400 mg/kg [QD5+R2]×4) also significantly reduces the tumor burden and prolongs survival of the tumor-bearing mice as compared to mice which receive no treatment and mice which receive the standard gemicitabine treatment.

### Example 8

### Combination of IIIg with Oxaliplatin on the Proliferation of Pancreatic Tumor Cells In Vitro

This example provides the assessment of IIIg in combination with an anti-tumor agent, oxaliplatin, on the proliferation of two human pancreatic tumor cell lines, COLO357FG and MiaPaCa-2, in vitro. Cell proliferation is measured by BrdU incorporation as described in detail hereinabove. The experiments are performed according to Chou and Talalay's method (Chou T C, Talalay P. Quantitative analysis of dose-effect relationships: the combined effects of multiple drugs or enzyme inhibitors. Adv Enzyme Regul 1984, 22: 27-55).

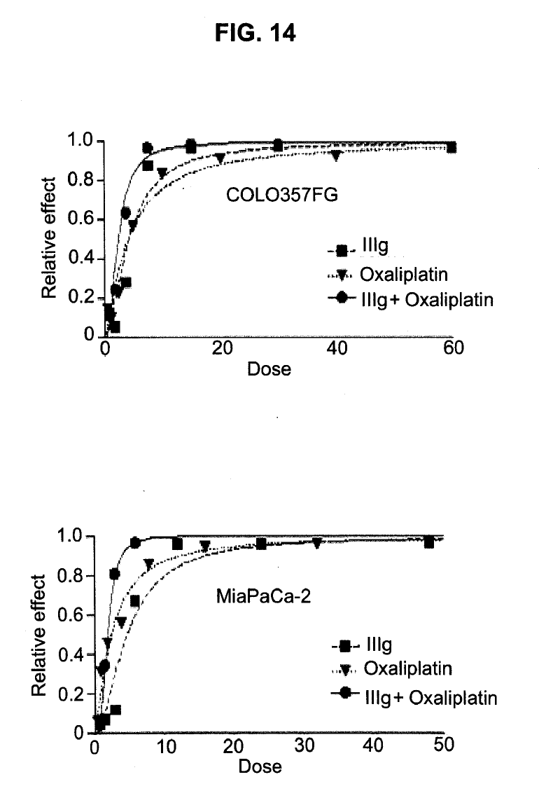

The results are shown in FIG. 14. It demonstrates that IIIg synergizes with oxaliplatin to inhibit the proliferation of the human metastatic pancreatic tumor cells.

### Example 9

### Combination of IIIg with Oxaliplatin in the Treatment of Pancreatic Tumor In Vivo

This example provides the assessment of IIIg in combination with an anti-tumor agent, oxaliplatin, on the proliferation of human pancreatic tumor cells (COLO357FG) in vivo.

Three days after the orthotopic implantation of 1.0×10⁶ COL0357FG tumor cells in 50 μL of HBSS, when bioluminescence imaging confirmed that tumors are well established, mice are randomly allocated into four groups to receive one of the following treatments: (a) Vehicle solution for 50 μL of sterile saline; (b) IIIg (400 mg/kg) once daily for 4 weeks [(QD5+R2)×4]; (c) oxaliplatin (10 mg/kg) biweekly for 4 weeks (ip); (d) combination of IIIg (400 mg/kg) once daily for 4 weeks [(QD5+R2)×4] with oxaliplatin (10 mg/kg biweekly, ip). Treatments are continued for 4 weeks. All mice are weighed weekly and observed for tumor growth. Tumor diameter is assessed with a Vernier caliper, and tumor volume (mm³) is calculated as d²×D/2, wherein d and D represent the shortest and longest diameters, respectively. Bulky disease is considered present when tumor burden is prominent in the mouse abdomen (tumor volume, >=2,000 mm³).

When at least 6 of 10 mice in a treatment group presented with bulky disease, the median survival duration for that group is considered to be reached. At the median survival duration of the control group, the tumor growth in mice in all groups is evaluated using the bioluminescence emitted by the tumor cells. Bioluminescence imaging is conducted using a cryogenically cooled IVIS 100 imaging system coupled to a data acquisition computer running Living Image software (Xenogen). The mice are sacrificed by carbon dioxide inhalation when evidence of advanced bulky disease is present. The day of sacrifice is considered the day of death for survival evaluation.

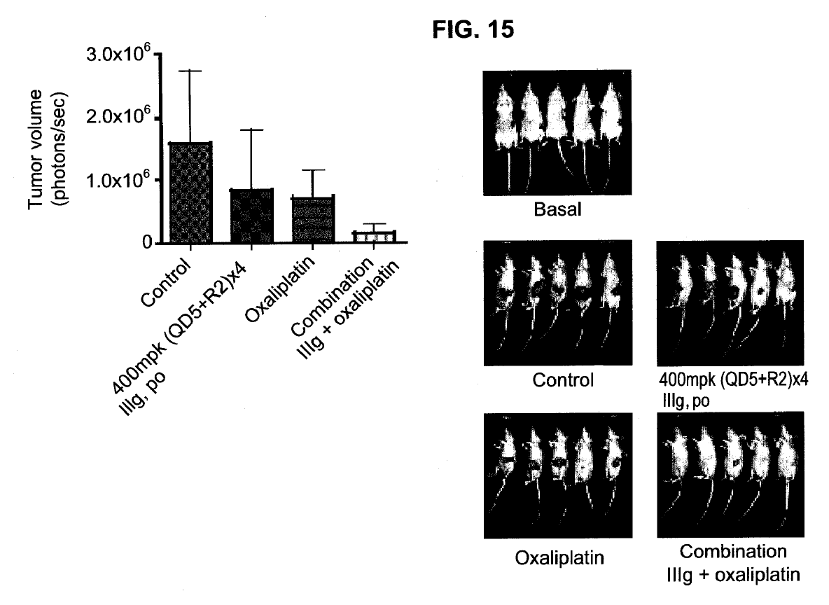

The results are shown in FIG. 15. It demonstrates that IIIg in combination with oxaliplatin has potent synergistic anti-tumor activity.

### Example 10

### Anti-Tumor Activity of IIIg on Human Breast Carcinoma Xenograft Model in Nude Mice

In this example, the anti-tumor activity of IIIg is assessed in a human MX-1 breast carcinoma xenograft model in nude mice. Varying drug regimens of IIIg with or without oxaliplatin are evaluated in a luciferase-expressing COLO357FG orthotopic nude mouse model using an IVIS 100 Imaging system. Mice are monitored daily for signs of toxicity including weight loss, diarrhea, inactivity, and general appearance.

### Mice

Female athymic nude mice (nu/nu, Harlan) are 10 weeks old, and have a body weight (BW) range of 18.1-27.0 g on D1 of the study. The animals are fed ad libitu water (reverse osmosis, 1 ppm Cl) and NIH 31 Modified and Irradiated Lab Diet® consisting of 18.0% crude protein, 5.0% crude fat, and 5.0% crude fiber. The mice are housed on irradiated ALPHA-dri® bed-o-cobs® Laboratory Animal Bedding in static microisolators on a 12-hour light cycle at 21-22° C. (70-72° F.) and 40-60% humidity. PRC specifically complies with the recommendations of the Guide for Care and Use of Laboratory Animals with respect to restraint, husbandry, surgical procedures, feed and fluid regulation, and veterinary care. The animal program at PRC is accredited by AAALAC International, which assures compliance with accepted standards for the care and use of laboratory animals.

### Tumor Implantation

The human MX-1 breast carcinoma utilized in the present study is maintained in nude mice by serial engraftment. A tumor fragment (1 mm³) is implanted s.c. into the right flank of each test mouse. Tumors are monitored twice weekly and then daily as their volumes approached 80-120 mm³. On D1 of the study, animals are sorted into treatment groups with tumor sizes of 63-144 mm³ and group mean tumor sizes of approximately 98 mm³. Tumor size, in mm3, is calculated from:

$$Tumor Volume = \frac{{w}^{2} \times l}{2}$$

where w=width and l=length in mm of the tumor. Tumor weight may be estimated with the assumption that 1 mg is equivalent to 1 mm³ of tumor volume.

### Test Articles

IIIg (experimental code for IIIg) dosing solutions are prepared fresh weekly by dissolving IIIg in 100% dimethylsulfoxide (DMSO). The compound and dosing solutions are stored at 4° C. 5-FU (positive control) (Adrucil®, Roche Diagnostics, 50 mg/mL, Lot # 200826) is diluted with 5% dextrose in water, pH 4.8. A fresh 5-FU dosing solution is prepared for each dose.

### Treatment

Mice are sorted into six groups (n=10), and treated in accordance with the protocol. Control Group 1 mice received DMSO, the IIIg vehicle, by intraperitoneal (i.p.) injection once daily for the duration of the study (qd to end). Group 2 mice received 100 mg/kg 5-FU i.p. once weekly for three weeks (qwk×3). In Group 2, the dosing volume of 0.2 mL/20-g mouse is scaled to the BW of each animal. In Groups 3, mice received IIIg twice a week (biweekly; biw) at 100 mg/kg.

### Endpoint

Tumors are calipered twice weekly for the duration of the study. Each animal is euthanized when its neoplasm reached the predetermined endpoint size (1200 mm³). The time to endpoint (TTE) for each mouse is calculated according to the following equation:

$$T T E = \frac{{log}_{10} ( endpoint volume ) - b}{m}$$

where TTE is expressed in days, endpoint volume is in mm3, b is the intercept, and m is the slope of the line obtained by linear regression of a log-transformed tumor growth data set. The data set is comprised of the first observation that exceeded the study endpoint volume and the three consecutive observations that immediately preceded the attainment of the endpoint volume. The calculated TTE is usually less than the day on which an animal is euthanized for tumor size. Animals that do not reach the endpoint are euthanized at the end of the study, and assigned a TTE value equal to the designated last day of the study (54 days). An animal classified as having died from treatment-related (TR) causes or non-treatment-related metastasis (NTRm) causes is assigned a TTE value equal to the day of death. An animal classified as having died from non-treatmentrelated (NTR) causes is excluded from TTE calculations. Treatment efficacy is determined from tumor growth delay (TGD), which is defined as the increase in the median TTE for a treatment group as compared to the control group:

TGD=T−C,

expressed in days, or as a percentage of the median TTE of the control group:

$$\% T G D = \frac{T - C}{C} \times 100$$

where:
T=median TTE for a treatment group,
C=median TTE for control Group 1.

### MTV and Criteria for Regression Responses

Treatment efficacy is also determined from the tumor volumes of animals remaining in the study on the last day, and from the number of regression responses. The MTV(n) is defined as the median tumor volume on D54 in the number of animals remaining, n, whose tumors have not attained the endpoint volume. Treatment may cause a partial regression (PR) or a complete regression (CR) of the tumor in an animal. A PR indicates that the tumor volume is 50% or less of its D1 volume for three consecutive measurements during the course of the study, and equal to or greater than 13.5 mm³ for one or more of these three measurements. A CR indicates that the tumor volume is less than 13.5 mm³ for three consecutive measurements during the course of the study. An animal with a CR at the termination of a study is additionally classified as a tumor-free survivor (TFS).

### Statistical and Graphical Analyses

Statistical and graphical analyses are conducted using Prism 3.03 (GraphPad) for Windows. The Kiruskal-Wallis test, and post hoc analysis with Dunn's multiple comparison test, are employed to analyze for differences among the treatment groups, and between two treatment groups, respectively. The Kiruskal-Wallis, which tests for differences in distribution functions, is an extension of the Mann-Whitney test, and an analog of the F-test used in ANOVAs. The logrank test is employed to analyze for differences between the overall survival experiences of two groups. The Kiruskal-Wallis and logrank tests utilize the TTE data for all animals in a group, except the NTR deaths. The two-tailed statistical analyses are conducted at P=0.05.

Kaplan-Meier plots show the percentage of animals remaining in the study versus time. Kaplan-Meier plots use the same TTE data as the Kiruskal-Wallis and logrank tests. The tumor growth curves show the group median tumor volume as a function of time. When an animal exits the study due to tumor size or TR death, the final tumor volume recorded for the animal is included with the data used to calculate the median volume at subsequent time points. Therefore, the final median tumor volume shown by the curve may differ from the MTV, which is the median tumor volume for mice remaining in the study on the last day (excluding all with tumors that have attained the endpoint). If more than one TR death occurs in a group, the median tumor growth curve is customarily truncated at the time of the last measurement that precedes the second TR death. Tumor growth curves are also truncated when the tumors in more than 50% of the assessable animals in a group have attained the endpoint volume.

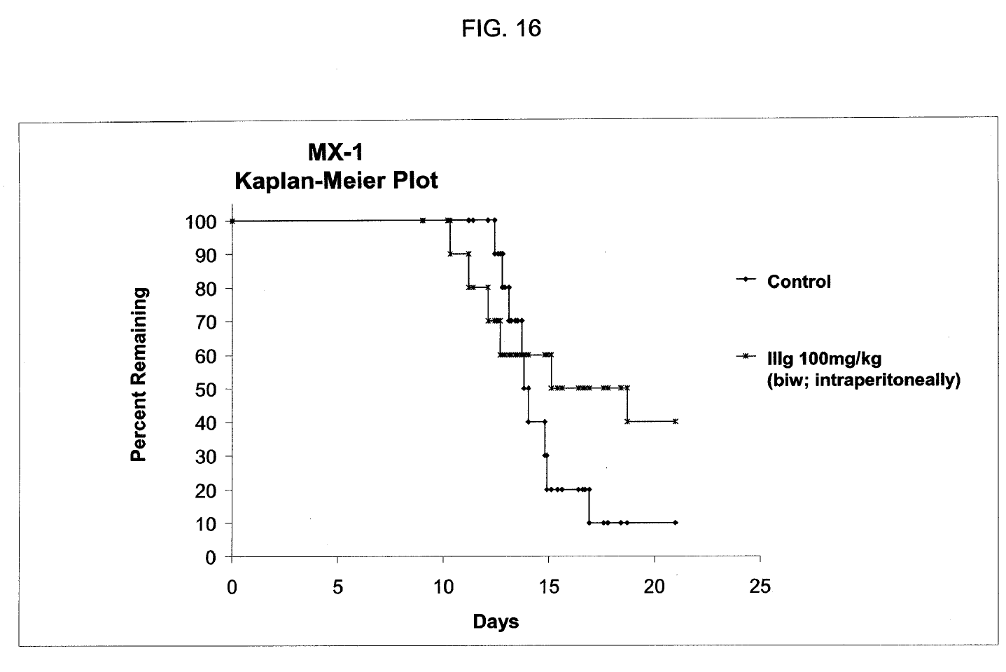

The results are shown in FIG. 16. Biweekly intraperitoneal injection of IIIg (100 mg/kg) prolongs survival of the tumor-bearing animals.

### Example 11

### Anti-Tumor Activity of IIIg on Human Colon Carcinoma Xenograft Model in Nude Mice

In this example, the anti-tumor activity of IIIg is assessed in a human SW620 colon adenocarcinoma xenograft model in nude mice. Varying drug regimens of IIIg with or without oxaliplatin are evaluated in a luciferase-expressing COLO357FG orthotopic nude mouse model using an IVIS 100 Imaging system. Mice are monitored daily for signs of toxicity including weight loss, diarrhea, inactivity, and general appearance.

### Mice

Female athymic nude mice (nu/nu, Harlan) are 10 weeks old, and had a body weight (BW) range of 18.1-27.0 g on D1 of the study. The animals are fed ad libitu water (reverse osmosis, 1 ppm Cl) and NIH 31 Modified and Irradiated Lab Diet® consisting of 18.0% crude protein, 5.0% crude fat, and 5.0% crude fiber. The mice are housed on irradiated ALPHA-dri® bed-o-cobs® Laboratory Animal Bedding in static microisolators on a 12-hour light cycle at 21-22° C. (70-72° F.) and 40-60% humidity. PRC specifically complies with the recommendations of the Guide for Care and Use of Laboratory Animals with respect to restraint, husbandry, surgical procedures, feed and fluid regulation, and veterinary care. The animal program at PRC is accredited by AAALAC International, which assures compliance with accepted standards for the care and use of laboratory animals.

### Tumor Implantation

The human SW620 colon adenocarcinoma utilized in the present study is maintained in nude mice by serial engraftment. A tumor fragment (1 mm³) is implanted s.c. into the right flank of each test mouse. Tumors are monitored twice weekly and then daily as their volumes approached 80-120 mm³. On D1 of the study, animals are sorted into treatment groups with tumor sizes of 63-144 mm³ and group mean tumor sizes of approximately 98 mm³. Tumor size, in mm3, is calculated from:

$$Tumor Volume = \frac{{w}^{2} \times l}{2}$$

where w=width and l=length in mm of the tumor. Tumor weight may be estimated with the assumption that 1 mg is equivalent to 1 mm³ of tumor volume.

### Test Articles

IIIg (experimental code for IIIg) dosing solutions are prepared fresh weekly by dissolving IIIg in 100% dimethylsulfoxide (DMSO). The compound and dosing solutions are stored at 4° C. 5-FU (positive control) (Adrucil®, Roche Diagnostics, 50 mg/mL, Lot # 200826) is diluted with 5% dextrose in water, pH 4.8. A fresh 5-FU dosing solution is prepared for each dose.

### Treatment

Mice are sorted into six groups (n=10), and treated in accordance with the protocol. Control Group 1 mice received DMSO, the IIIg vehicle, by intraperitoneal (i.p.) injection once daily for the duration of the study (qd to end). Group 2 mice received 100 mg/kg 5-FU i.p. once weekly for three weeks (qwk×3). In Group 2, the dosing volume of 0.2 mL/20-g mouse is scaled to the BW of each animal. In Groups 3, mice received IIIg biweekly for the duration of the study (biwk to end) at 50 mg/kg.

### Endpoint

Tumors are calipered twice weekly for the duration of the study. Each animal is euthanized when its neoplasm reached the predetermined endpoint size (1200 mm³). The time to endpoint (TTE) for each mouse is calculated according to the following equation:

$$T T E = \frac{{log}_{10} ( endpoint volume ) - b}{m}$$

where TTE is expressed in days, endpoint volume is in mm3, b is the intercept, and m is the slope of the line obtained by linear regression of a log-transformed tumor growth data set. The data set is comprised of the first observation that exceeded the study endpoint volume and the three consecutive observations that immediately preceded the attainment of the endpoint volume. The calculated TTE is usually less than the day on which an animal is euthanized for tumor size. Animals that do not reach the endpoint are euthanized at the end of the study, and assigned a TTE value equal to the designated last day of the study (54 days). An animal classified as having died from treatment-related (TR) causes or non-treatment-related metastasis (NTRm) causes is assigned a TTE value equal to the day of death. An animal classified as having died from non-treatment-related (NTR) causes is excluded from TTE calculations. Treatment efficacy is determined from tumor growth delay (TGD), which is defined as the increase in the median TTE for a treatment group as compared to the control group:

TGD=T−C,

expressed in days, or as a percentage of the median TTE of the control group:

$$\% T G D = \frac{T - C}{C} \times 100$$

where:
T=median TTE for a treatment group,
C=median TTE for control Group 1.

### MTV and Criteria for Regression Responses

Treatment efficacy is also determined from the tumor volumes of animals remaining in the study on the last day, and from the number of regression responses. The MTV(n) is defined as the median tumor volume on D54 in the number of animals remaining, n, whose tumors have not attained the endpoint volume. Treatment may cause a partial regression (PR) or a complete regression (CR) of the tumor in an animal. A PR indicates that the tumor volume is 50% or less of its D1 volume for three consecutive measurements during the course of the study, and equal to or greater than 13.5 mm³ for one or more of these three measurements. A CR indicates that the tumor volume is less than 13.5 mm³ for three consecutive measurements during the course of the study. An animal with a CR at the termination of a study is additionally classified as a tumor-free survivor (TFS).

### Statistical and Graphical Analyses

Statistical and graphical analyses are conducted using Prism 3.03 (GraphPad) for Windows. The Kiruskal-Wallis test, and post hoc analysis with Dunn's multiple comparison test, are employed to analyze for differences among the treatment groups, and between two treatment groups, respectively. The Kiruskal-Wallis, which tests for differences in distribution functions, is an extension of the Mann-Whitney test, and an analog of the F-test used in ANOVAs. The logrank test is employed to analyze for differences between the overall survival experiences of two groups. The Kiruskal-Wallis and logrank tests utilize the TTE data for all animals in a group, except the NTR deaths. The two-tailed statistical analyses are conducted at P=0.05.

Kaplan-Meier plots show the percentage of animals remaining in the study versus time. Kaplan-Meier plots use the same TTE data as the Kiruskal-Wallis and logrank tests. The tumor growth curves show the group median tumor volume as a function of time. When an animal exits the study due to tumor size or TR death, the final tumor volume recorded for the animal is included with the data used to calculate the median volume at subsequent time points. Therefore, the final median tumor volume shown by the curve may differ from the MTV, which is the median tumor volume for mice remaining in the study on the last day (excluding all with tumors that have attained the endpoint). If more than one TR death occurs in a group, the median tumor growth curve is customarily truncated at the time of the last measurement that precedes the second TR death. Tumor growth curves are also truncated when the tumors in more than 50% of the assessable animals in a group have attained the endpoint volume.

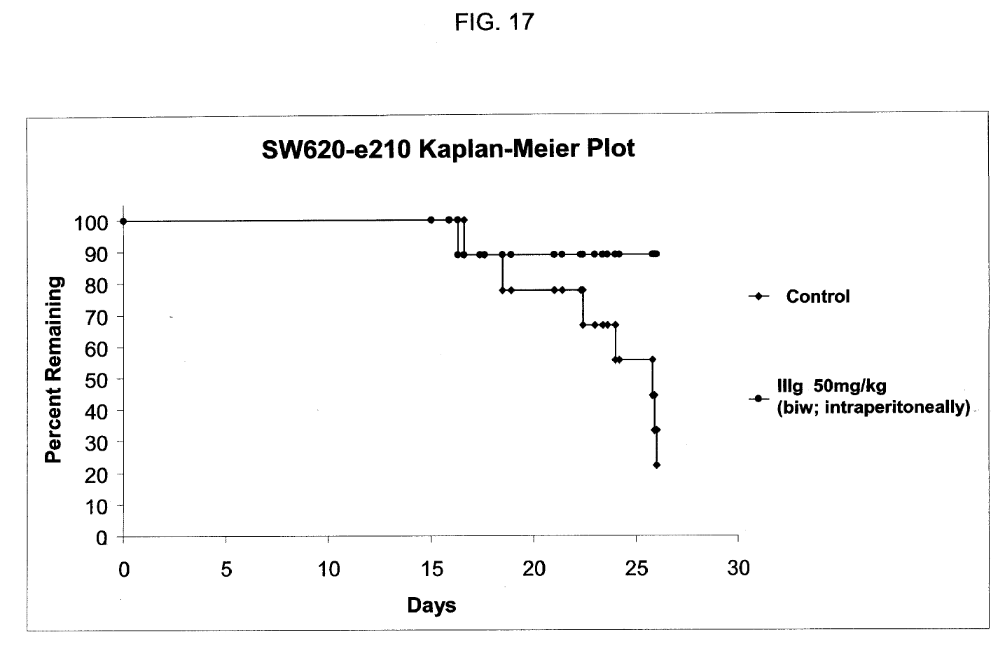

The results are shown in FIG. 17. Biweekly intraperitoneal injection of IIIg (50 mg/kg) prolongs survival of the tumor-bearing animals.

### Example 12

### Effect of IIIg in Combination with Irradiation on Proliferation of Pancreatic Cancer Cells

BrdU assay is well known in the art. Briefly, cells are cultured in the presence of the respective test substances in an appropriate 96-well MP at 37° C. for a certain period of time (1 to 5 days, depending on the individual assay system). Subsequently, BrdU is added to the cells and the cells are reincubated (usually 2-24 h). During this labeling period, the pyrimidine analogue BrdU is incorporated in place of thymidine into the DNA of proliferating cells. After removing the culture medium, the cells are fixed and the DNA is denatured in one step by adding FixDenat (the denaturation of the DNA is necessary to improve the accessibility of the incorporated BrdU for detection by the antibody). The anti-BrdU-POD antibody is added and the antibody binds to the BrdU incorporated in newly synthesized, cellular DNA. The immune complexes are detected by the subsequent substrate reaction via chemiluminescent detection (based on Cell Proliferation ELISA, BrdU Chemiluminescence Protocol from Roche).

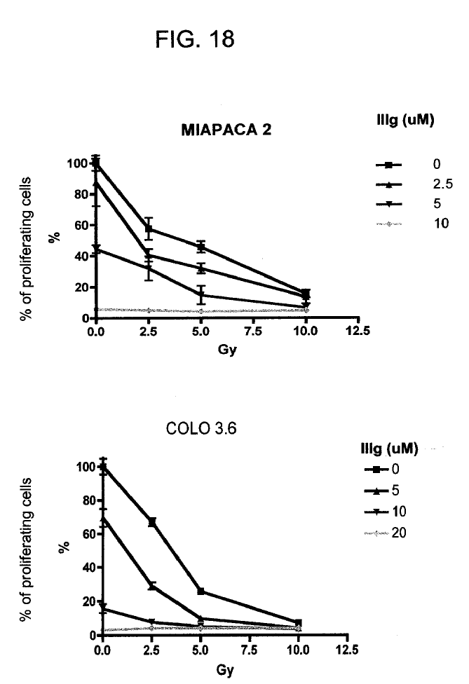

Results are shown in FIG. 18.

### Example 13

### Effects of Compound IIIc in Combination with Oxaliplatin on Cell Cycle Distribution of Metastatic Pancreatic Adenocarcinoma Cells

Colo375FG metastatic pancreatic adenocarcinoma cells are obtained from ATCC and cultured in Dulbecco Modified Eagle Medium with 10% fetal bovine serum. Cells are plated at 105 cells per P100 cell culture dish or at 104 cells per P60 cell culture dish in the presence of different concentrations compounds or DMSO control. Following treatment, the number of attached cells is measured using Coulter counter, and by staining with 1% methylene blue. Methylene blue is dissolved in 50%-50% mixture of Methanol and water. Cells are plated in 24- or 96-well plates and treated as planned, media are aspirated, cells are washed with PBS, fixed in methanol for 5-10 min, methanol is aspirated and plates are allowed to dry completely. Methylene blue solution is added to wells and plates are incubated for 5 min. Staining solution is removed and plates are washed with dH2O until washes are no longer blue. After plates are completely dry, a small amount of 1N HCl is added to each well to extract the methylene blue. The OD readout at 600 nm and a calibration curve are used to determine cell number.

Compounds of the invention are dissolved directly from dry powder to 10 mM stock solution in DMSO for each separate experiment. Control experiments are carried out with the matching volume/concentration of the vehicle (DMSO). In these controls, the cells show no changes in their growth or cell cycle distribution.

### PI Exclusion, Cell Cycle and TUNEL Assays

After the addition of drugs and incubation, cells are trypsinized and aliquots of the samples are taken for counting and PI (Propidium Iodide) exclusion assay. One part of the cells is centrifuged and resuspended in 0.5 ml ice-cold PBS containing 5 ug/ml of PI. The other part of the cells is fixed in ice-cold 70% ethanol and stored in a freezer overnight. For cell cycle analysis, cells are stained with propidium iodide (PI) by standard procedures. Cellular DNA content is determined by flow cytometry using BD LSRII FACS, and the percentages of cells in G1, S or G2/M are determined using ModFit software.

The cells are labeled for apoptosis with the “In Situ Cell Death Detection Kit, Fluorescein” (Roche Diagnostics Corporation, Roche Applied Science, Indianapolis, Ind.). Briefly, fixed cells are centrifuged and washed once in phosphate-buffered saline (PBS) containing 1% bovine serum albumin (BSA), then resuspended in 2 ml permeabilization buffer (0.1% Triton X-100 and 0.1% sodium citrate in PBS) for 25 minutes at room temperature and washed twice in 0.2 ml PBS/1% BSA. The cells are resuspended in 50 μl TUNEL reaction mixture (TdT enzyme and labeling solution) and incubated for 60 min at 37° C. in a humidified dark atmosphere in an incubator. The labeled cells are washed once in PBS/1% BSA, then resuspended in 0.5 ml ice-cold PBS containing 1 μg/ml 4,6-diamidino-2-phenylindole (DAPI) for at least 30 min. All cell samples are analyzed with a BD LSR II (BD Biosciences, San Jose, Calif.).

### Bromodeoxyuridine (BrdU) Labeling Assay

50 μl of BrdU (Sigma Chemical Co., St. Louis, Mo.) stock solution (1 mM) is added to give 10 μM BrdU final concentration. The cells are incubated for 30 min at 37□ C and fixed in ice-cold 70% ethanol and stored in a cold room (4□ C) overnight. Fixed cells are centrifuged and washed once in 2 ml PBS, then resuspended in 0.7 ml of denaturation solution (0.2 mg/ml pepsin in 2 N HCl) for 15 min at 37° C. in the dark and suspended with 1.04 ml 1M Tris buffer (Trizma base, Sigma Chemical Co.) and washed in 2 ml PBS. Then cells are resuspended in 100-μl anti-BrdU antibody (DakoCytomation, Carpinteria, Calif.) with 1:100 dilution in TBFP permeable buffer (0.5% Tween-20, 1% bovine serum albumin and 1% fetal bovine serum in PBS) and incubated for 25 min at room temperature in the dark and washed in 2 ml PBS. The primary antibody-labeled cells are resuspended in 100 μl Alexa Fluor F(ab′)2 fragment of goat anti-mouse IgG (H+L) (2 mg/mL) (Molecular Probes, Eugene, Oreg.) with 1:200 dilution in TBFP permeable buffer and incubated for 25 min at room temperature in the dark and washed in 2 ml PBS, then resuspended in 0.5 ml ice-cold PBS containing 1 μg/ml 4′,6-diamidino-2-phenylindole (DAPI) for at least 30 min. All cell samples are analyzed with a BD LSR II (BD Biosciences, San Jose, Calif.).

Results are summarized in Table 6.

TABLE 6

| | | | | Dead | Cells in S- |
|---|---|---|---|---|---|
| | | | Live | cells | phase of cell |
| | Compound | OxaliPt | Cell | TUNEL | cycle |
| | IIIc (mM) | (mM) | (%) | (%) | (% BrdU+) |
| Oxaliplatin | 0 | 0 | 100 | 2.6 | |
| (OxaliPt) | 0 | 10 | 54 | 21.0 | 81.74 |
| | 0 | 20 | 29 | 48.3 | |
| 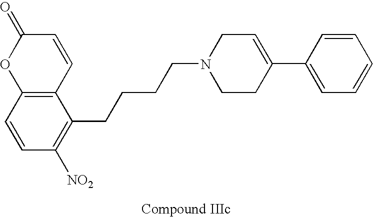 | 12.5 | 0 10 20 | 54 33 25 | 2.7 16.1 28.2 | 73.87 |

### Example 14

### Human Clinical Trials

Title: A Phase 1, first in human, open-label, dose escalation study evaluating the safety and pharmacokinetics of benzopyrone compound, more specifically, 5-iodo-6-nitro-benzopyrone (IIIg) in subjects with advanced solid tumors.

Study Phase: 1

Indication: Treatment of advanced solid tumors

Primary Objective: To assess the safety, establish the maximum tolerated dose (MTD) and generate pharmacokinetic profiles of IIIg after IV administration in adult subjects with histologically documented advanced solid tumors that are refractory to standard therapy or for which no standard therapy is available.

Secondary Objective(s): To evaluate the response in study subjects (per RECIST criteria) with measurable disease. To assess safety profiles: significant laboratory changes and adverse events (AEs) not defined as a dose limiting toxicity (DLT).

Exploratory Objective(s): To assess the effect of treatment on biological markers of tumor status.

Study Design: A phase 1, first in human, open-label, sequential dose escalation study designed to determine safety, MTD and PK profile of IIIg. IIIg will be administered intravenously twice weekly (days 1 and 4 of each week) for 3 weeks, followed by a one week IIIg treatment free period per one 28-day cycle. Cycle one (day 1 thru day 28) will be defined as the safety phase of the study during which the MTD will be determined. The remainder of the study will be termed the maintenance phase. Subjects may participate in this study until a subject experiences a drug intolerance or disease progression.

Safety assessment will follow the guidelines provided in the Cancer Therapy Evaluation Program Common Terminology Criteria for Adverse Events (CTCAE) Version 3.0 dated December, 2003. The first assessment of tumor response, for measurable disease, will be performed during week 8 of the study, and approximately every 8 weeks thereafter. The modified Response Evaluation Criteria in Solid Tumors (RECIST) criteria will be used to establish disease progression. For non-measurable disease, best medical practices will be used to determine time of disease progression

Primary Endpoint and Secondary Endpoints: Primary endpoints being safety/tolerability to characterize DLT and PK profiles: BA half life (t½), maximum observed concentration (Cₘₐₓ), area under the plasma concentration-time curve (AUC), and clearance (CL). Secondary endpoints being tumor response per RECIST criteria; safety profiles: significant laboratory changes and other AEs (not defined as a DLT). Exploratory being reduction in circulating tumor cell (CTC) levels.

Sample Size: As many as 36 subjects are expected to participate in this study. Study subjects will be assigned to sequential cohorts of 1, 3, or 6 subjects at varying dose levels. As many as 10 dose cohorts may be needed to define the MTD.

Summary of Subject Eligibility Criteria:

Inclusion criteria include: (a) ≧18 years old with a pathologically documented, advanced solid tumor that is refractory to standard treatment or for which no standard therapy is available, (b) Eastern Oncology Cooperative Group (ECOG) performance status of ≦2, and (c) absolute neutrophil count (ANC)≧1.5×109/L (without GCF support within 2 weeks of study day 1); platelet count≧100.0×109/L (without transfusion within 2 weeks of study day 1); and hemoglobin ≧9.0 g/dL (erythropoietic agents allowed).

Exclusion Criteria include: subject enrolled in another investigational device or drug trial, or is receiving other investigational agents; hematological malignancies; symptomatic or untreated brain metastases requiring concurrent treatment, inclusive of but not limited to surgery, radiation, and corticosteroids; history of seizure disorder; MI within 6 months of study day 1, unstable angina, congestive heart failure (CHF) with New York Heart Association (NYHA)>class II, uncontrolled hypertension; concurrent or prior (within 7 days of study day 1) anticoagulation therapy; specified concomitant medications (see Section 4.2.3); serum creatinine >1.5×ULN; elevated liver enzymes (AST/ALT)>2.5×ULN, or >5.0 if secondary to liver metastases, alkaline phosphatase >2.5×ULN or >5.0 if secondary to liver or bone metastases; total bilirubin >1.5×ULN; systemic chemotherapy within 28 days of study day 1 (42 day washout period for BCNU or mitomycin C); radiation therapy within 28 days of study day 1; antibody therapy for the treatment of an underlying malignancy within 1 month of study day 1, and; concurrent chemotherapy with any agent other than BA or radiation therapy is not permitted throughout the course of the study.

Investigational Product Dosage and Administration: IIIg will be provided in 10 mL vials of 10 mg/mL concentration. t is estimated that as many as 10 subject cohorts may be necessary to determine the MTD.

Starting Dose (Cohort A): In cohort A, a single subject will receive IIIg twice weekly at a dose level of 0.5 mg/kg based on weight measured at screening. If this subject experiences a grade 2 toxicity or higher, then 3 additional subjects will be enrolled in this cohort. If no additional subjects dosed in this cohort experience a DLT, then dose escalation will occur as below. If no DLT occurs in the initial subject, dose escalation will occur as below.

Dose Escalation Prior to Grade 2 Toxicity (Potential Cohorts B-J): Until a subject experiences a grade 2 toxicity or higher, one subject will be initially enrolled in all subsequent cohorts at planned 100% dose level increases, with possible cohort expansion as described for cohort A. Safety data will be reviewed after 6 doses of IIIg, and a decision to escalate to the next cohort will be made if no subject experiences a grade 2 toxicity or higher. If 1 subject in this cohort experiences a grade 2 toxicity or higher, then 3 additional subjects will be enrolled in this cohort. If none of these three additional subjects dosed in this cohort experience a DLT, then further dose escalation will occur. If 1 of 3 subjects experience a DLT, then 3 additional subjects will be enrolled in the same cohort with the same dose. If 0 of these 3 subjects experience a DLT then escalation will occur. If one or more of the additional subjects in a cohort experience a DLT, then the previous lower dose level will be defined as the MTD. Additional subjects may be accrued at the MTD if needed to ensure at least 18 subjects receive BA in the study.

Dose Escalation After Grade 2 Toxicity Level (Potential Cohorts B-J): After the dose associated with the initial grade 2 toxicity is expanded and cleared for dose escalation to the next level, then three subjects will be initially enrolled in all future cohorts (cohorts B, C, D, E, F, G, H, I, or J). If 0 of the 3 initial subjects experience a DLT, then dose escalation to the next cohort will proceed. If 1 of 3 subjects experience a DLT, then 3 additional subjects will be enrolled in the same cohort with the same dose. If 0 of these 3 subjects experience a DLT, then escalation will occur. If one or more of the additional subjects in a cohort experience a DLT, then the previous lower dose level will be defined as the MTD. Additional subjects may be accrued at the MTD if needed to ensure that at least 18 subjects receive IIIg in the study.

Intra-subject Dose Escalation: Once a IIIg dose level has been declared safe and tolerable based on the criteria defined above all subjects currently on lower doses may be escalated to the highest safe dose as appropriate (determined by the principal investigator). Once a MTD is determined, all subjects in the study may be escalated as appropriate to receive the MTD.

Overall Dose Escalation Limitations: When a grade 2 toxicity has been observed and that dose level subsequently cleared, individual dose escalations between cohorts will be more conservative, and will be limited to approximately a maximum 40% increase from the previous dose level until a grade 3 toxicity is seen, with subsequent escalations limited to approximately 25% dose increases. Absolute dose escalation will be decided by the safety review group after review of all available data.

Control Group: None

Procedures:

Screening: Pre-enrollment screening tests and evaluation will be performed only after a signed, written Institutional Review Board (IRB) approved informed consent is obtained from each subject. Procedures will be performed within 2 weeks of study day 1 unless otherwise noted. Clinical evaluation includes complete history, physical examination, ECOG status, height, weight, vital signs, and documentation of concomitant medications. Laboratory studies include hematology (with differential, reticulocyte count, and platelets); prothrombin time (PT) and partial thromboplastin time (PTT); comprehensive chemistry panel (sodium, potassium, chloride, CO2, creatinine, calcium, phosphorous, magnesium, BUN, uric acid, albumin, AST, ALT, alkaline phosphatase, total bilirubin, and cholesterol, HDL, and LDL), urinalysis with microscopic examination, serum tumor markers, serum or urine pregnancy test for women of child bearing potential. Cardiac studies include creatine kinase (CK), and 12-lead electrocardiogram (EKG). Clinical staging includes imaging for measurable disease by computed tomography (CT) or magnetic resonance (MRI) within 4 weeks of study day 1. Documentation of clinical staging for non-measurable disease will occur.

Treatment: Eligible subjects will be enrolled into the study and receive study drug on Day 1. Pre-dose and post-dose tests will be performed as outlined in the study protocol. Dosing of BA will occur twice weekly at days 1, 4, 8, 11, 15, and 18 of each 28 day cycle; and administered over an infusion period as long as 2 hours. On day 29, subjects will start cycle 2 and resume dosing at days 1, 4, 8, 11, 15, and 18 of that and each subsequent cycle. Subjects may participate in this study until they experience a drug intolerance or disease progression or withdraw consent. Subjects meeting the modified RECIST criteria of disease progression may continue in the study if they are demonstrating clinical benefit.

The first scheduled tumor response measurement for measurable disease will be performed during week 8 (study day 50±5 days) of the study, and every 8 weeks thereafter. Tumor response according to the modified Response Evaluation Criteria in Solid Tumors (RECIST) criteria will be used to establish disease progression by CT or MRI (the same technique used during screening must be used). For non-measurable disease, best medical practices will be used to determine time of disease progression.

End of Study: All subjects should have the end of study procedures as described in the protocol completed no more than 30 days after the last dose of IIIg. Additionally, subjects will have overall tumor response assessed via clinical imaging if not done within 30 days prior to the last dose of IIIg.

Statistical Considerations: Descriptive statistics will be calculated for safety, PK, and PD endpoints. Response data, to establish time to progression, will be reported descriptively in the form of listings. Tumor progression data will be categorized using the modified RECIST criteria.

PK parameters will be estimated using non-compartmental methods. PK parameters will be summarized by the arithmetic mean, standard deviation, coefficient of variation, maximum, minimum, median, and geometric mean. Summary statistics will be calculated with SPlus version 5.1 (or later).

If appropriate, data may also be analyzed by a non-linear mixed-effects modeling approach (population approach) to compartmental analysis. Other analyses will be done descriptively as appropriate.

Results will be analyzed after all subjects have received at least one cycle (6 doses) of IIIg at the MTD dose level (or their highest dose level received in the study). This will coincide

10 with the completion of the safety phase of the study. Additional analyses will be performed on an ongoing basis as necessary to provide information for design of future trials.

### Example 15

### Identification of IIIg Metabolites in Whole Blood Samples

### Materials and Methods

Samples are prepared for HPLC injection by precipitating whole blood (50 μL) with 3× volumes (150 μL) of acetonitrile. Following centrifugation, 190 μL of each supernatant is evaporated to dryness, reconstituted in 50 μl of 0.2% formic acid in 95% water 5% methanol and analyzed by the following LC/MS/MS conditions:

| | |
|---|---|
| HPLC: | Shimadzu VP System |
| Mobile Phase: | 0.2% formic acid in water (A) and 0.18% formic |
| | acid in methanol (B) |
| Column: | 1 × 50 mm Thermo BetaBasic C18 column |
| Injection Volume: | 25 μl |
| Gradient: | 5-75% B in 30 minutes |
| Flow Rate: | 100 μl/min |
| Mass Spectrometer: | Applied Biosystems/MDS SCIEX Q-STAR |
| Interface: | TurboIonSpray |
| Parent Ion Scan: | TOF Positive from 200-1200 amu |
| Product Ion Scan: | TOF Product Ion from 60-1200 amu of most |
| | intense ion in Parent Ion Scan |
| TOF Calibration: | Externally calibrated using Renin Substrate |

### Results:

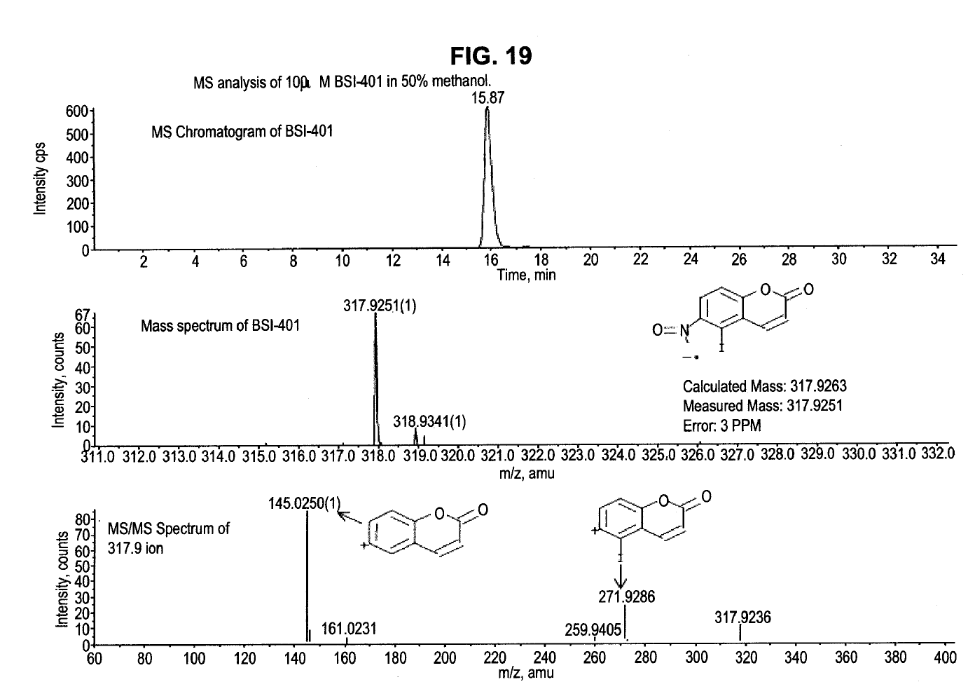

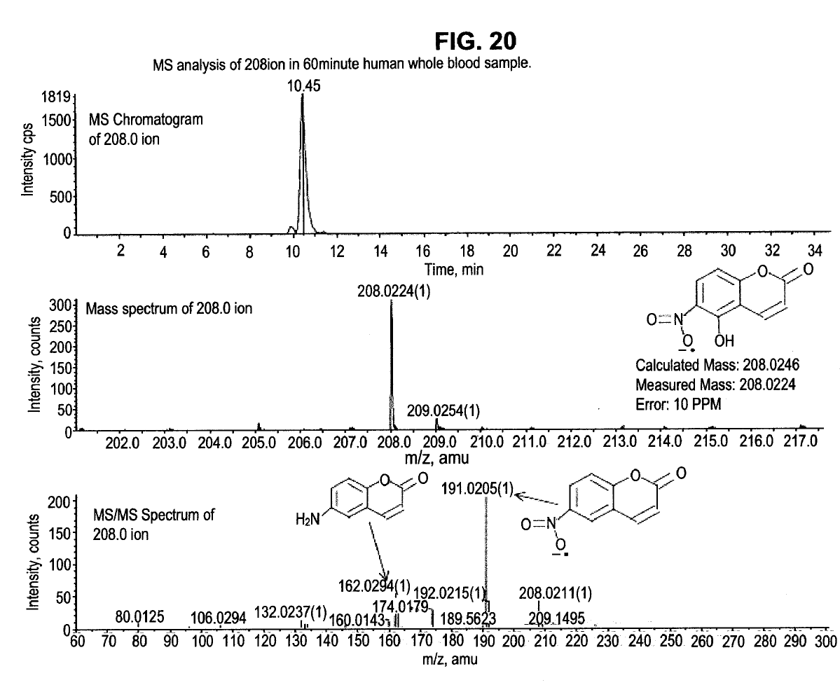

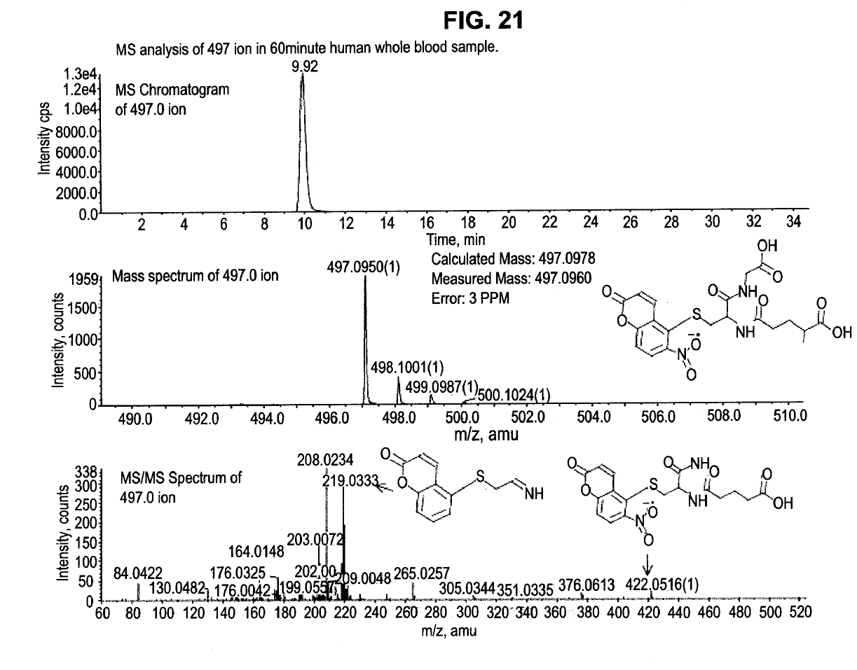

The UV and MS analysis of IIIg and potential metabolites are shown below in FIGS. 19-21. The 208 and 497 m/z ions are present in both the 0 and 60 minute time points. The 497 m/z ion appears to be a glutathione conjugate of the 208 m/z ion. Figures contain the extracted ion chromatogram, the MS (parent ion) spectrum and the MS/MS spectrum of the corresponding analyte.

The above examples are in no way intended to limit the scope of the instant invention. Further, it can be appreciated to one of ordinary skill in the art that many changes and modifications can be made thereto without departing from the spirit or scope of the appended claims, and such changes and modifications are contemplated within the scope of the instant invention.

It will be apparent to one of ordinary skill in the art that many changes and modifications can be made thereto without departing from the spirit or scope of the appended claims.

What is claimed is:

1. A method of treating a cancer comprising administering to a subject in need thereof an effective amount of a compound of formula (I), or a metabolite, a pharmaceutically acceptable salt or prodrug thereof: 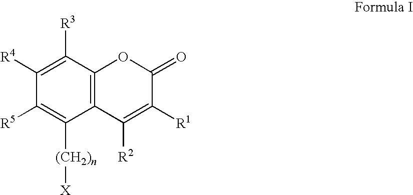
wherein n=0-10; R¹, R², R³, R⁴, R⁵ and X are independently selected from the group consisting of hydrogen, hydroxy, optionally substituted amine, amino, carboxyl, ester, nitroso, nitro, halogen, optionally substituted (C₁-C₆) alkyl, optionally substituted (C₁-C₆) alkoxy, optionally substituted (C₃-C₇) cycloalkyl, optionally substituted (C₃-C₇) heterocyclic, phenyl, and optionally substituted aryl; and wherein at least two of the R¹, R², R³, R⁴, and R⁵ substituents are always hydrogen;
wherein the cancer is selected from the group consisting of adrenal cortical cancer, anal cancer, aplastic anemia, bile duct cancer, bladder cancer, bone cancer, bone metastasis, central nervous system (CNS) cancers, peripheral nervous system (PNS) cancers, Castleman's disease, cervical cancer, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's disease, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, uterine cancer (e.g. uterine sarcoma), vaginal cancer, vulvar cancer, and Waldenstrom's macroglobulinemia.

2. A method of treating a cancer comprising administering to a subject in need thereof an effective amount of a compound of formula (I), or a metabolite, a pharmaceutically acceptable salt or prodrug thereof: 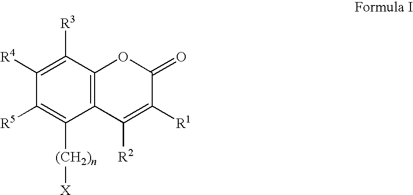
wherein n=0-10; R¹, R², R³, R⁴, R⁵ and X are independently selected from the group consisting of hydrogen, hydroxy, optionally substituted amine, amino, carboxyl, ester, nitroso, nitro, halogen, optionally substituted (C₁-C₆) alkyl, optionally substituted (C₁-C₆) alkoxy, optionally substituted (C₃-C₇) cycloalkyl, optionally substituted (C₃-C₇) heterocyclic, phenyl, and optionally substituted aryl; and wherein at least two of the R¹, R², R³, R⁴, and R⁵ substituents are always hydrogen;
wherein said cancer is a cancer formed at a different site of a body as a result of migration of a cell from a cancer selected from the group consisting of adrenal cortical cancer, anal cancer, aplastic anemia, bile duct cancer, bladder cancer, bone cancer, bone metastasis, central nervous system (CNS) cancers, peripheral nervous system (PNS) cancers, Castleman's disease, cervical cancer, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's disease, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, uterine cancer (e.g. uterine sarcoma), vaginal cancer, vulvar cancer, and Waldenstrom's macroglobulinemia.

3. The method of claim 1 or 2, wherein the compound is of formula II or a metabolite, a pharmaceutically acceptable salt or prodrug thereof: 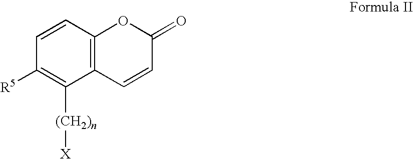
wherein R⁵ is selected from the group consisting of hydrogen, carboxyl, amino, nitroso, nitro, hydroxylamino, and hydroxy; and X is selected from the group consisting of halogen, hydroxy, optionally substituted (C₁-C₇) alkyl, optionally substituted (C₁-C₆) alkoxy, optionally substituted (C₃-C₇) cycloalkyl, optionally substituted (C₃-C₇) heterocyclic, phenyl, and optionally substituted aryl.

4. The method of claim 3, wherein X is a halogen selected from the group consisting of F, Cl, Br and I.

5. The method of claim 3, wherein X is iodine (I) and R⁵ is nitro, nitroso, hydroxylamino, hydroxyl, or amino.

6. The method of claim 3, wherein n is 0.

7. The method of claim 3, wherein the optionally substituted alkyl is substituted with a substituent selected from the group consisting of alkylamine, pyrrole, dihydropyrrole, and pyrrolidene.

8. The method of claim 3, wherein the compound is of the formula IIIa, IIIb, IIIc, IIId, IIIe, IIIf, IIIg, IIIh, IIIk, IIIl, IIIm, or IIIn, or one of their pharmaceutically acceptable salts or prodrugs:

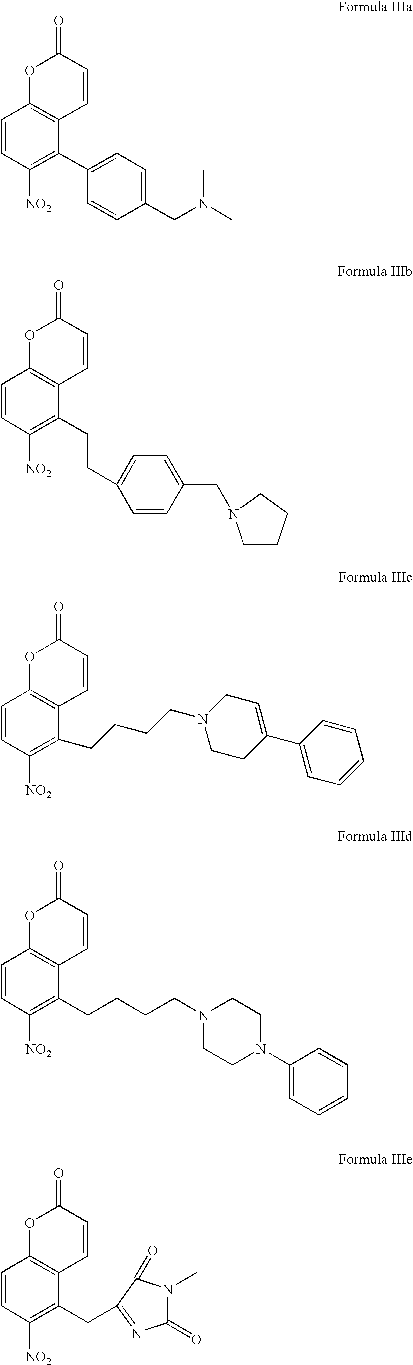

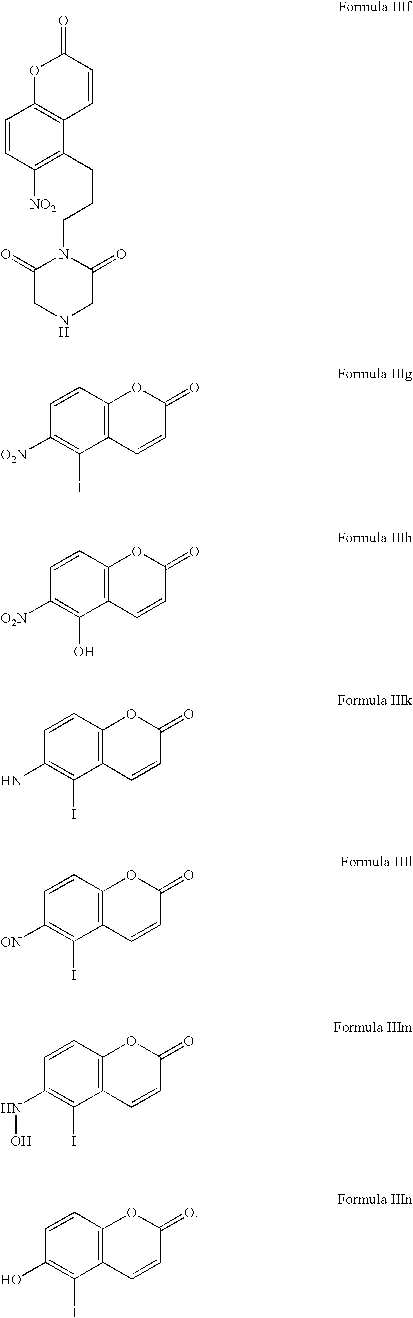

9. The method of claim 8, wherein the compound is 5-iodo-6-nitro-benzopyrone of Formula IIIg, or a metabolite, a pharmaceutically acceptable salt or prodrug thereof.

10. The method of claim 8, wherein the compound is 5-iodo-6-amino-benzopyrone of Formula IIIk, or a metabolite, a pharmaceutically acceptable salt or prodrug thereof.

11. The method of claim 8, wherein the compound is 5-iodo-6-nitroso-benzopyrone of Formula IIIl, or a metabolite, a pharmaceutically acceptable salt or prodrug thereof.

12. The method of claim 8, wherein the compound is 5-iodo-6-hydroxylamino-benzopyrone of Formula IIIm, or a metabolite, a pharmaceutically acceptable salt or prodrug thereof.

13. The method of claim 3, wherein the optionally substituted (C₃-C₇) heterocyclic is a five membered heterocyclic ring or a six membered heterocyclic ring.

14. The method of claim 13, wherein the optionally substituted (C₃-C₇) heterocyclic contains at least one nitrogen.

15. The method of claim 13, wherein the optionally substituted (C₃-C₇) heterocyclic is selected from the group consisting of azeridine, azetidine, pyrrole, dihydropyrrole, pyrrolidene, pyrazole, pyrazoline, pyrazolidine, imidazole, benzimidazole, triazole, tetrazole, oxazole, isoxazole, benzoxazole, oxadiazole, oxazoline, oxazolidine, thiazole, isothiazole, pyridine, dihydropyridine, tetrahydropyridine, quinazoline, pyrazine, pyrimidine, pyridazine, quinoline, isoquinoline, triazine, tetrazine, and piperazine.

16. The method of claim 13, wherein the optionally substituted (C₃-C₇) heterocyclic is substituted with a substituent selected from the group consisting of optionally substituted (C₁-C₆) alkyl, optionally substituted (C₁-C₆) alkoxy, optionally substituted (C₃-C₇) cycloalkyl, optionally substituted (C₃-C₇) heterocyclic, and optionally substituted aryl.

17. The method of claim 1 or 2 further comprising surgery, radiation therapy, chemotherapy, gene therapy, RNA therapy, immunotherapy, nanotherapy or a combination thereof.

18. The method of claim 1 or 2 further comprising administering an effective amount of an anti-tumor agent.

19. The method of claim 1 or 2 further comprising administering an effective amount of an organoplatinum compound.

20. The method of claim 1 or 2 further comprising administering an effective amount of an anti-metabolite compound.

21. The method of claim 1 or 2 further comprising administering an effective amount of oxaliplatin (OX).

22. The method of claim 1 or 2 further comprising administering an effective amount of gemcitabine (GEM).

23. The method of claim 1 or 2 further comprising administering an effective amount of OX and GEM.

24. The method of claim 1 or 2 wherein the administration is intravenous or intraperitoneal.

25. The method of claim 1 or 2 wherein the administration is orally.

26. The method of claim 1 or 2 wherein a poly-ADP-ribose polymerase (PARP) is inhibited by the compound in the subject.

27. The method of claim 1 or 2 wherein mono-ADP ribosylation and poly-ADP ribosylation are inhibited.

28. The method of claim 1 or 2 wherein a tumor cell undergoes apoptosis, cell cycle arrest, and/or necrosis in the subject.

29. The method of claim 1 or 2 wherein the subject expresses a detectable level of PARP protein.

30. The method of claim 1 or 2 wherein the subject has a detectable level of mono-ADP ribosylation and poly-ADP ribosylation.

31. A method of treating a cancer comprising administering to a subject in need thereof an effective amount of a compound of formula (I), or a metabolite, a pharmaceutically acceptable salt or prodrug thereof: 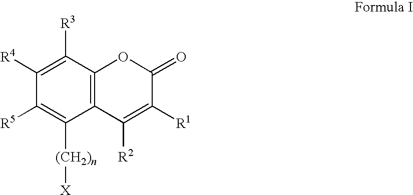
wherein n=0-10; R¹, R², R³, R⁴, R⁵ and X are independently selected from the group consisting of hydrogen, hydroxy, optionally substituted amine, amino, carboxyl, ester, nitroso, nitro, halogen, optionally substituted (C₁-C₆) alkyl, optionally substituted (C₁-C₆) alkoxy, optionally substituted (C₃-C₇) cycloalkyl, optionally substituted (C₃-C₇) heterocyclic, phenyl, and optionally substituted aryl; wherein at least two of the R¹, R², R³, R⁴, and R⁵ substituents are always hydrogen; and wherein at least one of the R¹, R², R³, R⁴, and R⁵ substituents is always a substituted cycloalkyl, a substituted heterocyclic, or a substituted phenyl;
wherein the cancer is selected from the group consisting of adrenal cortical cancer, anal cancer, aplastic anemia, bile duct cancer, bladder cancer, bone cancer, bone metastasis, central nervous system (CNS) cancers, peripheral nervous system (PNS) cancers, breast cancer, Castleman's Disease, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, uterine cancer (e.g. uterine sarcoma), vaginal cancer, vulvar cancer, and Waldenstrom's macroglobulinemia.

32. The method of claim 31, wherein the cancer is breast cancer, ovarian cancer, uterine cancer, pancreatic cancer, lung cancer, brain cancer, skin cancer, colon cancer, or a cancer derived from cancer stem cells.

33. The method of claim 31, wherein the breast cancer is negative for at least one of: ER, PR or HER2.

34. The method of claim 31, wherein the breast cancer is negative for at least one of: ER, PR or HER2; and wherein the breast cancer is positive for at least one of ER, PR or HER2.

35. The method of claim 31, wherein the breast cancer is negative for two of: ER, PR or HER2.

36. The method of claim 31, wherein the breast cancer is ER-negative and PR-negative.

37. The method of claim 31, wherein the breast cancer is ER-negative and HER2-negative.

38. The method of claim 31, wherein the breast cancer is PR-negative and HER2-negative.

39. The method of claim 31, wherein the breast cancer is an ER-negative breast cancer.

40. The method of claim 31, wherein the breast cancer is an HER²-negative breast cancer.

41. The method of claim 31, wherein X is a halogen selected from the group consisting of F, Cl, Br and I.

42. The method of claim 31, wherein X is iodine (I) and R⁵ is nitro, nitroso, hydroxylamino, hydroxyl, or amino.

43. The method of claim 31, wherein n is 0.

44. The method of claim 31, wherein the optionally substituted alkyl is substituted with a substituent selected from the group consisting of alkylamine, pyrrole, dihydropyrrole, and pyrrolidene.

45. The method of claim 31, wherein the compound is of the formula IIIa, IIIb, IIIc, IIId, IIIe, or IIIf, or one of their pharmaceutically acceptable salts or prodrugs:

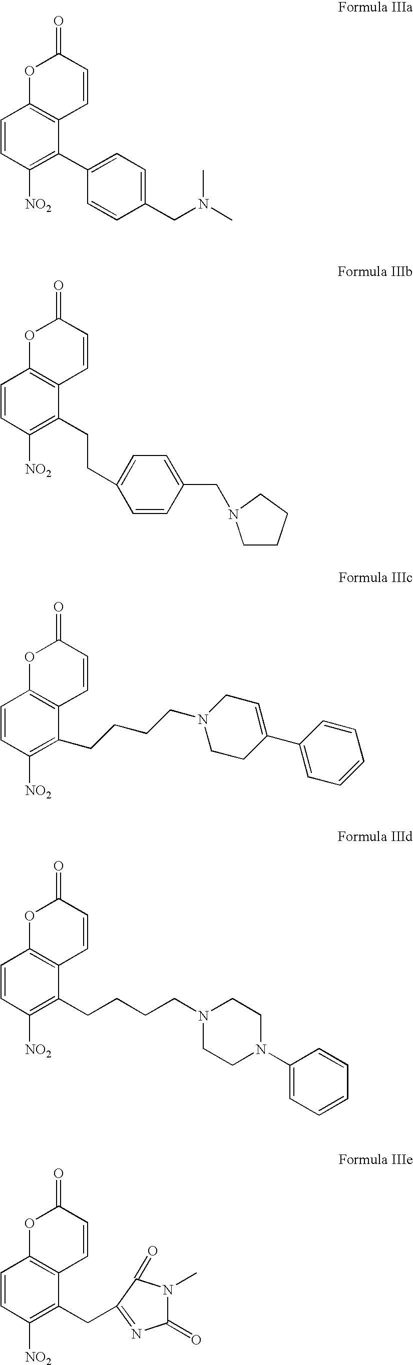

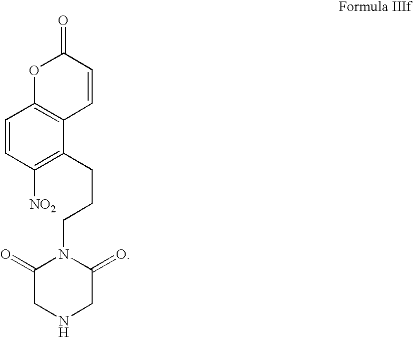

46. The method of claim 31, wherein the optionally substituted (C₃-C₇) heterocyclic is a five membered heterocyclic ring or a six membered heterocyclic ring.

47. The method of claim 31, wherein the optionally substituted (C₃-C₇) heterocyclic contains at least one nitrogen.

48. The method of claim 31, wherein the optionally substituted (C₃-C₇) heterocyclic is selected from the group consisting of azeridine, azetidine, pyrrole, dihydropyrrole, pyrrolidene, pyrazole, pyrazoline, pyrazolidine, imidazole, benzimidazole, triazole, tetrazole, oxazole, isoxazole, benzoxazole, oxadiazole, oxazoline, oxazolidine, thiazole, isothiazole, pyridine, dihydropyridine, tetrahydropyridine, quinazoline, pyrazine, pyrimidine, pyridazine, quinoline, isoquinoline, triazine, tetrazine, and piperazine.

49. The method of claim 31, wherein the optionally substituted (C₃-C₇) heterocyclic is substituted with a substituent selected from the group consisting of optionally substituted (C₁-C₆) alkyl, optionally substituted (C₁-C₆) alkoxy, optionally substituted (C₃-C₇) cycloalkyl, optionally substituted (C₃-C₇) heterocyclic, and optionally substituted aryl.

50. The method of claim 31 further comprising surgery, radiation therapy, chemotherapy, gene therapy, RNA therapy, immunotherapy, nanotherapy or a combination thereof.

51. The method of claim 31 further comprising administering an effective amount of an anti-tumor agent.

52. The method of claim 31 further comprising administering an effective amount of an organoplatinum compound.

53. The method of claim 31 further comprising administering an effective amount of an anti-metabolite compound.

54. The method of claim 31 further comprising administering an effective amount of oxaliplatin (OX).

55. The method of claim 31 further comprising administering an effective amount of gemcitabine (GEM).

56. The method of claim 31 further comprising administering an effective amount of OX and GEM.

57. The method of claim 31, wherein the administration is intravenous or intraperitoneal.

58. The method of claim 31, wherein the administration is orally.

59. The method of claim 31, wherein a poly-ADP-ribose polymerase (PARP) is inhibited by the compound in the subject.

60. The method of claim 31, wherein mono-ADP ribosylation and poly-ADP ribosylation are inhibited.

61. The method of claim 31, wherein a tumor cell undergoes apoptosis, cell cycle arrest, and/or necrosis in the subject.

62. The method of claim 31, wherein the subject expresses a detectable level of PARP protein.

63. The method of claim 31, wherein the subject has a detectable level of mono-ADP ribosylation and poly-ADP ribosylation.

64. A method of treating a cancer comprising administering to a subject in need thereof an effective amount of a composition comprising an anti-tumor agent and a compound of formula (I), or a metabolite, a pharmaceutically acceptable salt or prodrug thereof: 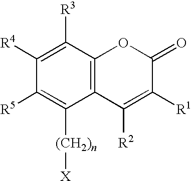
wherein n=0-10; R¹, R², R⁴, R⁵ and X are independently selected from the group consisting of hydrogen, hydroxy, optionally substituted amine, amino, carboxyl, ester, nitroso, nitro, halogen, optionally substituted (C₁-C₆) alkyl, optionally substituted (C₁-C₆) alkoxy, optionally substituted (C₃-C₇) cycloalkyl, optionally substituted (C₃-C₇) heterocyclic, phenyl, and optionally substituted aryl; and wherein at least two of the R¹, R², R³, R⁴, and R⁵ substituents are always hydrogen.

65. The method of claim 64, wherein the compound is of formula II or a metabolite, a pharmaceutically acceptable salt or prodrug thereof: 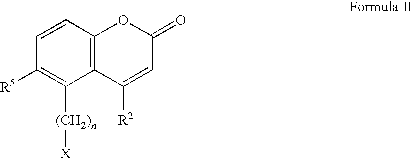
wherein R⁵ is selected from the group consisting of hydrogen, carboxyl, amino, nitroso, nitro, hydroxylamino, and hydroxy; and X is selected from the group consisting of halogen, hydroxy, optionally substituted (C₁-C₇) alkyl, optionally substituted (C₁-C₆) alkoxy, optionally substituted (C₃-C₇) cycloalkyl, optionally substituted (C₃-C₇) heterocyclic, phenyl, and optionally substituted aryl.

66. The method of claim 65, wherein the compound is of the formula IIIa, IIIb, IIIc, IIId, IIIe, IIIf, IIIg, IIIh, IIIk, IIIl, IIIm, or IIIn, or a metabolite, a pharmaceutically acceptable salt or prodrug thereof:

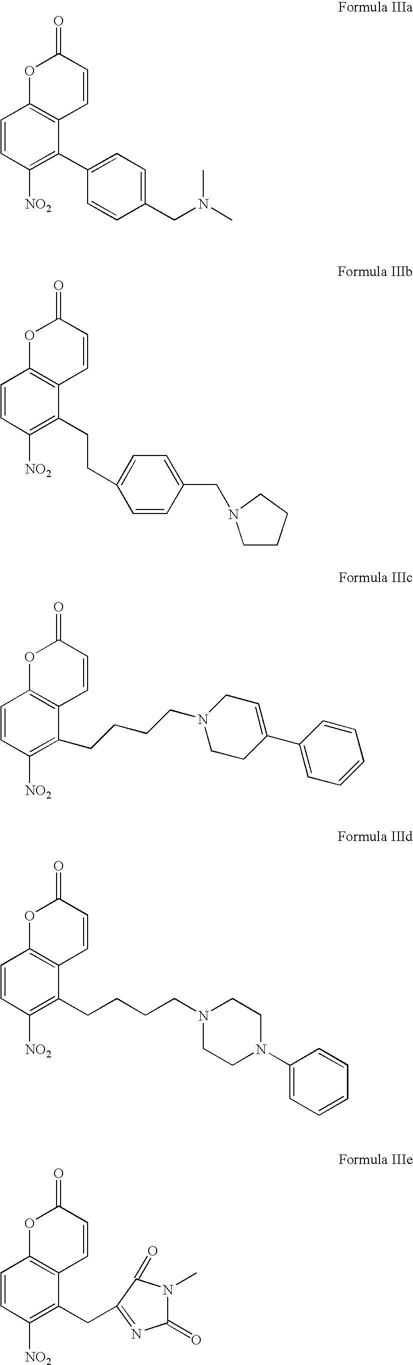

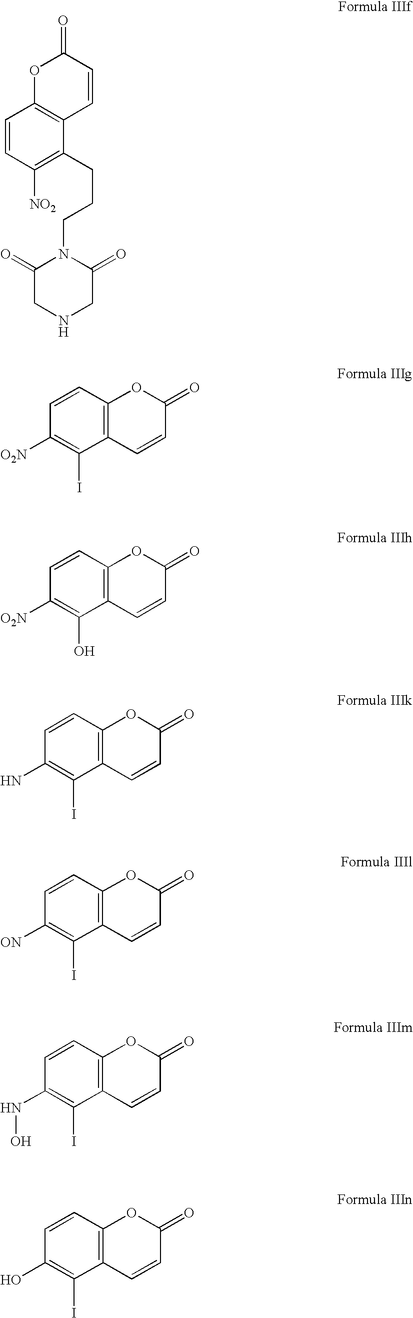

67. The method of claim 66, wherein the compound is 5-iodo-6-nitro-benzopyrone of Formula IIIg, or a metabolite, a pharmaceutically acceptable salt or prodrug thereof.

68. The method of claim 66, wherein the compound is 5-iodo-6-amino-benzopyrone of Formula IIIk, or a metabolite, a pharmaceutically acceptable salt or prodrug thereof.

69. The method of claim 66, wherein the compound is 5-iodo-6-nitroso-benzopyrone of Formula IIIl, or a metabolite, a pharmaceutically acceptable salt or prodrug thereof.

70. The method of claim 66, wherein the compound is 5-iodo-6-hydroxylamino-benzopyrone of Formula IIIm, or a metabolite, a pharmaceutically acceptable salt or prodrug thereof.

71. The method of claim 64, wherein the cancer is selected from the group consisting of adrenal cortical cancer, anal cancer, aplastic anemia, bile duct cancer, bladder cancer, bone cancer, bone metastasis, central nervous system (CNS) cancers, peripheral nervous system (PNS) cancers, breast cancer, Castleman's Disease, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, uterine cancer (e.g. uterine sarcoma), vaginal cancer, vulvar cancer, and Waldenstrom's macroglobulinemia.

72. The method of claim 64, wherein the cancer is breast cancer, ovarian cancer, uterine cancer, pancreatic cancer, lung cancer, brain cancer, skin cancer, colon cancer, or a cancer derived from cancer stem cells.

73. The method of claim 64, wherein the breast cancer is negative for at least one of: ER, PR or HER2.

74. The method of claim 64, wherein the breast cancer is negative for at least one of: ER, PR or HER2; and wherein the breast cancer is positive for at least one of ER, PR or HER2.

75. The method of claim 64, wherein the breast cancer is negative for two of: ER, PR or HER2.

76. The method of claim 64, wherein the breast cancer is ER-negative and PR-negative.

77. The method of claim 64, wherein the breast cancer is ER-negative and HER2-negative.

78. The method of claim 64, wherein the breast cancer is PR-negative and HER2-negative.

79. The method of claim 64, wherein the breast cancer is an ER-negative breast cancer.

80. The method of claim 64, wherein the breast cancer is an HER2-negative breast cancer.

81. The method of claim 64, wherein the anti-tumor agent is selected from the group consisting of antitumor alkylating agents, antitumor antimetabolites, antitumor antibiotics, plant-derived antitumor agents, antitumor organoplatinum compounds, antitumor campthotecin derivatives, antitumor tyrosine kinase inhibitors, monoclonal antibodies, interferons, biological response modifiers, and other agents having antitumor activities, or a pharmaceutically acceptable salt thereof.

82. The method of claim 81, wherein the antitumor alkylating agents are nitrogen mustard N-oxide, cyclophosphamide, ifosfamide, melphalan, busulfan, mitobronitol, carboquone, thiotepa, ranimustine, nimustine, temozolomide, and carmustine; the antitumor antimetabolites are methotrexate, 6-mercaptopurine riboside, mercaptopurine, 5-fluorouracil, tegafur, doxifluridine, carmofur, cytarabine, cytarabine ocfosfate, enocitabine, S-1, gemcitabine, fludarabine, and pemetrexed disodium; the antitumor antibiotics are actinomycin D, doxorubicin, daunorubicin, neocarzinostatin, bleomycin, peplomycin, mitomycin C, aclarubicin, pirarubicin, epirubicin, zinostatin stimalamer, idarubicin, sirolimus, and valrubicin; the plant-derived antitumor agents are vincristine, vinblastine, vindeshine, etoposide, sobuzoxane, docetaxel, paclitaxel, and vinorelbine; the antitumor platinum-complex compounds are cisplatin, carboplatin, nedaplatin, and oxaliplatin; the antitumor campthotecin derivatives are irinotecan, topotecan, and campthotecin; the antitumor tyrosine kinase inhibitors are gefitinib, imatinib, and erlotinib; the monoclonal antibodies are abciximab, adalimumab, alemtuzumab, basiliximab, bevacizumab, cetuximab, daclizumab, eculizumab, efalizumab, ibritumomab, tiuxetan, infliximab, muromonab-CD3, natalizumab, omalizumab, palivizumab, panitumumab, ranibizumab, gemtuzumab ozogamicin, rituximab, tositumomab, and trastuzumab; the interferons are interferon α, interferon α-2a, interferon α-2b, interferon β, interferon γ-1a, and interferon γ-n1, the biological response modifiers are krestin, lentinan, sizofuran, picibanil, or ubenimex, and the other antitumor agents are mitoxantrone, L-asparaginase, procarbazine, dacarbazine, hydroxycarbamide, pentostatin, tretinoin, alefacept, darbepoetin alfa, anastrozole, exemestane, bicalutamide, leuprorelin, flutamide, fulvestrant, pegaptanib octasodium, denileukin diftitox, aldesleukin, thyrotropin alfa, arsenic trioxide, bortezomib, capecitabine, and goserelin.

83. The method of claim 64, wherein the anti-tumor agent is an organoplatinum compound.

84. The method of claim 83, wherein the anti-tumor agent is oxaliplatin (OX), cisplatin, or carboplatin.

85. The method of claim 84, wherein the anti-tumor agent oxaliplatin (OX).

86. The method of claim 64, wherein the anti-tumor agent is an anti-metabolite agent.

87. The method of claim 86, wherein the anti-tumor agent is gemcitabine (GEM).

88. The method of claim 64 further comprising more than one anti-tumor agent.

89. The method of claim 88, wherein the anti-tumor agents are an organoplatinum compound and an anti-metabolite agent.

90. The method of claim 88, wherein the anti-tumor agents are OX and GEM.

91. The method of claim 64 further comprising surgery, radiation therapy, gene therapy, immunotherapy, RNA therapy, nanotherapy or a combination thereof.

92. The method of claim 64, wherein the administration is intravenous or intraperitoneal.

93. The method of claim 64, wherein the administration is orally.

94. The method of claim 64, wherein a poly-ADP-ribose polymerase (PARP) is inhibited by the compound in the subject.

95. The method of claim 64, wherein mono-ADP ribosylation and poly-ADP ribosylation are inhibited.

96. The method of claim 64, wherein a tumor cell undergoes apoptosis, cell cycle arrest, and/or necrosis in the subject.

97. The method of claim 64, wherein the subject expresses a detectable level of PARP protein.

98. The method of claim 64, wherein the subject has a detectable level of mono-ADP ribosylation and poly-ADP ribosylation.

99. A method of treating a cancer comprising administering to a subject in need thereof an effective amount of a composition comprising an organoplatinum compound and a compound of formula (I), or a metabolite, a pharmaceutically acceptable salt or prodrug thereof: 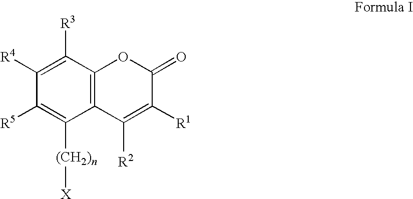
wherein n=0-10; R¹, R², R³, R⁴, R⁵ and X are independently selected from the group consisting of hydrogen, hydroxy, optionally substituted amine, amino, carboxyl, ester, nitroso, nitro, halogen, optionally substituted (C₁-C₆) alkyl, optionally substituted (C₁-C₆) alkoxy, optionally substituted (C₃-C₇) cycloalkyl, optionally substituted (C₃-C₇) heterocyclic, phenyl, and optionally substituted aryl; and wherein at least two of the R¹, R², R³, R⁴, and R⁵ substituents are always hydrogen.

100. The method of claim 99, wherein the compound is of formula II or a metabolite, a pharmaceutically acceptable salt or prodrug thereof: 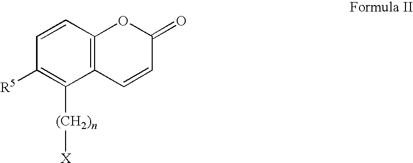
wherein R⁵ is selected from the group consisting of hydrogen, carboxyl, amino, nitroso, nitro, hydroxylamino, and hydroxy; and X is selected from the group consisting of halogen, hydroxy, optionally substituted (C₁-C₇) alkyl, optionally substituted (C₁-C₆) alkoxy, optionally substituted (C₃-C₇) cycloalkyl, optionally substituted (C₃-C₇) heterocyclic, phenyl, and optionally substituted aryl.

101. The method of claim 100, wherein the compound is of the formula IIIa, IIIb, IIIc, IIId, IIIe, IIIf, IIIg, IIIh, IIIk, IIIl, IIIm, or IIIn, or a metabolite, a pharmaceutically acceptable salt or prodrug thereof:

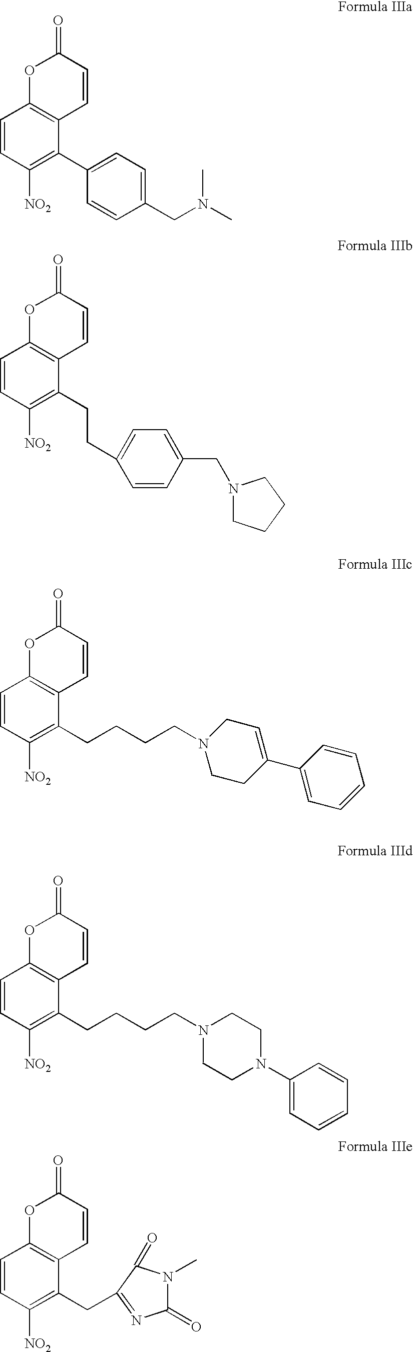

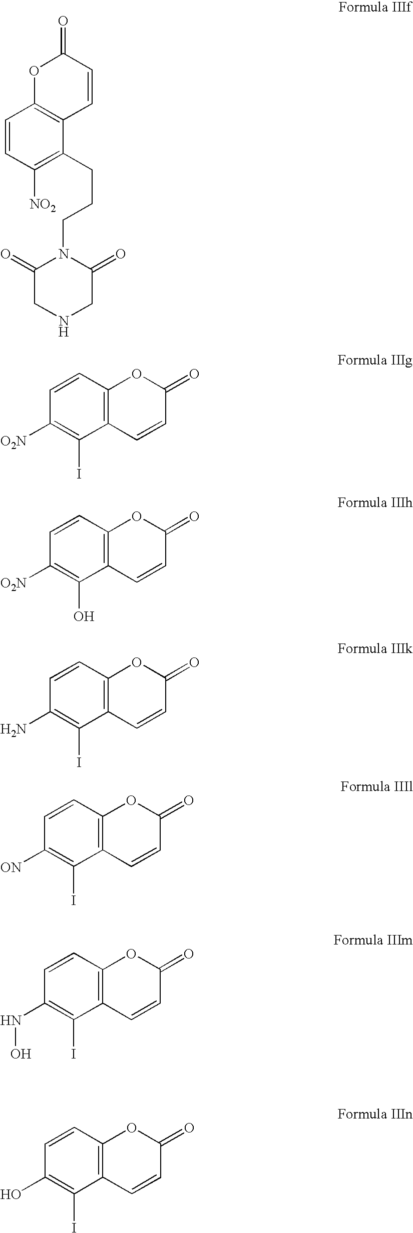

102. The method of claim 101, wherein the compound is 5-iodo-6-nitro-benzopyrone of Formula IIIg, or a metabolite, a pharmaceutically acceptable salt or prodrug thereof.

103. The method of claim 101, wherein the compound is 5-iodo-6-amino-benzopyrone of Formula IIIk, or a metabolite, a pharmaceutically acceptable salt or prodrug thereof.

104. The method of claim 101, wherein the compound is 5-iodo-6-nitroso-benzopyrone of Formula IIIl, or a metabolite, a pharmaceutically acceptable salt or prodrug thereof.

105. The method of claim 101, wherein the compound is 5-iodo-6-hydroxylamino-benzopyrone of Formula IIIm, or a metabolite, a pharmaceutically acceptable salt or prodrug thereof.

106. The method of claim 99, wherein the cancer is selected from the group consisting of adrenal cortical cancer, anal cancer, aplastic anemia, bile duct cancer, bladder cancer, bone cancer, bone metastasis, central nervous system (CNS) cancers, peripheral nervous system (PNS) cancers, breast cancer, Castleman's Disease, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, uterine cancer (e.g. uterine sarcoma), vaginal cancer, vulvar cancer, and Waldenstrom's macroglobulinemia.

107. The method of claim 99, wherein the cancer is breast cancer, ovarian cancer, uterine cancer, pancreatic cancer, lung cancer, brain cancer, skin cancer, colon cancer, or a cancer derived from cancer stem cells.

108. The method of claim 99, wherein the breast cancer is negative for at least one of: ER, PR or HER2.

109. The method of claim 99, wherein the breast cancer is negative for at least one of: ER, PR or HER2; and wherein the breast cancer is positive for at least one of ER, PR or HER2.

110. The method of claim 99, wherein the breast cancer is negative for two of: ER, PR or HER2.

111. The method of claim 99, wherein the breast cancer is ER-negative and PR-negative.

112. The method of claim 99, wherein the breast cancer is ER-negative and HER2-negative.

113. The method of claim 99, wherein the breast cancer is PR-negative and HER2-negative.

114. The method of claim 99, wherein the breast cancer is an ER-negative breast cancer.

115. The method of claim 99, wherein the breast cancer is an HER2-negative breast cancer.

116. The method of claim 99, wherein the organoplatinum compound is oxaliplatin (OX).

117. The method of claim 99 further comprising administering an effective amount of OX and 5-iodo-6-nitro-benzopyrone (IIIg).

118. The method of claim 99 further comprising administering an effective amount of OX and 5-iodo-6-amino-benzopyrone (IIIk).

119. The method of claim 99 further comprising administering an effective amount of an anti-metabolite.

120. The method of claim 99 further comprising administering an effective amount of gemcitabine (GEM).

121. The method of claim 99 further comprising surgery, radiation therapy, gene therapy, immunotherapy, RNA therapy, nanotherapy or a combination thereof.

122. The method of claim 99, wherein the administration is intravenous or intraperitoneal.

123. The method of claim 99, wherein the administration is orally.

124. The method of claim 99, wherein a poly-ADP-ribose polymerase (PARP) is inhibited by the compound in the subject.

125. The method of claim 99, wherein mono-ADP ribosylation and poly-ADP ribosylation are inhibited.

126. The method of claim 99, wherein a tumor cell undergoes apoptosis, cell cycle arrest, and/or necrosis in the subject.

127. The method of claim 99, wherein the subject expresses a detectable level of PARP protein.

128. The method of claim 99, wherein the subject has a detectable level of mono-ADP ribosylation and poly-ADP ribosylation.

129. A method of treating a cancer comprising administering to a subject in need thereof an effective amount of a composition comprising an anti-metabolite agent and a compound of formula (I), or a metabolite, a pharmaceutically acceptable salt or prodrug thereof: 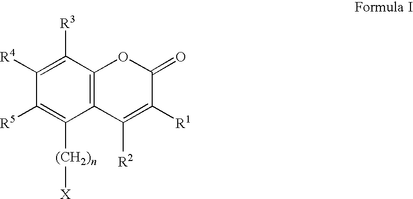
wherein n=0-10; R¹, R², R³, R⁴, R⁵ and X are independently selected from the group consisting of hydrogen, hydroxy, optionally substituted amine, amino, carboxyl, ester, nitroso, nitro, halogen, optionally substituted (C₁-C₆) alkyl, optionally substituted (C₁-C₆) alkoxy, optionally substituted (C₃-C₇) cycloalkyl, optionally substituted (C₃-C₇) heterocyclic, phenyl, and optionally substituted aryl; and wherein at least two of the R¹, R², R³, R⁴, and R⁵ substituents are always hydrogen.

130. The method of claim 129, wherein the compound is of formula II or its pharmaceutically acceptable salts or prodrugs: 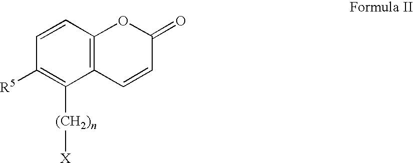
wherein R⁵ is selected from the group consisting of hydrogen, carboxyl, amino, nitroso, nitro, hydroxylamino, and hydroxy; and X is selected from the group consisting of halogen, hydroxy, optionally substituted (C₁-C₇) alkyl, optionally substituted (C₁-C₆) alkoxy, optionally substituted (C₃-C₇) cycloalkyl, optionally substituted (C₃-C₇) heterocyclic, phenyl, and optionally substituted aryl.

131. The method of claim 130, wherein the compound is of the formula IIIa, IIIb, IIIc, IIId, IIIe, IIIf, IIIg, IIIh, IIIk, IIIl, IIIm, or IIIn, or a metabolite, a pharmaceutically acceptable salt or prodrug thereof:

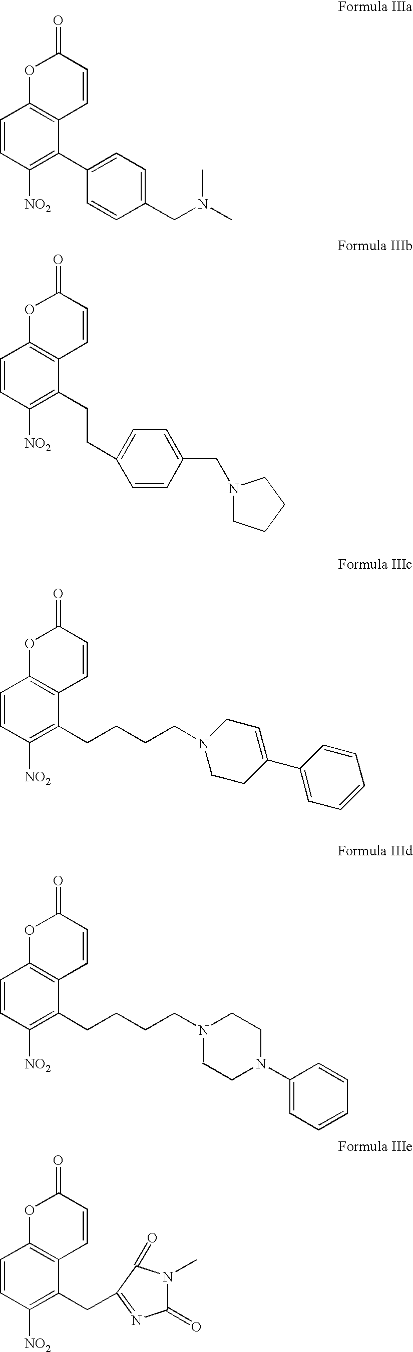

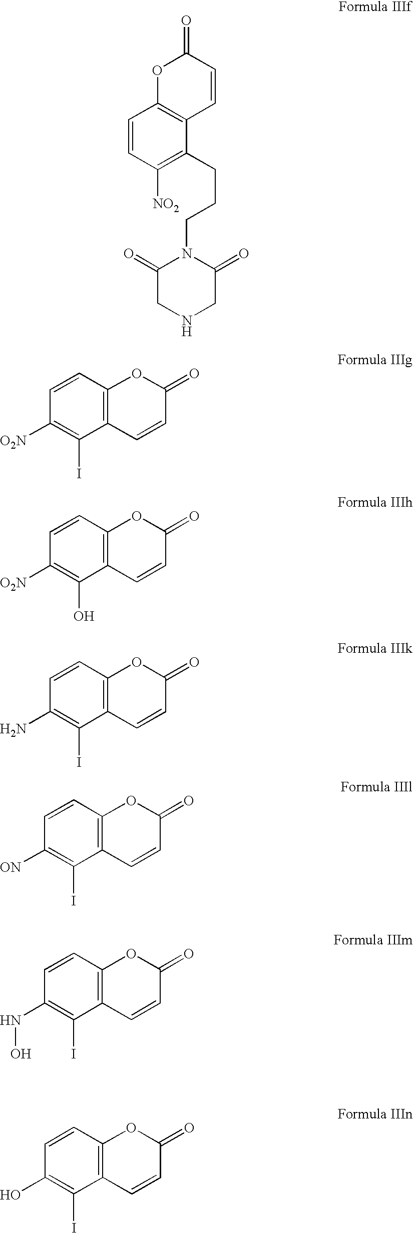

132. The method of claim 131, wherein the compound is 5-iodo-6-nitro-benzopyrone of Formula IIIg, or a metabolite, a pharmaceutically acceptable salt or prodrug thereof.

133. The method of claim 131, wherein the compound is 5-iodo-6-amino-benzopyrone of Formula IIIk, or a metabolite, a pharmaceutically acceptable salt or prodrug thereof.

134. The method of claim 131, wherein the compound is 5-iodo-6-nitroso-benzopyrone of Formula IIIl, or a metabolite, a pharmaceutically acceptable salt or prodrug thereof.

135. The method of claim 131, wherein the compound is 5-iodo-6-hydroxylamino-benzopyrone of Formula IIIm, or a metabolite, a pharmaceutically acceptable salt or prodrug thereof.

136. The method of claim 129, wherein the cancer is selected from the group consisting of adrenal cortical cancer, anal cancer, aplastic anemia, bile duct cancer, bladder cancer, bone cancer, bone metastasis, central nervous system (CNS) cancers, peripheral nervous system (PNS) cancers, breast cancer, Castleman's Disease, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, uterine cancer (e.g. uterine sarcoma), vaginal cancer, vulvar cancer, and Waldenstrom's macroglobulinemia.

137. The method of claim 129, wherein the cancer is breast cancer, ovarian cancer, uterine cancer, pancreatic cancer, lung cancer, brain cancer, skin cancer, colon cancer, or a cancer derived from cancer stem cells.

138. The method of claim 129, wherein the breast cancer is negative for at least one of: ER, PR or HER2.

139. The method of claim 129, wherein the breast cancer is negative for at least one of: ER, PR or HER2; and wherein the breast cancer is positive for at least one of ER, PR or HER2.

140. The method of claim 129, wherein the breast cancer is negative for two of: ER, PR or HER2.

141. The method of claim 129, wherein the breast cancer is ER-negative and PR-negative.

142. The method of claim 129, wherein the breast cancer is ER-negative and HER2-negative.

143. The method of claim 129, wherein the breast cancer is PR-negative and HER2-negative.

144. The method of claim 129, wherein the breast cancer is an ER-negative breast cancer.

145. The method of claim 129, wherein the breast cancer is an HER2-negative breast cancer.

146. The method of claim 129 wherein the anti-metabolite agent is gemcitabine (GEM).

147. The method of claim 129 further comprising administering an effective amount of GEM and 5-iodo-6-nitro-benzopyrone (IIIg).

148. The method of claim 129 further comprising administering an effective amount of GEM and 5-iodo-6-amino-benzopyrone (IIIk).

149. The method of claim 129 further comprising administering an effective amount of an organoplatinum compound.

150. The method of claim 129 further comprising administering an effective amount of oxaliplatin (OX).

151. The method of claim 129 further comprising surgery, radiation therapy, gene therapy, immunotherapy, RNA therapy, nanotherapy or a combination thereof.

152. The method of claim 129, wherein the administration is intravenous or intraperitoneal.

153. The method of claim 129, wherein the administration is orally.

154. The method of claim 129, wherein a poly-ADP-ribose polymerase (PARP) is inhibited by the compound in the subject.

155. The method of claim 129, wherein mono-ADP ribosylation and poly-ADP ribosylation are inhibited.

156. The method of claim 129, wherein a tumor cell undergoes apoptosis, cell cycle arrest, and/or necrosis in the subject.

157. The method of claim 129, wherein the subject expresses a detectable level of PARP protein.

158. The method of claim 129, wherein the subject has a detectable level of mono-ADP ribosylation and poly-ADP ribosylation.

159. A method of treating a cancer comprising administering to a subject in need thereof an effective amount of a composition comprising an organoplatinum compound and an anti-metabolite agent, and a compound of formula (I), or a metabolite, a pharmaceutically acceptable salt or prodrug thereof: 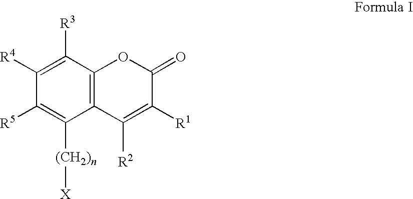
wherein n=0-10; R¹, R², R³, R⁴, R⁵ and X are independently selected from the group consisting of hydrogen, hydroxy, optionally substituted amine, amino, carboxyl, ester, nitroso, nitro, halogen, optionally substituted (C₁-C₆) alkyl, optionally substituted (C₁-C₆) alkoxy, optionally substituted (C₃-C₇) cycloalkyl, optionally substituted (C₃-C₇) heterocyclic, phenyl, and optionally substituted aryl; and wherein at least two of the R¹, R², R³, R⁴, and R⁵ substituents are always hydrogen.

160. The method of claim 159, wherein the compound is of formula II or a metabolite, a pharmaceutically acceptable salt or prodrug thereof: 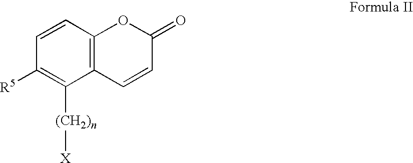
wherein R⁵ is selected from the group consisting of hydrogen, carboxyl, amino, nitroso, nitro, hydroxylamino, and hydroxy; and X is selected from the group consisting of halogen, hydroxy, optionally substituted (C₁-C₇) alkyl, optionally substituted (C₁-C₆) alkoxy, optionally substituted (C₃-C₇) cycloalkyl, optionally substituted (C₃-C₇) heterocyclic, phenyl, and optionally substituted aryl.

161. The method of claim 160, wherein the compound is of the formula IIIa, IIIb, IIIc, IIId, IIIe, IIIf, IIIg, IIIh, IIIk, IIIl, IIIm, or IIIn, or a metabolite, a pharmaceutically acceptable salt or prodrug thereof:

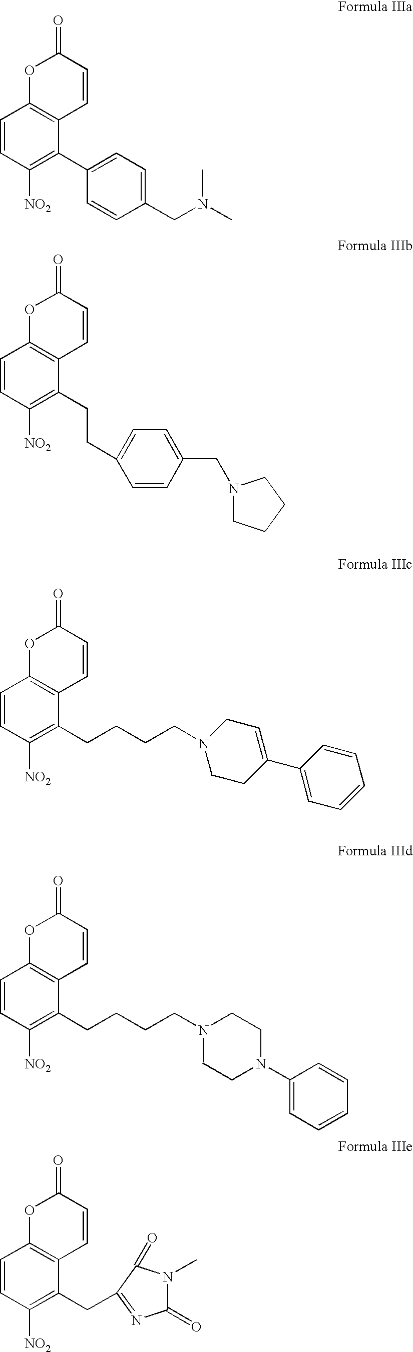

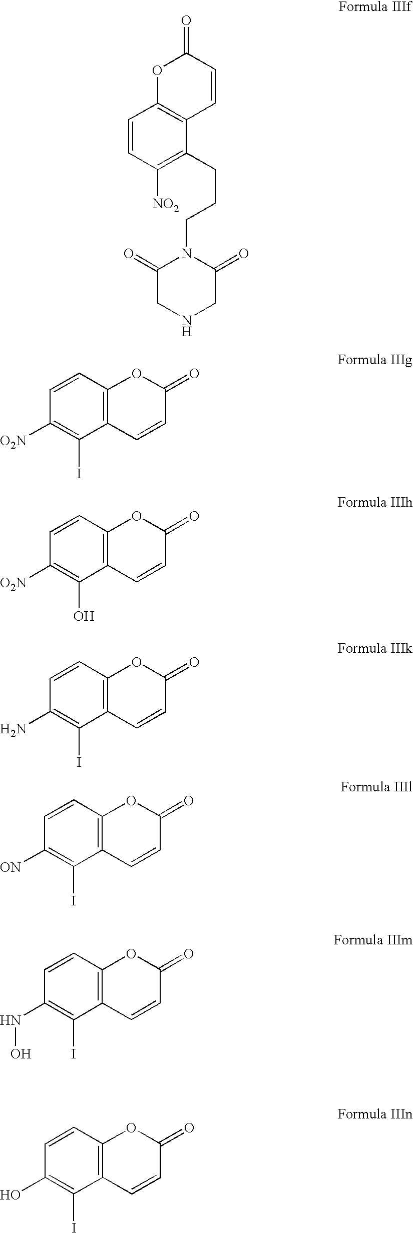

162. The method of claim 161, wherein the compound is 5-iodo-6-nitro-benzopyrone of Formula IIIg, or a metabolite, a pharmaceutically acceptable salt or prodrug thereof.

163. The method of claim 161, wherein the compound is 5-iodo-6-amino-benzopyrone of Formula IIIk, or a metabolite, a pharmaceutically acceptable salt or prodrug thereof.

164. The method of claim 161, wherein the compound is 5-iodo-6-nitroso-benzopyrone of Formula IIIl, or a metabolite, a pharmaceutically acceptable salt or prodrug thereof.

165. The method of claim 161, wherein the compound is 5-iodo-6-hydroxylamino-benzopyrone of Formula IIIm, or a metabolite, a pharmaceutically acceptable salt or prodrug thereof.

166. The method of claim 159, wherein the cancer is selected from the group consisting of adrenal cortical cancer, anal cancer, aplastic anemia, bile duct cancer, bladder cancer, bone cancer, bone metastasis, central nervous system (CNS) cancers, peripheral nervous system (PNS) cancers, breast cancer, Castleman's Disease, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, uterine cancer (e.g. uterine sarcoma), vaginal cancer, vulvar cancer, and Waldenstrom's macroglobulinemia.

167. The method of claim 159, wherein the cancer is breast cancer, ovarian cancer, uterine cancer, pancreatic cancer, lung cancer, brain cancer, skin cancer, colon cancer, or a cancer derived from cancer stem cells.

168. The method of claim 159, wherein the breast cancer is negative for at least one of: ER, PR or HER2.

169. The method of claim 159, wherein the breast cancer is negative for at least one of: ER, PR or HER2; and wherein the breast cancer is positive for at least one of ER, PR or HER2.

170. The method of claim 159, wherein the breast cancer is negative for two of: ER, PR or HER2.

171. The method of claim 159, wherein the breast cancer is ER-negative and PR-negative.

172. The method of claim 159, wherein the breast cancer is ER-negative and HER2-negative.

173. The method of claim 159, wherein the breast cancer is PR-negative and HER2-negative.

174. The method of claim 159, wherein the breast cancer is an ER-negative breast cancer.

175. The method of claim 159, wherein the breast cancer is an HER2-negative breast cancer.

176. The method of claim 159, wherein the organoplatinum compound is OX.

177. The method of claim 159, wherein the anti-metabolite agent is GEM.

178. The method of claim 159 further comprising administering an effective amount of OX, GEM and 5-iodo-6-nitro-benzopyrone (IIIg).

179. The method of claim 159 further comprising administering an effective amount of OX, GEM and 5-iodo-6-amino-benzopyrone (IIIk).

180. The method of claim 159 further comprising surgery, radiation therapy, gene therapy, immunotherapy, RNA therapy, nanotherapy or a combination thereof.

181. The method of claim 159, wherein the administration is intravenous or intraperitoneal.

182. The method of claim 159, wherein the administration is orally.

183. The method of claim 159, wherein a poly-ADP-ribose polymerase (PARP) is inhibited by the compound in the subject.

184. The method of claim 159, wherein mono-ADP ribosylation and poly-ADP ribosylation are inhibited.

185. The method of claim 159, wherein a tumor cell undergoes apoptosis, cell cycle arrest, and/or necrosis in the subject.

186. The method of claim 159, wherein the subject expresses a detectable level of PARP protein.

187. The method of claim 159, wherein the subject has a detectable level of mono-ADP ribosylation and poly-ADP ribosylation.

188. A composition for the treatment of a cancer, the composition comprising compound of formula I, or a metabolite, a pharmaceutically acceptable salt or prodrug thereof: 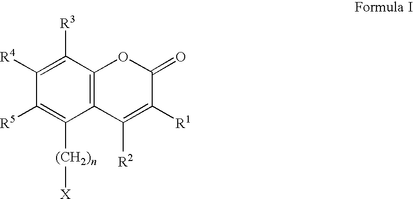
wherein n=0-10; R¹, R², R³, R⁴, R⁵ and X are independently selected from the group consisting of hydrogen, hydroxy, optionally substituted amine, amino, carboxyl, ester, nitroso, nitro, halogen, optionally substituted (C₁-C₆) alkyl, optionally substituted (C₁-C₆) alkoxy, optionally substituted (C₃-C₇) cycloalkyl, optionally substituted (C₃-C₇) heterocyclic, phenyl, and optionally substituted aryl; and wherein at least two of the R¹, R², R³, R⁴, and R⁵ substituents are always hydrogen; and
wherein the compound is not one of the following:

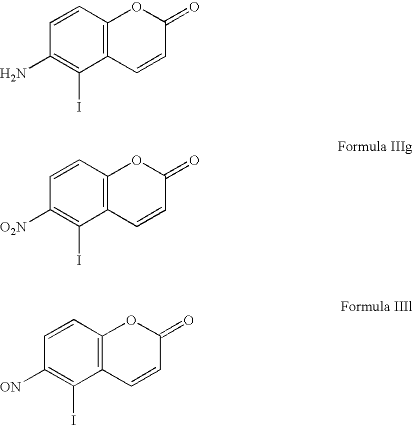

189. The composition of claim 188, wherein the compound is of the formula IIIa, IIIb, IIIc, IIId, IIIe, IIIf, IIIh, IIIm, or IIIn, or a metabolite, a pharmaceutically acceptable salt or prodrug thereof:

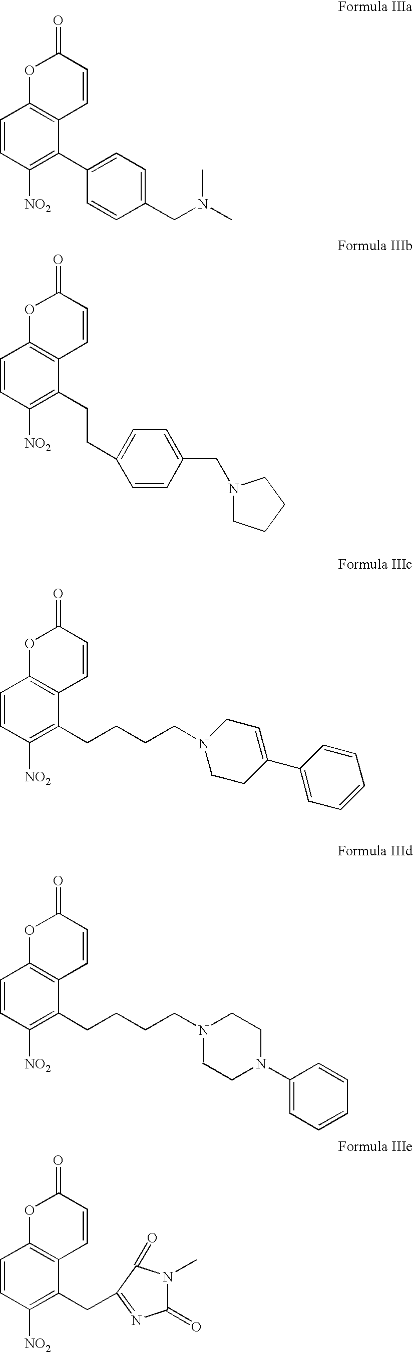

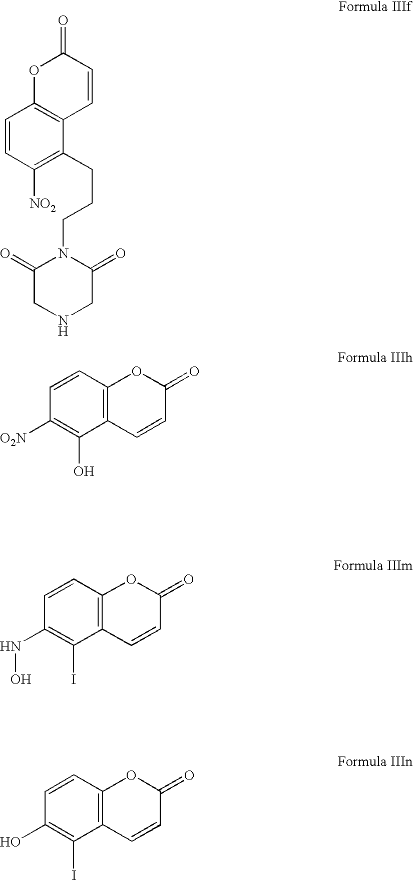

190. The composition of claim 188, wherein the cancer is selected from the group consisting of adrenal cortical cancer, anal cancer, aplastic anemia, bile duct cancer, bladder cancer, bone cancer, bone metastasis, central nervous system (CNS) cancers, peripheral nervous system (PNS) cancers, breast cancer, Castleman's Disease, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, uterine cancer (e.g. uterine sarcoma), vaginal cancer, vulvar cancer, and Waldenstrom's macroglobulinemia.

191. The composition of claim 188, wherein the cancer is breast cancer, ovarian cancer, uterine cancer, pancreatic cancer, lung cancer, brain cancer, skin cancer, colon cancer, or a cancer derived from cancer stem cells.

192. The composition of claim 188, wherein the breast cancer is negative for at least one of: ER, PR or HER2.

193. The composition of claim 188, wherein the breast cancer is negative for at least one of: ER, PR or HER2; and wherein the breast cancer is positive for at least one of ER, PR or HER2.

194. The composition of claim 188, wherein the breast cancer is negative for two of: ER, PR or HER2.

195. The composition of claim 188, wherein the breast cancer is ER-negative and PR-negative.

196. The composition of claim 188, wherein the breast cancer is ER-negative and HER2-negative.

197. The composition of claim 188, wherein the breast cancer is PR-negative and HER2-negative.

198. The composition of claim 188, wherein the breast cancer is an ER-negative breast cancer.

199. The composition of claim 188, wherein the breast cancer is an HER2-negative breast cancer.

200. The composition of claim 188 further comprises an anti-tumor agent.

201. The composition of claim 200, wherein the anti-tumor agent is selected from the group consisting of antitumor alkylating agents, antitumor antimetabolites, antitumor antibiotics, plant-derived antitumor agents, antitumor organoplatinum compounds, antitumor campthotecin derivatives, antitumor tyrosine kinase inhibitors, monoclonal antibodies, interferons, biological response modifiers, and other agents having antitumor activities, or a pharmaceutically acceptable salt thereof

202. The composition of claim 201, wherein the antitumor alkylating agents are nitrogen mustard N-oxide, cyclophosphamide, ifosfamide, melphalan, busulfan, mitobronitol, carboquone, thiotepa, ranimustine, nimustine, temozolomide, and carmustine; the antitumor antimetabolites are methotrexate, 6-mercaptopurine riboside, mercaptopurine, 5-fluorouracil, tegafur, doxifluridine, carmofur, cytarabine, cytarabine ocfosfate, enocitabine, S-1, gemcitabine, fludarabine, and pemetrexed disodium; the antitumor antibiotics are actinomycin D, doxorubicin, daunorubicin, neocarzinostatin, bleomycin, peplomycin, mitomycin C, aclarubicin, pirarubicin, epirubicin, zinostatin stimalamer, idarubicin, sirolimus, and valrubicin; the plant-derived antitumor agents are vincristine, vinblastine, vindeshine, etoposide, sobuzoxane, docetaxel, paclitaxel, and vinorelbine; the antitumor platinum-complex compounds are cisplatin, carboplatin, nedaplatin, and oxaliplatin; the antitumor campthotecin derivatives are irinotecan, topotecan, and campthotecin; the antitumor tyrosine kinase inhibitors are gefitinib, imatinib, and erlotinib; the monoclonal antibodies are abciximab, adalimumab, alemtuzumab, basiliximab, bevacizumab, cetuximab, daclizumab, eculizumab, efalizumab, ibritumomab, tiuxetan, infliximab, muromonab-CD3, natalizumab, omalizumab, palivizumab, panitumumab, ranibizumab, gemtuzumab ozogamicin, rituximab, tositumomab, and trastuzumab; the interferons are interferon α, interferon α-2a, interferon α-2b, interferon β, interferon γ-1a, and interferon γ-n1, the biological response modifiers are krestin, lentinan, sizofuran, picibanil, or ubenimex, and the other antitumor agents are mitoxantrone, L-asparaginase, procarbazine, dacarbazine, hydroxycarbamide, pentostatin, tretinoin, alefacept, darbepoetin alfa, anastrozole, exemestane, bicalutamide, leuprorelin, flutamide, fulvestrant, pegaptanib octasodium, denileukin diftitox, aldesleukin, thyrotropin alfa, arsenic trioxide, bortezomib, capecitabine, and goserelin.

203. The composition of claim 200, wherein the anti-tumor agent is an organoplatinum compound.

204. The composition of claim 203, wherein the anti-tumor agent is oxaliplatin (OX), cisplatin, or carboplatin.

205. The composition of claim 200, wherein the anti-tumor agent is an anti-metabolite agent.

206. The composition of claim 205, wherein the anti-tumor agent is gemcitabine (GEM).

207. The composition of claim 188 further comprising more than one anti-tumor agent.

208. The composition of claim 188, wherein the anti-tumor agents are an organoplatinum compound and an anti-metabolite agent.

209. The composition of claim 188, wherein the anti-tumor agents are OX and GEM.

210. The composition of claim 188 may be used in combination with surgery, radiation therapy, gene therapy, immunotherapy, RNA therapy, nanotherapy or a combination thereof.

211. The composition of claim 188, wherein a poly-ADP-ribose polymerase (PARP) is inhibited by the compound of formula (I).

212. The composition of claim 188, wherein mono-ADP ribosylation and poly-ADP ribosylation are inhibited.

213. The composition of claim 188, wherein a cancer cell expresses a detectable level of PARP protein.

214. The composition of claim 188, wherein the subject has a detectable level of mono-ADP ribosylation and poly-ADP ribosylation.

215. A kit for the treatment of a cancer, the kit comprising an effective amount of a composition of claim 188, or a pharmaceutically acceptable salt or prodrug thereof.

216. The kit of claim 215, wherein the cancer is selected from the group consisting of adrenal cortical cancer, anal cancer, aplastic anemia, bile duct cancer, bladder cancer, bone cancer, bone metastasis, central nervous system (CNS) cancers, peripheral nervous system (PNS) cancers, breast cancer, Castleman's disease, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, uterine cancer (e.g. uterine sarcoma), vaginal cancer, vulvar cancer, and Waldenstrom's macroglobulinemia.

217. A composition for the treatment of a cancer, the composition comprising a combination of an anti-tumor agent and a compound of formula I, or a metabolite, a pharmaceutically acceptable salt or prodrug thereof: 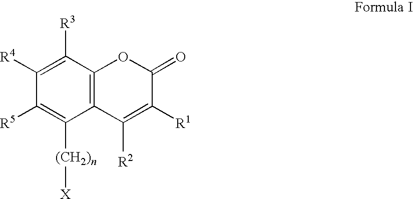
wherein n=0-10; R¹, R², R³, R⁴, R⁵ and X are independently selected from the group consisting of hydrogen, hydroxy, optionally substituted amine, amino, carboxyl, ester, nitroso, nitro, halogen, optionally substituted (C₁-C₆) alkyl, optionally substituted (C₁-C₆) alkoxy, optionally substituted (C₃-C₇) cycloalkyl, optionally substituted (C₃-C₇) heterocyclic, phenyl, and optionally substituted aryl; and wherein at least two of the R¹, R², R³, R⁴, and R⁵ substituents are always hydrogen.

218. The composition of claim 217, wherein the compound is of formula II or a metabolite, a pharmaceutically acceptable salt or prodrug thereof: 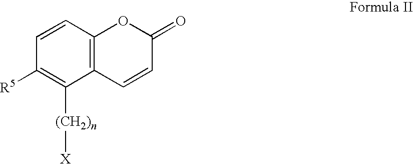
wherein R⁵ is selected from the group consisting of hydrogen, carboxyl, amino, nitroso, nitro, hydroxylamino, and hydroxy; and X is selected from the group consisting of halogen, hydroxy, optionally substituted (C₁-C₇) alkyl, optionally substituted (C₁-C₆) alkoxy, optionally substituted (C₃-C₇) cycloalkyl, optionally substituted (C₃-C₇) heterocyclic, phenyl, and optionally substituted aryl.

219. The composition of claim 218, wherein the compound is of the formula IIIa, IIIb, IIIc, IIId, IIIe, IIIf, IIIg, IIIh, IIIk, IIIl, IIIm, or IIIn, or a metabolite, a pharmaceutically acceptable salt or prodrug thereof:

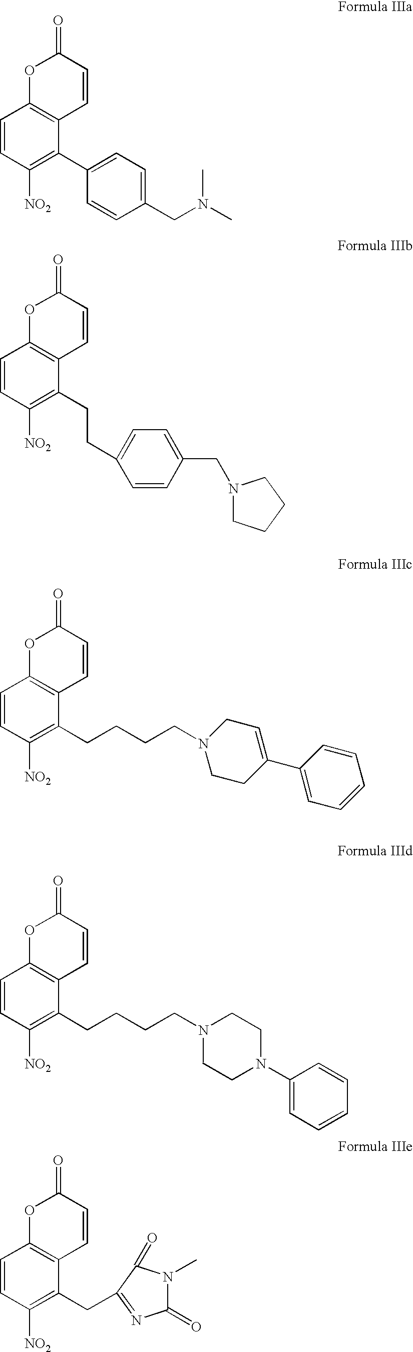

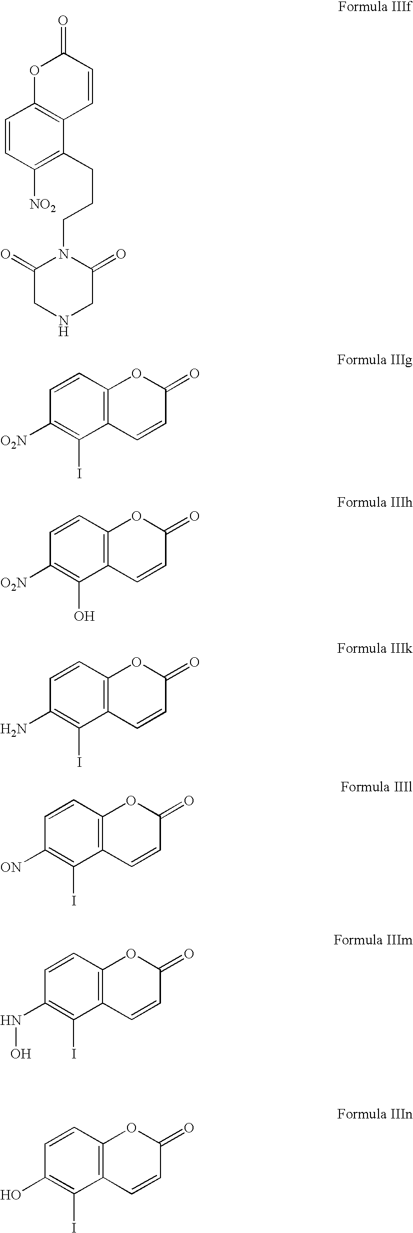

220. The composition of claim 219, wherein the compound of formula (I) is 5-iodo-6-nitro-benzopyrone of Formula IIIg, or a metabolite, a pharmaceutically acceptable salt or prodrug thereof.

221. The composition of claim 219, wherein the compound of formula (I) is 5-iodo-6-amino-benzopyrone of Formula IIIk, or a metabolite, a pharmaceutically acceptable salt or prodrug thereof.

222. The composition of claim 219, wherein the compound is 5-iodo-6-nitroso-benzopyrone of Formula IIIl, or a metabolite, a pharmaceutically acceptable salt or prodrug thereof.

223. The composition of claim 219, wherein the compound is 5-iodo-6-hydroxylamino-benzopyrone of Formula IIIm, or a metabolite, a pharmaceutically acceptable salt or prodrug thereof.

224. The composition of claim 217, wherein the cancer is selected from the group consisting of adrenal cortical cancer, anal cancer, aplastic anemia, bile duct cancer, bladder cancer, bone cancer, bone metastasis, central nervous system (CNS) cancers, peripheral nervous system (PNS) cancers, breast cancer, Castleman's disease, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, uterine cancer (e.g. uterine sarcoma), vaginal cancer, vulvar cancer, and Waldenstrom's macroglobulinemia.

225. The composition of claim 217, wherein the cancer is breast cancer, ovarian cancer, uterine cancer, pancreatic cancer, lung cancer, brain cancer, skin cancer, colon cancer, or a cancer derived from cancer stem cells.

226. The composition of claim 217, wherein the breast cancer is negative for at least one of: ER, PR or HER2.

227. The composition of claim 217, wherein the breast cancer is negative for at least one of: ER, PR or HER2; and wherein the breast cancer is positive for at least one of ER, PR or HER2.

228. The composition of claim 217, wherein the breast cancer is negative for two of: ER, PR or HER2.

229. The composition of claim 217, wherein the breast cancer is ER-negative and PR-negative.

230. The composition of claim 217, wherein the breast cancer is ER-negative and HER2-negative.

231. The composition of claim 217, wherein the breast cancer is PR-negative and HER2-negative.

232. The composition of claim 217, wherein the breast cancer is an ER-negative breast cancer.

233. The composition of claim 217, wherein the breast cancer is an HER2-negative breast cancer.

234. The composition of claim 217, wherein the anti-tumor agent is selected from the group consisting of antitumor alkylating agents, antitumor antimetabolites, antitumor antibiotics, plant-derived antitumor agents, antitumor organoplatinum compounds, antitumor campthotecin derivatives, antitumor tyrosine kinase inhibitors, monoclonal antibodies, interferons, biological response modifiers, and other agents having antitumor activities, or a pharmaceutically acceptable salt thereof.

235. The composition of claim 234, wherein the antitumor alkylating agents are nitrogen mustard N-oxide, cyclophosphamide, ifosfamide, melphalan, busulfan, mitobronitol, carboquone, thiotepa, ranimustine, nimustine, temozolomide, and carmustine; the antitumor antimetabolites are methotrexate, 6-mercaptopurine riboside, mercaptopurine, 5-fluorouracil, tegafur, doxifluridine, carmofur, cytarabine, cytarabine ocfosfate, enocitabine, S-1, gemcitabine, fludarabine, and pemetrexed disodium; the antitumor antibiotics are actinomycin D, doxorubicin, daunorubicin, neocarzinostatin, bleomycin, peplomycin, mitomycin C, aclarubicin, pirarubicin, epirubicin, zinostatin stimalamer, idarubicin, sirolimus, and valrubicin; the plant-derived antitumor agents are vincristine, vinblastine, vindeshine, etoposide, sobuzoxane, docetaxel, paclitaxel, and vinorelbine; the antitumor platinum-complex compounds are cisplatin, carboplatin, nedaplatin, and oxaliplatin; the antitumor campthotecin derivatives are irinotecan, topotecan, and campthotecin; the antitumor tyrosine kinase inhibitors are gefitinib, imatinib, and erlotinib; the monoclonal antibodies are abciximab, adalimumab, alemtuzumab, basiliximab, bevacizumab, cetuximab, daclizumab, eculizumab, efalizumab, ibritumomab, tiuxetan, infliximab, muromonab-CD3, natalizumab, omalizumab, palivizumab, panitumumab, ranibizumab, gemtuzumab ozogamicin, rituximab, tositumomab, and trastuzumab; the interferons are interferon α, interferon α-2a, interferon α-2b, interferon β, interferon γ-1a, and interferon γ-n1, the biological response modifiers are krestin, lentinan, sizofuran, picibanil, or ubenimex, and the other antitumor agents are mitoxantrone, L-asparaginase, procarbazine, dacarbazine, hydroxycarbamide, pentostatin, tretinoin, alefacept, darbepoetin alfa, anastrozole, exemestane, bicalutamide, leuprorelin, flutamide, fulvestrant, pegaptanib octasodium, denileukin diftitox, aldesleukin, thyrotropin alfa, arsenic trioxide, bortezomib, capecitabine, and goserelin.

236. The composition of claim 217, wherein the anti-tumor agent is an organoplatinum compound.

237. The composition of claim 236, wherein the anti-tumor agent is oxaliplatin (OX), cisplatin, or carboplatin.

238. The composition of claim 217, wherein the anti-tumor agent is an anti-metabolite agent.

239. The composition of claim 238, wherein the anti-tumor agent is gemcitabine (GEM).

240. The composition of claim 217 further comprising more than one anti-tumor agent.

241. The composition of claim 217, wherein the anti-tumor agents are an organoplatinum compound and an anti-metabolite agent.

242. The composition of claim 217, wherein the anti-tumor agents are OX and GEM.

243. The composition of claim 217, wherein a poly-ADP-ribose polymerase (PARP) is inhibited by the compound of formula (I).

244. The composition of claim 217, wherein mono-ADP ribosylation and poly-ADP ribosylation are inhibited.

245. The composition of claim 217, wherein a cancer cell expresses a detectable level of PARP protein.

246. The composition of claim 217, wherein the subject has a detectable level of mono-ADP ribosylation and poly-ADP ribosylation.

247. A kit for the treatment of a cancer, the kit comprising an effective amount of a composition of claim 217, or a pharmaceutically acceptable salt or prodrug thereof.

248. The kit of claim 247, wherein the cancer is selected from the group consisting of adrenal cortical cancer, anal cancer, aplastic anemia, bile duct cancer, bladder cancer, bone cancer, bone metastasis, central nervous system (CNS) cancers, peripheral nervous system (PNS) cancers, breast cancer, Castleman's disease, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, uterine cancer (e.g. uterine sarcoma), vaginal cancer, vulvar cancer, and Waldenstrom's macroglobulinemia.
